(19) United States
(12) Patent Application Publication
(10) Pub. No.: US 2026/0062745 A1
(43) Pub. Date: Mar. 5, 2026

(54) FLOW CELL DEVICES AND USE THEREOF

(71) Applicant: Element Biosciences, Inc., San Diego, CA (US)

(72) Inventors: Bill Kengli LIN, San Digo, CA (US); Yingxian YU, San Diego, CA (US); Jingzhi LU, San Diego, CA (US); Michael Charles RAY, San Diego, CA (US); Siyuan XING, San Diego, CA (US); Chiting CHANG, San Diego, CA (US); Omid KHANDAN, San Diego, CA (US); Jeffrey YEH, San Diego, CA (US); Daniel HASTINGS, San Diego, CA (US); Michael Thomas DANGELO, San Diego, CA (US); Chueh-Yu WU, San Diego, CA (US)

(21) Appl. No.: 19/261,197

(22) Filed: Jul. 7, 2025

Related U.S. Application Data
(63) Continuation of application No. PCT/US2024/010760, filed on Jan. 8, 2024.
(60) Provisional application No. 63/479,158, filed on Jan. 9, 2023.
(60) Provisional application No. 63/502,896, filed on May 17, 2023.

(51) Int. Cl. C12Q 1/6874; B01L 3/00

(52) U.S. Cl. CPC C12Q 1/6874; B01L 3/502761; B01L 2200/0647; B01L 2200/16; B01L 2300/0877; B01L 2300/165; B01L 2400/082; B01L 2400/088

(57) ABSTRACT

The present disclosure provides flow cell devices, systems, and methods for facilitating and performing DNA sequencing analysis with reduced system complexity and cost, significant COGS saving and reduced contamination level. The flow cell devices and systems can comprise: a support comprising one or more substrates; one or more channels defined by the one or more substrates and configured to allow fluids to flow therethrough; an inlet in fluidic connection with the one or more channels, the inlet comprising an open landing area in one substrate; and an outlet.

### CROSS REFERENCE

This application is a continuation of International Application No. PCT/US2024/010760, filed Jan. 8, 2024, which claims priority to, and the benefit of, U.S. Provisional Application No. 63/479,158, filed Jan. 9, 2023, and U.S. Provisional Application No. 63/502,896, filed May 17, 2023, each of which is incorporated herein by reference in its entirety.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BACKGROUND

Flow cell devices are used in chemistry and biotechnology applications. In next-generation sequencing (NGS) systems, flow cell devices are used to immobilize template nucleic acid molecules derived from biological samples and then introduce a repetitive flow of sequencing reagents to attach labeled nucleotides to specific positions in the template sequences. A series of label signals are detected and decoded to reveal the nucleotide sequences of the template molecules, e.g., immobilized, or amplified, or combinations thereof, nucleic acid template molecules attached to a surface of the flow cell.

Existing NGS flow cells are multi-layered structures fabricated from planar surface substrates and other flow cell components, which are then bonded to form fluid flow channels. Such flow cells may require costly, multi-step precision fabrication techniques to achieve the required design specifications. On the other hand, inexpensive and off-the-shelf, single channel capillaries are available in a variety of sizes and shapes but are generally not suited for ease of handling and compatibility with the repetitive switching between reagents that are required for application such as NGS.

### SUMMARY

Disclosed herein, in one aspect, is a flow cell device comprising a support comprising one or more substrates, wherein the one or more substrates comprise an inlet and an outlet, wherein the inlet comprises an open landing area; and one or more channels defined by the one or more substrates, wherein the one or more channels are in fluidic connection with the inlet and the outlet, wherein the one or more channels are configured to allow a fluid or a gas gap between the fluid and another fluid to flow through the one or more channels. In some embodiments, the open landing area is at least partly covered with a surface coating. In some embodiments, the one or more channels extend from the inlet to the outlet. In some embodiments, the one or more channels extend along a first direction and between the inlet and the outlet. In some embodiments, the one or more channels are configured to allow the gas gap to flow through the one or more channels, wherein the fluid comprises a first reagent and the another fluid comprises a second reagent. In some embodiments, the one or more channels are configured to allow the gas gap to flow through the one or more channels during a DNA sequencing run. In some embodiments, the one or more channels are configured to allow the gas gap to flow through the one or more channels from the inlet. In some embodiments, the one or more channels are configured to allow the gas gap to flow through the one or more channels to facilitate reducing contamination of the second reagent by the first reagent in the DNA sequencing run. In some embodiments, the one or more channels are configured to allow the gas gap to flow through the one or more channels to reduce a minimum amount of the first reagent, the second reagent, or a washing reagent used for the DNA sequencing run. In some embodiments, the one or more channels comprise one or more surfaces. In some embodiments, the one or more surfaces comprises an inner surface. In some embodiments, the one or more surfaces comprises an exterior surface. In some embodiments, the one or more surfaces comprises an interior top surface, an interior bottom surface, or both. In some embodiments, the one or more surfaces comprises an exterior top surface, an exterior bottom surface, or both. In some embodiments, the one or more surfaces comprises a planar surface. In some embodiments, the one or more surfaces is passivated. In some embodiments, the one or more surfaces is passivated with a coating that immobilizes a surface capture primer, a nucleic acid template molecule, or both, for capturing a polynucleotide. In some embodiments, the one or more surfaces comprises the polynucleotide coupled thereto. In some embodiments, the gas gap is configured to remove moisture or a liquid from at least part of the one or more surfaces of the one or more channels. In some embodiments, the gas gap does not impair a chemical function of the one or more surfaces. In some embodiments, the coating of the one or more surfaces comprises at least one hydrophilic polymer coating layer. In some embodiments, the coating of the one or more surfaces comprises a plurality of oligonucleotide molecules attached to at least one hydrophilic polymer coating layer. In some embodiments, the one or more surfaces comprises at least one discrete region that comprises a plurality of clonally-amplified sample nucleic acid molecules that have been annealed to a plurality of attached oligonucleotide molecules. In some embodiments, the at least one hydrophilic polymer coating layer has a water contact angle of no more than about 50 degrees. In some embodiments, at least one of the plurality of clonally-amplified sample nucleic acid molecules comprises a concatemer annealed to at least one of the plurality of attached oligonucleotide molecules. In some embodiments, the at least one hydrophilic polymer coating layer comprises polyethylene glycol (PEG). In some embodiments, the one or more surfaces further comprises a second hydrophilic polymer coating layer. In some embodiments, the at least one hydrophilic polymer coating layer comprises a branched hydrophilic polymer. In some embodiments, the branched hydrophilic polymer comprises at least 8 branches. In some embodiments, the at least one of the plurality of the clonally-amplified sample nucleic acid molecules comprises a single-stranded multimeric nucleic acid molecule comprising repeats of a regularly occurring monomer unit. In some embodiments, the single-stranded multimeric nucleic acid molecule is at least 10 kilobases in length. In some embodiments, the at least one of the plurality of the clonally-amplified sample nucleic acid molecules further comprises a double-stranded monomeric copy of the regularly occurring monomer unit. In some embodiments, the plurality of oligonucleotide molecules is present at about a uniform surface density across the one or more surfaces. In some embodiments, the plurality of oligonucleotide molecules is present at a local surface density of at least 100,000 molecules/μm² at a first position on the one or more surfaces, and at a second local surface density at a second position on the one or more surfaces. In some embodiments, the coating comprises: a first layer comprising a monolayer of polymer molecules tethered to a surface of a substrate of the one or more substrates; a second layer comprising a second monolayer of polymer molecules tethered to the polymer molecules of the first layer; and a third layer comprising a third monolayer of polymer molecules tethered to the polymer molecules of the second layer, wherein at least one of the first layer, the second layer, or the third layer comprises branched polymer molecules. In some embodiments, the third layer further comprises oligonucleotides tethered to the polymer molecules of the third layer. In some embodiments, the oligonucleotides tethered to the polymer molecules of the third layer are distributed at a plurality of depths throughout the third layer. In some embodiments, the coating further comprises: a fourth layer comprising branched polymer molecules tethered to the polymer molecules of the third layer, and a fifth layer comprising polymer molecules tethered to the branched polymer molecules of the fourth layer. In some embodiments, the polymer molecules of the fifth layer further comprise oligonucleotides tethered to the polymer molecules of the fifth layer. In some embodiments, the oligonucleotides tethered to the polymer molecules of the fifth layer are distributed at a plurality of depths throughout the fifth layer. In some embodiments, the at least one hydrophilic polymer coating layer comprises polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxyethyl methacrylate) (PHEMA), poly(oligo (ethylene glycol) methyl ether methacrylate) (POEGMA), polyglutamic acid (PGA), poly-lysine, poly-glucoside, streptavidin, or dextran.

In some embodiments, when the plurality of clonally-amplified sample nucleic acid molecules or complementary sequences thereof are labeled with Cyanine dye-3, an image of the one or more surfaces exhibits a ratio of fluorescence intensities for the plurality of clonally-amplified sample nucleic acid molecules or complementary sequences thereof labeled with Cyanine dye-3, and nonspecific Cyanine dye-3 dye adsorption background (Bᵢₙₜₑᵣ) of at least 3:1. In some embodiments, the image of the one or more surfaces exhibits a ratio of fluorescence intensities for the plurality of clonally-amplified sample nucleic acid molecules or complementary sequences thereof labeled with Cyanine dye-3, and a combination of nonspecific Cyanine dye-3 dye adsorption background and nonspecific amplification background (Bᵢₙₜₑᵣ+Bᵢₙₜᵣₐ) of at least 3:1. In some embodiments, when the plurality of clonally-amplified sample nucleic acid molecules or complementary sequences thereof are labeled with Cyanine dye-3, the image of the one or more surfaces exhibits a ratio of fluorescence intensities for the plurality of clonally-amplified sample nucleic acid molecules or complementary sequences thereof labeled with Cyanine dye-3, and nonspecific dye adsorption background (Bᵢₙₜₑᵣ) of at least 5:1. In some embodiments, the image of the one or more surfaces exhibits a ratio of fluorescence intensities for the plurality of clonally-amplified sample nucleic acid molecules or complementary sequences thereof labeled with Cyanine dye-3, and a combination of nonspecific Cyanine dye-3 dye adsorption background and nonspecific amplification background (Bᵢₙₜₑᵣ+Bᵢₙₜᵣₐ) of at least 5:1. In some embodiments, when the plurality of clonally-amplified sample nucleic acid molecules or complementary sequences thereof are labeled with Cyanine dye-3, a fluorescence image of the one or more surfaces exhibits a contrast-to-noise ratio (CNR) of at least 20 when the fluorescence image is acquired using an inverted microscope equipped with a 20× objective, NA=0.75, dichroic mirror optimized for 532 nm light, a bandpass filter optimized for Cyanine dye-3 emission, and a camera under non-signal saturating conditions, while the one or more surfaces is immersed in a buffer. In some embodiments, the plurality of oligonucleotide molecules is present at a surface density of at least 1,000 molecules/m². In some embodiments, the first reagent is configured to wet the one or more surfaces of the one or more channels. In some embodiments, the second reagent is configured to rewet the one or more surfaces of the one or more channels after removal of moisture or the liquid from the at least part of the one or more surfaces of the one or more channels. In some embodiments, the gas gap comprises air. In some embodiments, the gas gap comprises dry air. In some embodiments, the gas gap comprises one or more inert gases. In some embodiments, the gas gap comprises one or more active gases. In some embodiments, the first or the second reagent comprise a liquid. In some embodiments, the first or the second reagent does not contain an air bubble that is greater than a predetermined size. In some embodiments, the coating comprises a liquid-repelling coating. In some embodiments, the coating comprises an omniphobic coating. In some embodiments, the coating comprises a slippery liquid-infused porous surface (SLIPS). In some embodiments, the coating comprises a slippery omniphobic covalently attached liquid (SOCAL) coating. In some embodiments, the coating comprises a liquid-like polymer brush surface that is covalently attached to the one or more substrates. In some embodiments, the coating is formed by impregnating lubricants in one or more porous surfaces. In some embodiments, the lubricants comprise a liquid with a surface energy below about 20 mJ/m². In some embodiments, the lubricants comprise a silicone oil. In some embodiments, the coating comprises a surface energy that is below about 20 mJ/m². In some embodiments, the coating is formed by acid-catalyzed graft polycondensation of one or more saline monomers. In some embodiments, the one or more saline monomers comprise dimethyldimethoxysilane. In some embodiments, the open landing area is in fluidic connection with the one or more channels. In some embodiments, the open landing area is in fluidic connection with one channel of the one or more channels. In some embodiments, the open landing area is in fluidic connection with two or more of the one or more channels. In some embodiments, the open landing area is on a bottom substrate of the one or more substrates. In some embodiments, the inlet comprises a hole in a top substrate of the one or more substrates. In some embodiments, the hole in the top substrate is positioned above at least part of the open landing area. In some embodiments, the flow cell device is configured to allow a dispenser to openly dispense one or more reagents through the hole to the open landing area. In some embodiments, the dispenser is configured to openly dispense the one or more reagents from a tip of the dispenser to the open landing area. In some embodiments, the dispenser is configured to openly dispense the one or more reagents from the tip of the dispenser to the open landing area without tubing in between the dispenser and the open landing area. In some embodiments, at least part of the tip of the dispenser is in contact with the open landing area. In some embodiments, the tip of the dispenser is not in contact with the open landing area. In some embodiments, the flow cell device further comprises a cleaning outlet in the one or more substrates. In some embodiments, the cleaning outlet is in fluidic connection with the inlet. In some embodiments, the cleaning outlet is in fluidic connection with the open landing area. In some embodiments, the cleaning outlet is positioned underneath the open landing area. In some embodiments, the cleaning outlet is in a top or bottom substrate of the one or more substrates. In some embodiments, the cleaning outlet comprises a side port on the one or more substrates, wherein the side port: extends at least along a direction that is perpendicular or nearly perpendicular to an x direction; extends at least along a direction that is perpendicular or nearly perpendicular to a y direction; extends at least along a direction that is perpendicular or nearly perpendicular to a z direction; extends at least along a direction that is oblique to an x direction; extends at least along a direction that is oblique to a y direction; or extends at least along a direction that is oblique to a z direction. In some embodiments, the cleaning outlet is configured to be coupled with a first pump or a second pump. In some embodiments, the one or more channels comprise one or more microfluidic channels. In some embodiments, the one or more surfaces is coated with fluorescent beads that are chemically immobilized to the one or more surfaces. In some embodiments, the fluorescent beads are covalently attached to the one or more surfaces. In some embodiments, a gap between the interior top surface and the interior bottom surface is about 150 μm, 130 μm, 120 μm, 110 μm, 100 μm, 90 μm, 80 μm, 70 μm, 60 μm, 50 μm, or 40 μm. In some embodiments, a height of the one or more channels is about 150 μm, 130 μm, 120 μm, 110 μm, 100 μm, 90 μm, 80 μm, 70 μm, 60 μm, 50 μm, or 40 μm. In some embodiments, the polynucleotide captured thereon is configured to be imaged in a sequencing cycle. In some embodiments, the one or more substrates comprise a top substrate and a bottom substrate. In some embodiments, the one or more channels are defined between the top substrate and the bottom substrate. In some embodiments, the one or more channels are defined at least partly in a top surface of the bottom substrate. In some embodiments, the one or more channels are defined at least partly in a bottom surface of the top substrate. In some embodiments, the one or more substrates further comprise a middle substrate. In some embodiments, the one or more channels are defined at least partly in the middle substrate. In some embodiments, the one or more substrates comprise glass or plastic. In some embodiments, at least part of the support is transparent. In some embodiments, at least part of the one or more substrates is transparent. In some embodiments, the support is solid. In some embodiments, the one or more channels comprise 1, 2, 3, 4, 5, 6, 7, or 8 channels. In some embodiments, the one or more channels comprise 2, 4, 6, 8, or 10 channels. In some embodiments, each channel of the one or more channels comprises a lane length of less than about 70 mm, 75 mm, 80 mm, or 90 mm. In some embodiments, each channel of the one or more channels comprises a lane width of less than about 10 mm, 8 mm, 7 mm, 6 mm, 5 mm, 4 mm, or 3 mm. In some embodiments, at least a portion of the open landing area is covered with a second surface coating comprising a slippery coating. In some embodiments, at least a portion of the open landing area is covered with a second surface coating comprising a liquid-repelling coating. In some embodiments, at least a portion of the open landing area is covered with a second surface coating comprising an omniphobic coating. In some embodiments, at least a portion of the open landing area is covered with a second surface coating comprising a slippery liquid-infused porous surface (SLIPS). In some embodiments, at least a portion of the open landing area is covered with a second surface coating comprising a slippery omniphobic covalently attached liquid (SOCAL) coating. In some embodiments, at least a portion of the open landing area is covered with a second surface coating comprising a liquid-like polymer brush surface that is covalently attached to the one or more substrates. In some embodiments, at least a portion of the open landing area is covered with a second surface coating comprising impregnating a lubricant in a porous surface to generate the second surface coating with a surface energy below about 20 mJ/m². In some embodiments, at least a portion of the open landing area is covered with a second surface coating comprising acid-catalyzed graft polycondensation of one or more saline monomers. In some embodiments, the one or more saline monomers comprise dimethyldimethoxysilane. In some embodiments, the flow cell device is configured to allow cleaning at least part of the first reagent from at least part of the one or more channels during a DNA sequencing run. In some embodiments, the flow cell device is configured to allow at least part of the first reagent to remain in the one or more channels. In some embodiments, the first reagent and the second reagent are different. In some embodiments, at least part of the one or more channels comprise more than about 40% of a corresponding volume or length of each of the one or more channels. In some embodiments, at least part of the one or more channels comprise more than about half of a corresponding volume or length of each of the one or more channels. In some embodiments, at least part of the one or more channels comprise more than about 60% of a corresponding volume or length of each of the one or more channels. In some embodiments, at least part of the one or more channels comprises more than about 70% of a corresponding volume or length of each of the one or more channels. In some embodiments, at least part of the one or more channels comprises more than about 80% of a corresponding volume or length of each of the one or more channels. In some embodiments, the cleaning outlet is configured to allow a residual amount of the first reagent on the open landing area to flow through the cleaning outlet. In some embodiments, the residual amount of the first reagent on the open landing area comprises meniscus of the first reagent. In some embodiments, the flow cell device further comprises one or more seals positioned on the one or more substrate. In some embodiments, a first portion of a channel of the one or more channels comprises a first z location and a second portion of the channel comprises a second z location that is different from the first z location. In some embodiments, the first portion of the channel comprises one or more first imaging surfaces. In some embodiments, the second portion of the channel comprises one or more second imaging surfaces. In some embodiments, the top substrate or the bottom substrate comprises one or more substrate layers. In some embodiments, the top substrate comprises a first thickness above the first portion of the channel and a second thickness about the second portion of the channel. In some embodiments, the second thickness is greater than the first thickness. In some embodiments, the second thickness is 20%, 50%, 80%, 100%, 120%, 150%, or 200% more than the first thickness. In some embodiments, the bottom substrate comprises a third thickness above the first portion of the channel and a fourth thickness above the second portion of the channel. In some embodiments, the fourth thickness is greater than the third thickness. In some embodiments, the fourth thickness is 20%, 50%, 80%, 100%, 120%, 150%, or 200% more than the third thickness. In some embodiments, the one or more seals comprise one or more mechanical seals. In some embodiments, the one or more seals comprise one or more gaskets. In some embodiments, the cleaning outlet is configured to remove a fluid from the one or more channels. In some embodiments, the cleaning outlet is in sealed fluidic connection with a pump or vacuum. In some embodiments, the cleaning outlet is configured to direct a fluid or a gas to the one or more channels. In some embodiments, the bottom substrate comprises glass, plastic, or both. In some embodiments, the one or more seals comprise a first seal with a thickness along a z direction that is comparable to a thickness of the top substrate in the second portion. In some embodiments, the one or more seals comprise a second seal with a thickness along a z direction that is comparable to a thickness of the bottom substrate in the second portion. In some embodiments, the second seal has a thickness along the z direction that is greater than the thickness of the bottom substrate in the first portion.

In some embodiments, the flow cell device further comprises a frame covering at least a portion of the one or more substrates. In some embodiments, the frame is mechanically fixed to the one or more seals. In some embodiments, the frame comprises plastic. In some embodiments, the one or more seals interface with a manifold or a connector to allow sealed fluidic communication between the manifold or connector with the one or more channels. In some embodiments, the manifold or the connector comprises one or more fluidic pathways. In some embodiments, the one or more fluidic pathways are in fluidic communication with the one or more channels. In some embodiments, the one or more fluidic pathways are in fluidic communication with the open landing area. In some embodiments, the manifold or the connector is configured to be in sealed fluidic communication with the one or more channels by applying a pressure satisfying a predetermined threshold thereon. In some embodiments, the one or more fluidic pathways extend along a y axis and wherein the pressure is applied along the y axis. In some embodiments, the one or more fluidic pathways extend along a x axis and wherein the pressure is applied along the x axis. In some embodiments, the manifold or connector comprises a bonding interface that directly contacts an end of the flow cell device. In some embodiments, the manifold or connector comprises a bonding interface that contacts an end of the flow cell device with the one or more seals in between. In some embodiments, the manifold or connector comprises a bonding interface that contacts an end of the flow cell device with adhesive in between. In some embodiments, the manifold or connector comprises an open area at an end of a fluidic pathway of the one or more fluidic pathways. In some embodiments, the open area fluidically connects to the open landing area. In some embodiments, the flow cell device further comprises one or more reference features configured to position the flow cell device relative to the manifold or connector, a sample stage, or a sequencing system. In some embodiments, the one or more reference features comprise at least one alignment feature located at a central point along the x axis. In some embodiments, the one or more reference features comprise at least one alignment feature located at or near an end of the one or more substrate along the y axis. In some embodiments, the one or more reference features comprise a cavity running through the one or more substrate and configured to be coupled to a pin. In some embodiments, the one or more reference features comprise a grove extending through the one or more substrates that is configured to be coupled to a pin. In some embodiments, the manifold or connector comprises a top portion or a bottom portion that extends beyond the one or more substrates along the z axis and covers at least part of one or more substrates in a x-y plane. In some embodiments, the top portion or bottom portion is at the first portion, the second portion, or both of the one or more channels. In some embodiments, the top portion or bottom portion comprises one or more alignment features configured to align the top portion or bottom portion to the flow cell device. In some embodiments, the top portion or bottom portion comprises one or more alignment features configured to align the top portion or bottom portion to the flow cell device along z axis or along y axis. In some embodiments, the flow cell device further comprises one or more tubes that interface with the manifold or connector and the flow cell device. In some embodiments, each of the one or more tubes comprises a wall surrounding a lumen. In some embodiments, the lumen is in fluidic communication with the one or more channels of the flow cell device and the one or more fluidic pathways of the manifold or connector. In some embodiments, at least part of the one or more tubes are embedded in the one or more substrates. In some embodiments, each of the one or more tubes is coupled to the manifold or connector thereby enabling fluidic communication therebetween. In some embodiments, the one or more seals comprise a sock seal that covers at least a portion of the flow cell device in the x-y plane and one end of the flow cell device in the x-z plane. In some embodiments, the one or more seals comprise a flexible material that deforms under a pressure satisfying a predetermine threshold. In some embodiments, the one or more seals comprise a L-shaped seal that extends along the z axis and y axis. In some embodiments, the L-shaped seal extends along the y axis and into a corresponding channel of the one or more channels. In some embodiments, a pressure or force is applied to the L-shaped seal along y axis to enable sealed fluidic communication between the flow cell device and the manifold. In some embodiments, the one or more seals are configured to interface with the manifold or a connector thereby allowing sealed fluidic communication between the flow cell device and the manifold. In some embodiments, the one or more seals comprise a membrane seal that covers at least part of the flow cell device and at least part of the manifold or connector thereby sealing fluidic communication therebetween. In some embodiments, the membrane seal comprises a flat gasket placed on top of a top surface of the top substrate, a flat gasket placed beneath a bottom surface of the bottom substrate, or both. In some embodiments, the membrane seal extends in the x-y plane. In some embodiments, the manifold or connector comprises a finger cut-out area between two channels of the one or more channels of the flow cell device. In some embodiments, the manifold or connector comprises a seal placed in the finger cut-out area and configured to seal fluidic communication between the two channels. In some embodiments, the manifold or connector comprises a fluidic pathway with an outlet exiting the manifold on a plane that is orthogonal to the y axis, to the x axis, or to the z axis. In some embodiments, the top substrate or bottom substrate comprises one or more ramped ends. In some embodiments, a tip of one of the one or more ramped ends presses on the one or more seals. In some embodiments, each of the one or more ramped ends interfaces with a ramped manifold or connector. In some embodiments, the one or more ramped ends comprise a first acute ramp angle to a y axis. In some embodiments, the ramped manifold or connector comprises a second acute ramp angle to the y axis. In some embodiments, the first acute ramp angle is different from the second acute ramp angle. In some embodiments, the first acute ramp angle is identical to the second acute ramp angle. In some embodiments, the ramped manifold or connector comprises a complementary ramp to the ramped end of the flow cell device. In some embodiments, the one or more seals comprise a diagonal gasket with a fluidic pathway running in an y-z plane. In some embodiments, the diagonal gasket, manifold, or connector interfaces with an end of the top substrate and a top surface of the bottom substrate. In some embodiments, the diagonal gasket, manifold, or connector interfaces with an end of the bottom substrate and a top interior surface of the top substrate. In some embodiments, the diagonal gasket manifold, or connector allows sealed fluidic communication from the fluidic pathway to the one or more channels when a force or pressure comprises a y-axis component satisfying a first threshold and a z axis component satisfying a second threshold. In some embodiments, the top substrate and the bottom substrate are laterally offset from each other at least along the y axis. In some embodiments, at least part of the manifold or connector is fixedly attached to a bottom interior surface of the bottom substrate. In some embodiments, the flow cell device further comprises an interposer configured to define the one or more channels between the top substrate and the bottom substrate. In some embodiments, the top substrate and the bottom substrate are not fixedly attached to each other directly. In some embodiments, at least part of the manifold or connector is fixedly attached to a top interior surface of the top substrate. In some embodiments, the fluidic pathway of the diagonal gasket, manifold, or connector runs at least along the y axis. In some embodiments, the manifold or connector further comprises an open well leading to a second open landing area, and wherein the second open landing area is configured for receiving reagents from a dispensing tip. In some embodiments, the open well of the manifold or connector is in fluidic communication with the one or more channels. In some embodiments, the second open landing area of the manifold or connector is in fluidic communication with the inlet of the one or more channels. In some embodiments, the one or more seals comprise a thermoplastic connector and a thermoplastic seal mounted on the thermoplastic connector. In some embodiments, the thermoplastic seal is deformable under a pressure change, a temperature change, or both. In some embodiments, the thermoplastic seal comprises one or more materials that are different from one or more materials of the thermoplastic connector. In some embodiments, the one or more seals comprise a first connector having a top portion that is slidable on a top surface of the top substrate. In some embodiments, the one or more seals comprise a second connector having a bottom portion that is slidable on a bottom surface of the bottom substrate. In some embodiments, the top portion connects to a first side portion of the first connector that is configured to interface with an end of the flow cell device in the x-z plane. In some embodiments, the bottom portion connects to a second side portion of the second connector that is configured to interface with an end of the flow cell device in the x-z plane. In some embodiments, a pressure or force on the first and second side portion, satisfying a predetermined threshold, is configured to slide the first and second connector relative to the flow cell device with deformation thereby enabling sealed communication between the one or more channels and a fluidic pathway defined between the top and bottom connector. In some embodiments, the inlet comprises a port that opens at the bottom surface of the bottom substrate. In some embodiments, the port is in fluidic communication with the one or more channels and the fluidic pathway of the connector. In some embodiments, the one or more seals comprise a semi-rigid or deformable material that deforms under pressure or force. In some embodiments, the semi-rigid or deformable material is configured to restore its shape before deformation when the pressure or force is removed. In some embodiments, the one or more seals comprises a gasket, a second connector, a second manifold, or a part thereof, or their combinations. In some embodiments, the flow cell device further comprises a force-applying mechanism that is controlled by computer readable instructions executable on a computer processor. In some embodiments, the second manifold, the second connector, or the one or more seals are connected to the force-applying mechanism thereby allowing connection to or disconnection from the flow cell device.

Disclosed herein, in another aspect, is a flow cell system comprising: a flow cell device disclosed herein; a fluidic control device. In some embodiments, the fluidic control device comprises the first pump, the second pump, or both. In some embodiments, the fluidic control device comprises: a third pump coupled with the outlet of the flow cell device; and the dispenser that is configured to openly dispense the one or more reagents to the inlet of the flow cell device. In some embodiments, the fluidic control device comprises: a fourth pump in fluidic connection with the cleaning outlet of the flow cell device; a fifth pump, wherein the fourth pump or fifth pump is in fluidic connection with the outlet of the flow cell device; and the dispenser that is configured to openly dispense the one or more reagents to the inlet of the flow cell device. In some embodiments, the first pump or second pump is configured to introduce the gas gap via the inlet and flow the gas gap at least partly through the one or more channels. In some embodiments, the flow cell system further comprises a third manifold or connector with the fluidic pathway running in a y-z plane. In some embodiments, the first pump is configured to clean the open landing area by driving the residual amount of the first reagent off the open landing area to flow through the cleaning outlet.

Disclosed herein, in another aspect, is a method for preparing a flow cell for DNA sequencing reactions, comprising: (a) providing the flow cell comprising (i) an inlet and an outlet, wherein the inlet comprises an open landing area for receiving one or more reagents, and (ii) one or more channels disposed between the inlet and the outlet for performing the sequencing reactions; (b) openly dispensing a first reagent of the one or more reagents to the open landing area to flow at least part of the first reagent from the open landing area to the one or more channels; (c) introducing a gas into the one or more channels; (d) openly dispensing a second reagent of the one or more reagents to the open landing area to flow at least part of the second reagent from the open landing area to the one or more channels, thereby removing a residual amount of the first reagent from the one or more channels.

Disclosed herein, in another aspect, is a method for preparing a flow cell for DNA sequencing reactions, comprising: (a) providing the flow cell comprising (i) an inlet and an outlet, wherein the inlet comprises an open landing area for receiving one or more reagents, and (ii) one or more channels disposed between the inlet and the outlet for performing the sequencing reactions; (b) openly dispensing a first reagent of the one or more reagents to the open landing area to flow at least part of the first reagent from the open landing area to the one or more channels; wherein at least part of the open landing area comprises a surface coating to facilitate removal of a residual amount of the first reagent from the open landing area; and (c) openly dispensing a second reagent of the one or more reagents to the open landing area to flow at least part of the second reagent from the open landing area to the one or more channels.

Disclosed herein, in another aspect, is a method for sequencing with a flow cell device, comprising: (a) providing the flow cell comprising (i) an inlet and an outlet, wherein the inlet comprises an open landing area for receiving one or more reagents, and (ii) one or more channels disposed between the inlet and the outlet for performing the sequencing reactions; (b) openly dispensing a first reagent of the one or more reagents to the open landing area to flow at least part of the first reagent from the open landing area to the one or more channels; (c) removing a residual amount of the first reagent from at least part of the open landing area by flowing the residual amount of the first reagent through a cleaning outlet of the flow cell device; and (d) openly dispensing a second reagent of the one or more reagents to the open landing area to flow at least part of the second reagent from the open landing area to the one or more channels.

Disclosed herein, in another aspect, is a method for manufacturing a flow cell device, comprising: obtaining one or more substrates; generating one or more channels in the one or more substrates, wherein the one or more channels are configured to allow a fluid or a gas gap between the fluid and another fluid to flow through the one or more channels; forming an inlet comprising a hole in one of the one or more substrates and an open landing area, wherein the inlet is in fluidic connection with the one or more channels; forming an outlet that is in fluidic connection with the one or more channels; coating at least a portion of a surface of the one or more channels with a first coating, wherein the surface is configured to be dried and rewet during a DNA sequencing run; and fixedly coupling the one of one or more substrates together.

Disclosed herein, in another aspect, is a method for manufacturing a flow cell device, comprising: obtaining one or more substrates; generating one or more channels in the one or more substrates; forming an inlet comprising a hole in one of the one or more substrates and an open landing area, wherein the inlet is in fluidic connection with the one or more channels; coating at least a portion of a surface of the one or more channels with a first coating; covering at least a portion of the open landing area with a second coating; and fixedly coupling the one of one or more substrates together.

Disclosed herein, in another aspect, is a method for manufacturing a flow cell device, comprising: obtaining one or more substrates; forming an inlet comprising a hole in one of the one or more substrates and an open landing area; generating one or more channels in the one or more substrates; forming an outlet in the one or more substrates, wherein the inlet and outlet are in fluidic connection with the one or more channels; forming a cleaning outlet in the one or more substrates, wherein the cleaning outlet is in fluidic connection with the inlet, and wherein the cleaning outlet is closer to the inlet than to the outlet; and fixedly coupling the one of one or more substrates together.

In some embodiments, the one or more channels are configured to allow the gas gap to flow through the one or more channels between allowing the first reagent and the second reagent to flow through the one or more channels. In some embodiments, the one or more channels are configured to allow the gas gap to flow through the one or more channels during a DNA sequencing run. In some embodiments, the one or more channels are configured to allow the gas gap to flow through the one or more channels from the inlet. In some embodiments, the one or more channels are configured to allow the gas gap to flow through the one or more channels to facilitate reducing contamination of the second reagent by the first reagent in a DNA sequencing run. In some embodiments, the one or more channels are configured to allow the gas gap to flow through the one or more channels to reduce a minimum amount of the first reagent, the second reagent, or a washing reagent required for a DNA sequencing run. In some embodiments, the one of the one or more channels comprises one or more surfaces. In some embodiments, the one or more surfaces comprises an inner surface. In some embodiments, the one or more surfaces comprises an exterior surface. In some embodiments, the one or more surfaces comprises an interior top surface, an interior bottom surface, or both. In some embodiments, the one or more surfaces comprises an exterior top surface, an exterior bottom surface, or both. In some embodiments, the one or more surfaces comprises a planar surface. In some embodiments, the one or more surfaces is passivated. In some embodiments, the one or more surfaces is passivated with a coating that immobilizes a surface capture primer, a nucleic acid template molecule, or both, for capturing a polynucleotide. In some embodiments, the one or more surfaces comprises a polynucleotide captured thereon. In some embodiments, the gas gap is configured to dry at least part of the one or more surfaces of the one or more channels. In some embodiments, the gas gap does not impair a chemical function of the one or more surfaces. In some embodiments, the coating of the one or more surfaces comprises at least one hydrophilic polymer coating layer. In some embodiments, the coating of the one or more surfaces comprises a plurality of oligonucleotide molecules attached to at least one hydrophilic polymer coating layer. In some embodiments, the one or more surfaces comprises at least one discrete region that comprises a plurality of clonally-amplified sample nucleic acid molecules that have been annealed to a plurality of attached oligonucleotide molecules. In some embodiments, the at least one hydrophilic polymer coating layer has a water contact angle of no more than about 50 degrees. In some embodiments, at least one of the plurality of clonally-amplified sample nucleic acid molecules comprises a concatemer annealed to at least one of the plurality of attached oligonucleotide molecules. In some embodiments, the at least one hydrophilic polymer coating layer comprises PEG. In some embodiments, the one or more surfaces further comprises a second hydrophilic polymer coating layer. In some embodiments, the at least one hydrophilic polymer coating layer comprises a branched hydrophilic polymer. In some embodiments, the branched hydrophilic polymer comprises at least 8 branches. In some embodiments, the at least one of the plurality of the clonally-amplified sample nucleic acid molecules comprises a single-stranded multimeric nucleic acid molecule comprising repeats of a regularly occurring monomer unit. In some embodiments, the single-stranded multimeric nucleic acid molecule is at least 10 kilobases in length. In some embodiments, at least one of the plurality of the clonally-amplified sample nucleic acid molecules further comprises a double-stranded monomeric copy of the regularly occurring monomer unit. In some embodiments, the plurality of oligonucleotide molecules is present at about a uniform surface density across the one or more surfaces. In some embodiments, the plurality of oligonucleotide molecules is present at a local surface density of at least 100,000 molecules/μm² at a first region on the one or more surfaces, and at a second local surface density at a second region on the one or more surfaces. In some embodiments, the coating comprises: a first layer comprising a monolayer of polymer molecules tethered to the one or more surfaces of the substrate; a second layer comprising a second monolayer of polymer molecules tethered to the polymer molecules of the first layer; and a third layer comprising a third monolayer of polymer molecules tethered to the polymer molecules of the second layer, wherein at least one of the first layer, the second layer, or the third layer comprises branched polymer molecules. In some embodiments, the third layer further comprises oligonucleotides tethered to the polymer molecules of the third layer. In some embodiments, the oligonucleotides tethered to the polymer molecules of the third layer are distributed at a plurality of depths throughout the third layer. In some embodiments, the coating further comprises: a fourth layer comprising branched polymer molecules tethered to the polymer molecules of the third layer, and a fifth layer comprising polymer molecules tethered to the branched polymer molecules of the fourth layer. In some embodiments, the polymer molecules of the fifth layer further comprise oligonucleotides tethered to the polymer molecules of the fifth layer. In some embodiments, the oligonucleotides tethered to the polymer molecules of the fifth layer are distributed at a plurality of depths throughout the fifth layer. In some embodiments, the at least one hydrophilic polymer coating layer comprises polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxyethyl methacrylate) (PHEMA), poly(oligo (ethylene glycol) methyl ether methacrylate) (POEGMA), polyglutamic acid (PGA), poly-lysine, poly-glucoside, streptavidin, or dextran. In some embodiments, when the clonally-amplified sample nucleic acid molecules or complementary sequences thereof are labeled with Cyanine dye-3, an image of the one or more surfaces exhibits a ratio of fluorescence intensities for the clonally-amplified, Cyanine dye-3-labeled sample nucleic acid molecules, or complementary sequences thereof, and nonspecific Cyanine dye-3 dye adsorption background (Bᵢₙₜₑᵣ) of at least 3:1. In some embodiments, the image of the one or more surfaces exhibits a ratio of fluorescence intensities for clonally amplified, Cyanine dye-3-labeled sample nucleic acid molecules, or complementary sequences thereof, and a combination of nonspecific Cyanine dye-3 dye adsorption background and nonspecific amplification background (Bᵢₙₜₑᵣ+Bᵢₙₜᵣₐ) of at least 3:1. In some embodiments, when the clonally-amplified sample nucleic acid molecules or complementary sequences thereof are labeled with Cyanine dye-3, the image of the one or more surfaces exhibits a ratio of fluorescence intensities for clonally-amplified, Cyanine dye-3-labeled sample nucleic acid molecules, or complementary sequences thereof, and nonspecific dye adsorption background (Bᵢₙₜₑᵣ) of at least 5:1. In some embodiments, the image of the one or more surfaces exhibits a ratio of fluorescence intensities for clonally-amplified, Cyanine dye-3-labeled sample nucleic acid molecules, or complementary sequences thereof, and a combination of nonspecific Cyanine dye-3 dye adsorption background and nonspecific amplification background (Bᵢₙₜₑᵣ+Bᵢₙₜᵣₐ) of at least 5:1. In some embodiments, when the clonally-amplified sample nucleic acid molecules or complementary sequences thereof are labeled with Cyanine dye-3, the fluorescence image of the one or more surfaces exhibits a contrast-to-noise ratio (CNR) of at least 20 when the fluorescence image is acquired using an inverted microscope equipped with a 20× objective, NA=0.75, dichroic mirror optimized for 532 nm light, a bandpass filter optimized for Cyanine dye-3 emission, and a camera under non-signal saturating conditions, while the one or more surfaces is immersed in a buffer. In some embodiments, the plurality of oligonucleotide molecules is present at a surface density of at least 1,000 molecules/m². In some embodiments, the first reagent is configured to wet the one or more surfaces of the one or more channels. In some embodiments, the second reagent is configured to rewet the one or more surfaces of the one or more channels after at least partly drying the one or more surfaces by the gas gap. In some embodiments, a flow cell system comprises the flow cell device, wherein the flow cell system further comprises: a fluidic control device comprising: a first pump coupled with the outlet; and a dispenser that is configured to openly dispense one or more reagents to the inlet. In some embodiments, the first pump or a second pump is configured to introduce the gas gap via the inlet and flow the gas gap at least partly through the one or more channels. In some embodiments, the gas gap comprises air. In some embodiments, the gas gap comprises dry air. In some embodiments, the gas gap comprises one or more inert gases. In some embodiments, the gas gap comprises one or more active gases. In some embodiments, the first or second reagent comprise a liquid. In some embodiments, the first or the second reagent lacks an air bubble that is greater than a predetermined size. In some embodiments, the coating comprises a liquid-repelling coating. In some embodiments, the coating comprises an omniphobic coating. In some embodiments, the coating comprises a slippery liquid-infused porous surface (SLIPS). In some embodiments, the coating comprises a slippery omniphobic covalently attached liquid (SOCAL) coating. In some embodiments, the coating comprises a liquid-like polymer brush surface that is covalently attached to the one or more substrates. In some embodiments, the coating is formed by impregnating a lubricant in one or more porous surfaces. In some embodiments, the lubricant comprises a liquid with a surface energy below about 20 mJ/m². In some embodiments, the lubricant comprises a silicone oil. In some embodiments, the coating comprises a surface energy that is below about 20 mJ/m². In some embodiments, the coating is formed by acid-catalyzed graft polycondensation of one or more saline monomers. In some embodiments, the one or more saline monomers comprises dimethyldimethoxysilane. In some embodiments, the open landing area is in fluidic connection with the one or more channels. In some embodiments, the open landing area is in fluidic connection with one of the one or more channels. In some embodiments, the open landing area is on a bottom substrate of the one or more substrates. In some embodiments, the inlet comprises a hole in a top substrate of the one or more substrates. In some embodiments, the hole in the top substrate is positioned above at least part of the open landing area. In some embodiments, the dispenser is configured to openly dispense the one or more reagents through the hole to the open landing area. In some embodiments, the dispenser is configured to openly dispense the one or more reagents from a tip of the dispenser to the open landing area. In some embodiments, the dispenser is configured to openly dispense the one or more reagents from the tip of the dispenser to the open landing area without tubing in between the dispenser and the open landing area. In some embodiments, at least part of the tip of the dispenser is in contact with the open landing area. In some embodiments, the tip of the dispenser is not in contact with the open landing area. In some embodiments, the flow cell device further comprises a cleaning outlet in the one or more substrates. In some embodiments, the cleaning outlet is in fluidic connection with the inlet. In some embodiments, the cleaning outlet is in fluidic connection with the open landing area. In some embodiments, the cleaning outlet is in a top or a bottom substrate of the one or more substrates. In some embodiments, the cleaning outlet comprises a side port on the one or more substrates, wherein the side port: extends at least along a direction that is perpendicular or nearly perpendicular to an x direction; extends at least along a direction that is perpendicular or nearly perpendicular to a y direction; extends at least along a direction that is perpendicular or nearly perpendicular to a z direction; extends at least along a direction that is oblique to an x direction; extends at least along a direction that is oblique to a y direction; or extends at least along a direction that is oblique to a z direction. In some embodiments, the cleaning outlet is configured to be coupled with the first pump or the second pump. In some embodiments, the one or more channels comprises a microfluidic channel. In some embodiments, the one or more surfaces is coated with a fluorescent bead that is chemically immobilized to the one or more surfaces. In some embodiments, the fluorescent bead is covalently attached to the one or more surfaces. In some embodiments, a gap between the interior top surface and the interior bottom surface is about 150 μm, 130 μm, 120 μm, 110 μm, 100 μm, 90 μm, 80 μm, 70 μm, 60 μm, 50 μm, or 40 μm. In some embodiments, a height of the one or more channels is about 150 μm, 130 μm, 120 μm, 110 μm, 100 μm, 90 μm, 80 μm, 70 μm, 60 μm, 50 μm, or 40 μm. In some embodiments, the polynucleotide captured thereon is configured to be imaged in a sequencing cycle. In some embodiments, the one or more substrates comprises a top substrate and a bottom substrate. In some embodiments, the one or more channels are defined between the top substrate and the bottom substrate. In some embodiments, the one or more channels are defined at least partly in a top surface of the bottom substrate. In some embodiments, the one or more channels are defined at least partly in a bottom surface of the top substrate. In some embodiments, the one or more substrates further comprises a middle substrate. In some embodiments, the one or more channels are defined at least partly in the middle substrate. In some embodiments, the one or more substrates comprise glass or plastic. In some embodiments, at least part of the support is transparent. In some embodiments, at least part of the one or more substrates is transparent. In some embodiments, the support is solid. In some embodiments, the one or more channels comprise 1, 2, 3, 4, 5, 6, 7, or 8 channels. In some embodiments, the one or more channels comprise 2, 4, 6, 8, or 10 channels. In some embodiments, each channel of the one or more channels comprises a lane length of less than about 70 mm, 75 mm, 80 mm, or 90 mm. In some embodiments, each channel of the one or more channels comprises a lane width of less than about 10 mm, 8 mm, 7 mm, 6 mm, 5 mm, 4 mm, or 3 mm. In some embodiments, at least a portion of the open landing area comprises a second coating comprising a slippery coating. In some embodiments, at least a portion of the open landing area comprises a second coating comprising a liquid-repelling coating. In some embodiments, at least a portion of the open landing area comprises a second coating comprising an omniphobic coating. In some embodiments, at least a portion of the open landing area comprises a second coating comprising a slippery liquid-infused porous surface (SLIPS). In some embodiments, at least a portion of the open landing area comprises a second coating comprising a slippery omniphobic covalently attached liquid (SOCAL) coating. In some embodiments, at least a portion of the open landing area comprises a second coating comprising a liquid-like polymer brush surface that is covalently attached to the one or more substrates. In some embodiments, at least a portion of the open landing area comprises a second coating comprising impregnating a lubricant in porous surfaces to generate the coating with a surface energy below about 20 mJ/m². In some embodiments, at least a portion of the open landing area comprises a second coating comprising impregnating acid-catalyzed graft polycondensation of one or more saline monomers. In some embodiments, the one or more saline monomers comprise dimethyldimethoxysilane. In some embodiments, a process of using the flow cell device comprises removing at least part of the first reagent from at least part of the one or more channels during a DNA sequencing run. In some embodiments, the at least part of the first reagent remains in the one or more channels during the DNA sequencing run. In some embodiments, the first reagent and the second reagent are different. In some embodiments, at least part of the one or more channels comprises more than about 40% of a corresponding volume or length of each of the one or more channels. In some embodiments, at least part of the one or more channels comprises more than about half of a corresponding volume or length of each of the one or more channels. In some embodiments, at least part of the one or more channels comprises more than about 60% of a corresponding volume or length of each of the one or more channels. In some embodiments, at least part of the one or more channels comprises more than about 70% of a corresponding volume or length of each of the one or more channels. In some embodiments, at least part of the one or more channels comprises more than about 80% of a corresponding volume or length of each of the one or more channels. In some embodiments, a process of using the flow cell device comprises driving a residual amount of the first reagent or the second reagent off the open landing area via a cleaning outlet of the flow cell device. In some embodiments, the cleaning outlet is configured to allow a residual amount of the first reagent on the open landing area to flow through the cleaning outlet. In some embodiments, a flow cell system comprises the flow cell device, wherein the flow cell system further comprises: a fluidic control device comprising: a first pump in fluidic connection with the cleaning outlet, wherein the first pump or a second pump is in fluidic connection with the outlet; and a dispenser that is configured to openly dispense the one or more reagents to the inlet. In some embodiments, the first pump is configured to clean the open landing area by driving a residual amount of the first reagent off the open landing area to flow through the cleaning outlet. In some embodiments, the process further comprises: removing at least part of the first reagent from at least part of the one or more channels by driving the gas gap between fluids from the inlet and through at least part of the one or more channels. In some embodiments, the residual amount of the first reagent on the open landing area comprises meniscus of the first reagent.

### DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIG. 1 is a non-limiting example of a block diagram of a computer-implemented system for performing operations in DNA sequencing and sequencing analysis using the flow cell devices herein, according to some embodiments.

FIG. 2A is a schematic showing of a non-limiting example of the flow cell device, according to some embodiments.

FIG. 2B is a schematic showing of a non-limiting example of the flow cell device, according to some embodiments.

FIG. 2C is a schematic showing of a non-limiting example of the flow cell device, according to some embodiments.

FIG. 2D is a schematic showing of a non-limiting example of the flow cell device, according to some embodiments.

FIG. 2E is a schematic showing of a non-limiting example of the flow cell device, according to some embodiments.

FIG. 2F is a schematic showing of a non-limiting example of the flow cell device, according to some embodiments.

FIG. 2G is a schematic showing of a non-limiting example of the flow cell device, according to some embodiments.

FIG. 3A is a schematic showing of a non-limiting example of the flow cell device, according to some embodiments.

FIG. 3B is a schematic showing of a non-limiting example of the flow cell device, according to some embodiments.

FIG. 4A is a non-limiting example of the flow cell device, according to some embodiments.

FIG. 4B is a non-limiting example of the flow cell device, according to some embodiments.

FIG. 4C is a non-limiting example of the flow cell device, according to some embodiments.

FIG. 4D is a non-limiting example of the flow cell device, according to some embodiments.

FIG. 5A is a schematic showing of a non-limiting example of the flow cell device, according to some embodiments.

FIG. 5B is a schematic showing of a non-limiting example of the flow cell device, according to some embodiments.

FIG. 5C is a schematic showing of a non-limiting example of the flow cell device, according to some embodiments.

FIG. 5D is a schematic showing of a non-limiting example of the flow cell device, according to some embodiments.

FIG. 6A is a non-limiting example of the flow cell device, according to some embodiments.

FIG. 6B is a non-limiting example of the flow cell device, according to some embodiments.

FIG. 6C is a non-limiting example of the flow cell device, according to some embodiments.

FIG. 7A is a schematic showing of a non-limiting example of the flow cell device with the embedded tube(s), according to some embodiments.

FIG. 7B is a schematic showing of a non-limiting example of the flow cell device with the embedded tube(s), according to some embodiments.

FIG. 7C is a schematic showing of a non-limiting example of the flow cell device with the embedded tube(s), according to some embodiments.

FIG. 7D is a schematic showing of a non-limiting example of the flow cell device with the embedded tube(s), according to some embodiments.

FIG. 8 is a schematic showing of a non-limiting example of the flow cell device, according to some embodiments.

FIG. 9A is a schematic showing of a non-limiting example of the flow cell device, according to some embodiments.

FIG. 9B is a non-limiting example of the flow cell device, according to some embodiments.

FIG. 9C is a non-limiting example of the flow cell device, according to some embodiments.

FIG. 9D is a non-limiting example of the flow cell device, according to some embodiments.

FIG. 9E is a non-limiting example of the flow cell device, according to some embodiments.

FIG. 10 is a schematic showing of a non-limiting example of the flow cell device, according to some embodiments.

FIG. 11 is a schematic showing of a non-limiting example of the flow cell device, according to some embodiments.

FIG. 12A is a non-limiting example of the flow cell device, according to some embodiments.

FIG. 12B is a non-limiting example of the flow cell device, according to some embodiments.

FIG. 12C is a non-limiting example of the flow cell device, according to some embodiments.

FIG. 12D is a non-limiting example of the flow cell device, according to some embodiments.

FIG. 12E is a non-limiting example of the flow cell device, according to some embodiments.

FIG. 12F is a non-limiting example of the flow cell device, according to some embodiments.

FIG. 12G is a non-limiting example of the flow cell device, according to some embodiments.

FIG. 12H is a non-limiting example of the flow cell device, according to some embodiments.

FIG. 13 is a schematic showing of a non-limiting example of the flow cell device, according to some embodiments.

FIG. 14A is a schematic showing of a non-limiting example of the flow cell device, according to some embodiments.

FIG. 14B is a schematic showing of a non-limiting example of the flow cell device, according to some embodiments.

FIG. 14C is a schematic showing of a non-limiting example of the flow cell device, according to some embodiments.

FIG. 14D is a schematic showing of a non-limiting example of the flow cell device, according to some embodiments.

FIG. 15 is a schematic showing of a non-limiting example of a linear single stranded library molecule.

FIG. 16 is a schematic showing of a non-limiting example of a linear single stranded library molecule.

FIG. 17 is a schematic of non-limiting examples of various configurations of multivalent molecules.

FIG. 18 is a schematic of a non-limiting example of a multivalent molecule comprising a generic core attached to a plurality of nucleotide-arms.

FIG. 19 is a schematic of a non-limiting example of a multivalent molecule comprising a dendrimer core attached to a plurality of nucleotide-arms.

FIG. 20 is a schematic of a non-limiting example of a multivalent molecule comprising a core attached to a plurality of nucleotide-arms, where the nucleotide arms comprise biotin, spacer, linker and a nucleotide unit.

FIG. 21 is a schematic of a non-limiting example of a nucleotide-arm comprising a core attachment moiety, spacer, linker and nucleotide unit.

FIG. 22 is a schematic of non-limiting examples of the chemical structures of a spacer (top), and the chemical structures of various linkers, including an 11-atom Linker, a 16-atom Linker, a 23-atom Linker and an N3 Linker (bottom).

FIG. 23 is a schematic of non-limiting examples of the chemical structures of various linkers, including Linkers 1-9.

FIG. 24 is a schematic of non-limiting examples of the chemical structures of various linkers joined/attached to nucleotide units.

FIG. 25 is a schematic of non-limiting examples of the chemical structures of various linkers joined/attached to nucleotide units.

FIG. 26 is a schematic of non-limiting examples of the chemical structures of various linkers joined/attached to nucleotide units.

FIG. 27 is a schematic of non-limiting examples of the chemical structures of various linkers joined/attached to nucleotide units.

FIG. 28 is a schematic of a non-limiting example of the chemical structure of a biotinylated nucleotide-arm.

FIG. 29 is a schematic of a non-limiting example of the flow cell devices.

FIG. 30 is a schematic of a non-limiting example of a flow cell system, according to some embodiments.

FIG. 31 is a schematic of a non-limiting example of a flow cell device, according to some embodiments.

FIG. 32 is a schematic of a non-limiting example of a flow cell device, according to some embodiments.

FIGS. 33A-33F are non-limiting examples of flow cell devices, according to some embodiments. FIG. 33A is a prospective view of the substrates of an embodiment of the flow cell device disclosed herein. FIG. 33B is a top view of the flow cell device in FIG. 33A. FIG. 33C is a cross-sectional view of the flow cell device at D-D′ in FIG. 33B. FIG. 33D is a prospective view of the substrates of another embodiment of the flow cell device disclosed herein. FIG. 33E is a prospective view of the substrates of yet another embodiment of the flow cell device disclosed herein. FIG. 33F shows a prospective view and a top view of yet another embodiment of the flow cell device disclosed herein.

FIGS. 34A-34C are non-limiting examples of fluidic control devices of the flow cell systems for delivery of reagents to the flow cell devices, according to some embodiments. FIG. 34A shows a fluidic control device comprising a dispenser (680a) and a continuous track (691a). FIG. 34B shows a fluidic control device comprising a dispensing plate (692a) with an electrowetting surface. FIG. 34C shows a fluidic control device comprising a reagent reservoir (694a) and a sipper (693a).

FIG. 35 is a graph illustrating contamination levels achieved by flow cell systems disclosed herein in comparison to existing flow cell systems.

FIG. 36 is a non-limiting example of a block diagram of a computer system for fluidic control and for performing sequencing and sequencing analysis, according to some embodiments.

FIGS. 37A-37E are non-limiting examples of the flow cell device in FIGS. 33A-33D. FIG. 37A is a perspective view of the flow cell device. FIG. 37B is a perspective view of the flow cell device showing the top, middle and bottom substrates. FIG. 37C is a top view of the top substrate of the flow cell device. FIG. 37D is a top view of the middle substrate of the flow cell device. FIG. 37E is a top view of the bottom substrate of the flow cell device.

FIGS. 38A-38E are non-limiting examples of the flow cell device disclosed herein. FIG. 38A is a perspective view of the flow cell device. FIG. 38B is a perspective view of the flow cell device showing the top, middle and bottom substrates. FIG. 38C is a top view of the top substrate of the flow cell device. FIG. 38D is a top view of the middle substrate of the flow device. FIG. 38E is a top view of the bottom substrate of the flow cell device.

FIGS. 39A-39C are non-limiting examples of the flow cell device disclosed herein. FIG. 39A is a top view of an embodiment of the flow cell device. FIG. 39B is a top view of another embodiment of the flow cell device. FIG. 39C is a top view of yet another embodiment of the flow cell device.

FIGS. 40A-40G are non-limiting examples of the flow cell device disclosed herein. FIG. 40A is a side view of the flow cell device. FIG. 40B shows a cross-sectional view at A-A in FIG. 40A. FIG. 40C is a top view of the flow cell device. FIG. 40D is a cross-sectional view at B-B in FIG. 40B. FIG. 40E shows an expanded view of area A in FIG. 40B. FIG. 40F shows an expanded view of area C in FIG. 40C. FIG. 40G shows an expanded view of area B in FIG. 40D.

FIGS. 41A-41C are an embodiment of the flow cell device in FIG. 33E in a top view (FIG. 41A), a prospective view (FIG. 41B), and a prospective view of the bottom, middle, and top substrates (FIG. 41C).

FIG. 42 is a graph illustrating contamination levels of individual tiles and average contamination level across multiple tiles of the flow cell device achieved by flow cell systems disclosed herein.

FIG. 43A a non-limiting example of an embodiment of the flow cell device with a filter. In this particular embodiment, the filter reduces or eliminates contamination that may enter the channel from the open landing area.

FIG. 43B a non-limiting example of an embodiment of the flow cell device with a filter. In this particular embodiment, the filter reduces or eliminates contamination that may enter the channel from the open landing area.

### DETAILED DESCRIPTION

Described herein are systems and devices to analyze different nucleic acid sequences e.g., from amplified nucleic acid arrays in flow cells or from an array of immobilized nucleic acids. The systems and devices described herein can also be useful in, e.g., sequencing for comparative genomics, tracking gene expression, microRNA sequence analysis, epigenomics, and aptamer and phage display library characterization, and other sequencing applications. The systems and devices herein comprise various combinations of optical, mechanical, fluidic, thermal, electrical, and computing devices/aspects.

The advantages of the disclosed flow cell devices, fluidic control devices, and systems include, but are not limited to: significantly lower consumable costs (e.g., as compared to those for currently available nucleic acid sequencing systems); efficient and effective cleaning of flow cell devices thereby reducing contamination of sequencing processes, e.g., by residual reagent(s); reduced delivery time of reagents, reduced washing time, and increased homogeneity of reagents on the flow cells; reduced device and system manufacturing/maintenance complexity and cost; flexible system throughput and flexible adaptation of the systems to different sequencing applications.

The advantages of the disclosed flow cell devices and systems may also include: reduced bending stress on the flow cell substrate, which may lead to improved optical flatness and performance; improved thermal contact between the flow cell and the thermal controlling devices, which may improve chemistry performance on the flow cell; improved spatial clearance for open fluidics dispensing to the flow cell device; increased surface area for the development and imaging of polonies, which may improve the throughput of sequencing applications; and improved fluid interface sealing than existing flow cell devices.

The design features of some disclosed flow cell devices, cartridges, and systems include, but are not limited to: an open dispensing tip in the fluidic control device and an open landing area on the flow cell device to allow open delivery of reagents and/or washing liquid(s) without the complexity and cost of existing tubing and to enable flexibility in the systems to adapt to different sequencing applications; a slippery coating that facilitates fluidic transfer and residual cleaning from the opening landing area; a cleaning outlet in fluidic connection to the open landing area to facilitate cleaning of liquid meniscus that cannot be effectively cleaned using washing reagent(s) or washing buffer(s) alone; a location of the cleaning outlet(s) that allow effective cleaning during sequencing processes and convenient expansion of the flow cell device to increase sequencing throughput without the need to change the cleaning outlet(s); a channel coating that allows purging of an air gap between two fluidic reagents without damaging subsequent sequencing reactions; and compatibility with a wide variety of detection methods such as fluorescence imaging.

Although the disclosed flow cell devices and systems are described primarily in the context of their use for nucleic acid sequencing applications, various aspects of the disclosed devices and systems may be applied not only to nucleic acid sequencing but also to any other type of chemical analysis, biochemical analysis, nucleic acid analysis, cell analysis, or tissue analysis application. It shall be understood that different aspects of the disclosed devices and systems can be appreciated individually, collectively, or in combination with each other.

### Sequencing Systems

Disclosed herein, in some embodiments, are flow cell devices and systems that can be employed for performing or facilitating DNA sequencing analysis using sequencing systems, e.g., next generation sequencing (NGS) systems. The sequencing systems may utilize various sequencing techniques including but not limited to the sequencing techniques disclosed herein.

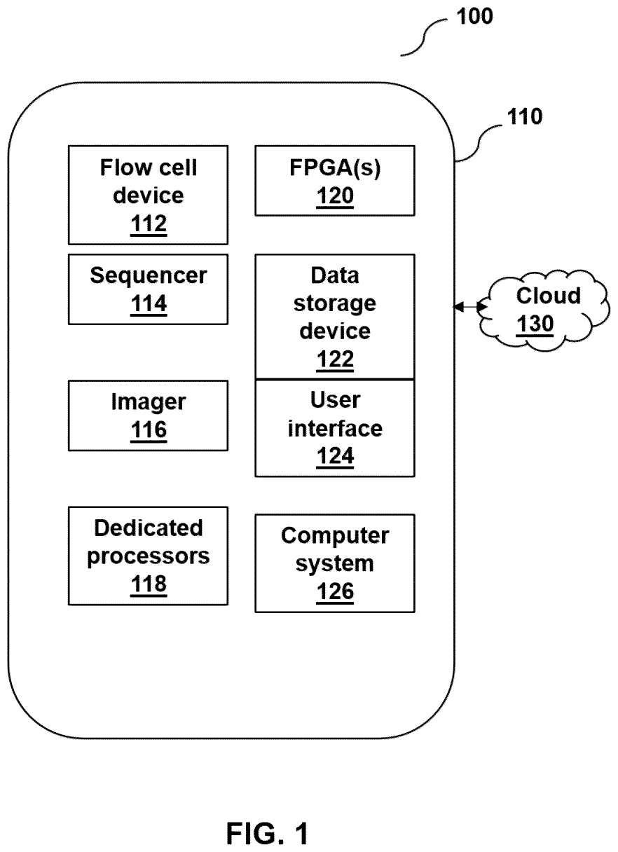

FIG. 1 illustrates a block diagram of a computer-implemented system 100 for performing sequencing and sequencing analysis, according to one or more embodiments disclosed herein. The system 100 has a sequencing system 110 that includes a flow cell device 112, a sequencer 114, an imager 116, a data storage device 122, and a user interface 124. The sequencing system 110 may be connected to a cloud 130. The sequencing system 110 may include one or more of dedicated processors 118, Field-Programmable Gate Array(s) (FPGAs) 120, and a computer system 126.

In some embodiments, the flow cell device 112 is configured to capture DNA fragments and form DNA sequences for base-calling on the flow cell device. The flow cell device 112 can include a support as disclosed herein. The support can be a solid support. The support can include a surface coating thereon as disclosed herein. The surface coating can be a polymer coating as disclosed herein. The surface coating can be on a surface of the one or more channels of the flow cell device. A different or identical surface can be placed on a surface of the inlet of the flow cell device.

A flow cell device 112 can include multiple tiles or imaging areas thereon, and each tile may be separated into a grid of subtiles. Each subtile can include a plurality of clusters or polonies thereon. As a nonlimiting example, a flow cell can have 424 tiles, and each tile can be divided into a 6×9 grid, therefore 54 subtiles. The flow cell image as disclosed herein can be an image including signals of a plurality of clusters or polonies. The flow cell image can include one or more tiles of signals or one or more subtiles of signals. In some embodiments, a flow cell image can be an image that includes all the tiles and approximately all signals thereon. The flow cell image can be acquired from a channel during an imaging or sequencing cycle using the imager 116. In some embodiments, each tile may include millions of polonies or clusters. As a nonlimiting example, a tile can include about 1 to 10 million clusters or polonies. Each polony can be a collection of many copies of DNA fragments.

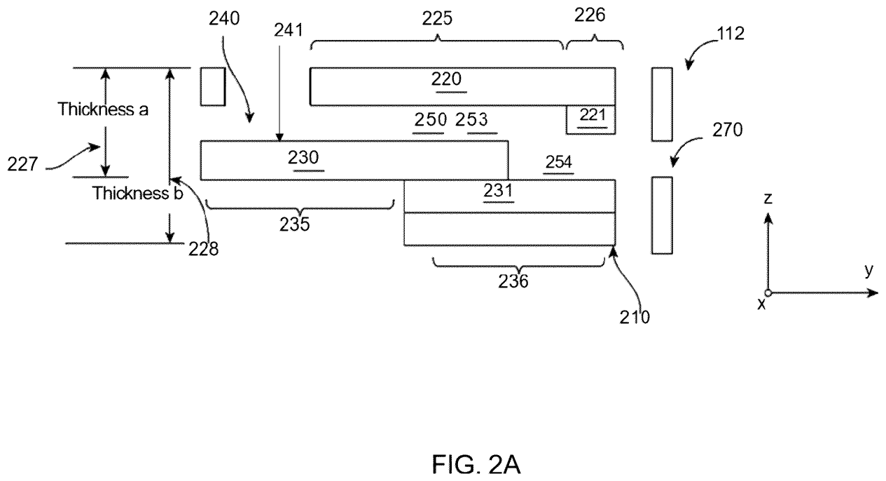

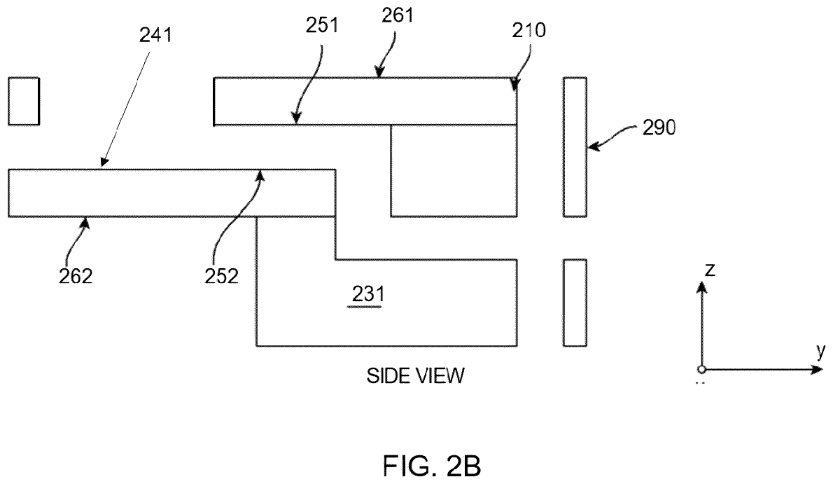

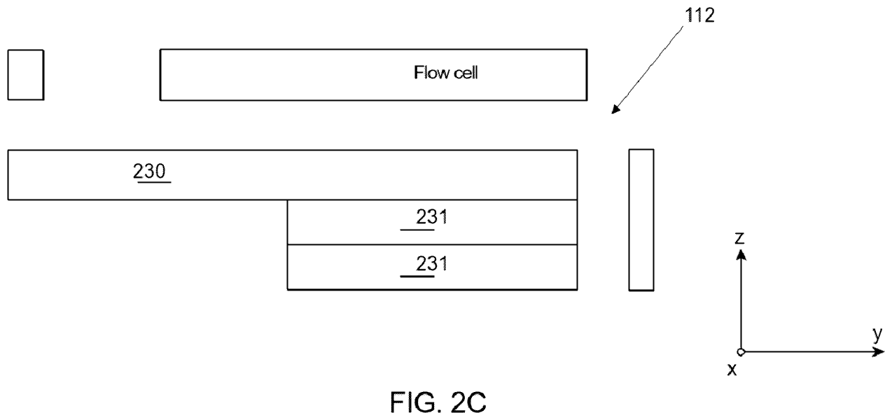

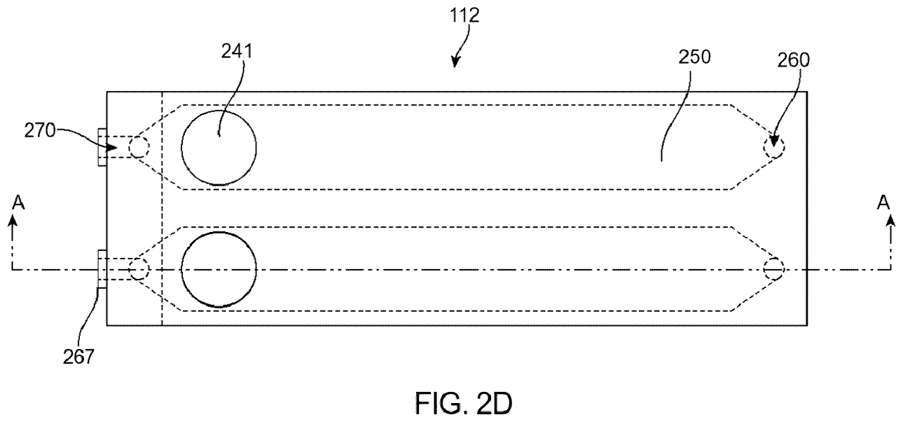

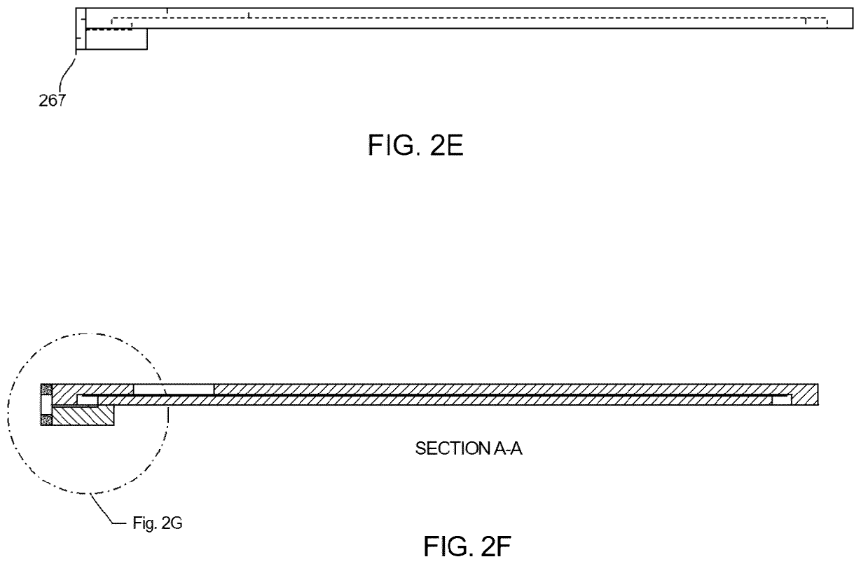

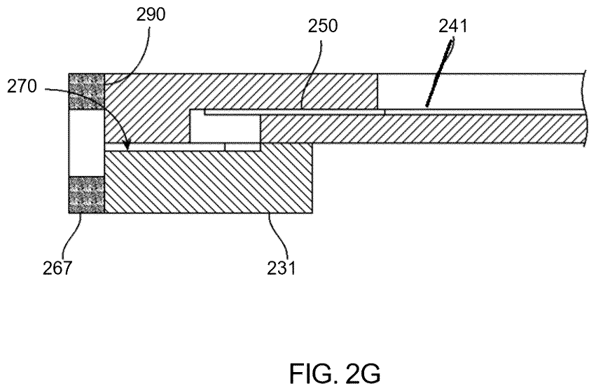

More details of the flow cell device 112 and its functional and structural elements are disclosed herein in relation to figures, e.g., FIGS. 2A-2G, FIGS. 3A-3B, FIGS. 4-14, FIGS. 30-32, 33A-33F, 34A-34C, and 35.

The sequencer 114 may be configured to flow a nucleotide mixture onto the flow cell device 112, cleave blockers from the nucleotides in between flowing steps, and perform other steps for the formation of the DNA sequences on the flow cell device 112. The nucleotides may have fluorescent elements attached that emit light or energy in a wavelength that indicates the type of nucleotide. Each type of fluorescent element may correspond to a particular nucleotide base (e.g., A, G, C, T). The fluorescent elements may emit light in visible wavelengths. In some embodiments, the sequencer 114 and the flow cell device 112 may be configured to perform various sequencing methods disclosed herein, for example, sequencing-by-avidite or sequencing-by-synthesis.

For example, each nucleotide base may be assigned a color. Different types of nucleotides can have different colors. Adenine (A) may be red, cytosine (C) may be blue, guanine (G) may be green, and thymine (T) may be yellow, for example. The color or wavelength of the fluorescent element for each nucleotide may be selected so that the nucleotides are distinguishable from one another based on the wavelengths of light emitted by the fluorescent elements.

The imager 116 may be configured to capture images of the flow cell device 112 after each flowing step. In an embodiment, the imager 116 is a camera configured to capture digital images, such as a CMOS or a CCD camera. The camera may be configured to capture images at the wavelengths of the fluorescent elements bound to the nucleotides. The images can be called flow cell images.

In some embodiments, the imager 116 can include one or more optical systems disclosed herein. The optical system(s) can be configured to capture optical signals from the flow cell and generate corresponding digital images thereof. The digital images can then be used for base calling.

In an embodiment, the images of the flow cell may be captured in groups, where each image in the group is taken at a wavelength or in a spectrum that matches or includes one of the fluorescent elements. In another embodiment, the images may be captured as single images that capture all of the wavelengths of the fluorescent elements.

The resolution of the imager 116 controls the level of detail in the flow cell images, including pixel size. In existing systems, this resolution is very important, as it controls the accuracy with which a spot-finding algorithm identifies the polony centers. In some embodiments, the image resolution of flow cell images disclosed herein can be about 10 nanometers (nms) to a couple of hundreds of nms or greater. One way to increase the accuracy of spot finding is to improve the resolution of the imager 116, or improve the processing performed on images taken by imager 116. Detecting polony centers in pixels other than those detected by a spot-finding algorithm can be performed. These methods can allow for improved accuracy in detection of polony centers without increasing the resolution of the imager 116. The resolution of the imager may even be less than existing systems with comparable performance, which may reduce the cost of the sequencing system 110.

The image quality of the flow cell images controls the base calling quality. One way to increase the accuracy of base calling is to improve the imager 116, or improve the processing performed on images taken by imager 116 to result in a better image quality.

After base calling is performed, with the option of certain processing on base calling results, sequencing reads can be outputted from the system to the cloud 130 or to a computer system 126. The sequencing read(s) herein can be a forward read (R1), a reverse read (R2), or both. The sequencing reads herein can be any orderly sequence of bases of A, T, C, and G.

In some embodiments, the sequencing reads can be directly communicated to the computer system 126 for subsequent analysis such as adaptor trimming.

These sequencing analysis methods, including primary analysis, or secondary analysis, or combinations thereof, can be advantageously performed in parallel in the computer system 126, without interference with or delay of existing sequencing workflow of the system 100. The results of sequencing analysis can be made available for generating sequencing results for users. Some or all operations of the sequencing process can be advantageously performed by the FPGA(s) and data can be communicated between the CPU(s) and FPGA(s) to reduce the total operational time from methods operating without the FPGA(s).

The operations or actions disclosed herein may be performed by the dedicated processors 118, the FPGA(s) 120, the computer system 126, or a combination thereof. One or more operations or actions in methods disclosed herein may be performed by the dedicated processors 118, the FPGA(s) 120, the computer system 126, or a combination thereof. In some embodiments, which operations or actions are to be performed by the dedicated processors 118, the FPGA(s) 120, the computer system 126, or their combinations can be determined based on one or more of: a computation time for the specific operation(s), the complexity of computation in the specific operation(s), the need for data transmission between the hardware devices, or combinations thereof.

The computer system 126 can include one or more general purpose computers or computer processors that provide interfaces to run a variety of programs in an operating system, such as Windows™ or Linux™. Such an operating system may provide great flexibility to a user.

In some embodiments, the computer processor may control various structural elements of a flow cell system or flow cell device as disclosed herein. For example, the computer processor may execute computer instructions to control a force-applying mechanism that applies force or pressure on a connector, a manifold, a seal, or a combination thereof to enable seal fluidic connection to the flow cell device.

The dedicated processors 118 may not be general-purpose processors, but instead custom processors with specific hardware or instructions for performing method steps. In some embodiments, the dedicated processors may include various processing units. In some embodiments, the dedicated processors may include: application specific integrated circuits (ASIC) chips, neural processing units (NPUs), artificial intelligence chips (AI chips), tensor processing units (TPUs), graphic processing units (GPU). Dedicated processors may include integrated circuits that may be reconfigurable or non-configurable but optimized for specific computational tasks, e.g., making predictions using neural networks. Dedicated processors may directly run specific software without an operating system. The lack of an operating system reduces overhead, at the cost of the flexibility in what the processor may perform. A dedicated processor may make use of a custom programming language, which may be designed to operate more efficiently than the software run on general-purpose computers. This may increase the speed at which the steps are performed and allow for real time processing.

In some embodiments, the FPGA(s) 120 may be configured to perform operations of the sequencing analysis methods described herein. An FPGA is programmed as hardware that may perform a specific task. A special programming language may be used to transform software steps into hardware componentry. Once an FPGA is programmed, the hardware directly processes digital data that is provided to it without running software. The FPGA instead uses logic gates and registers to process the digital data. Because there is no overhead required for an operating system, an FPGA may process data faster than a general-purpose computer. Similar to dedicated processors, this is at the cost of flexibility.

The lack of software overhead may also allow an FPGA to operate faster than a general processor, e.g., a CPU, although this will depend on the exact processing to be performed and the specific FPGA and the processor.

A group of FPGA(s) 120 may be configured to perform the steps in parallel. For example, a number of FPGA(s) 120 may be configured to perform a processing step for an image, a set of images, a subtile, or a select region in one or more images. Each FPGA(s) 120 may perform its own part of the processing step at the same time, reducing the time needed to process data. This may allow the processing steps to be completed in real time. Further discussion of the use of FPGAs is provided below.

Performing the processing steps in real time may allow the system to use less memory, as the data may be processed as it is received. This improves over existing systems, which may need to store the data before it may be processed, and which may require more memory or access of a computer system located in the cloud 130.

In some embodiments, the data storage device 122 is used to store information used in or obtained from sequencing analysis. For example, the DNA sequences determined after adaptor trimming may be stored in the data storage device 122. Compressed, or uncompressed, or combinations thereof, sequencing data may be stored in the data storage device 122. The FASTQ file may also be stored in the data storage device 122.

The user interface 124 may be used by a user to operate the sequencing system or access data stored in the data storage device 122 or the computer system 126.

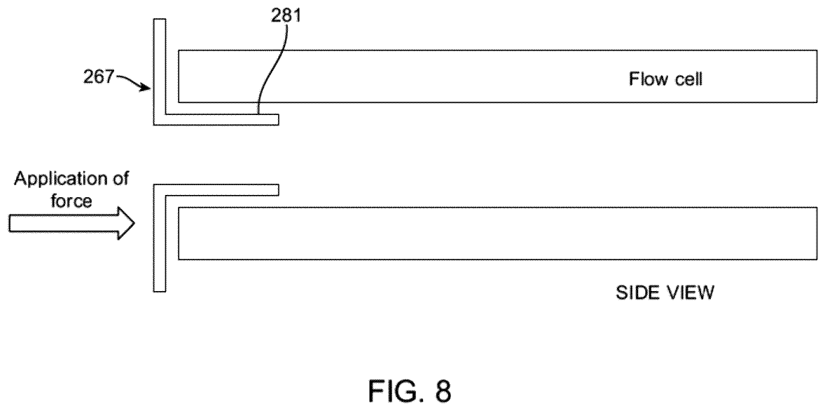

The computer system 126 may control the general operation of the sequencing system and may be coupled to the user interface 124. The computer system 126 may also perform steps in sequencing analysis, such as image registration, color correction, base calling, adaptor trimming, demultiplexing, etc. In some embodiments, the computer system 126 is a computer system 800, as described in more detail in FIG. 8. The computer system 126 may store information regarding the operation of the sequencing system 110, such as configuration information, instructions for operating the sequencing system 110, or user information. The computer system 126 may be configured to pass information between the sequencing system 110 and the cloud 130.

As discussed above, the sequencing system 110 may have dedicated processors 118, FPGA(s) 120, or the computer system 126. The sequencing system may use one, two, or all of these elements to accomplish the processing described above. In some embodiments, when these elements are present together, the processing tasks are split between them. For example, the FPGA(s) 120 may be used to perform some portion or all of sequencing analysis operations, optionally, the dedicated processor, 118 may be used to perform some other portion of the sequencing analysis, e.g., predicting polony locations of in situ samples. while the computer system 126 may perform other processing functions for the sequencing system 110. Various combinations of these elements may allow various system embodiments that balance efficiency and speed of processing with cost of processing elements.

The cloud 130 may be a network, remote storage, or some other remote computing system separate from the sequencing system 110. The connection to cloud 130 may allow access to data stored externally to the sequencing system 110 or allow for updating of software in the sequencing system 110.

### Flow Cell Devices

Disclosed herein, in some embodiments, are flow cell devices and systems that can be employed for performing or facilitating DNA sequencing analysis. Flow cell devices herein can be used to immobilize template nucleic acid molecules derived from biological samples and introduce a repetitive flow of sequencing reagents (e.g., sequencing-by-binding, sequencing-by-synthesis, or sequencing-by-avidite, or combinations thereof) to attach labeled nucleotides to specific positions in the template sequences. A series of labeled signals are detected and decoded to reveal the nucleotide sequences of the template molecules, e.g., immobilized, or amplified, or combinations thereof, nucleic acid template molecules attached to a surface of the flow cell.

In some embodiments, the samples herein can be traditional 2D DNA sequencing samples. In some embodiments, the samples herein can be 3D volumetric samples, e.g., in situ samples of cell(s) or tissue(s).

The flow cell device may include a support comprising one or more substrates; one or more channels defined by the one or more substrates and extending along a first direction, along y axis, and between an inlet and outlet; and one or more seals positioned on the one or more substrate to improve sealing of the fluidic communication between the flow cell device to the flow control device or any other part of the sequencing system. In some embodiments, the one or more channels are in fluidic communication with fluidic pathway(s) of the manifold or connectors. Such fluidic communication between the channels and the fluidic pathway can be direct or indirect via a cleaning outlet. The open landing area may also be in fluidic communication with fluidic pathway(s) of the manifold or connectors, directly or via the cleaning outlet. The fluidic pathway(s) of the manifold or connector can then be in fluidic communication with a fluid control device like a pump or a vacuum thereby allowing clearing the reagents from the open landing area and/or the channels. The fluidic pathway(s) may also be used for introducing liquid or gas into the open landing area or the one or more channels when needed.

In some embodiments, a flow cell device 112 disclosed herein can comprise a support 210 having one or more substrates, a number of channels, an inlet, a cleaning outlet, and an outlet. FIGS. 2-14 show non-limiting examples of flow cell devices 112.

In some embodiments, the flow cell device 112 disclosed herein can include a support 210. The support can be solid. At least part of the support 210 can be transparent so that light transmitting from a light source of the imager (116 in FIG. 1) can travel through the transparent portion of the support and reach the samples located on the flow cell device 112.

The support 210 can comprise one or more substrates 220, 230.

As shown in FIGS. 2A-2B, the one or more substrates can include a top substrate 220 and a bottom substrate 230. When the flow cell device 112 is placed in the sequencing system 110 for imaging, the top substrate 220 can be closer to the camera of the imager 116, along the z direction, than the bottom substrate 230. The bottom substrate 330 can be closer to a translation stage of the sequencing system 110 for holding and supporting the flow cell device 112 during sequencing than the top substrate 220. The z direction can be orthogonal to the image plane. In some embodiments, the top and/or bottom substrate can include one or more layers. For example, the top substrate can include a second layer, a third layer, or even more layers 221, and the bottom substrate can include one or multiple layers 231 that are mechanically fixed together (e.g., glued or attached with adhesion) with at least some area of overlap in the x-y plane. 227 represents the thickness a, and 228 represents the thickness b as seen in FIG. 2A.

In some embodiments, the flow cell device 112 can further include a middle substrate in between the top and the bottom substrate.

The top substrate (including 220 and 221) may comprise a first thickness above the first portion 253 of the channel and a second thickness about the second portion 254 of the channel 250. The second thickness may be greater than the first thickness. In some embodiments, the second thickness may be 20%, 50%, 80%, 100%, 120%, 150%, or 200% more than the first thickness.

The bottom substrate (including 230 and 231) may comprise a third thickness above the first portion 253 of the channel and a fourth thickness above the second portion 254 of the channel. The fourth thickness may be greater than the third thickness. The fourth thickness may be 20%, 50%, 80%, 100%, 120%, 150%, or 200% more than the third thickness. The thicknesses herein may be along the z direction.

In some embodiments, the second thickness may be identical to the first thickness. The fourth thickness may be identical to or greater than the third thickness. In other words, the top substrate and/or bottom substrate may have uniform thickness from one end to another end of the flow cell device along the y axis. Having a uniform thickness in the top substrate may advantageously facilitate homogenous light transmission from the light source to the sample immobilized on the flow cell device.

In some embodiments, the one or more substrates can include 2, 3, 4, 5, 6, or even more substrates. In some embodiments, the one or more substrates when assembled together into the flow cell device may form 1, 2, 3, 4, or more surfaces that samples may be immobilized thereon. For example, the one or more substrates may form 2 surfaces, e.g., a top and a bottom surface of a fluidic channel that are displaced from each other along z axis. As another example, the one or more substrates may form 4 surfaces, e.g., a top and a bottom surface of a first fluidic channel that are displaced from each other along z axis and a top and a bottom surface of a second fluidic channel that are displaced from each other along z axis, where the first and second fluidic channel are at different z locations along the z axis.

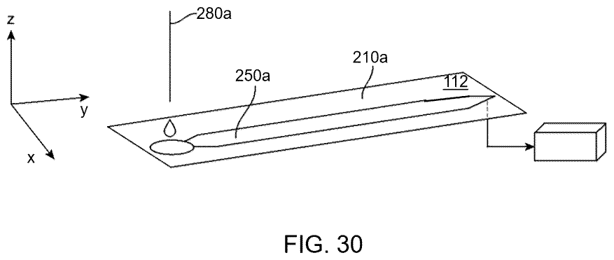

In some embodiments, a flow cell device 112 disclosed herein can comprise a support having one or more substrates, a number of channels, an inlet, and an outlet. FIGS. 30-32, and 33A-33F show additional embodiments of flow cell devices.

In some embodiments, the flow cell device 112 disclosed herein can include a support 210, 510a. The support 210, 510a can be solid. At least part of the support 210, 510a can be transparent so that light transmitting from a light source of the imager (116 in FIG. 1) can travel through the transparent portion of the support and reach the samples located on the flow cell device 112.

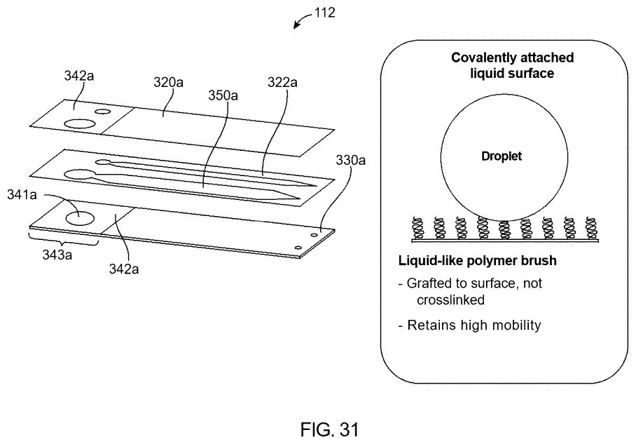

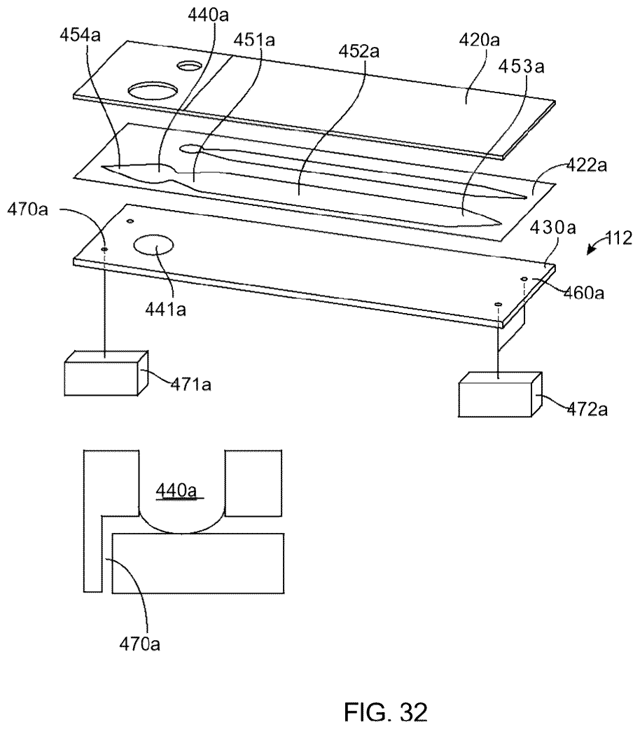

The support 210, 510a can comprise one or more substrates 220, 230, 320a, 322a, 330a, 520a, 420a, 422a, 430a, 522a, 530a, 722a. As shown in FIGS. 31-32, the one or more substrates can include a top substrate 320a, 420a and a bottom substrate 330a, 430a. When the flow cell device 112 is placed in the sequencing system 110 for imaging, the top substrate 320a, 420a can be closer to the camera of the imager 116, along the z direction, than the bottom substrate 330a, 430a. The bottom substrate 330a, 430a can be closer to a translation stage of the sequencing system 110 for holding and supporting the flow cell device 112 during sequencing than the top substrate 320a, 420a.

In some embodiments, the flow cell device 112 can further include a middle substrate 322a, 422a, 522a, 722a in between the top 320a, 420a, 520a and the bottom substrate 330a, 430a, 530a as shown in FIGS. 31-32, 33A and 33C.

Each substrate can have a predetermined thickness, and different substrates can have different thicknesses. In some embodiments, each substrate can have a uniform thickness along the z direction. In some embodiments, each substrate can have a uniform thickness along the z direction in at least a portion of the substrate (e.g., in the first portion 225, or second portion 235). In some embodiments, the portion with uniform thickness can encompass the channel(s) or the imaging areas of the flow cell device 112.

In some embodiments, the top or bottom substrate can have a thickness of about 0.2 mm to about 5 mm. In some embodiments, the top or bottom substrate can have a thickness of about 0.6 mm to about 3 mm. In some embodiments, the top or bottom substrate can have a thickness of about 0.8 mm to about 2 mm. In some embodiments, the top or bottom substrate can have a thickness of about 0.8 mm to about 1.5 mm. In some embodiments, the top or bottom substrate can have a thickness of about 0.8 mm to about 1.2 mm. In some embodiments, the top or bottom substrate can have a thickness of about 0.9 mm to about 1.1 mm.

In some embodiments, the top or bottom substrate can have a thickness of 0.2 mm to 5 mm. In some embodiments, the top or bottom substrate can have a thickness of 0.6 mm to 3 mm. In some embodiments, the top or bottom substrate can have a thickness of 0.8 mm to 2 mm. In some embodiments, the top or bottom substrate can have a thickness of 0.8 mm to 1.5 mm. In some embodiments, the top or bottom substrate can have a thickness of 0.8 mm to about 1.2 mm. In some embodiments, the top or bottom substrate can have a thickness of 0.9 mm to 1.1 mm. In some embodiments, the top or bottom substrate can have a thickness of 0.95 mm to 1.05 mm.

In some embodiments, the middle substrate can have a thickness of about 40 μm to 200 μm. In some embodiments, the middle substrate can have a thickness of about 40 μm to 150 μm. In some embodiments, the middle substrate can have a thickness of about 40 μm to 70 μm. In some embodiments, the middle substrate can have a thickness of about 80 μm to 120 μm. In some embodiments, the middle substrate can have a thickness of about 60 μm to 90 μm.

In some embodiments, the middle substrate can have a thickness of 40 μm to 200 μm. In some embodiments, the middle substrate can have a thickness of 40 μm to 150 μm. In some embodiments, the middle substrate can have a thickness of 40 μm to 70 μm. In some embodiments, the middle substrate can have a thickness of 80 μm to 120 μm. In some embodiments, the middle substrate can have a thickness of 60 μm to 90 μm.

In some embodiments, the thickness a of the flow cell device 112 can be in a range from 1 mm to 5 mm. In some embodiments, the thickness a of the flow cell device 112 can be in a range from 1.5 mm to 3.5 mm. In some embodiments, the thickness of the flow cell device 112 can be in a range from 2 mm to 6 mm.

In some embodiments, the substrate(s) can have an elongate shape extending along the y axis. In some embodiments, the substrate(s) can have various shapes such as rectangular, square, etc.

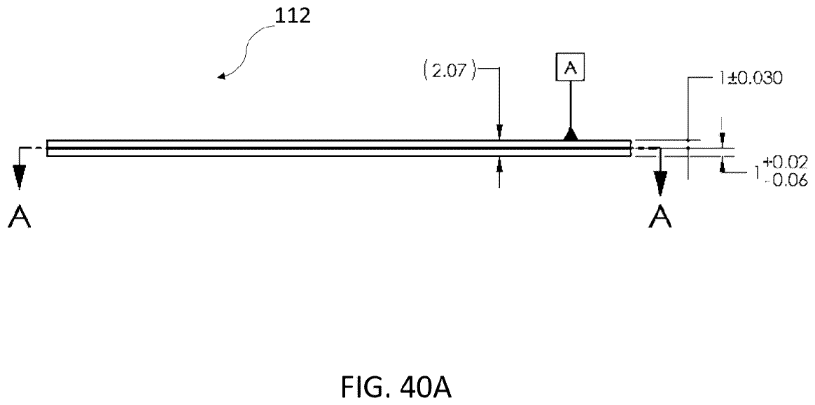

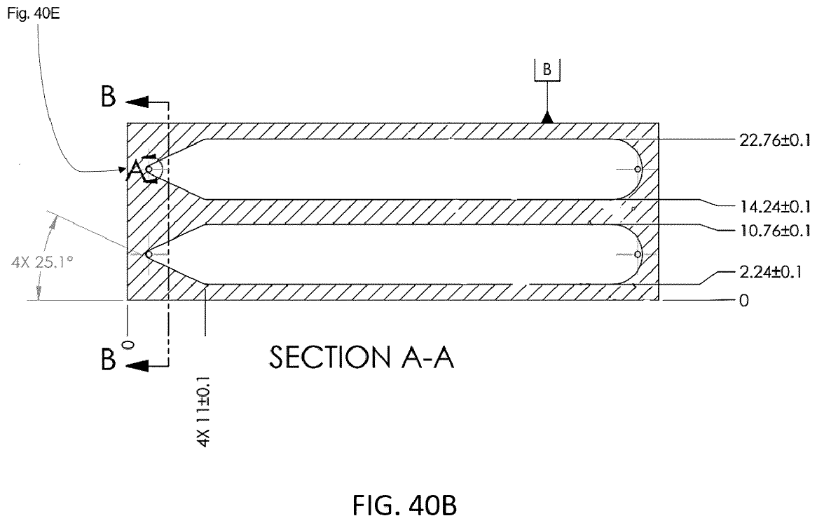

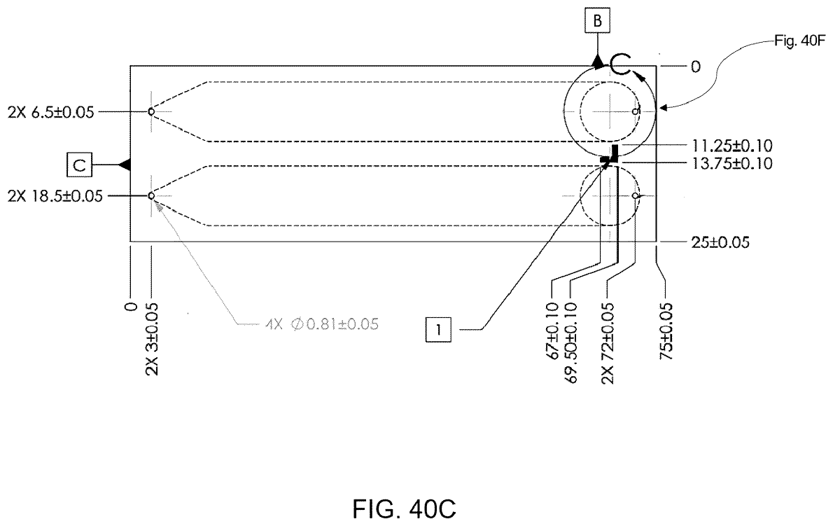

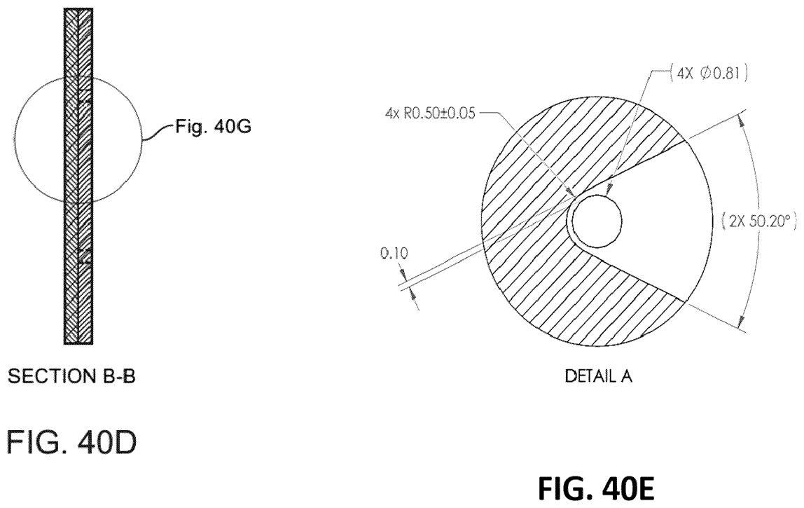

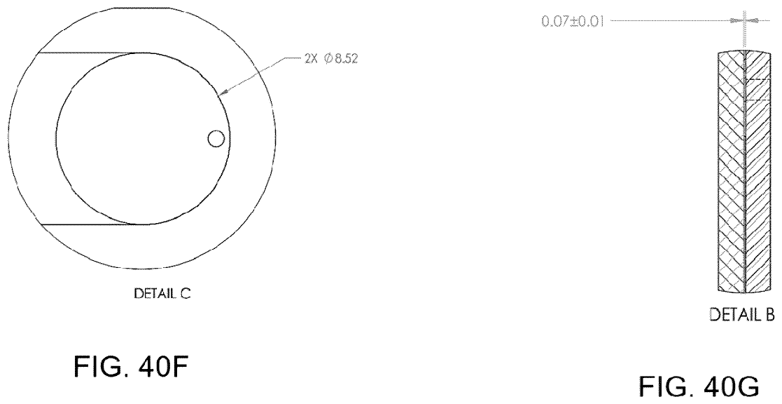

In some embodiments, the one or more substrates can have one or more surfaces that are planar. In some embodiments, the one or more substrates contains no curvature perceivable to naked eyes, e.g., as shown in FIGS. 2A, 30-32, so that the one or more substrates can have planar surfaces. In some embodiments, the flatness of the surface(s) of the substrates can be measured as the height from its peak to valley in an direction orthogonal to the surface(s). The height can be less than about 0.01 mm, 0.02 mm, 0.03 mm, 0.04 mm, 0.05 mm, 0.06 mm, 0.07 mm, 0.08 mm, or 0.09 mm, e.g., along a direction orthogonal to the surface. In other words, the flat surface(s) of the substrates may fit between two parallel planar 2D planes that are less than 0.01 mm, 0.02 mm, 0.03 mm, 0.04 mm, 0.05 mm, 0.06 mm, 0.07 mm, 0.08 mm, or 0.09 mm apart from each other. In some embodiments, the flatness of the surface(s) of the substrates can include a height from its peak to valley that is less than 0.01 mm, 0.02 mm, 0.03 mm, 0.04 mm, 0.05 mm, 0.06 mm, 0.07 mm, 0.08 mm, or 0.09 mm. However, the substrates do not have to be planar in certain embodiments. Alternatively, a part or the entirety of one or more substrates can be curved. In some embodiments, the flatness of the surface(s) of the substrates from its peak to valley can be less than about 0.01 mm, 0.02 mm, 0.03 mm, 0.04 mm, 0.05 mm, 0.06 mm, 0.07 mm, 0.08 mm, or 0.09 mm. In some embodiments, the flatness of the surface(s) of the substrates from its peak to valley can be less than 0.01 mm, 0.02 mm, 0.03 mm, 0.04 mm, 0.05 mm, 0.06 mm, 0.07 mm, 0.08 mm, or 0.09 mm. However, the substrates do not have to be planar in certain embodiments. Alternatively, a part or the entirety of one or more substrates can be curved. As an example, surfaces A, B, and C in FIGS. 40A-40G can have a flatness from peak to valley of about 0.02 mm or 0.03 mm.

In some embodiments, the support or the one or more substrates can comprise glass or plastic. In some embodiments, the support or one or more substrates are all-glass, all-plastic, or partly glass and partly plastic. FIG. 2B shows one or more layers 231 of the bottom substrate that can comprise bonded plastic at the second portion 236. In some embodiments, the support or the one or more substrates can comprise a tape such as a pressure sensitive adhesive (PSA) tape. In some embodiments, the support or one or more substrates are all-glass or all-plastic. For example, the middle substrate as shown in FIG. 31 can be made from PSA tape and can conveniently tape the top and bottom substrates to it fixedly.

In some embodiments, the substrate(s) can define one or more channels of the flow cell devices 112. In some embodiments, the channels 250 can allow fluid, e.g., liquid or gas, to flow therethrough. In some embodiments, the substrates 320a, 322a, 330a, 420a, 422a, 430a, 520a, 522a, and/or 530a can define one or more channels 250, 350a, 450a, 550a of the flow cell devices 112. In some embodiments, the channels 250, 350a, 450a, 550a can allow fluid, e.g., liquid or gas, to flow therethrough.

The gas herein can comprise one type of gas or a combination of different types of gasses. In some embodiments, the gas comprises air. The gas can comprise dry air. In some embodiments, the gas comprises one or more inert gasses. In some embodiments, the gas comprises one or more active gasses.

The reagents herein can comprise liquid. In some embodiments, the reagents are deprived of air bubbles that are greater than a predetermined size. In some embodiments, the first reagent is configured to wet the first coating of the surface of the one or more channels. In some embodiments, the second reagent is configured to rewet the surface of the one or more channels after at least partly drying the surface by the gas gap.

In some embodiments, the first portion of the channel 253 of the one or more channels 250 comprises a first z location and a second portion 254 of the channel comprises a second z location that is different from the first z location, e.g., as shown in FIG. 2A.

In some embodiments, the channel(s) can be defined by a top interior surface 251 and a bottom interior surface 252 of the substrates. In specific, the channels 250 can each include a lumen defined by a top interior surface 251 and a bottom interior surface 252 of the substrates surrounding the lumen. In some embodiments, the channel comprises a first portion that is at a first z location and a second portion that is at a second z location offsetting from the first z location, as shown in FIGS. 2A-2B. The top interior surface 251 and bottom interior surface 252 may extend from the cleaning outlet 270 and/or the inlet 240 to the channel and then to the outlet 260.

In some embodiments, the channel(s) 250a, 350a, 450a, 550a can be defined by a top interior surface 521a and a bottom interior surface 521a of the substrates. In specific, the channels 250a, 550a can each include a lumen 551a defined by a top interior surface 521a and a bottom interior surface 521a of the substrates surrounding the lumen 551a, and a grove in either the top, bottom, or both surfaces, without a middle substrate.

In some embodiments, the one or more substrates can include 2, 3, 4, 5, 6, or even more substrates. In some embodiments, the one or more substrates when assembled together into the flow cell device may form 1, 2, 3, 4, or more surfaces that samples may be immobilized thereon. For example, the one or more substrates may form 2 surfaces, e.g., a top and a bottom surface of a fluidic channel that are displaced from each other along z axis. As another example, the one or more substrates may form 4 surfaces, e.g., a top and a bottom surface of a first fluidic channel that are displaced from each other along z axis and a top and a bottom surface of a second fluidic channel that are displaced from each other along z axis, where the first and second fluidic channel are at different z locations along the z axis.

In some embodiments, the channels 350a, 450a, 550a can be defined by the top and bottom substrates with an addition of a middle substrate 322a, 422a, 522a. The middle substrate can include a void, e.g., an elongated void, extending along a longitudinal axis, or y axis, of the middle substrate. The void's width can define the width of the channel 350a, 450a, 550a, along the x axis, and the void's length, along the y direction, can define the length of the channel. FIGS. 31-32 and 33A show flow cell devices with channels 350a, 450a, 550a defined by the top, middle, and bottom substrates.

In some embodiments, the channels are microfluidic channels. In some embodiments, a gap or height between the top interior surface and the bottom interior surface of the substrates that defines the channels, along the z direction, is about 150 μm, 130 μm, 120 μm, 110 μm, 100 μm, 90 μm, 80 μm, 70 μm, 60 μm, 50 μm, or 40 μm. In some embodiments, the gap or height of the channel is no more than about 100 μm. In some embodiments, the gap or height of the channel is no more than about 80 μm, 70 μm, 60 μm, 50 μm, or 40 μm.

In some embodiments, a gap or height between the top interior surface 251 and the bottom interior surface 252 of the substrates that defines the channels, along the z direction, is 150 μm, 130 μm, 120 μm, 110 μm, 100 μm, 90 μm, 80 μm, 70 μm, 60 μm, 50 μm, or 40 μm. In some embodiments, the gap or height of the channel is no more than 100 μm. In some embodiments, the gap or height of the channel is no more than 80 μm, 70 μm, 60 μm, 50 μm, or 40 μm.

In some embodiments, a length of the channel, along the y direction, is about 120 mm, 100 mm, 90 mm, 80 mm, 70 mm, 60 mm, 50 mm, 40 mm, or 30 mm. In some embodiments, the length of the channel is no more than about 100 mm. In some embodiments, the length of the channel is no more than about 80 mm, 75 mm, 70 mm, 65 mm, 60 mm, 55 mm, 50 mm, 45 mm, or 40 mm.

In some embodiments, a length of the channel, along the y direction, is 120 mm, 100 mm, 90 mm, 80 mm, 70 mm, 60 mm, 50 mm, 40 mm, or 30 mm. In some embodiments, the length of the channel is no more than 100 mm. In some embodiments, the length of the channel is no more than 80 mm, 75 mm, 70 mm, 65 mm, 60 mm, 55 mm, 50 mm, 45 mm, or 40 mm.

In some embodiments, a width of the channel, along the x direction, is about 50 mm, 40 mm, 30 mm, 25 mm, 20 mm, 15 mm, 10 mm, 8 mm, or 5 mm. In some embodiments, the length of the channel is no more than about 10 mm or about 7 mm. In some embodiments, the width of the channel is no more than about 30 mm, 25 mm, 20 mm, 15 mm, 10 mm, or 5 mm.

In some embodiments, a width of the channel, along the x direction, is 50 mm, 40 mm, 30 mm, 25 mm, 20 mm, 15 mm, 10 mm, 8 mm, or 5 mm. In some embodiments, the width of the channel is no more than 10 mm or 7 mm. In some embodiments, the width of the channel is no more than 30 mm, 25 mm, 20 mm, 15 mm, 10 mm, or 5 mm.

In some embodiments, the distance between two adjacent channels or the distance from an edge of the channel to the edge of the flow cell device, along the x axis, is about 0.5 mm to about 15 mm. In some embodiments, the distance between two adjacent channels or the distance from an edge of the channel to the edge of the flow cell device, along the x axis, is about 1 mm to about 8 mm. In some embodiments, the distance between two adjacent channels or the distance from an edge of the channel to the edge of the flow cell device, along the x axis, is about 2 mm to 6 mm.

In some embodiments, the distance between two adjacent channels or the distance from an edge of the channel to the edge of the flow cell device, along the x axis, is 0.5 mm to 15 mm. In some embodiments, the distance between two adjacent channels or the distance from an edge of the channel to the edge of the flow cell device, along the x axis, is 1 mm to 8 mm. In some embodiments, the distance between two adjacent channels or the distance from an edge of the channel to the edge of the flow cell device, along the x axis, is 2 mm to 6 mm.

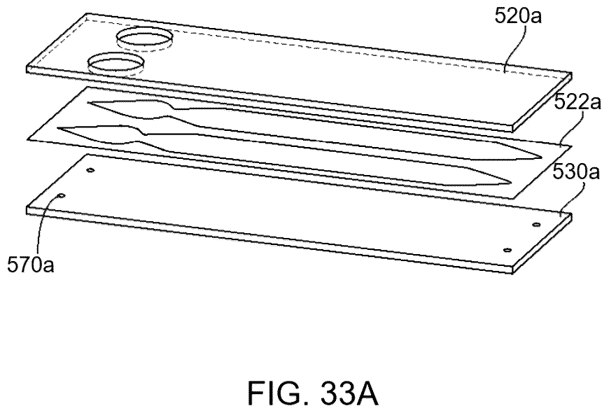

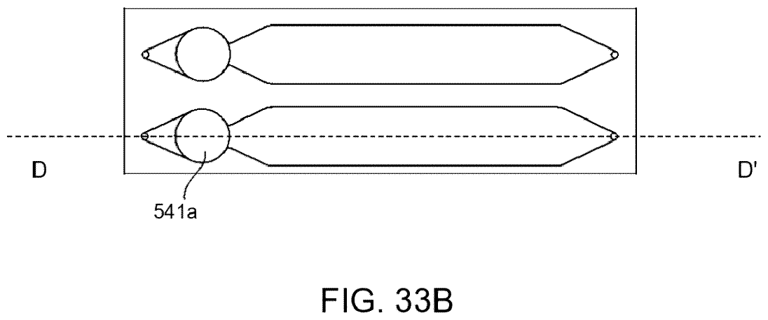

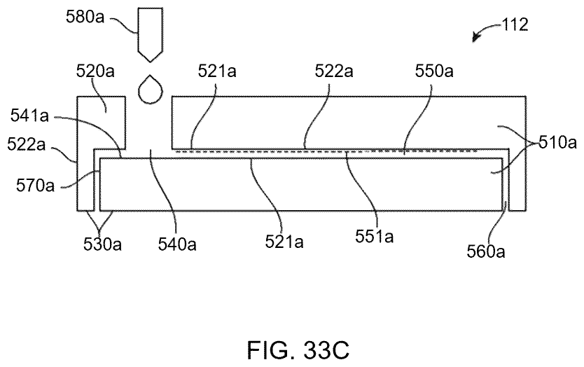

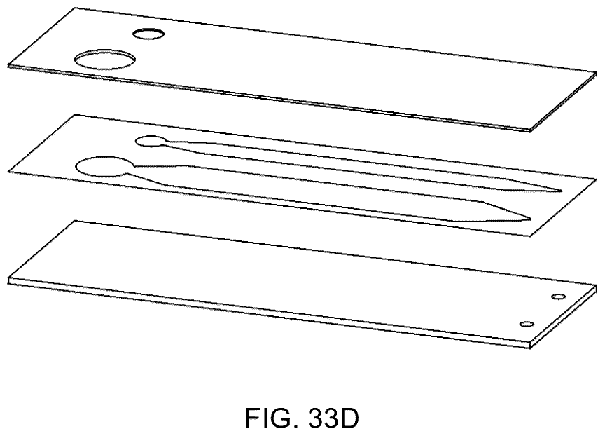

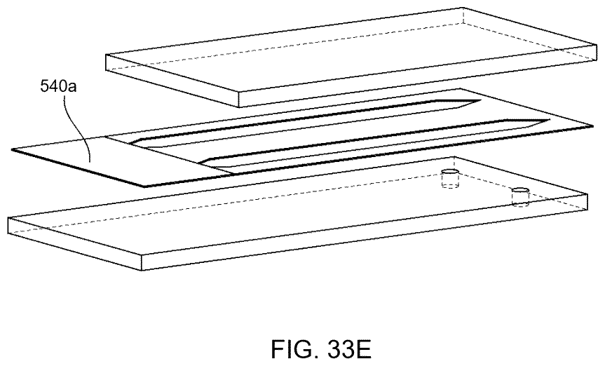

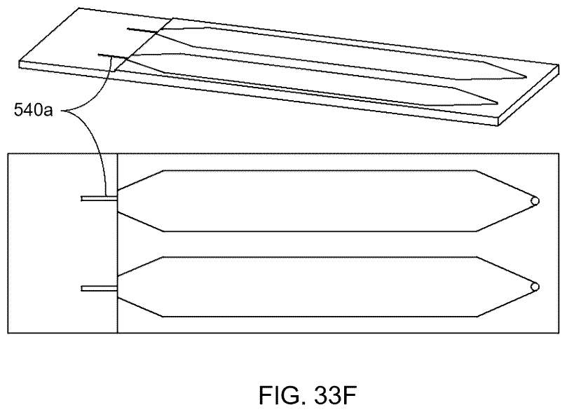

In some embodiments, the flow cell devices can have more than one channel, and all the channels can have a uniform size and shape. FIGS. 33A, 33E, and 33F show embodiments of flow cell devices with two channels of the identical size and shape. In some embodiments, the flow cell devices can have channels of different sizes, or shapes, or combinations thereof. FIGS. 31-32 show embodiments of flow cell devices with similar channel length but different channel widths

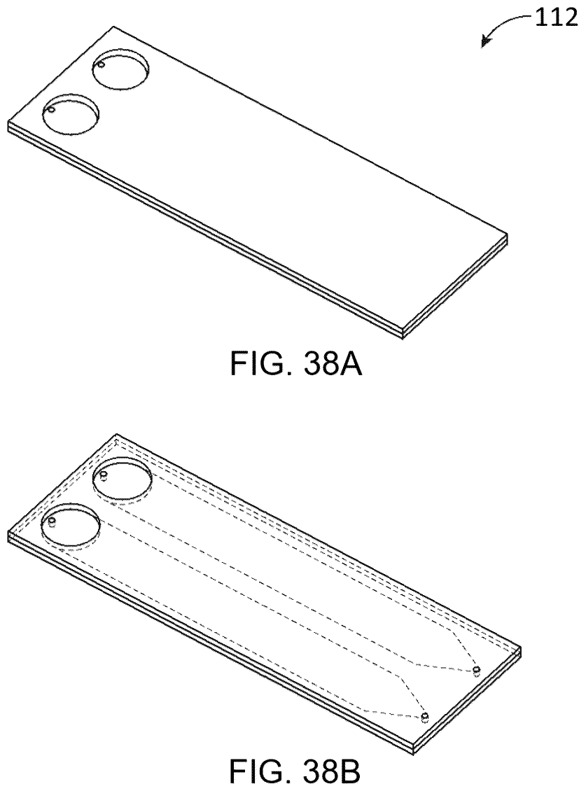

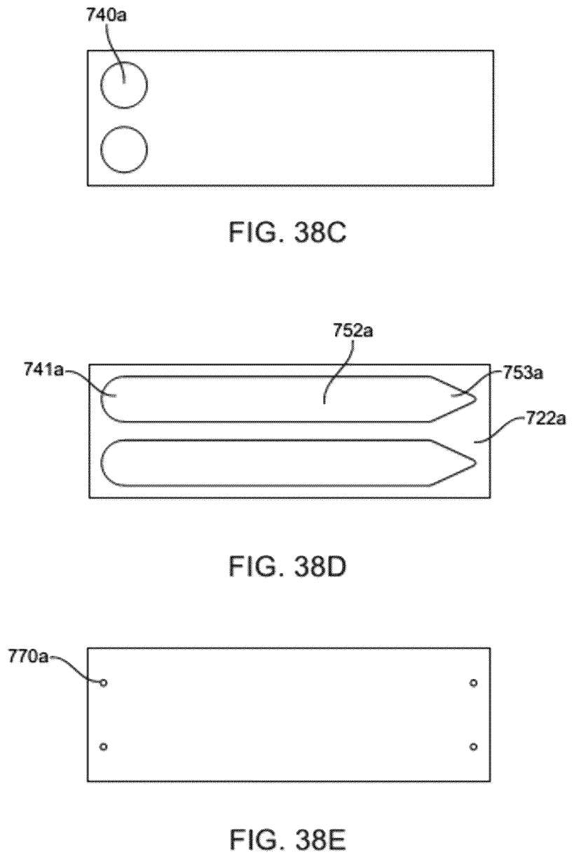

In some embodiments, the channels may include a tapered portion that connects the open landing area to the body of the channel (e.g., FIGS. 2D, 5A, 30-32). The tapered area and its taper angle can be determined by the size of the open landing area that it is connecting to, and also the width of the channel body. The size of the tapered area and its taper angle can be adjusted to facilitate efficient fluid transfer from the open landing area to the body of the channel. The tapered transition portion may connect the open landing area 241 to the body of the channel 250. A second tapered area can be used to connect the body of the channel 250 to the outlet 260. FIG. 32 shows an embodiment of the flow cell device with a tapered transition portion 451a connecting the open landing area 441a to the body of the channel 452a. A second tapered area 453a can be used to connect the body of the channel 452a to the outlet 460a. FIG. 38D shows the second tapered area 753a in a different embodiment. The size and shape of the tapered transition portion may be varied depending on the various sequencing applications of the flow cell device, e.g., type of sample, flow rate required during sequencing reactions, etc.

The size and shape of the tapered transition portion 451a may be varied depending on the applications of the flow cell device. FIGS. 40A-40G show an exemplary embodiment the tapered transition portion connecting the body of the channel to the outlet with the sizes and dimensions. The unit of the sizes of different parts of the flow cell device are in millimeters. FIGS. 40A-40G are non-limiting examples of the flow cell device disclosed herein. FIG. 40A is a side view of the flow cell device. FIG. 40B shows a cross-sectional view at A-A in FIG. 40A. FIG. 40C is a top view of the flow cell device. FIG. 40D is a cross-sectional view at B-B in FIG. 40B. FIG. 40E shows an expanded view of area A in FIG. 40B. FIG. 40F shows an expanded view of area C in FIG. 40C. FIG. 40G shows an expanded view of area B in FIG. 40D.

In some embodiments, the tapered transition portion from the outlet to the body of the channel can be about 3 mm to about 15 mm along the y axis. In some embodiments, the tapered transition portion from the outlet to the body of the channel can be about 5 mm to about 12 mm along the y axis. In some embodiments, the tapered transition portion from the outlet to the body of the channel can be about 6 mm to about 9 mm along the y axis. In some embodiments, the tapered transition portion from the outlet to the body of the channel can be 3 mm to 15 mm along the y axis. In some embodiments, the tapered transition portion from the outlet to the body of the channel can be 5 mm to 12 mm along the y axis. In some embodiments, the tapered transition portion from the outlet to the body of the channel can be 6 mm to 9 mm along the y axis.

In some embodiments, the tapered angle, e.g., the acute angle between an edge of the flow cell device and an edge of the tapered area is 25.1 degrees as shown in FIG. 40B. In some embodiments, the tapered angle can be in the range of about 15 degrees to about 40 degrees. In some embodiments, the tapered angle can be in the range of about 20 degrees to about 30 degrees. In some embodiments, the tapered angle can be in the range of 15 degrees to about 40 degrees. In some embodiments, the tapered angle can be in the range of 20 degrees to about 30 degrees.

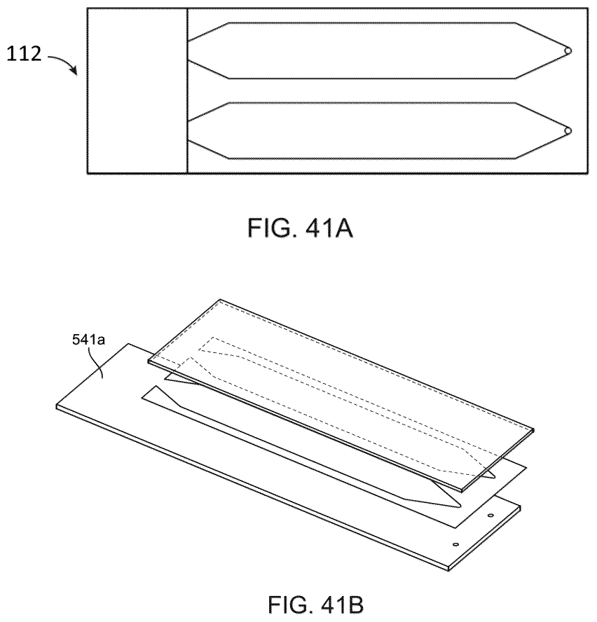

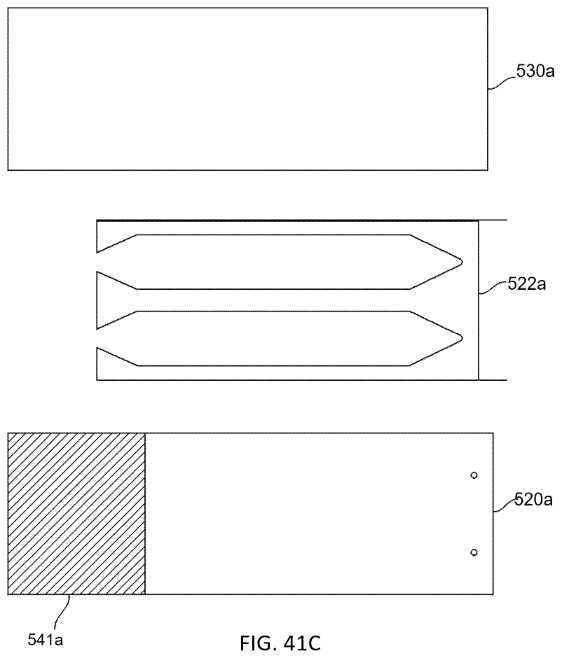

In some embodiments, each channel has its own corresponding open landing area, or inlet, or combinations thereof, e.g., in FIGS. 32, 33A, 33F and 38A-38E. In some embodiments, two or more channels share a single open landing area, or inlet, or combinations thereof, e.g., in FIGS. 33E and 41A-41C.

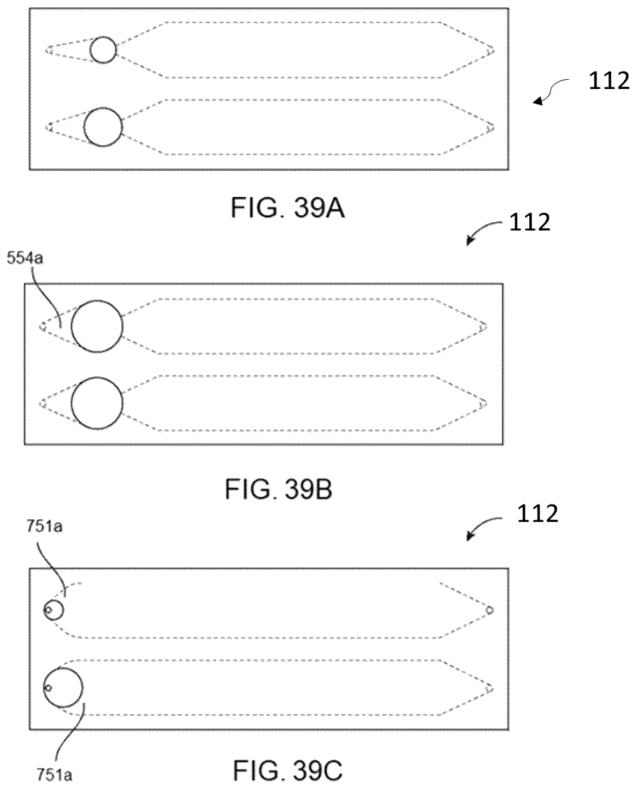

In some embodiments, the open landing area is directly connected to the body of the channel. In some embodiments, the open landing area is connected to the body of the channel without a tapered transition portion in between. FIGS. 38A-38E show an embodiment of the flow cell device disclosed herein. The flow cell device 112 includes a circular open landing area 741a that is directly connected to the body of the channel 752a without a tapered transition portion. In this particular embodiment, the channel 752a starts where the open landing area ends, and the channel width is substantially identical or exactly identical to the diameter of the opening landing area. As show in FIG. 39C, the size of the open landing area can be different from the embodiment in FIGS. 38A-38E either in one channel or in one or more channels so that the diameter of the open landing area is smaller than the width of the channel along the x axis. When the diameter of the open landing area is smaller, there can be a tapered transition region 751a between the open landing area and the body of the channel.

The flow cell device 112 can include one or more inlets 240, 340a, 440a, 540a, 740a and one or more outlets 260, 460a, 560a, 760a and/or one or more cleaning outlets 270, 470a, 570a, 770a. The flow cell device 112 can include one or more channels 250, 350a, 450a, 550a, 750a. A channel 250 can run from its corresponding inlet 240 to its corresponding outlet 260, thereby allowing fluidic communication from the inlet to the outlet. Sequencing reagents can be introduced to the flow cell device 112 via the inlet 240, flow through the channels 250 and interact with samples located therein, and exit from the outlet 260.

The flow cell device 112 can include one or more inlets 240, 340a, 440a, 540a, 740a and one or more outlets 560a. A channel 550a can run from its corresponding inlet 540a to its corresponding outlet 560a, thereby allowing fluidic communication from the inlet to the outlet. Sequencing reagents can be introduced to the flow cell device 112 via the inlet 540a, flow through the channels 550a and interact with samples located therein, and exit from the outlet 560a.

In some embodiments, the flow cell devices 112 further comprises a cleaning outlet 270, 470a, 570a, 770a. The cleaning outlet can be in fluidic communication with the open landing area for cleaning residuals left thereon.

The cleaning outlet 270, 470a, 570a, 770a can be located in the one or more substrates. In some embodiments, the cleaning outlet 270 may be located on a top substrate, bottom substrate, and/or middle substrate as a side port (e.g., FIG. 2G).

In some embodiments, the cleaning outlet comprises a side port on the one or more substrates. In some embodiments, the side port extends at least along a direction that is perpendicular or nearly perpendicular to an x direction. In some embodiments, the side port extends at least along a direction that is perpendicular or nearly perpendicular to a y direction. In some embodiments, the side port extends at least along a direction that is perpendicular or nearly perpendicular to a z direction. In some embodiments, the side port extends at least along a direction that is oblique to an x direction. In some embodiments, the side port extends at least along a direction that is oblique to a y direction. In some embodiments, the side port extends at least along a direction that is oblique to a z direction. In some embodiments, the side port extends at least along a direction that is perpendicular or nearly perpendicular to an x-y plane. In some embodiments, the side port extends at least along a direction that is perpendicular or nearly perpendicular to an x-z plane. In some embodiments, the side port extends at least along a direction that is perpendicular or nearly perpendicular to an x-y plane. In some embodiments, the side port extends at least along a direction that is perpendicular or nearly perpendicular to a y-z plane. In some embodiments, the side port extends at least along a direction that is oblique to an x-y plane. In some embodiments, the side port extends at least along a direction that is oblique to an x-z plane. In some embodiments, the side port extends at least along a direction that is oblique to a y-z plane.

The cleaning outlet 270 can be in fluidic connection with the inlet 240. In some embodiments, the cleaning outlet is configured to be coupled with a fluid driving device, e.g., a pump or vacuum of the fluidic control device, optionally via a connector, a manifold, or both. The pump may be in addition to a pump coupled to the outlet 260. In some embodiments, a same fluid driving device, e.g., pump, can be coupled to the outlet and the cleaning outlet.

The distance from the cleaning outlet 270, 470a, 570a, 770a to the inlet 240 can be shorter than that to the outlet 260. The distance can be within the x-y plane. The shorter distance from the cleaning outlet to the inlet is designed to facilitate transfer of liquid or gas from the open landing area to the cleaning outlet.

In some embodiments, the relative position of the cleaning outlet 270 to the inlet or open landing area 241, 341a, 441a, 541a, 741a can be different. In some embodiments, the cleaning outlet 270 may include a side port that is customized to fit to a connector or a manifold device. In some embodiments, the side port extends at least along a direction that is perpendicular or nearly perpendicular to a y direction. In some embodiments, the side port extends at least along a direction that is perpendicular or nearly perpendicular to a z direction. In some embodiments, the side port extends at least along a direction that is oblique to an x direction. In some embodiments, the side port extends at least along a direction that is oblique to a y direction. In some embodiments, the side port extends at least along a direction that is oblique to a z direction. In some embodiments, the side port extends at least along a direction that is perpendicular or nearly perpendicular to an x-y plane. In some embodiments, the side port extends at least along a direction that is perpendicular or nearly perpendicular to an x-z plane. In some embodiments, the side port extends at least along a direction that is perpendicular or nearly perpendicular to an x-y plane. In some embodiments, the side port extends at least along a direction that is perpendicular or nearly perpendicular to a y-z plane. In some embodiments, the side port extends at least along a direction that is oblique to an x-y plane. In some embodiments, the side port extends at least along a direction that is oblique to an x-z plane. In some embodiments, the side port extends at least along a direction that is oblique to a y-z plane.

In such embodiments, the cleaning outlet 270, 470a, 570a, 770a is not directly connected to the corresponding open landing area, but instead connected via a tapered transition portion therebetween, e.g., FIG. 2D.

The distance from the cleaning outlet (from the closer end of the cleaning outlet) to the closest edge or the center of the open landing area can be 0 mm or about 10 mm. The distance from the cleaning outlet to the closest edge or the center of the open landing area, when the cleaning outlet is not directly underneath the open landing area, can be from about 0 mm to about 20 mm. The distance from the cleaning outlet to the closest edge or the center of the open landing area, when the cleaning outlet is not directly underneath the open landing area, can be from about 0 mm to about 15 mm. The distance from the cleaning outlet to the closest edge or the center of the open landing area, when the cleaning outlet is not directly underneath the open landing area, can be from about 3 mm to about 10 mm.

In some embodiments, there can be residuals of reagents, such as meniscus, which remains on the open landing area, or on the wall(s) of the hole of the inlet, or combinations thereof. Such residuals, if not removed, may cause unintended mixing when a subsequent reagent is delivered to the open landing area and consequently contaminate sequencing reactions in the channels. Washing with liquid(s) alone may not be effective in removing such residual reagents as meniscus, so it may take multiple flushing of washing liquids to completely remove the residual in existing flow cell systems, with increased washing time and washing costs. The cleaning outlet 270 in fluidic connection can advantageously facilitate time- and cost-effective removal of such residuals. In some embodiments, a mechanical driving force can be applied, e.g., by a pump or an inlet vacuum, via the cleaning outlet, to completely remove such residual of reagents on the open landing area. As such, the required time and washing volume to remove the residuals to achieve a satisfactory contamination level can be effectively improved from existing flow cell devices.

The size and shape of the cleaning outlet may be customized to suit different sequencing applications. Although the cleaning outlet is shown as a cylindrical shape in FIGS. 2D-2F, it can be made in different shapes, such as a cone, an inverted cone, etc. In some embodiments, the size and shape of the cleaning outlet can be identical to that of the outlet. In some embodiments, the size of the cleaning outlet can be no more than about 10%, 20%, or 30% different from that of the outlet. In some embodiments, the diameter of the cleaning outlet in the x-y plane is about 0.3 mm to about 10 mm. In some embodiments, the height of the cleaning outlet in the z direction is the same as the height of the bottom substrate. In some embodiments, the height of the cleaning outlet is about 0.3 mm to about 3 mm. In some embodiments, the height of the cleaning outlet is about 0.5 mm to about 1 mm. In some embodiments, the diameter of the cleaning outlet in the x-y plane is 0.3 mm to 10 mm. In some embodiments, the height of the cleaning outlet in the z direction is the same as the height of the bottom substrate. In some embodiments, the height of the cleaning outlet is 0.3 mm to 3 mm. In some embodiments, the height of the cleaning outlet is 0.5 mm to 1 mm.

The size and shape of the inlet and outlet can be customized to suit various sequencing applications. For example, the size and shape can be determined based on the specific sequencing application(s), such as, a minimal flush volume, a contamination threshold, the parameters of the flow cell, e.g., the size of the flow cell channels, or the parameters of the dispenser, e.g., the size of the dispensing tip. As a nonlimiting example, the inlet can be cylindrical with walls extending along the z direction and orthogonal to the substrates, for example, such as shown in FIG. 33C. In some embodiments, at the bottom of the cylindrical void/hole, the inlet 240 can be connected to a cleaning outlet 270. In some embodiments, at the bottom of the cylindrical void/hole, the inlet 440a can be connected to a cleaning outlet 470a, 570a. The inlet can be shaped differently in different embodiments. For example, the inlet can have an inverted cone shape with wider openings at the top and narrows down toward the channel to reduce the residuals of reagents that can remain in the inlet. FIG. 33D shows an embodiment with an inlet as a cylindrical shape. In embodiments without the cleaning outlet, the flow cell device includes no connection extending from the inlet to the cleaning outlet. In another embodiment, as in FIG. 33E, the inlet 540a can be part or all of the open landing area. In yet another embodiment of the flow cell device, as in FIG. 33F, the inlet may be a grove of various sizes or shapes in the middle substrate, or in the middle and the bottom substrates, which is in fluidic connection to the channels.

In another embodiment, as in FIG. 33E, the inlet 540a can comprise the open landing area or a portion thereof but no other structural elements in the flow cell device. FIGS. 41A-41C shows the embodiment in FIG. 33E from different views. FIG. 41A is a top view of the flow cell device. FIG. 41B shows three different substrates in a prospective view, and FIG. 41C show the bottom, middle, and top substrates. In yet another embodiment of the flow cell device, as in FIG. 33F, the inlet may be a grove of various sizes or shape in the middle substrate, or in the middle and the bottom substrates that are in fluidic connection to the channels.

The diameter of the inlet, e.g., the widest dimension in the x-y plane, can be in the range of about 3 mm to about 11 mm. As another example, the height of the inlet, along the z direction, can be the total height of the top substrate and the middle substrate, and it can be in the range of about 1 mm to 12 mm.

The diameter of the outlet or the cleaning outlet, in the x-y plane, can be in the range of about 0.3 mm to about 4 mm. In some embodiments, the diameter of the outlet can be in the range of about 0.4 mm to about 2 mm, and the outlet can be a cylindrical shape.

The diameter of the inlet, e.g., the widest dimension in the x-y plane, can be in the range of 3 mm to 11 mm. As another example, the height of the inlet, along the z direction, can be the total height of the top substrate and the middle substrate, and it can be in the range of 1 mm to 12 mm.

The diameter of the outlet or the cleaning outlet, in the x-y plane, can be in the range of 0.3 mm to 4 mm. In some embodiments, the diameter of the outlet can be in the range of 0.4 mm to 2 mm, and the outlet can be a cylindrical shape.

The size and shape of the inlet and outlet can be customized to suit various sequencing applications. For example, the size and shape can be determined based on the specific sequencing application(s), such as, a minimal flush volume, a contamination threshold, the parameters of the flow cell, e.g., the size of the flow cell channels, or the parameters of the dispenser, e.g., the size of the dispensing tip.

FIGS. 30-32, and 33A-33F show flow cell devices with two to three substrates forming one or two channels, and each channel having a corresponding inlet and outlet. However, the number of substrates, channels, inlets, and outlets can vary in different embodiments. In some embodiments, the number of substrates, channels, inlets and outlets can be any integer number that is greater than 0. In some embodiments, the flow cell devices herein have 2, 4, 6, 8, 10, or even more channels.

In some embodiments, the flow cell device may include one or more seals 290 that help prevent leaking either between structural elements of the flow cell device or between the flow cell device and other devices connected thereto (e.g., manifold, connector, pump, etc.). The leaking may cause damage or contamination to the sequencing system or sample immobilized on the flow cell device. For example, leaking may occur between two adjacent layers in the top or bottom substrates without the one or more seals. As another example, leaking may occur between the connection of the flow cell device and its manifold or between the flow cell device and the connector connecting the flow cell device to the manifold.

The one or more seals may comprise one or more mechanical seals. The one or more seals can comprise: a gasket 267, a manifold or connector, a part of a manifold device, or their combinations. The one or more seals can include one or more gaskets. The one or more seals may comprise a flexible material that deforms under a pressure satisfying a predetermined threshold, e.g., rubber.

In some embodiments, the one or more seals can be positioned at one end of the substrates along y axis, as shown in FIGS. 2A-2C.

In some embodiments, the flow cell device may include increased thickness in the substrates (e.g., FIG. 2A-2C) at the second portion 226 and 236 thereby advantageously facilitating attachment of the one or more seals to the substrates and consequently enabling improved sealing of fluidic communication than flow cell devices without increased thickness in the substrates. In some embodiments, the increased substrate thickness in combination with the fluidic channel having z-offset in two portions also provides clearance to fluidic dispensing elements, e.g., dispensing tips to the open landing area. In other words, the flow cell devices (e.g., with variable thickness in the top substrate) herein can provide clearance above the top surface of the top substrate and avoid blocking dispensing tip movement at the same z level along the x-y plane.

In some embodiments, the one or more seals comprise a first seal with a thickness along a z direction that is comparable to a thickness of the top substrate in the second portion 226, e.g., FIG. 2A. The one or more seals may comprise a second seal with a thickness along a z direction that is comparable to a thickness of the bottom substrate in the second portion 236. In some embodiments, the second seal has a thickness along the z direction that is greater than the thickness of the bottom substrate in the first portion. The one or more seals may comprise various widths along the y axis and/or x axis.

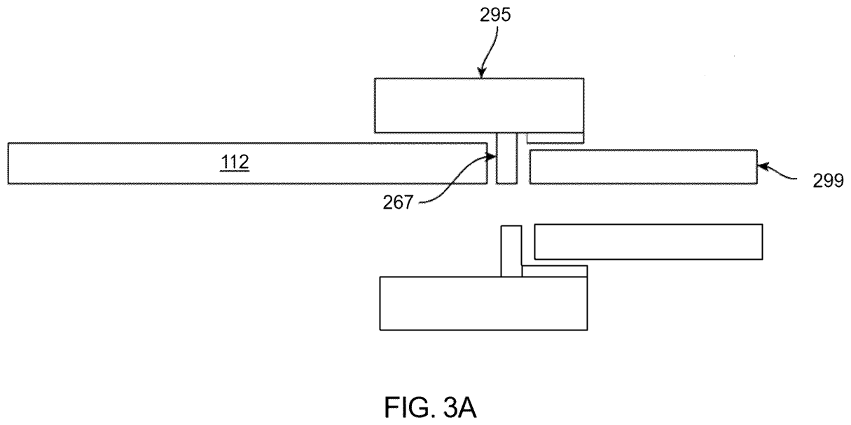

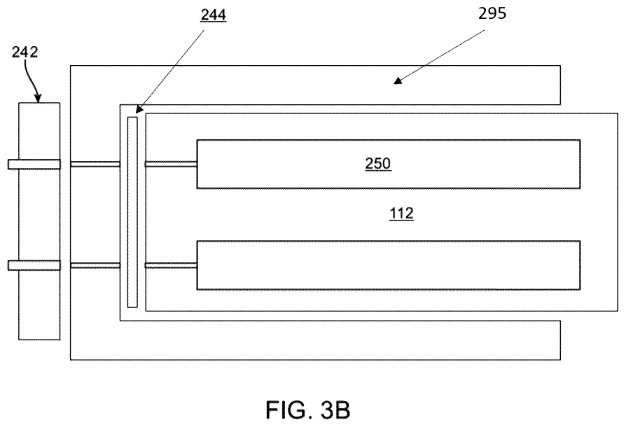

As shown in FIG. 3A, the flow cell device 112 may further comprise a frame 295 covering at least a portion of the one or more substrates. In some embodiments, the frame 295 is mechanically fixed to the one or more seals. The frame may comprise plastic, metal, polymer, glass, or a combination thereof. The frame may be configured to facilitate positioning of the one or more substrate relative to a connector or a manifold that is in fluidic communication with the flow cell device. In some embodiments, the frame may cover a portion of the top substrate and/or bottom substrate as shown in FIG. 3A. In some embodiments, the frame may keep the top substrate and/or bottom substrate exposed for imaging and heat transmission purposes, as shown in FIG. 3B. Also shown in FIG. 3B is a larger gasket that is part of instrument 242, an intermediate gasket that is part of the flow cell consumable 244, and a plastic frame that is part of the flow cell consumable 295.

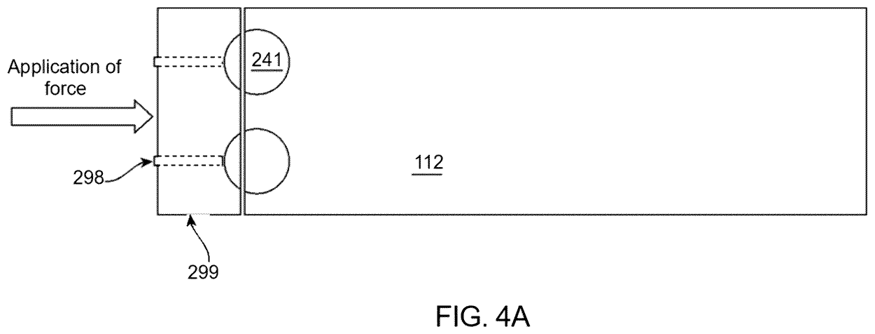

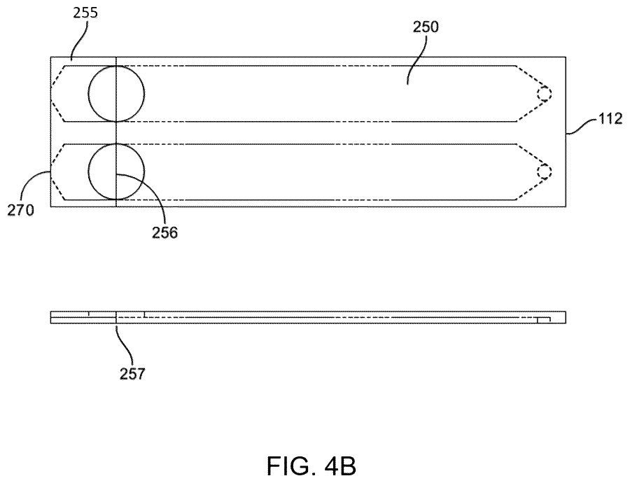

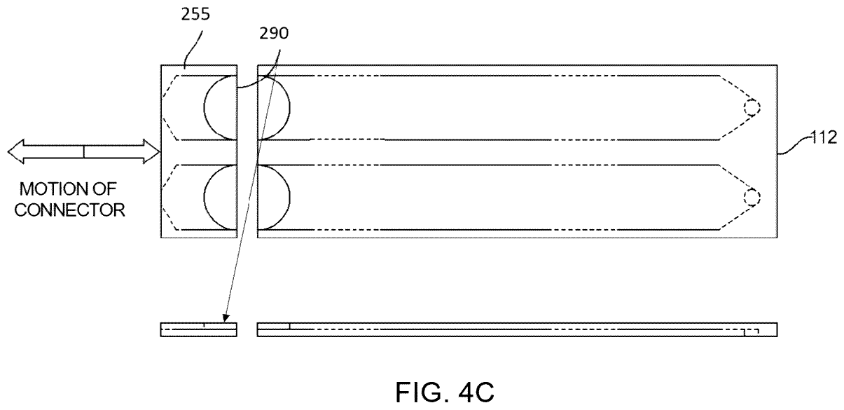

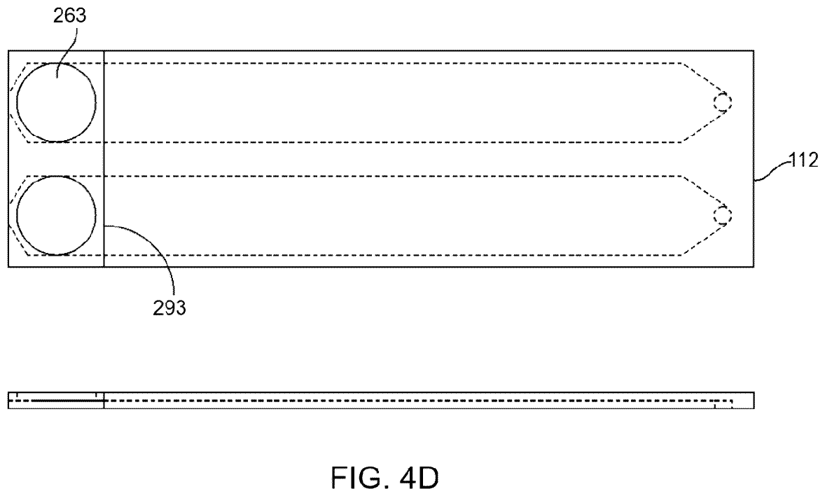

In some embodiments, the flow cell system may further comprise a manifold or connector 299 that interfaces with the flow cell device 112. The manifold or connector can comprise one or more fluidic pathways 298. The one or more fluidic pathways 298 may be in fluidic communication with the one or more channels 250, directly or indirectly. In some embodiments, the manifold or connector comprises one or more fluidic pathways 298 in fluidic communication with one or more open landing areas 241, 341a, 441a, 541a, 741a. For example, in FIGS. 4A-4D, the manifold or connector comprises a corresponding fluidic pathway that opens to an area that when coupled with the flow cell devices, becomes a complete circular open landing area 241. In other words, part of the entire open landing area 241, 341a, 441a, 541a, 741a may be comprised in the manifold or connector 299, while the other part of the entire open landing area 241 may be comprised in the substrates. Having the open landing area partly or completely off the flow cell device as shown in FIGS. 4A-4D may advantageously increase the length of the one or more channels along the y axis thus increasing imaging areas than flow cell devices with open landing areas that cannot be used as imaging areas. The open landing area that is partly or completely moved to the manifold or connector can be combined with other embodiments herein to increase imaging areas in various sequencing and imaging applications.

In some embodiments, the manifold or connector is configured to be in sealed fluidic communication with the one or more channels with application of a force or pressure satisfying a predetermined threshold thereon. The force or pressure may be along the y axis, at least. In some embodiments, the pressure applied on the structural elements of the flow cell system, e.g., the gasket or the manifold, can be in a range from 0 to 500 kPa, 0 to 280 kPa, 0 to 250 kPa, or 0 to kPa. In some embodiments, the force applied on the structural elements of the flow cell system, e.g., the gasket or the manifold, can be in a range from 0 Newton to 80 Newton, 0 to 60 N, 2 N to 50 N, or 5 to 30 N. In some embodiments, some or all of the structural elements of the flow cell system may be in a vacuum configuration, therefore exerting pressure or force satisfying the threshold for sealing fluidic communication between the flow cell device and the manifold. In some embodiments, the pressure threshold is in a range from 100 kPa to 500 kPa. In some embodiments, the pressure threshold is in a range from 150 kPa to 300 kPa. In some embodiments, the force threshold is in a range from 0.1N to 35N. In some embodiments, the force threshold is in a range from 1N to 25N.

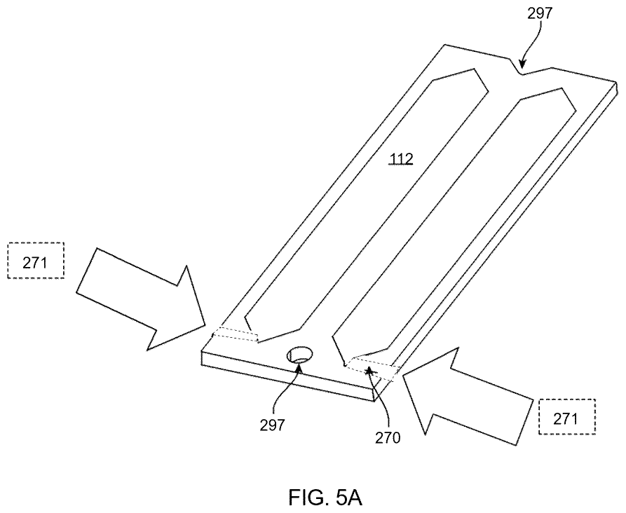

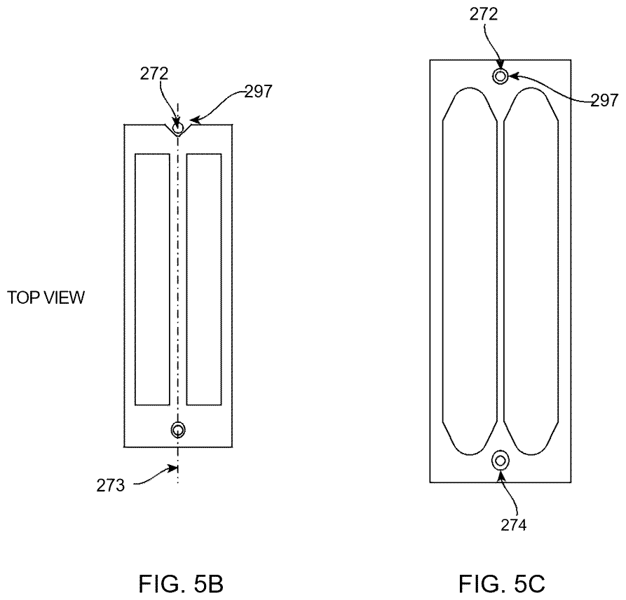

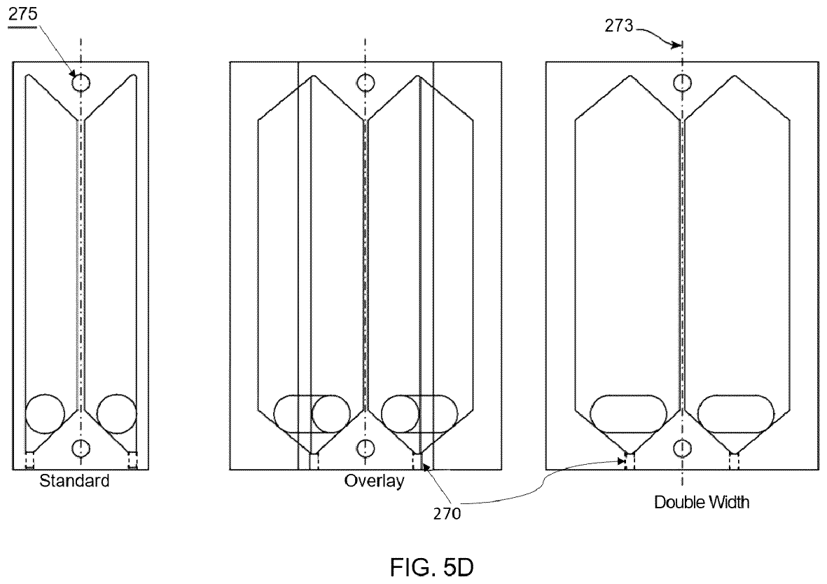

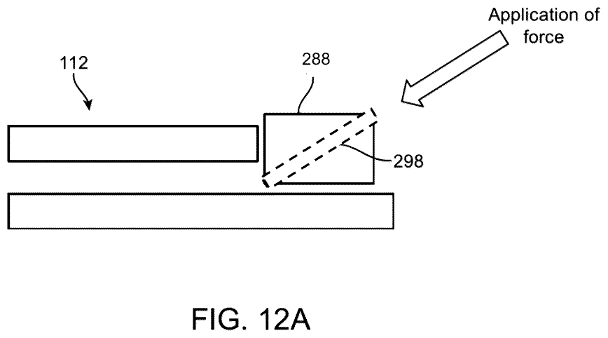

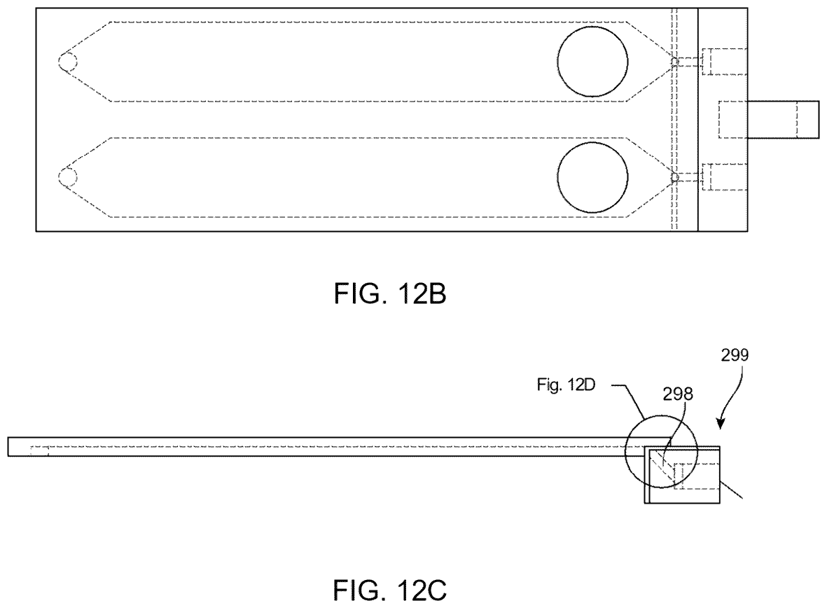

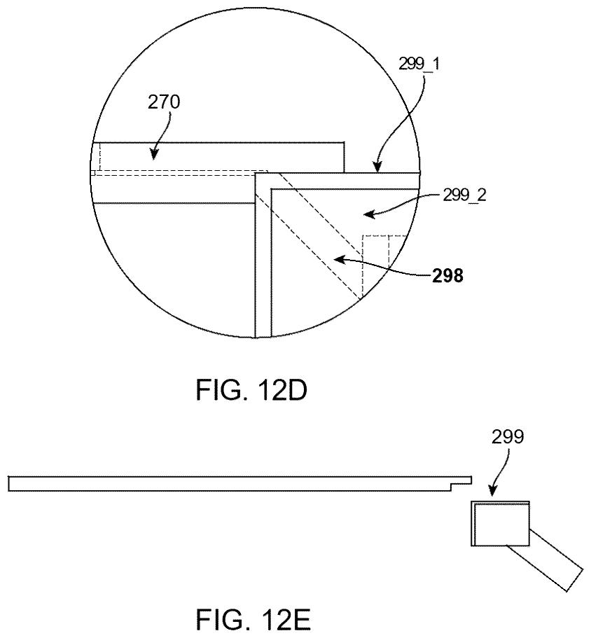

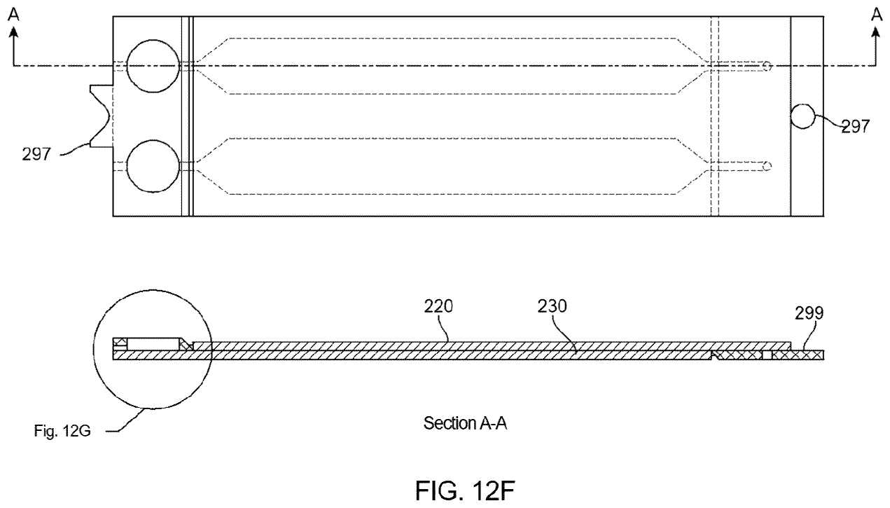

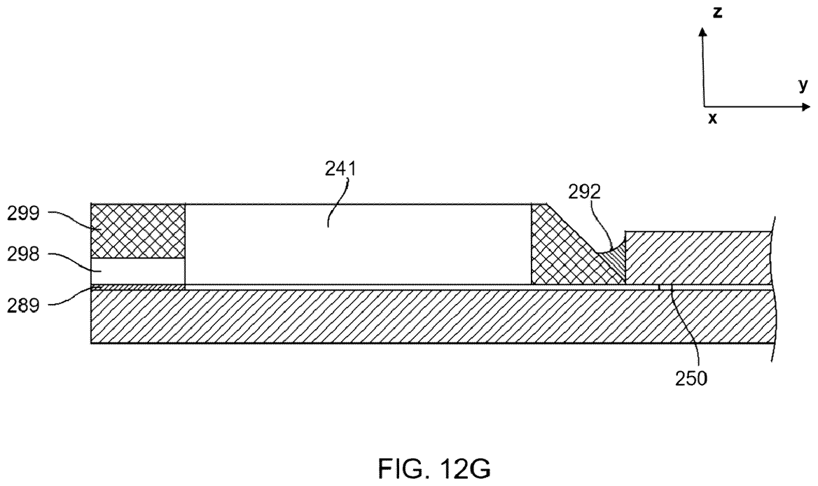

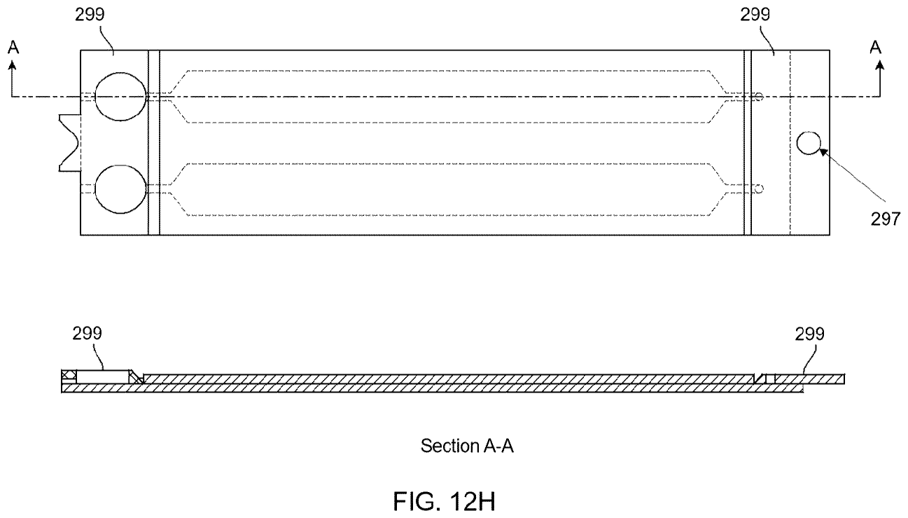

The one or more fluidic pathways 298 may extend along a y axis and wherein the pressure is applied along the y axis, e.g., FIG. 4A. The one or more fluidic pathways extend along a x axis and wherein the pressure is applied along the x axis, e.g., FIG. 5A. Also shown in FIG. 5A are one or more reference features 297 and a manifold interface 271. In some embodiments, the one or more fluidic pathways may extend in any direction in the x-y plane, in the y-z plane (FIG. 12A), or in three dimensions. Also shown are FIGS. 5B, 5C, and 5D. In FIG. 5B, also shown is a centering alignment pin 272, and datum established by centering the alignment features 273. Also shown in FIG. 5C is a hole in the slot alignment feature 274. In FIG. 5D, also shown is a pin-in-hole alignment feature 275.

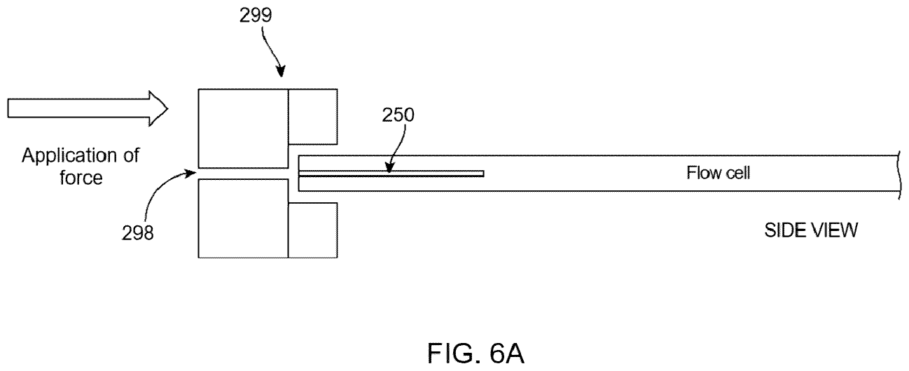

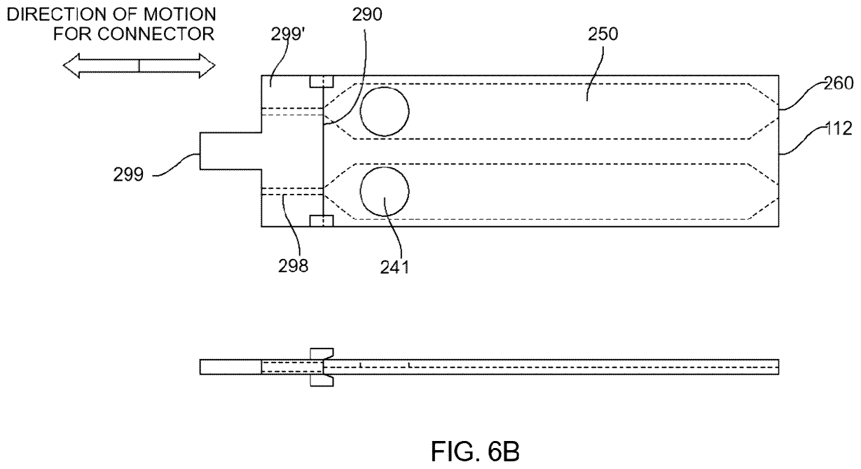

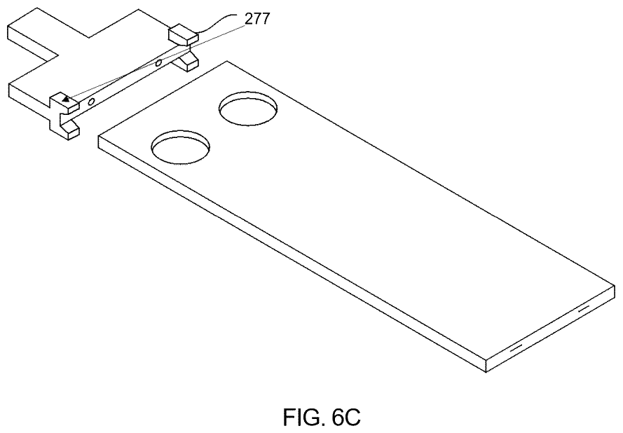

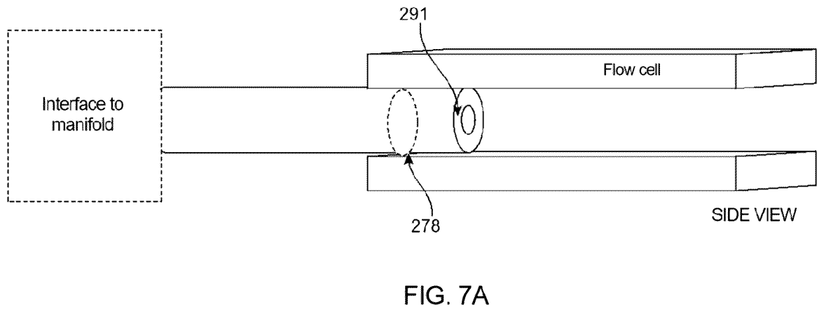

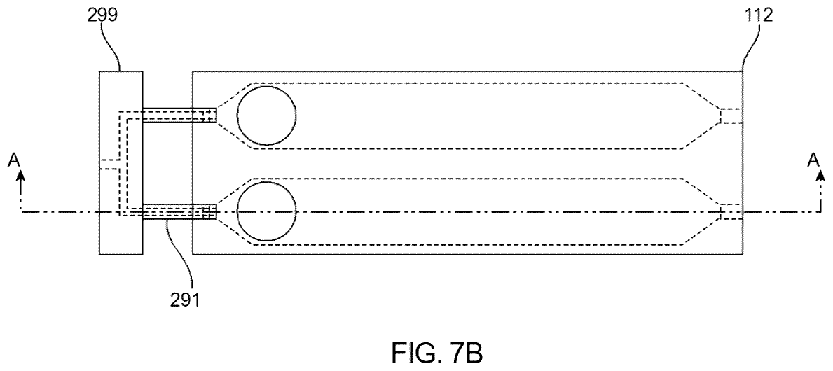

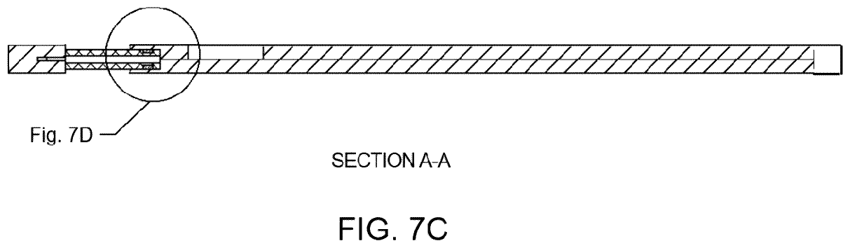

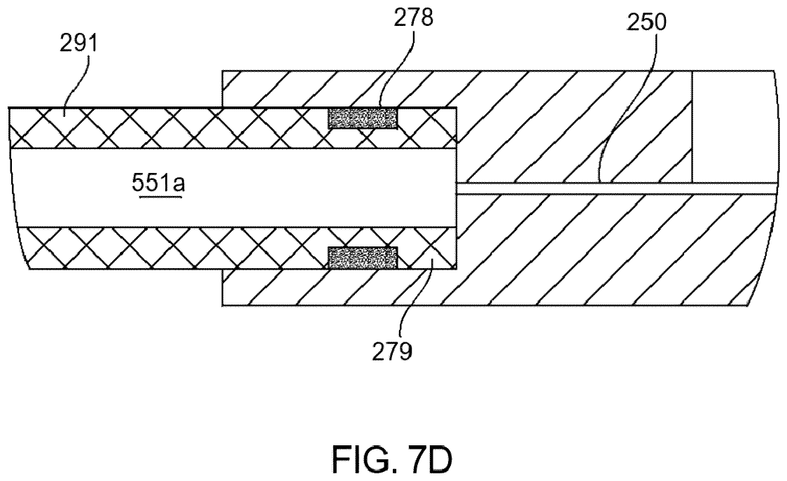

In some embodiments, a single fluidic pathway may correspond to and in fluidic communication with only a corresponding channel to minimize contamination across channels. In some embodiments, a single fluidic pathway may be in fluidic communication with multiple channels. It is advantageous that the flow cell devices herein can be connected (in sealed fluidic communication) with different connectors or manifolds having different configurations to optimize flexibility in utilizing the flow cell device for different sequencing application or chemistry protocols. For example, as shown in FIG. 7B, different channels of the flow cell devices can be in fluidic communication with a same reagent cartridge through the manifold or connector 299. Alternatively, as shown in FIG. 6B, different channels of the flow cell devices can be in fluidic communication with different reagent cartridges using a different manifold or connector 299.

In some embodiments, the one or more seals interface with the manifold, the connector, or a fluid control device to allow sealed fluidic communication to the one or more channels, as shown in FIGS. 3A-3B, 5A and 6A-6C. Such interfacing can be direct or indirect. With direct interfacing, the one or more seals directly connect the manifold or the flow control device. With indirect interfacing, there can be a connector or fitting therebetween.

In some embodiments, the one or more seals may function as a connector or part of a manifold. In some embodiments, the one or more seals may include: a connector, a manifold, part of a manifold, or their combinations.

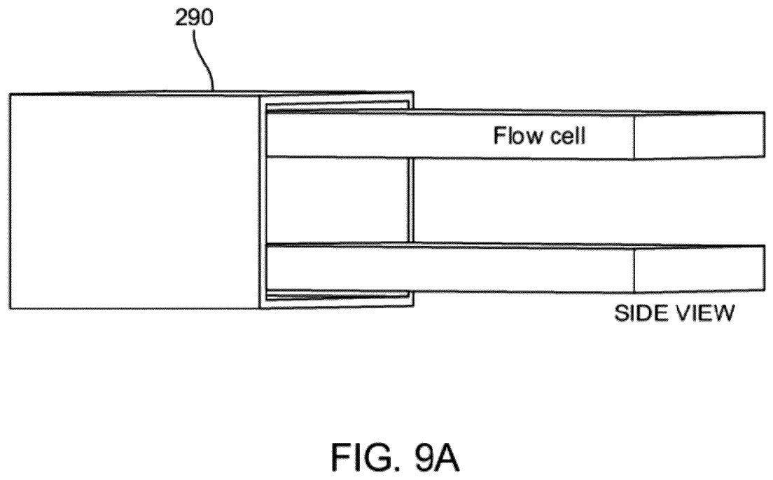

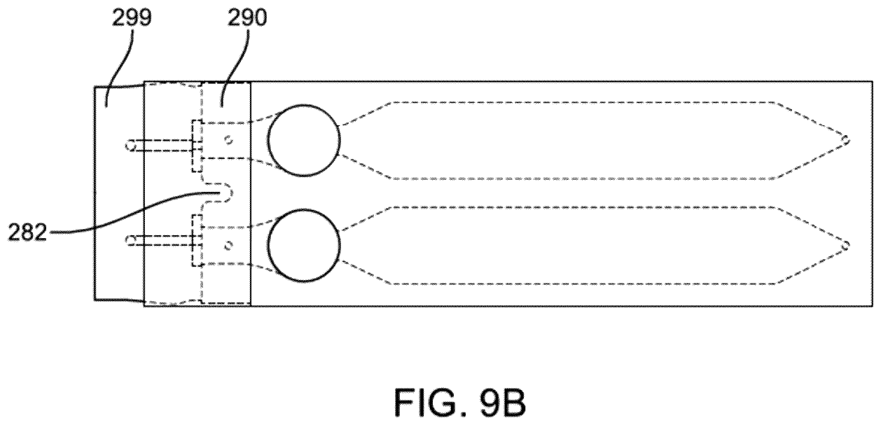

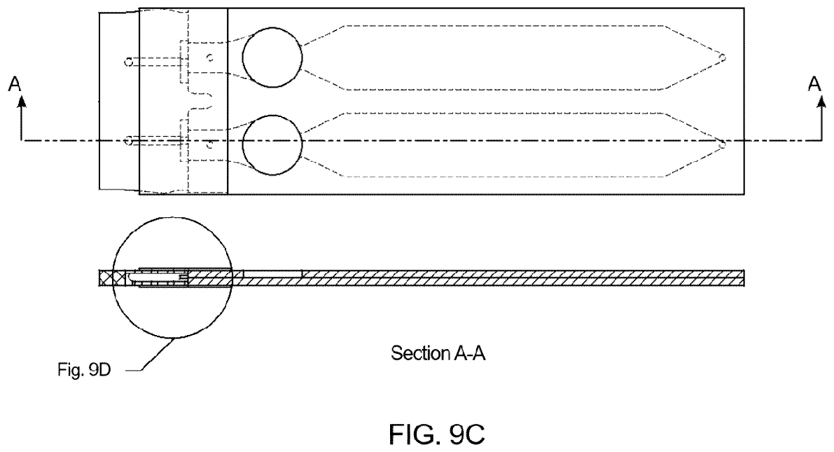

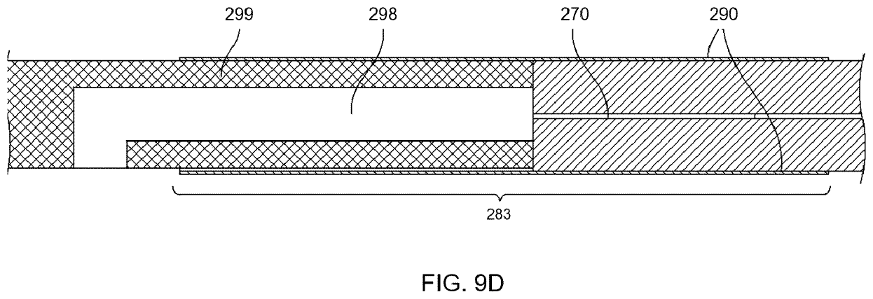

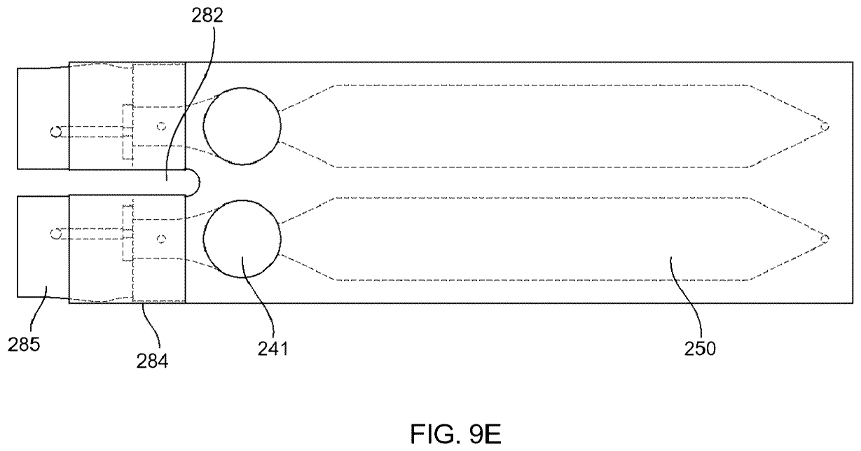

In some embodiments, the one or more seals comprises a sock seal 290 that covers at least a portion of the flow cell device 112 in the x-y plane. In some embodiments, the sock seal also covers one end of the flow cell device in the x-z plane, e.g., in FIG. 9A.

In some embodiments, the one or more seals comprises a membrane seal 290. In some embodiments, the membrane seal or sleeve may lap together two surfaces of the flow cell device and the manifold or connector at the top and/or bottom of the flow cell device. The membrane seal may be flat or conform to flatness of the surfaces it laps together. FIGS. 9B-9E show an embodiment of the flow cell device with the membrane seal 290. The membrane seal may advantageously require less sealing force or pressure than other seal geometries (e.g. an O-ring at the end of the substrate). In some embodiments, the membrane seal may enable a path length along y, covering at least part of the manifold and part of the substrates, e.g., the path length may be several millimeters or longer along y axis. The membrane seal may also include a thin cross section, along the z axis and/or x axis, thereby having a relatively high resistance to flow. Air leaking in from around the seal may have a negligible effect on the sealed fluidic communication. An additional advantage of the membrane seal is that the negative pressure inside acts over a larger area and the force multiplication of this large area can more effectively overcome any stiffness in a loose sleeve and maintains the thin cross section in vacuum applications. The membrane seal may reduce or eliminate flow between separate fluidic channels of the flow cell device, effectively sealing the two lanes independently. A gasket material can be included at the finger cut-out location to separate nearby channels and enable sealing therebetween, as shown in FIG. 9E. Also shown in FIG. 9B is a finger cut area that separates the fluidic paths 282. Also shown in FIG. 9E is a plastic gasket manifold on a per-lane basis 285, and a polymer gasket sleeve on a per-lane basis 284.

The material of the membrane seal or other seals disclosed herein may be compliant so that the sealing force increases, e.g., proportionally to, as increasing vacuum. The path length along y axis may be of various lengths ranging from 1 mm to 4 cm. The path length along y axis may be of various lengths ranging from 2 mm to 2 cm. The path length along y axis may be of various lengths ranging from 2 mm to 1 cm. As shown in FIGS. 9A, the path length may cover all the width of the flow cell device along x axis. FIG. 9D shows an expanded view of the interface of the manifold or connector 299 and the flow cell device 112 interfaced together with the membrane seal 290. In some embodiments, the one or more seals may additionally include adhesive or shrink-tight elements to enhance sealing. Also shown in FIG. 9D is the path length 283.

In some embodiments, the one or more seals comprises a L-shaped seal that extends along the z axis and y axis. As shown in FIG. 8, the L-shaped seal extends along the y axis and into a corresponding channel of the one or more channels. A pressure or force may be applied to the L-shaped seal along y axis to enable sealed fluidic communication between the flow cell device and the manifold. The one or more seals may be configured to interface with the manifold thereby allowing sealed fluidic communication between the flow cell device and the manifold. In some embodiments, the seal that extends along the z and y axis may be of various shapes that is similar to the L-shape, for example, a C-shape. The size of the arms of the L-shape also can be varied, for example to cover at least part of the thickness or all the thickness of the substrate. Also shown in FIG. 8 are flexible flaps inserted into the flow cell channel 281.

In some embodiments, the one or more seals comprise a diagonal gasket with a fluidic pathway running in an y-z plane. FIG. 12A shows a side view of an embodiment of the flow cell device and the diagonal gasket 288 connectable to the flow cell device. The diagonal gasket may interface with an end of the top substrate and a bottom interior surface 252 of the bottom substrate as shown in FIG. 12A. Alternatively, the diagonal gasket may interface with an end of the bottom substrate and a top interior surface 251 of the top substrate. The gasket

may have a thickness along z axis that does not block motion of the dispensing tip to the open landing area, e.g., in x-y plane. The acute angle between the channel and the fluidic pathway can be varied in a range from 0 degrees to 85 degrees. The acute angle between the channel and the fluidic pathway can be varied in a range from 10 degrees to 65 degrees.

FIG. 12B shows a top view of an embodiment of the flow cell device with a manifold or connector 299 that is structurally and functionally similar as the diagonal gasket in FIG. 12A. FIG. 12C shows a side view of the flow cell device in FIG. 12B. FIG. 12D is an expanded view of the manifold or connector 299 interfacing with the channel 250 of the flow cell device in FIG. 12C. FIG. 12E shows the manifold or connector that is not coupled to the flow cell device. The manifold or connector may interface with an end of the bottom substrate and a top interior surface 251 of the top substrate, e.g., FIG. 12C. Coupling the diagonal gasket, the manifold, or the connector to the flow cell device as shown in FIG. 12C may advantageously provide clearance above the top surface 261 of the top substrate thereby facilitating easy and efficient movement of the dispensing tools to the inlet or open landing area of the flow cell device. Alternatively, the manifold or connector may interface with an end of the top substrate and a bottom interior surface 252 of the bottom substrate. The diagonal gasket, manifold, or connector 299 may allow sealed fluidic communication from the fluidic pathway 298 to the one or more channels 250 when a force or pressure comprising a y-axis component satisfying a first threshold and a z axis component satisfying a second threshold is applied. In some embodiments, The diagonal gasket, the manifold, or connector, may include a handle for applying force or pressure on the diagonal gasket, as shown in FIG. 12C and FIG. 6C. The force or pressure may be applied along direction that the handle extends. The force or pressure may be applied in various 3 dimensional directions. The force or pressure may be at least along y and z directions. Also shown in FIG. 6C are optional self-alignment features 277.

In some embodiments, the manifold or connector 299 may include a connector core 299_1 and a gasket over-mold 299_2 on at least part of the connector core. For example, the over-mold 299_2 may cover the interfacing area that the connector 299 may have with the substrate(s) of the flow cell device 112, e.g., FIG. 12D. In some embodiments, the manifold or connector may include the connector core 299_1 and a separate piece of gasket, for example, in an “L” shape or various other shapes, which can be assembled together with the connector core 299_1.

In some embodiments, the top substrate and bottom substrate of the flow cell device may have a lateral offset along y axis from each other, the manifold or connector 299 may be positioned on top of the bottom substrate as shown in FIGS. 12F-12H. In such embodiments, the manifold or connector may be fixedly attached to the one or more substrate. For example, the manifold or connector 299 can be laminated onto the substrates with pressure sensitive adhesive or various bonding or adhesion methods. The fluidic pathway 298 in the manifold or connector can be in sealed fluidic communication with one or more channels of the flow cell device, directly or indirectly. As shown in FIG. 12G, the fluidic pathway 298 in the manifold or connector is in direct fluidic communication with the open landing area and the channels without a cleaning outlet 270. The fluidic pathway 298 may include an end that connects to a fluidic control device, e.g., a vacuum. That end of the fluidic pathway may be in a plane that is orthogonal to the x-z plane as shown in FIG. 12G. Alternatively, the fluidic pathway can run from the one or more channels and exit the manifold or connector from a side thereof in a plane that is orthogonal to y-z plane (not shown). Alternatively, the fluidic pathway can run from the one or more channels and exit the manifold or connector from a top or bottom thereof in a plane that is orthogonal to x-y plane (not shown). Also shown in FIG. 12G is an interposer 289 and an optional adhesive or seal 292.

In some embodiments, the flow cell system herein may include an interposer defining the one or more channel and the open landing area. The interpose can be between the manifold or connector and the bottom substrate as shown in FIG. 12G. In some embodiments, the interpose can be between the manifold or connector and the top substrate when the manifold or connector is positioned beneath the top substrate.

In some embodiments, at least some portion of the manifold can comprise plastics.

In some embodiments, adhesive seal may be used at some area(s) of interfaces between the manifold or connector and the substrate. In some embodiments, adhesive seal may be applied depending on materials and surface properties of the substrate and the manifold or connector. For example, optional adhesive or seal may be added when the manifold or connector has a ramped face facing the end of the top substrate as shown in FIG. 12G.

In some embodiments, the open landing area 241, 341a, 441a, 541a, 741a can be comprised in the manifold or connector instead of in the one or more substrates, as shown in FIG. 12F-12G.

In some embodiments, two substrates of similar sizes, the top and bottom substrates are laterally offset in y axis in order to create two bonding areas for manifold or connectors. One bonding area is close to the opening landing area as shown in FIG. 12G. The other bonding area is at the other end of the flow cell device near the outlet as shown in FIG. 12F, and it is configured to bind an underside manifold or connector 299. Alternatively, a larger bottom substrate can be used as shown in FIG. 12H, so instead of the underside manifold or connector, both bonding areas are configured for fixedly attaching a manifold or connector on top of the bottom substrate. In some embodiments, the thickness of the manifold along z axis can be maintained to be comparable to the thickness of the top substrate thereby providing clearance for dispensing tips to the open landing area.

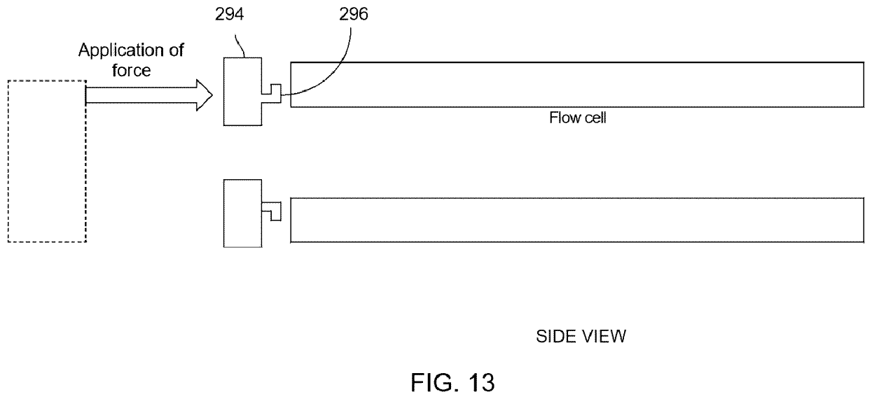

FIG. 13 shows a thermoplastic connector 294 and a thermoplastic seal 296.

In some embodiments, the bottom substrate may include a length along y axis in the range from 50 mm to 120 mm. In some embodiments, the bottom substrate may include a length along y axis in the range from 60 mm to 110 mm. In some embodiments, the bottom substrate may include a length along y axis in the range from 75 mm to 100 mm. In some embodiments, the bottom substrate may include a length along y axis in the range from 85 mm to 100 mm.

In some embodiments, the one or more seals comprises a thermoplastic connector and a thermoplastic seal mounted on the thermoplastic connector. The thermoplastic seal may be deformable under pressure changes, temperature changes, or both. The thermoplastic seal may comprise one or more materials different from the thermoplastic connector. The thermoplastic seal may enable sealed fluidic communication between the channels of the flow cell device and the manifold, when a force or pressure satisfying a predetermined threshold is applied. The force or pressure may include a y axis component, at least.

It is worth noting that the threshold force or pressure applying in different embodiments of the flow cell device to seal the fluidic communication may be different or identical. It is worth noting that various mechanisms may be used to maintain constant application of force or pressure for a predetermined period of time. The predetermined period of time may vary from less than 1 second to multiple hours. In some embodiments, the predetermined period of time includes the time window during which the reagents are being communicated between the flow cell devices and other structural elements of the flow cell system, e.g., the manifold or connector. In some embodiments, the predetermined period of time includes the time window during which a sequencing run is in progress.

The force or pressure may be along the y axis, at least. In some embodiments, the pressure applied to seal the fluidic communication between the flow cell device and other elements, e.g., the gasket or the manifold, can be in a range from 0 to 500 kPa, 0 to 280 kPa, 0 to 250 kPa, or 0 to 220 kPa. In some embodiments, the pressure applied to seal the fluidic communication between the flow cell device and other elements, e.g., the gasket or the manifold, can be not greater than 100 kPa, 150 kPa, 180 kPa, 200 kPa, 300 kPa, or 400 kPa. In some embodiments, the force applied on the flow cell system, e.g., the gasket or the manifold, can be in a range from 0 Newton to 80 Newton, 0 to 60 N, 2 N to 50 N, or 5 to 30 N. In some embodiments, the force applied on the flow cell system, e.g., the gasket or the manifold, can be not greater than 20N, 25N, 20N, 35N, 40N, 45N, 50N, 55N, 60N, 70N, 80N, 100N, or 200N. In some embodiments, some or all of the structural elements of the flow cell system may be in a vacuum configuration, therefore exerting pressure or force satisfying the threshold for sealing fluidic communication between the flow cell device and the manifold. In some embodiments, the pressure threshold is in a range from 50 kPa to 500 kPa. In some embodiments, the pressure threshold is in a range from 150 kPa to 300 kPa. In some embodiments, the force threshold is in a range from 0.1N to 35N. In some embodiments, the force threshold is in a range from 1N to 25N.

In some embodiments, the one or more seals may comprise a first connector having a top portion that is slidable on a top surface 261 of the top substrate. The one or more seals comprises a second connector having a bottom portion that is slidable on the bottom surface 262 of the bottom substrate. The top portion may connect to a first side portion of the first connector that is configured to interface with an end of the flow cell device in the x-z plane. The bottom portion may connect to a second side portion of the second connector that is configured to interface with an end of the flow cell device in the x-z plane. The top portion or bottom portion connected with its corresponding side portion into an integrated connector. A pressure or force on the first and second side portion, satisfying a predetermined threshold, may be configured to slide the first and second connector relative to the flow cell device with deformation thereby enabling sealed communication between the one or more channels and a fluidic pathway defined between the top and bottom connector.

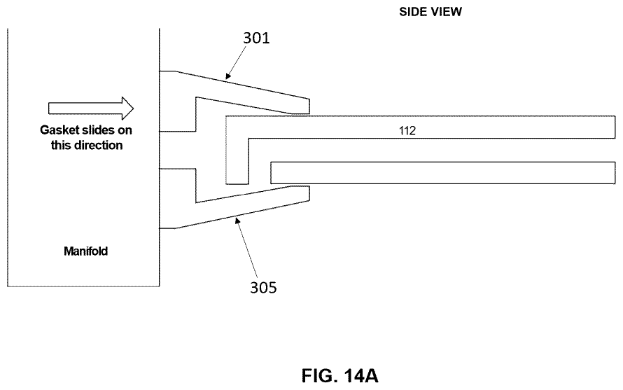

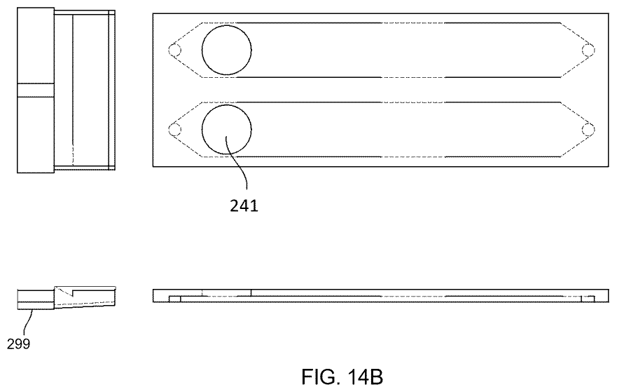

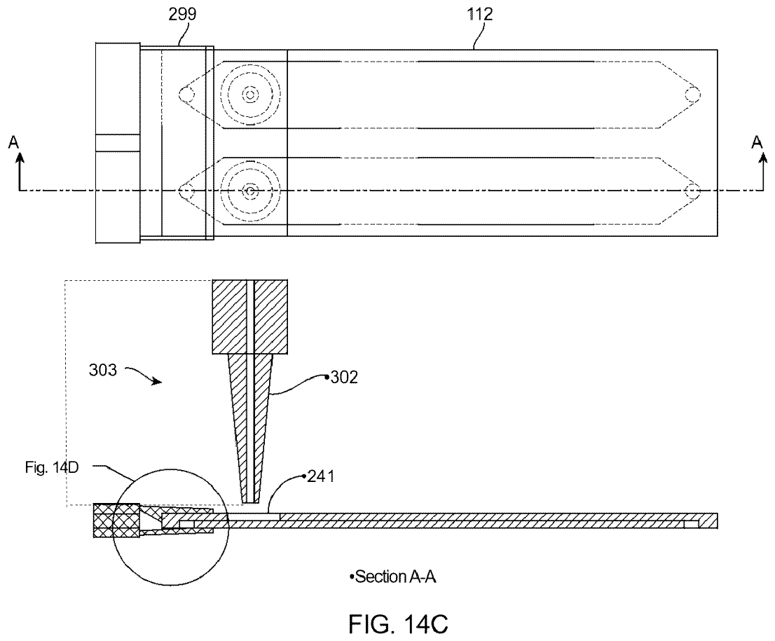

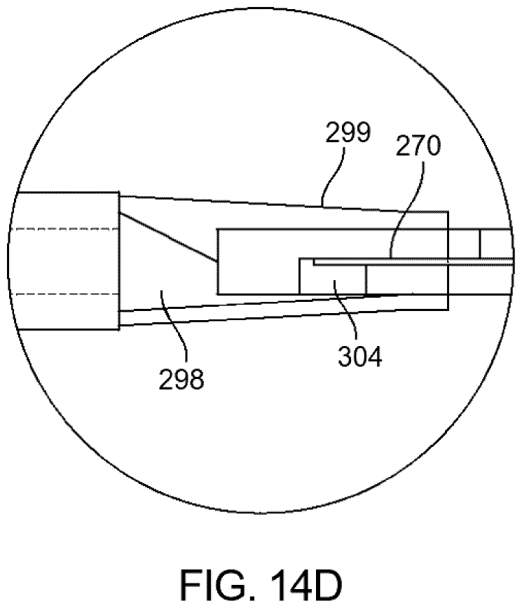

FIGS. 14A-14D show a non-limiting example of the first connector 301 having a top portion slidable on the top surface of the top substrate and a second connector 305 having a bottom portion slidable on the bottom surface of the bottom substrate. The specific geometry of the first and second connector can vary in different applications. The sliding surface of the top portion and the bottom portion may be approximately flat to allow smooth sliding relative to the flow cell device. In this particular embodiment, the flow cell device includes a port opening at the bottom surface of the flow cell to enable fluidic communication between the fluidic pathway and the one or more channels. In some embodiments, the port can open at the top surface, at one end of the flow cell device along the y axis (or in the x-z plane), or at either side of the flow cell device along x axis (or in the y-z plane).

The first connector may include a smooth top surface as shown in FIGS. 14C-14D to provide clearance for dispensing tips to travel to the open landing area. FIGS. 14B and 14C shows the connector in a disconnected and connected position to the flow cell device. The connector may be actuated using a force-applying mechanism to enable accurate connection or disconnection with sealed fluidic communication. force-applying mechanism may include a motor. The force-applying mechanism may be controlled by the sequencing system, e.g., using a software program executable on a computer processor of the sequencing system, to exert force or pressure via the motor for a predetermined period of time, e.g., during a sequencing run. Also shown in FIG. 14C is a pipettor 302 and a pipettor travel area 303. Also shown in FIG. 304 is a bottom port on the flow cell.

In some embodiments, the one or more seals comprises semi-rigid or deformable materials that deform under pressure or force. In some embodiments, the semi-rigid or deformable materials are configured to restore its shape before deformation when the pressure or force is removed.

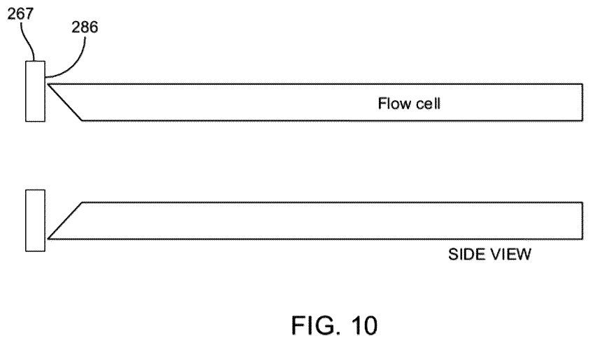

In some embodiments, the top substrate or bottom substrate comprises one or more ramped ends. In some embodiments, a tip of the ramped end may press on the one or more seals, e.g., FIG. 10. The ramped end may facilitate sealing of the fluidic communication by requiring less pressure or force. The ramp may be within the y-z plane. The tip pressing on the one or more seals may be at a top surface or bottom surface, e.g., 251, 252, 261, 262, of the top or bottom substrate. Also shown in FIG. 10 are the semi-sharp corners of the flow cell pressed into the gasket 286.

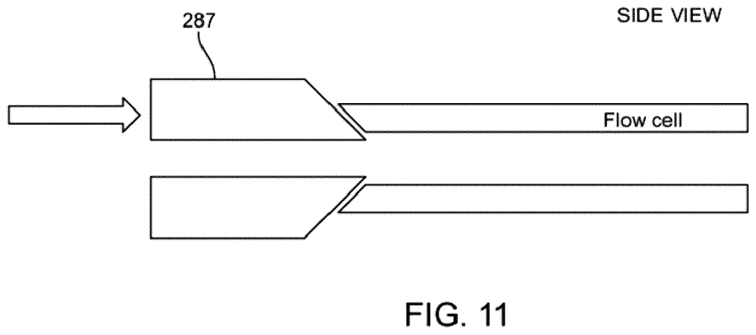

In some embodiments, each of the ramped ends may interface with a ramped manifold or connector, e.g., FIG. 11. The one or more ramped ends may comprise a first acute ramp angle to a y axis and wherein the ramped manifold or connector comprise a second acute ramp angle to the y axis. The first acute ramp angle may be different from the second acute ramp angle. The first acute ramp angle may be about identical to the second acute ramp angle. In that case, the ramped manifold or connector comprises a complementary ramp to the ramped end of the flow cell device. Also shown in FIG. 11 is a rigid flared tube manifold 287.

In some embodiments, the flow cell device 112 further comprises one or more reference features configured for positioning the flow cell device relative to the manifold or connector, a sample stage, or a sequencing system. The one or more reference features may comprise at least one alignment feature located at or near a central point along the x axis. The one or more reference features comprises at least one alignment feature located at or near an end of the one or more substrate along the y axis. As shown in FIGS. 5A-5D, the one or more reference features 297 comprises a cavity running through the one or more substrates and couplable to a pin or a post. In some embodiments, the one or more reference features comprises a grove (e.g., FIG. 5A) extending through the one or more substrates that is couplable to a pin or a post. In some embodiments, the one or more reference features may comprise various features that may couple together for alignment purposes, including but not limited to a recess, a clamp, a side arm, etc.

In some embodiments, the cleaning outlet may extend, at least, in the x-y plane. For example, the cleaning outlet 270 extends in the x-y plane as a side port, e.g., as shown in FIGS. 2D, 4, and 5A. In some embodiments, the side port extends at least along a direction that is perpendicular or nearly perpendicular to a y direction. In some embodiments, the side port extends at least along a direction that is perpendicular or nearly perpendicular to a z direction. In some embodiments, the side port extends at least along a direction that is oblique to an x direction. In some embodiments, the side port extends at least along a direction that is oblique to a y direction. In some embodiments, the side port extends at least along a direction that is oblique to a z direction. In some embodiments, the side port extends at least along a direction that is perpendicular or nearly perpendicular to an x-y plane. In some embodiments, the side port extends at least along a direction that is perpendicular or nearly perpendicular to an x-z plane. In some embodiments, the side port extends at least along a direction that is perpendicular or nearly perpendicular to an x-y plane. In some embodiments, the side port extends at least along a direction that is perpendicular or nearly perpendicular to a y-z plane. In some embodiments, the side port extends at least along a direction that is oblique to an x-y plane. In some embodiments, the side port extends at least along a direction that is oblique to an x-z plane. In some embodiments, the side port extends at least along a direction that is oblique to a y-z plane.

Such orientation of the cleaning outlet may be advantageously compatible with different flow cell shape, size and/or different shape or size of channels. For example, FIG. 5D shows how different flow cell devices can be aligned to the same sample stage using the reference feature(s) disclosed herein, and how the flow cell device with two channels can be expanded in the x-y plane, especially along the x-axis without the need to change existing cleaning outlets or add additional cleaning outlets for additional channels or wider channels. The ramped portion of individual channels may also be shaped to enable using shared cleaning outlet among two or more channels. In some embodiments, the location and orientation of the cleaning outlet, e.g., as shown in FIGS. 2D, 4, and 5A advantageously allow different flow cell devices to be sequenced without the need to change or add fluidic connections to the manifold/connector and/or fluidic control devices thereby enabling convenient and efficient switching between different sequencing applications. Further, the location and orientation of the cleaning outlet, e.g., as shown in FIGS. 2D, 4, and 5A advantageously allow convenient and efficient scaling of the flow cell device to increase the number of channels and samples to be sequenced, thereby allowing improved sequencing throughput using the same sequencing systems. FIG. 2E shows a side view of the flow cell device in FIG. 2D, and FIG. 2E shows a cross-sectional view of the flow cell device in FIG. 2D at AA. FIG. 2G shows the detailed view of FIG. 2F.

In some embodiments, the manifold or connector 299 comprises a top portion or bottom portion that extends beyond the one more substrate along the z axis and covers at least part of one or more substrate in a x-y plane. The top portion or bottom portion of the manifold can be at the first portion, the second portion, or both of the one or more channels 250. FIG. 6A-6C show non-limiting examples of the flow cell device 112 with the manifold or connector 299. The manifold may include a connector 299′ that connects the flow cell device 112 and the other elements of the manifold or connector 299. Alternatively, the connector 299′ may be a structurally separate piece from the manifold that functions to connect the flow cell device and the other elements of the manifold.

The connector 299′ may include the top and bottom portions defining an opening therebetween. The opening can be in direct sealed fluidic communication with the channel(s) 250. In some embodiments, each channel may be in fluidic communication with a fluidic pathway 298 of the manifold in a connected position, as shown in FIG. 6B. FIG. 6C shows the connector 299′ in a separated position from the flow cell device 112. The connector 299′ The flow cell device may include an optional gasket at the interface between the flow cell device and the manifold/connector 299.

The flow cell system may include a force-applying mechanism comprising but not limited to motors, electromagnetic actuators, springs, linkages, or their combinations. In some embodiments, the manifold or connector 299 may be actuated by the force-applying mechanism to connect or disconnect from the flow cell device. The force-applying mechanism may be controlled by the sequencing system to enable connection or disconnection to the flow cell device to enable sealed fluidic communication between the flow cell device and the fluidic control device. In some embodiments, the manifold or connector 299 may be part of a manifold device or fluid control device of the flow cell system.

In some embodiments, the flow cell system further comprises one or more tubes that interfaces with the manifold or connector 299 and the flow cell device 112. The tube(s) can be positioned therebetween. Each of the one or more tubes 291 may comprise a wall surrounding a lumen 551a. The lumen 551a may be in fluidic communication with the one or more channels 250 of the flow cell device and the one or more fluidic pathways 298 of the manifold or connector 299. The embedded tube(s) may advantageously provide improved sealing when compared with sealings at the end face of the substrates. An optional O-ring may be included to further improve sealing between the embedded tube and the flow cell device. FIGS. 7A-7D show non-limiting examples of the flow cell device with embedded tube(s). In the embodiments shown in FIGS. 7B-7D, the flow cell device 112 can be integrated with the manifold or connector 299 so that they are fixedly attached to each other with sealed fluidic communication therebetween to facilitate convenient handling of the flow cell device as well as easy and leak-proof connection to the fluidic control devices. For example, the integrated flow cell and connector may interface to an instrument-side connector. Such interface may include easy disconnect interfaces, e.g., as shown in FIGS. 6B-6C. Also shown in FIG. 7D is an optional O-ring 278 and a tube embedded into the flow cell 279.

In some embodiments, the pressure or force applied may be customized depending on different size, shape, material, or other characteristics of the flow cell system. In some embodiments, the pressure applied on the structural elements of the flow cell system, e.g., the gasket or the manifold, can be in a range from 0 to 320 kPa, 0 to 280 kPa, 0 to 250 kPa, or 0 to 220 ka. In some embodiments, some or all of the structural elements of the flow cell system may be in a vacuum configuration, therefore exerting pressure satisfying the threshold for sealing fluidic communication between the flow cell device and the manifold. In some embodiments, the pressure threshold is in a range from 150 kPa to 300 kPa. In some embodiments, the force threshold is in a range from 0.1N to 35N. In some embodiments, the force threshold is in a range from 1N to 25N.

In some embodiments, the force applied on the structural elements of the flow cell system, e.g., the gasket or the manifold, can be in a range from 0 to 50 N, 0 to 40 N, 5 to 30 N, 5 to 25N, 1 to 25N, or 5 to 15 N. In some embodiments, some or all of the structural elements of the flow cell system may be in a vacuum configuration, therefore exerting force satisfying the threshold for sealing fluidic communication between the flow cell device and the manifold.

It is worth noting that the different embodiments of flow cell devices and features disclosed corresponding to such embodiments are not limited to the corresponding embodiments they are disclosed in. Instead, embodiments and their corresponding features may be combined together for various customized needs. As a non-limiting example, FIG. 12H includes the reference features 297 as shown in the embodiments in FIGS. 5A-5D. As another example, the one or more substrates in embodiments shown in FIG. 12A-12H may include a bottom or top substrate with one or more layers 221, 231 as shown in FIGS. 2A-2C. As yet in an example, the one or more substrates in FIG. 12A-12H may include a ramped end as shown in FIG. 11. As yet in another example, the open landing area may be completely on the flow cell device, partly on the flow cell device, or completely on the manifold or connector as shown in FIGS. 2D, 4A-4B, and 12F. As shown in FIG. 4B, the connector includes a bonded end cap 255 that can interface with the flow cell device, and the open landing area may be split between the end cap and the flow cell device. Also shown in FIG. 4B is a pipettor landing pad split between the end cap and the flow cell 256, and a bonding interface 257. Shown in FIG. 4C is a connectorized end cap 255 shown in a disconnected position, as well as the presence of a face seal gasket 290. Shown in FIG. 4D is a pipettor landing pad located entirely on the end cap 263, as well as a bonded or connectorized interface 293.

As shown in FIG. 33C, one or more of the interior surfaces 521a can be coated with a first coating 522a.

In some embodiments, the channels are configured to allow fluids, e.g., liquid reagents, and an air gap between the fluids to flow therethrough. In some embodiments, the air gap can comprise a bolus of gas. The air gap can be introduced similarly as the liquid reagents, e.g., via the inlet to the channels, and then exit from the outlet and/or from the cleaning outlet. Alternatively, the air gap can be introduced from other openings such as the outlet or the cleaning outlet of the flow cell device. The air gap can be driven mechanically by one or more structural elements of the fluidic control device herein. As an example, the air gap can be sucked into the channels via the inlet by a mechanical force applied at the outlet, e.g., by a pump or a vacuum. As another example, the air gap may be purged by a pump or the like via the inlet.

The volume of the air gap can vary depending on the geometry, or size, or combinations thereof, of the flow cells and channels. For example, the volume of air gap can be selected to fill up about 30%, 40%, 50%, 60%, or 70% of the entire volume of each channel. As another example, the volume of the air gap can be adjusted based on the subsequent reagent to be administered, e.g., air gap can be increased if higher cleaning or reduction in contamination is desired.

The air gap that flows through the one or more channels can be configured to push existing reagents in the channel(s) toward the outlet and exit from outlet. As a result, subsequent delivery of sequencing reagent(s) can achieve high homogeneity in the flow cells. In existing flow cells relying solely on washing buffer(s) between the delivery of sequencing reagents, mixing of the sequencing reagents with washing buffer or liquid(s) is inevitable, and there can be a concentration gradient of the sequencing reagent(s) with higher concentration at one end closer to the landing area or inlet, and lower concentration at the opposite end closer to the outlet. Such gradient or inhomogeneity can be gradually reduced by repeated washing but remains difficult to be completely eliminated. The gradient of concentration or inhomogeneity in concentration of reagents may cause sequencing analysis of tiles toward the opposite end of the flow cell to be less accurate and unreliable at least partly due to inhomogeneous reactions, or attachment, or combinations thereof, of compounds in the reagent to the polonies. In addition, introduction of air bubbles into existing flow cells between reagents may damage the channel coating, or the polonies, or combinations thereof, tethered thereon and being imaged, thereby impairing the sequencing process. The flow cell devices herein may advantageously utilize the air gap between administration of sequencing reagents to minimize or eliminate reagent concentration gradient or inhomogeneity in the flow cells, along the y axis, with no or minimal damages to the samples immobilized thereon in sequencing processes.

In some embodiments, the air gap and washing liquid(s) can be combined to achieve optimal cleaning of the channel(s). In some embodiments, the air gap can be used alone to achieve optimal cleaning of the channels. In some embodiments, the washing scheme, using air gap, washing liquid(s), or both can be determined based on the contamination level of the reagent to be delivered. In some embodiments, the washing scheme, using air gap, washing liquid(s), or both can be determined based on the cost of the reagent, along or in combination with other factors such as contamination levels. In some embodiments, the order of using air gap and washing liquid(s) can vary when the two are combined in the washing scheme. The air gap can be applied after or before any number of flushing with washing liquid(s). In some embodiments, the air gap can be purged in between any selected flushing of washing liquids.

The air gap that flows through the one or more channels may dry the coating of the one or more channels, but the functionality of the coating can remain unaltered after one or more air gaps flow therethrough. In some embodiments, the air gap that flows through the one or more channels may dry the polonies tethered thereon the channel coating. However, the air gap does not damage the polonies and ensures proper sequencing reaction of the polonies when a subsequent liquid reagent is flushed through the channel(s). As such, the flow cell devices with such channels can be cleaned by pursing air gaps into the channels, alone or in combination with washing with reagents. Usage of the air gap for cleaning can increase the efficiency and effectiveness of cleaning the channels while simultaneously reducing the costs of reagents that is required for washing and performing sequencing analysis, while satisfying a predetermined contamination requirement.

In some embodiments, the surface can be passivated for the first coating 522a. In some embodiments, the surface is passivated with the first coating 522a that immobilizes surface capture primers, nucleic acid template molecules, or both for capturing polynucleotides thereon. In some embodiments, during sequencing, the surface can comprise polynucleotides captured thereon. In some embodiments, the polynucleotides captured thereon are configured to be imaged in a sequencing cycle.

In some embodiments, the first coating 522a of the surface comprises one or more hydrophilic polymer coating layers. The first coating can comprise a plurality of oligonucleotide molecules attached to at least one hydrophilic polymer coating layer. The hydrophilic polymer coating layer(s) can comprise PEG. The hydrophilic polymer layer(s) can comprise a branched hydrophilic polymer and the branched hydrophilic polymer can comprise at least 8 branches. In some embodiments, the hydrophilic polymer coating layer(s) has a water contact angle of no more than about 50 degrees.

In some embodiments, the surface comprises at least one discrete region that comprises a plurality of clonally-amplified sample nucleic acid molecules that have been annealed to the plurality of attached oligonucleotide molecules. In some embodiments, at least one of the plurality of the clonally-amplified sample nucleic acid molecules comprises a concatemer annealed to at least one of the plurality of attached oligonucleotide.

In some embodiments, the at least one of the plurality of sample nucleic acid molecules comprises a single-stranded multimeric nucleic acid molecule comprising repeats of a regularly occurring monomer unit. The single-stranded multimeric nucleic acid molecules can be at least 10 kilobases in length. In some embodiments, the at least one of the plurality of sample nucleic acid molecules further comprises a double-stranded monomeric copy of the regularly occurring monomer unit. The plurality of oligonucleotide molecules can be present at about a uniform surface density across the surface. The plurality of oligonucleotide molecules can be present at a local surface density of at least about 100,000 molecules/μm² at a first position on the surface, and at a second local surface density at a second position on the surface. In some embodiments, the plurality of oligonucleotide molecules is present at a surface density of at least about 1,000 molecules/m².

In some embodiments, the first coating can comprise multiple hydrophilic polymer coating layers. The first coating can include a first layer comprising a monolayer of polymer molecules tethered to the surface of the substrate. The first coating can further include a second layer comprising a second monolayer of polymer molecules tethered to the polymer molecules of the first layer; and a third layer comprising a third monolayer of polymer molecules tethered to the polymer molecules of the second layer, wherein at least one of the first layer, the second layer, or the third layer comprises branched polymer molecules.

In some embodiments, the third layer can comprise oligonucleotides tethered to the polymer molecules of the third layer. The oligonucleotides tethered to the polymer molecules of the third layer can be distributed at a plurality of depths throughout the third layer.

In some embodiments, the first coating can comprise a fourth layer comprising branched polymer molecules tethered to the polymer molecules of the third layer, and a fifth layer comprising polymer molecules tethered to the branched polymer molecules of the fourth layer. In some embodiments, the polymer molecules of the fifth layer further comprise oligonucleotides tethered to the polymer molecules of the fifth layer. The oligonucleotides tethered to the polymer molecules of the fifth layer are distributed at a plurality of depths throughout the fifth layer.

In some embodiments, the hydrophilic polymer coating layer of the first coating can comprise a molecule selected from the group consisting of: polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo (ethylene glycol) methyl ether methacrylate) (POEGMA), polyglutamic acid (PGA), poly-lysine, poly-glucoside, streptavidin, and dextran.

In some embodiments, when the clonally-amplified sample nucleic acid molecules or complementary sequences thereof are labeled with Cyanine dye-3, an image of the surface exhibits a ratio of fluorescence intensities for the clonally-amplified, Cyanine dye-3-labeled sample nucleic acid molecules, or complementary sequences thereof, and nonspecific Cyanine dye-3 dye adsorption background (Bᵢₙₜₑᵣ) of at least 3:1.

In some embodiments when the image of the surface exhibits a ratio of fluorescence intensities for clonally amplified, Cyanine dye-3-labeled sample nucleic acid molecules, or complementary sequences thereof, and a combination of nonspecific Cyanine dye-3 dye adsorption background and nonspecific amplification background (Bᵢₙₜₑᵣ+Bᵢₙₜᵣₐ) of at least 3:1.

In some embodiments, when the clonally-amplified sample nucleic acid molecules or complementary sequences thereof are labeled with Cyanine dye-3, the image of the surface exhibits a ratio of fluorescence intensities for clonally-amplified, Cyanine dye-3-labeled sample nucleic acid molecules, or complementary sequences thereof, and nonspecific dye adsorption background (Bᵢₙₜₑᵣ) of at least 5:1.

In some embodiments, when the image of the surface exhibits a ratio of fluorescence intensities for clonally-amplified, Cyanine dye-3-labeled sample nucleic acid molecules, or complementary sequences thereof, and a combination of nonspecific Cyanine dye-3 dye adsorption background and nonspecific amplification background (Bᵢₙₜₑᵣ+Bᵢₙₜᵣₐ) of at least 5:1.

In some embodiments, when the clonally-amplified sample nucleic acid molecules or complementary sequences thereof are labeled with Cyanine dye-3, the fluorescence image of the surface exhibits a contrast-to-noise ratio (CNR) of at least 20 when the fluorescence image is acquired using an inverted microscope equipped with a 20× objective, NA=0.75, dichroic mirror optimized for 532 nm light, a bandpass filter optimized for Cyanine dye-3 emission, and a camera under non-signal saturating conditions, while the surface is immersed in a buffer.

In some embodiments, one or more of the interior surfaces 521a can be coated, in combination with the first coating 522a, a third coating of fluorescent beads (not shown).

The fluorescent beads can be chemically immobilized to the surface. The fluorescent beads can be covalently immobilized to the surface. The fluorescent beads can be immobilized or fixedly attached to the surface by forming a coating thereon, e.g., a third coating, so that the fluorescent beads remain fixed or immobilized relative to the surface 521a. The coating can be applied directly to and in contact with the surface 521a. Alternatively, the third coating can be applied indirectly to or not in direct contact with the surface 521a. In some embodiments, the third coating can be applied in between the surface 521a and the first coating 522a.

In some embodiments, the fluorescent beads are chemically immobilized to the surface. In some embodiments, the fluorescent beads are covalently immobilized to the surface. In some embodiments, the fluorescent beads are pre-activated to enable chemical attachment to the surface. In some embodiments, the fluorescent beads are pre-activated to enable covalent attachment to the surface. In some embodiments, the clusters or polonies of polynucleotides captured thereon and the fluorescent beads are imaged simultaneously in one or more sequencing cycles using the sequencing system 110.

In some embodiments, reagent(s) may be administered to the flow cell device through the channels to improve the wettability of the sample on the surface, e.g., the cultured cells or tissue. In some embodiments, such reagents may include various buffers used in sample preparation of DNA sequencing samples, e.g., the PBS buffer. In some embodiments, such reagent may include various surfactants. In some embodiments, reagent(s) may be administered to the flow cell device to improve the wettability of the sample on the surface by 1%, 2%, 5%, 10%, 15%, 20%, 25%, 30%, 50%, 80%, 100%, 120%, 150%, 180%, 200% or more in comparison to the wettability before administration of such reagent(s). In some embodiments, reagent(s) may be administered to the flow cell device through the microfluidic channels to reduce the surface tension of air/liquid interface(s) (e.g., the air/liquid interface of bubbles). In some embodiments, reagent(s) may be administered to the flow cell device to reduce surface tension by 1%, 2%, 5%, 10%, 15%, 20%, 25%, 30% 50%, 80%, 100%, 120%, 150%, 180%, 200% or more in comparison to the surface tension before administration of the reagent(s). In some embodiments, reagent(s) may be administered to the flow cell device to increase bubble size by 1%, 2%, 5%, 10%, 15%, 20%, 25%, 30%, 50%, 80%, 100%, 120%, 150%, 180%, 200% or more in comparison to the bubble size before administration of the reagent(s). In some embodiments, the reagents that improves the wettability of the sample may be administered during sample preparation. In some embodiments, the reagents that improves the wettability of the sample may be administered before flow cell assembly, e.g., assembly of the one or more substrates into the cell device. The flow cell assembly may include generating the flow cell device with sample(s) immobilized thereon to be sequenced during a sequencing run. In some embodiments, the reagents that improves the wettability of the sample may be administered during rolling circle amplification (RCA). In some embodiments, the reagents that improves the wettability of the sample may be administered before the start of the sequencing run to generate sequencing results. In some embodiments, the reagents that reduces the surface tension of air/liquid interface(s) (e.g., the air/liquid interface of bubbles) may be administered during sample preparation. In some embodiments, the reagents that reduces the surface tension of air/liquid interface(s) (e.g., the air/liquid interface of bubbles) may be administered before flow cell assembly. In some embodiments, the reagents that reduces the surface tension of air/liquid interface(s) (e.g., the air/liquid interface of bubbles) may be administered during rolling circle amplification (RCA). In some embodiments, the reagents that the reagents that reduces the surface tension of air/liquid interface(s) may be administered before the start of the sequencing run to generate sequencing results.

In some embodiments, the flow rate of reagent(s) during sample preparation may be within a range from 1 uL/sec to 5000 uL/sec. In some embodiments, the flow rate of reagent(s) during sample preparation may be within a range from 10 uL/sec to 1000 uL/sec. In some embodiments, the flow rate of reagent(s) during sample preparation may be within a range from 10 uL/sec to 500 uL/sec. In some embodiments, the flow rate of reagent(s) during sample preparation may be within a range from 20 uL/sec to 500 uL/sec. In some embodiments, the flow rate of reagent(s) during sample preparation may be within a range so that the shear stress on the sample and/or reagent(s) may be increased by 1%, 2%, 5%, 10%, 15%, 20%, 25%, 30%, 50%, 80%, 100%, 120%, 150%, 180%, 200%, 250%, 200%, 350%, 400%, 450%, 500%, 600%, or more in comparison to the shear stress of a previous flow rate of the reagent(s). In some embodiments, the reagents and/or sample may be flowed with a rate that within the predetermined flow rate range during rolling circle amplification (RCA).

In some embodiments, a negative pressure is applied to the flow cell device, and more particularly, to the lumen of the channels. The negative pressure may be applied during sample preparation and/or RCA. The negative pressure may be applied before the start of the sequencing run to generate sequencing results. In some embodiments, the negative pressure may be in the range of −1 kPa to −450 kPa. In some embodiments, the negative pressure may be in the range of −10 kPa to −350 kPa. In some embodiments, the negative pressure may be in the range of −10 kPa to −150 kPa. In some embodiments, the shear stress increase can be obtained by elevating the temperature to be within a temperature range of 30° C. to 80° C. and applying the negative pressure in the range of −10 kPa to −150 kPa.

In some embodiments, the reagents and/or sample may be heated to be within a temperature range of 30° C. to 80° C. for a predetermined duration during sample preparation so that the shear stress on the sample and/or reagent(s) may be increased by 1%, 2%, 5%, 10%, 15%, 20%, 25%, 30%, 50%, 80%, 100%, 120%, 150%, 180%, 200%, 250%, 200%, 350%, 400%, 450%, 500%, 600%, or more in comparison to the shear force before heating of the reagent(s) and/or samples. In some embodiments, the reagents and/or sample may be heated during rolling circle amplification (RCA).

### Open Landing Areas

The flow cell devices and systems can include one or more open landing areas 241, 341a, 441a, 541a, 741a. FIGS. 30-32, and 33A-33F show flow cell devices with the open landing area(s) for one or more channels.

The open landing area can be part of the inlet. The open landing area can be on a bottom substrate. The open landing area can be in fluidic connection with its corresponding channel(s). The open landing area can be in fluidic connection with a manifold or a connector. The open landing area 341a, 441a, 541a can be part of the inlet 340a, 440a, 540a. The open landing area 341a, 441a, 541a can be on a bottom substrate 330a, 430a, 530a. The open landing area 341a, 441a, 541a can be in fluidic connection with its corresponding channel(s) 350a, 450a, 550a.

The inlet can comprise a void or hole in the top substrate that is located above at least part of the open landing area 241, 341a, 441a, 541a, 741a. The air gap, or liquid reagents, or combinations thereof, can be introduced via the void or hole of the inlet to reach the open landing area, and then transfer from the open landing area into the corresponding channel(s). In some embodiments, the void or hole can have a cross section area in the x-y plane that is substantially identical or identical to the area of the open landing area. In some embodiments, the void or hole can have a cross section area in the x-y plane that is greater than the area of the open landing area. In some embodiments, the void or hole can be considered to have a rectangular-shaped cross section in the x-y plane which is as wide as the flow cell device along the x axis. The inlet 340a, 440a, 540a can comprise a void or hole in the top substrate 320a, 420a, 520a that is located above at least part of the open landing area 341a, 441a, 541a. The air gap, or liquid reagents, or combinations thereof, can be introduced via the void or hole of the inlet 340a, 440a, 540a to reach the open landing area 341a, 441a, 541a, and then transfer from the open landing area 341a, 441a, 541a into the corresponding channel(s) 350a, 450a, 550a. In some embodiments, the void or hole can have a cross section area in the x-y plane that is substantially identical or identical to the area of the open landing area, e.g., as in FIGS. 31-32, 33A and 33F. In some embodiments, the void or hole can have a cross section area in the x-y plane that is greater than the area of the open landing area, e.g., in FIG. 33E. The void or hole in FIG. 33E can be considered to have a rectangular shaped cross section in the x-y plane which is as wide as the flow cell device along the x axis.

The inlet and the open landing area can advantageously enable open administration of liquids or gas to the flow cell devices. The inlet 340a, 440a, 540a and the open landing area 341a, 441a, 541a can advantageously enable open administration of liquids or gas to the flow cell devices. The open administration via the open landing area enabled by the flow cell devices herein can advantageously remove series of closed tubing or locked-in tubing thereby greatly reducing system complexity and cost and allowing more flexible adaptation of the systems and devices for various sequencing applications. The open administration via the open landing area may also advantageously improve compatibility of the fluidic control and fluidic dispensing with different flow cell devices without the need to alter the closed tubing in existing sequencing systems. For example, each dispensing tip may be used for only a corresponding reagent without contamination with other reagents. As another example, multiple dispensing tips can be used for simultaneously administration of identical or different reagents to different channels to increase sequencing efficiency and reduce the sequencing time.

The size and shape of the hole or void, and the size and shape of the open landing area can vary in different embodiments. The sizes and shapes may be determined based on parameters in the specific sequencing application(s), such as, a flush volume, a contamination threshold, the dimensions of the flow cell, e.g., the width of the flow cell channels, or the parameters of the dispenser, e.g., the size of the dispensing tip. As a nonlimiting example, the hole or void is cylindrical with walls extending along the z direction and orthogonal to the substrates. As a nonlimiting example, the hole or void is cylindrical as shown in FIG. 33C with walls extending along the z direction and orthogonal to the substrates. However, the hole or void can be shaped, or sized, or combinations thereof, differently. For example, the hole or void can have an inverted cone shape with wider openings at the top and narrows down toward the channel to reduce the residuals of reagents that can remain in the inlet. In some embodiments, a larger open landing area may better facilitate reagent transfer into the channels and keeping the open landing area's size to the width of the channels in a predetermined ratio range may also better facilitate reagent transfer into the channels. As a nonlimiting example, the diameter of the opening area, e.g., the widest dimension in the x-y plane, can be in the range of about 3 mm to about 40 mm. In some embodiments, the diameter of the opening area is about identical to the width of the corresponding channel. In some embodiments, the diameter of the opening area is about 10%, 20%, 30%, 40% or 50% less than the width of the corresponding channel. As a nonlimiting example, the diameter of the hole or void, e.g., the widest dimension in the x-y plane, can be in the range of about 3 mm to about 40 mm. In some embodiments, the diameter of the hole or void is about identical to the width of the corresponding channel. In some embodiments, the diameter of the hole or void is about 10%, 20%, 30%, 40% or 50% less than the width of the corresponding channel.

To work with the open landing area, the flow cell system may include a fluidic control device which may comprise a dispenser that is configured to openly dispense one or more reagents to the inlet. To work with the open landing area, the flow cell system may include a fluidic control device which may comprise a dispenser 280a, 580a that is configured to openly dispense one or more reagents to the inlet 540a. The dispenser can openly dispense from a tip, via the void or hole of the inlet, to the open landing area. The dispenser can openly dispense from a tip, via the void or hole of the inlet, to the open landing area 341a, 541a. In some embodiments, there is no tubing connecting the dispenser and the inlet. In some embodiments, the dispenser directly contacts part of the inlet, e.g., the landing area, or a wall of the void, to openly dispense the reagents. In some embodiments, the dispenser does not directly contact any physical part of the inlet, but its tip may extend into the void or hole of the inlet. In some embodiments, at least part of the tip of the dispenser is in contact with the open landing area. In some embodiments, the tip of the dispenser is not in direct physical contact with the open landing area.

The dispenser may include more than one dispensing tip, e.g., pipette tips, so that each different reagent can have its own dispensing tip without mixing of reagents occurring in the dispenser or the dispensing tips. In a sense, the dispenser disclosed herein removes the common line in existing flow cell systems and reduces the dead volume in the common line in existing flow cell systems so that the required consumption of reagents for identical sequencing process can be significantly reduced. Further, removal of common line and usage of separate dispensing tips reduces mixing of reagents and the resulting contamination of reagents dispensed to the flow cell devices.

In some embodiments, the dispenser and its tip(s) may be manually operated for moving, or dispensing, or combinations thereof. In some embodiments, the dispenser and its tip(s) may be automatically operated for moving, or dispensing, or combinations thereof. For example, the dispenser may include an array of dispensing tips, each in fluidic communication with a reagent reservoir in a cartridge, and a robotic arm moves the array to position a corresponding tip above the landing area and then controls the dispensing. When a next reagent needs to be delivered, the robotic arm can withdraw the previous dispensing tip and locate the next reagent tip in the array for dispensing. The automatic operation of the dispenser and its tips may be controlled by a software executable on the hardware processor of the sequencing system herein. In some embodiments, multiple dispensing tips may be controlled to dispense simultaneously. In some embodiments, the same dispensing tip may be controlled to dispense to a first open landing area, and subsequently move to a second open landing area for dispensing.

### Filters

In some embodiments, the open landing area may be associated with the risk of being contaminated by external environment, e.g., dust, fiber, and debris. The contamination may enter the microfluidic channels from the open landing area. When the contamination goes into the microfluidic channel, it is possible that the contamination may stay in the microfluidic channel and cause changes to the surface, the flow pattern, as well as liquid exchange efficiency, reducing sequencing quality of areas nearby the contamination, e.g., fibers.

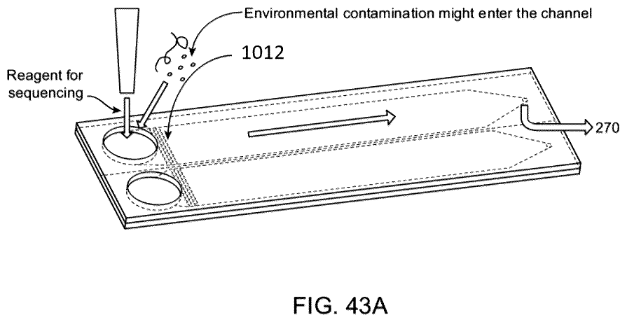

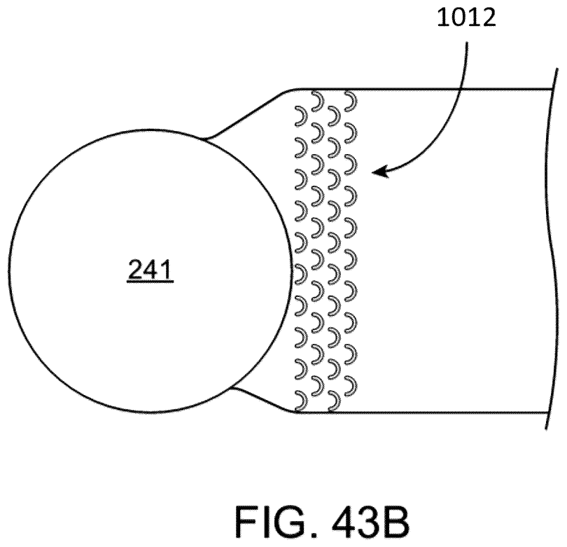

In some embodiments, the flow cell device may include one or more filters that are configured to capture or trap the contamination that may otherwise enter the microfluidic channel. The filter(s) may be installed at various positions between the open landing area and the microfluidic channel. FIGS. 43A-43B show an exemplary embodiment of the filter. In some embodiments, the filter is installed to capture the contamination from the environment. The filter(s) may advantageously facilitate capturing of contamination, i.e., any undesired particles or residuals from external environment to enter the microfluidic channels. The filter(s) may facilitate avoiding spatial block-out of the surface(s) of the microfluidic channel and preventing change of the flow pattern through the microfluidic channel to ensure efficient liquid exchange, thereby allowing accurate and reliable sequencing reactions.

In some embodiments, the filter(s) may be configured to capture or trap solid contamination. In some embodiments, the filter(s) may be configured to capture or trap air bubbles, e.g., within a diameter limitation. In some embodiments, the filter installation does not change fluidic mechanics in the microfluidic channels because the flow cycles can be optimized, e.g., the flow rate, flow speed, etc., for the installed filters to achieve the identical flow mechanics as desired before installation of the filters. In some embodiments, the filter does not change the average flow rate in the microfluidic channels but it may change the local flow field immediately nearby. In some embodiments, sequencing reactions immediately nearby the filter(s) may or may not be considered for sequencing results.

In some embodiments, the filter(s) may comprise various materials. In some embodiments, the filter(s) may comprises one or more identical materials that have been used to build the flow cell device. In some embodiment, the filter(s) may comprise one or more of: glass, plastic, polymer, and hydrogel. In some embodiments, the filter(s) may comprise one or more micro-fabricated materials.

In some embodiments, the filter(s) may include more than one filters 1012 positioned in a 3D pattern, e.g., as shown in FIG. 43B. In some embodiment, the filter(s) may be installed at various location on the FC device along the fluidic pathway that the sequencing reagents may travel after arriving at the open landing area and before existing the flow cell device. In some embodiments, different filters may be installed at different locations of the flow cell device. For example, the first filter(s) may be installed as shown in FIGS. 43A-43B close to the opening landing area. The second additional filter(s) may be installed at or near the center of the microfluidic channels to the right of the first filter(s) (not shown).

In some embodiments, the flow cell devices 112 further comprises a cleaning outlet 470a, 570a, 770a. The cleaning outlet 470a, 570a, 770a can be located in the one or more substrates, for example, in the bottom substrate 430a, 530a. In some embodiments, the cleaning outlet 470a, 570a, 770a may be located on a top substrate or in a middle substrate as a side port (not shown). In some embodiments, the side port extends at least along a direction that is perpendicular or nearly perpendicular to a y direction. In some embodiments, the side port extends at least along a direction that is perpendicular or nearly perpendicular to a z direction. In some embodiments, the side port extends at least along a direction that is oblique to an x direction. In some embodiments, the side port extends at least along a direction that is oblique to a y direction. In some embodiments, the side port extends at least along a direction that is oblique to a z direction. In some embodiments, the side port extends at least along a direction that is perpendicular or nearly perpendicular to an x-y plane. In some embodiments, the side port extends at least along a direction that is perpendicular or nearly perpendicular to an x-z plane. In some embodiments, the side port extends at least along a direction that is perpendicular or nearly perpendicular to an x-y plane. In some embodiments, the side port extends at least along a direction that is perpendicular or nearly perpendicular to a y-z plane. In some embodiments, the side port extends at least along a direction that is oblique to an x-y plane. In some embodiments, the side port extends at least along a direction that is oblique to an x-z plane. In some embodiments, the side port extends at least along a direction that is oblique to a y-z plane.

The cleaning outlet 470a, 570a, 770a can be in fluidic connection with the inlet 440a, 540a. In some embodiments, the cleaning outlet 470a, 570a, 770a is configured to be coupled with a fluid driving device, e.g., a pump or vacuum 471a of the fluidic control device. The pump 471a may be in addition to the pump 472a coupled to the outlet 460a. In some embodiments, a same fluid driving device, e.g., pump, can be coupled to the outlet 460a, 560a and the cleaning outlet 470a.

The distance from the cleaning outlet 470a, 570a, 770a can be shorter to the inlet 440a, 540a than to the outlet 460a, 560a. The distance can be within the x-y plane. The shorter distance from the cleaning outlet to the inlet is designed to facilitate transfer of liquid or gas from the open landing area to the cleaning outlet.

In some embodiments, the relative position of the cleaning outlet 470a, 570a, 770a to the inlet 440a, 540a, 740a can be different. In some embodiments, the cleaning outlet 770a can be directly underneath the open landing area 741a, e.g., in FIGS. 38A-38E and 39C. In such embodiments, the cleaning outlet 770a is directly connected to the open landing area.

In some embodiments, the cleaning outlet 470a, 570a may not be directly beneath the open landing area but of a distance to the open landing area, e.g., in FIGS. 32, 33A, 37A-37E, and 39A-39B. In such embodiments, the cleaning outlet 470a, 570a is not directly connected to the corresponding open landing area, but instead connected via a tapered transition portion 454a, 554a therebetween.

The distance from the cleaning outlet to the closest edge or the center of the open landing area can be 0 mm or about 0 mm. The distance from the cleaning outlet to the closest edge or the center of the open landing area, when the cleaning outlet is not directly underneath the open landing area, can be from about 0 mm to about 20 mm. The distance from the cleaning outlet to the closest edge or the center of the open landing area, when the cleaning outlet is not directly underneath the open landing area, can be from about 0 mm to about 15 mm. The distance from the cleaning outlet to the closest edge or the center of the open landing area, when the cleaning outlet is not directly underneath the open landing area, can be from about 0 mm to about 10 mm. The distance from the cleaning outlet to the closest edge or the center of the open landing area, when the cleaning outlet is not directly underneath the open landing area, can be from about 3 mm to about 10 mm.

The distance from the cleaning outlet to the closest edge or the center of the open landing area, when the cleaning outlet is not directly underneath the open landing area, can be from 0 mm to 15 mm. The distance from the cleaning outlet to the closest edge or the center of the open landing area, when the cleaning outlet is not directly underneath the open landing area, can be from 0 mm to 10 mm. The distance from the cleaning outlet to the closest edge or the center of the open landing area, when the cleaning outlet is not directly underneath the open landing area, can be from 3 mm to 10 mm.

In some embodiments, there can be residuals of reagents, such as meniscus, as shown in the bottom panel of FIG. 32, that remains on the open landing area, or on the wall(s) of the hole of the inlet, or combinations thereof. Such residuals, if not removed, may cause unintended mixing when a subsequent reagent is delivered to the open landing area and consequently contaminate sequencing reactions in the channels. Washing with liquid(s) alone may not be effective in removing such residual reagents as meniscus, so it may take multiple flushing of washing liquids to completely remove the residual in existing flow cell systems, with increased washing time and washing costs. The cleaning outlet 470a, 570a in fluidic connection can advantageously facilitate time- and cost-effective removal of such residuals. In some embodiments, a mechanical driving force can be applied, e.g., by a pump or an inlet vacuum, via the cleaning outlet, to completely remove such residual of reagents on the open landing area. As such, the required time and washing volume to remove the residuals to achieve a satisfactory contamination level can be effectively improved from existing flow cell devices.

The size and shape of the cleaning outlet may be customized to suite different sequencing applications. Although the cleaning outlet is shown as a cylinder in FIG. 33C, it can be made in different shapes, such as a cone, an inverted cone, etc. In some embodiments, the size and shape of the cleaning outlet can be identical to that of the outlet. In some embodiments, the size of the cleaning outlet can be no more than about 10%, 20%, or 30% different from that of the outlet. In some embodiments, the diameter of the cleaning outlet in the x-y plane is about 0.3 mm to about 10 mm. In some embodiments, the height of the cleaning outlet in the z direction is the same as the height of the bottom substrate. In some embodiments, the height of the cleaning outlet is about 0.3 mm to about 3 mm. In some embodiments, the height of the cleaning outlet is about 0.5 mm to about 1 mm. In some embodiments, the diameter of the cleaning outlet in the x-y plane is 0.3 mm to 10 mm. In some embodiments, the height of the cleaning outlet in the z direction is the same as the height of the bottom substrate. In some embodiments, the height of the cleaning outlet is 0.3 mm to 3 mm. In some embodiments, the height of the cleaning outlet is 0.5 mm to 1 mm.

### Slippery Coatings

In some embodiments, part of the substrate, other than the interior surface of the channels, can be covered with a second coating, e.g., a slippery coating, to facilitate transfer of fluids on the coating, either alone or in combination with the first coating disclosed herein. The second coating can be different from the first coating of the channels. The second coating can be applied directly to the substrate(s) without application of the first coating. The second coating can be applied to the substrate(s) on top of the application of the first coating.

The thickness of the coating along the z axis may be customized so that it does not interfere or reduce fluidic communication speed, or other fluidic parameter(s), or combinations thereof, to the channels in comparison to flow cell devices without the coating. The thickness of the coating along the z axis may be customized so that it increases or facilitates fluidic communication speed, or other fluidic parameter(s), or combinations thereof, to the channels in comparison to flow cell devices without the coating.

In some embodiments, the open landing area is covered with a coating. In some embodiments, the coating can be applied to at least part of the open landing area. In some embodiments, the coating can be applied to any combination of surfaces of the substrates except the interior surfaces defining the lumen of the channels. The coating can effectively facilitate liquid transfer from the open landing area to the channels and/or to a cleaning outlet to exit the flow cell device. For example, the coating may help reduce the volume of residual reagents on the open landing area when the reagent(s) is transferred into the channels. As another example, the coating may facilitate complete removal of the residual reagents on the open landing area, when an inlet vacuuming force is applied via the cleaning outlet.

In some embodiments, the open landing area 341a is covered with the second coating 342a. FIG. 31 shows an embodiment of the second coating 342a on the open landing area 341a, the rest of the open landing area 343a, and the part of the top substrate that is above the open landing area 343a. The right panel of FIG. 31 shows a schematic drawing of the second coating 342a with a liquid droplet of a reagent thereon. In some embodiments, the second coating 342a can be applied to at least part of the open landing area 341a. In some embodiments, the second coating 342a can be applied to any combination of surfaces of the substrates except the interior surfaces defining the lumen of the channels. The second coating 342a can effectively facilitate liquid transfer from the open landing area 341a to the channels 350a, 550a or to a cleaning outlet 570a to exit the flow cell device. For example, the second coating 342a may help reduce the volume of residual reagents on the open landing area when the reagent(s) is transferred into the channels. As another example, the second coating 342a may facilitate complete removal of the residual reagents on the open landing area, when an inlet vacuuming force is applied via the cleaning outlet.

In some embodiments, the coating can be various liquid-repelling coating(s). In some embodiments, the coating can be an omniphobic coating. In some embodiments, the coating comprises a slippery omniphobic covalently attached liquid (SOCAL) coating. In some embodiments, the coating comprises a liquid-like polymer brush surface that is covalently attached to the one or more substrates. In some embodiments, the coating is formed by acid-catalyzed graft polycondensation of one or more saline monomers. The one or more saline monomers can comprise dimethyldimethoxysilane (PDMS). In some embodiments, the one or more saline monomers can have a low surface energy that is below about 10, 15, 20, 25, or 20 mJ/m².

In some embodiments, the second coating 342a can be any liquid-repelling coating. In some embodiments, the second coating can be an omniphobic coating. In some embodiments, the second coating comprises a slippery omniphobic covalently attached liquid (SOCAL) coating. In some embodiments, the second coating comprises a liquid-like polymer brush surface that is covalently attached to the one or more substrates. In some embodiments, the second coating is formed by acid-catalyzed graft polycondensation of one or more saline monomers. The one or more saline monomers can comprise dimethyldimethoxysilane (PDMS). In some embodiments, the one or more saline monomers can have a low surface energy that is below about 10, 15, 20, 25, or 20 mJ/m².

The coating or second coating can be formed using various methods. For example, it can be formed by impregnating lubricants in one or more porous surfaces. In some embodiments, the coating comprises a slippery liquid-infused porous surface (SLIPS). In some embodiments, the lubricants comprise a liquid with a low surface energy, where the low surface energy is below a predetermined threshold. The predetermined threshold can be about 20 milliJoule per square meter (mJ/m²). In some embodiments, the predetermined threshold can be about 10, 12, 14, 16, 18, 20, 22, 24, 26, 28, or 30 mJ/m². In some embodiments, the lubricants comprise a silicone oil. In some embodiments, the coating comprises low surface energy that is below about 10, 15, 20, 25, or 20 mJ/m².

### Contamination Levels and COGS Savings

In some embodiments, one or more of the open landing area with open dispensing, the channel coating, the slippery coating of the open landing area, and the cleaning outlet and vacuuming can be used alone or in different combinations to achieve cleaning of the flow cell devices. FIGS. 30-32, 33A-33F show nonlimiting embodiments of the combination of one or more of them in the flow cell devices.

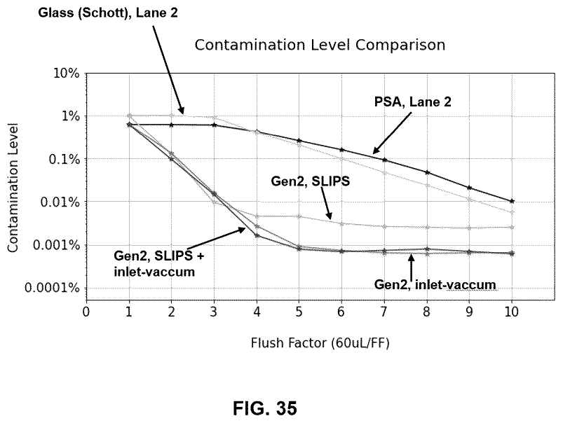

FIG. 35 shows the contamination level of flow cell devices disclosed herein, in comparison to existing flow cell devices. Images of the flow cell channels are acquired per flush volume. The flushing in this embodiment is about 60 microliter (μL), determined based on at least the channel size and geometry. The contamination of flow cell channels, on average, is about 1% for all three flow cell devices and two existing flow cells. The contamination level starts to decrease as the number of flush volume increases. When the flush factor reaches 5, the total volume of flushed reagents reaches about 300 μL. At this flushing factor, the contamination level of three flow cell devices disclosed herein, with SLIPS coating on the open landing read, with inlet vacuum from the cleaning outlet, or their combinations, exhibit a contamination level of lower than 0.01%, while the contamination level of existing flow cells are significantly higher, at above 0.1%. It takes more than 10 flush factors or over 600 μL of washing reagents for the existing flow cell devices to achieve similar contamination level as in the three flow cell devices disclosed herein. In this particular embodiment, flushing with more than 300 μL does not further decrease the contamination level to a significant level. The contamination level of about 0.001% can be satisfactory for all of the reagents used in the NGS sequencing application. The three flow cell devices advantageously achieved contamination levels for accurate and reliable sequencing process with a significant reduction in Cost of Goods Sold (COGS) than existing methods.

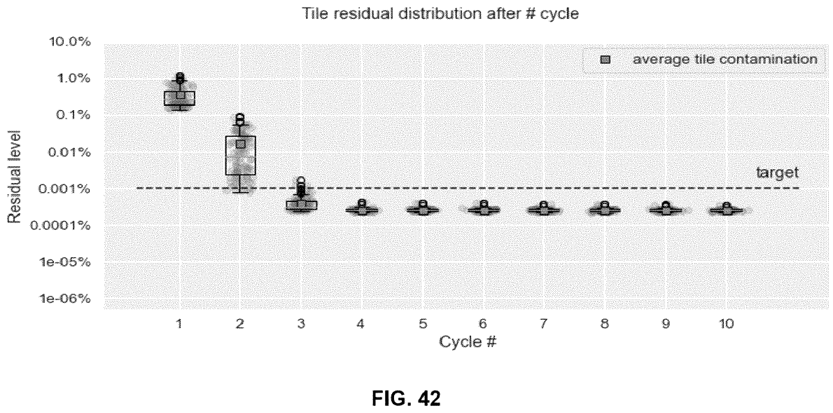

FIG. 42 shows residual level or contamination level that is averaged among different tiles of a flow cell device disclosed herein. Tile contamination variation across the flow cell device can be caused by the spatial location of the tile on the flow cell and its relative position to the inlet, or the outlet, or combinations thereof. Average contamination levels of different tiles of the flow cell devices disclosed herein are effectively reduced to be less than 1% by the first cycle of flush volume. By the third flush volume or cycle, the residual or contamination level of different tiles are all reduced to be below 0.001%. The average tile contamination is below the level of 0.001% by the third flush volume. The individual tile contaminations across the flow cell are below the level of 0.001% by the third or fourth flush volume. The flush volume is about 60 uL so that the contamination level for individual tiles regardless of its spatial location on the flow cell is reduced to be below 0.001% with a total flush volume of reagents or washing liquids of 240 uL.

In an embodiment, the required volume of sequencing-by-avidite reagents for stepping, cleaving, and imaging are all significantly reduced by using the flow cell devices disclosed herein. The stepping reagent requires a volume of about 430 uL, and it was reduced to about 90 uL with active volume reduction (AVR) to recycle a certain portion of the reagents. The AVR can be used in both existing flow cells systems and the flow cell systems disclosed herein. The AVR can be about 40%, 50%, 60%, 70%, 80%, or 90% of the total volume that is required with respect to a sequencing application. The total volume can be a volume without AVR. The reagents saving with AVR is about 5× in comparison to existing flow cell devices. Without AVR, the reduction can still be about 2.5× in comparison to existing flow cell devices. In some embodiments, the cleaving, trapping, and imaging reagents are reduced from about 300 uL to about 60 uL, with AVR. Table 1 below shows the volume of sequencing reagents required in using an existing flow cell system and COGS saving or reduced volume of reagents required using a flow cell device disclosed herein.

TABLE 1
Reduction of reagent consumption during a same sequencing
application achieved by a flow cell system disclosed herein
in comparison to an existing flow cell system.

| | | | Flow | | Baseline v6.0 | | COGS saving | |
|---|---|---|---|---|---|---|---|---|
| | | Temp | rate | Incubation | Volume | | Volume | |
| Steps | Reagent | (c.) | (μl/s) | (s) | (μl) | FF* | (μl) | FF* |
| Step | Removal | 42 | 56.3 | 0 | 300 | 5 | 60 | 1 |
| | UWB 1 | 52 | 98 | 0 | 600 | 10 | 180 | 1 |
| | Step Mix | 52 | 60 | 40 | 430 | 7.2 | 86 | 1.4 |
| Cleave | Removal | 51 | 103 | 0 | 300 | 5 | 60 | 1 |
| | Cleave | 51 | 65 | 30 | 300 | 5 | 60 | 1 |
| Trap | UWB 2 | 42 | 100 | 0 | 480 | 8 | 120 | 2 |
| | UWB 3 (PT 3) | 42 | 100 | 0 | 420 | 7 | 120 | 2 |
| | Trap Mix | 42 | 50 | 45 | 300 | 5 | 60 | 1 |
| | Imaging | 20 | 30.5 | 0 | 300 | 5 | 60 | 1 |

Image

### Fluidic Control Devices

Disclosed herein are fluidic control devices that can be coupled to the flow cell devices and actively apply mechanical forces for dispensing or collecting liquids, or gas, or combinations thereof, from the flow cell devices.

In some embodiments, the fluidic control devices can comprise a pump, a vacuum, or any other device that can actively apply a mechanical force to the lumen of the channels, or the open landing area, or combinations thereof, via the outlet or cleaning outlet. FIG. 32 shows a fluidic control device with a vacuum 472a that is coupled to all outlets 460a of the flow cell device 4112. FIG. 32 shows another vacuum 471a that is coupled to the cleaning outlet 470a of the flow cell device 112. The vacuums 471a and 472a can be the same vacuum or pump.

In some embodiments, the fluidic control devices can comprise a dispenser 280a, 580a with one or more dispensing tips. The dispenser 280a, 580a can dispense preset amounts of reagents within a certain time window to the inlet.

In some embodiments, the fluidic control devices can comprise a robotic arm that controls movement of the dispenser. In some embodiments, the robotic arm can move the dispenser in 3D space so that the dispensing tip can reach a specific location before it starts dispensing. In some embodiments, the robotic arm can retrieve a dispensing tip after one dispense and move a second dispending tip to a location for a subsequent one.

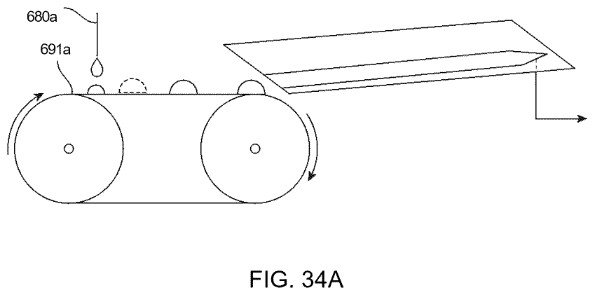

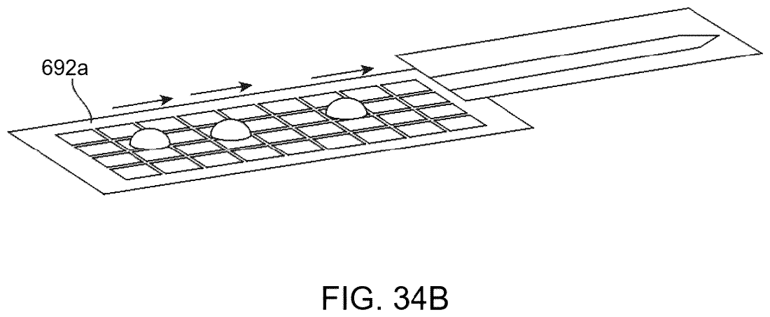

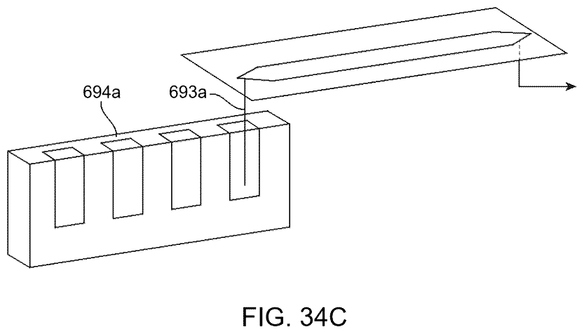

In some embodiments, the fluidic control devices can comprise a dispensing roller configured to dispense the reagents as shown in FIG. 34A. The reagents can be dispensed by the dispenser 680a to a continuous track 691a rolled on one or more wheels, and the wheels of the roller can roll the track 691a and the reagents to an open landing area of the flow cell. In this particular embodiment, the inlet can be a side-port at an edge of the substrates. There can be an active force applied at the outlet to facilitate delivery of the reagents from the track to the inlet.

In some embodiments, the fluidic control devices can comprise a dispensing plate with an electrowetting surface. As shown in FIG. 34B, the dispensing plate 692a can be translated, thereby translating the reagents dispensed thereon to the inlet, which in this embodiment, is a side-port at an edge of the substrates.

In some embodiments, the fluidic control devices can comprise a reagent reservoir and a sipper as shown in FIG. 34C. In this particular embodiment, one end of the sipper 693a can be inserted in a reagent reservoir 694a, and the other end of the sipper can point to or be in contact with the inlet. The reagent can be sucked out in a controlled fashion to the open landing area of the flow cell. The open landing area, in this embodiment, is facing downward, and the hole or void of the inlet is in the bottom substrate. Various mechanisms can be used to control the sipping action. For example, an active mechanical force can be applied from the outlet to sip a predetermined amount of reagent from the reservoir. A different sipper can be used for a different reagent to avoid unintended mixing of reagents in the sipper 693a.

### Methods

Disclosed herein are methods of using the flow cell devices 112 for performing, or facilitating, or combinations thereof, sequencing analysis using the sequencing system 110. Disclosed herein are also methods of manufacturing the flow cell devices 112 that can be used to perform, or to facilitate, or combinations thereof, sequencing analysis. The methods herein can include some or all of the operations disclosed herein. The operations may be performed in, but is not limited to, the order that is described herein.

The operations herein may be performed manually. The operations may be automatically performed by a robotic arm or the like (not shown). The robotic arm can be controlled by a computer system, e.g., 126 in FIG. 1, to automatically perform some or all of the operations disclosed herein. Alternatively, the computer system 126, dedicated processors, 118, the FPGA(s) 120, or their combinations, may be programmed to control the robotic arm. The computer system of the robotic arm can have installed on it software, firmware, hardware, or their combinations that in operation cause the computer system to perform the operations or actions disclosed herein.

The methods can be performed by one or more processors in the computer system, e.g., 126, disclosed herein. In some embodiments, the processor can include one or more of: a processing unit, an integrated circuit, or their combinations. For example, the processing unit can include a central processing unit (CPU), or a graphic processing unit (GPU), or combinations thereof. The integrated circuit can include a chip such as a field-programmable gate array (FPGA). In some embodiments, the processor can include the computing system. In some embodiments, some or all of the operations in the methods herein may be performed by one or more of: FPGAs, ASIC chips, neural processing units (NPUs), artificial intelligence chips (AI chips), tensor processing units (TPUs), graphic processing units (GPU).

In some embodiments, some or all operations in the methods can be performed by the FPGA(s). In embodiments when some operations are performed by FPGA(s), the data after an operation performed by the FPGA(s) can be communicated by the FPGA(s) s to the CPU(s) so that the CPU(s) can perform subsequent operation(s) in method using such data. Similarly, data can also be communicated from the CPU(s) to the FPGA(s) for processing by the FPGA(s). In some embodiments, all the operations in methods can be performed by CPU(s). Alternatively, the operations performed by CPU(s) can be performed by other processors such as the dedicated processors, or FPGAs. In some embodiments, all the operations in method can be performed by FPGA(s).

The methods of manufacturing the flow cell devices disclosed herein can comprise an operation of obtaining the one or more substrates. The operation of obtaining the one or more substrates can comprises obtaining the one or more substrates separately so that the one or more substrates are not physically coupled or bonded to each other yet.

The methods disclosed herein can comprise an operation of generating one or more channels in the one or more substrates. In some embodiments, the channels are generated as holes completely in the middle substrates. In some embodiments, generating a channel comprises generating a grove in the top or bottom substrates and generating a hole in the middle substrate, and the channel can be formed by stacking the grove and the hole together. In some embodiments, generating a channel comprises generating a grove in each of the two adjacent substrates and combining the groves together to form the channel, via etching or any other mechanisms. The present disclosure does not limit the mechanisms by which the hole, groove, or cavity, can be formed in the substrates. The hole, groove, or cavity can form a lumen that allows fluids and a gas gap between the fluids to flow therethrough, when the substrates are fixedly coupled together, e.g., bonded.

The methods disclosed herein can comprise an operation of forming an inlet. The operation of forming an inlet can comprise forming a hole or a void in at least one of the one or more substrates and forming an open landing area. The hole or void can be at or near one end of the substrates, or the channels, or combinations thereof. For example, forming the inlet can comprise forming a cylinder hole in the top substrate and forming an open landing area in the middle substrate that matches the location of the cylinder hole, e.g., at the same location, so that when the two substrates are stacked together, the hole is directly above the landing area or at least partly above the landing area.

The methods disclosed herein can comprise an operation of forming an outlet. The operation of forming an outlet can comprise forming a hole or a void in at least one of the one or more substrates. For example, forming the outlet can comprise forming a cylinder hole in the bottom substrate at or near the opposite end of the substrates, or channels, or combinations thereof, from the inlet.

In some embodiments, the inlet and outlet are in fluidic connection with the one or more channels.

The methods disclosed herein can comprise an operation of fixedly coupling the substrates together, e.g., bonding the substrate with pressure sensitive adhesive. The coupling operation can be achieved via chemical, mechanical, or laser bonding, but is not limited to such bonding techniques.

The methods disclosed herein can comprise coating at least a portion of a surface of the one or more channels with a first coating, as disclosed herein. The surface can be interior surface defining the lumen(s) of the one or more channels. For example, the surface can include a top or bottom interior surface.

The methods disclosed herein can comprise coating at least a portion of a surface of the one or more channels with an additional coating to the first coating, e.g., a third coating of fluorescent beads.

The methods disclosed herein can comprise an operation of covering at least a portion of the open landing area with a second coating as disclosed herein. The second coating can be different from or identical to the first coating in the channels. In some embodiments, the process of applying the second coating can be different from or identical to applying the first coating in the channels. In some embodiments, at least some actions in the entire process of applying the second coating can be different from or identical to applying the first coating in the channels.

In some embodiments, coating the open landing area comprises impregnating lubricants in one or more porous surfaces. In some embodiments, coating the open landing area comprises acid-catalyzed graft polycondensation of one or more saline monomers.

In some embodiments, the methods of manufacturing the flow cell devices further comprises an operation of forming a cleaning outlet in the one or more substrates. The operation of forming the cleaning outlet can comprise forming the cleaning outlet in fluidic connection with the inlet and positioning the cleaning outlet so that it is closer to the inlet than to the outlet. The operation of forming the cleaning outlet can further comprise forming the cleaning outlet in a predetermined size and shape. For example, the size and shape of the cleaning outlet can be approximately the same as the outlet. The operation of forming the cleaning outlet can further comprise forming the cleaning outlet in the bottom substrate, the top substrate, the middle substrate, or their combinations. As an example, the cleaning outlet can be a side port formed by a half grove in the middle substrate and the bottom substrate. In some embodiments, the side port extends at least along a direction that is perpendicular or nearly perpendicular to a y direction. In some embodiments, the side port extends at least along a direction that is perpendicular or nearly perpendicular to a z direction. In some embodiments, the side port extends at least along a direction that is oblique to an x direction. In some embodiments, the side port extends at least along a direction that is oblique to a y direction. In some embodiments, the side port extends at least along a direction that is oblique to a z direction. In some embodiments, the side port extends at least along a direction that is perpendicular or nearly perpendicular to an x-y plane. In some embodiments, the side port extends at least along a direction that is perpendicular or nearly perpendicular to an x-z plane. In some embodiments, the side port extends at least along a direction that is perpendicular or nearly perpendicular to an x-y plane. In some embodiments, the side port extends at least along a direction that is perpendicular or nearly perpendicular to a y-z plane. In some embodiments, the side port extends at least along a direction that is oblique to an x-y plane. In some embodiments, the side port extends at least along a direction that is oblique to an x-z plane. In some embodiments, the side port extends at least along a direction that is oblique to a y-z plane.

The methods of using the flow cells disclosed herein can comprise an operation of dispensing a first reagent openly to an open landing area of an inlet of the flow cell device. The dispensing operation can be performed manually by a user or automatically by a robotic arm. The dispensing of the first reagent can be from a dispensing tip of a dispenser of the fluidic control device disclosed herein. In some embodiments, a dispensing tip is used for dispensing the first reagent but not any other reagents to avoid unintended mixing of reagents in the dispensing tip.

The methods can further comprise an operation of moving the dispensing tip to a specific location before dispensing. The specific location can be above the hole of the inlet of the flow cell. The specific location can be that the tip is at least part inside the hole of the inlet. In some embodiments, at least part of the tip is in contact with the wall of the hole or the open landing area at the bottom of the hole. In some embodiments, the dispensing tip may comprise a shock absorbing portion that contacts the open landing area without exerting damaging force to the open landing area or the substrate(s). The dispending operation can last for a predetermined period of time to ensure a predetermined amount of first reagent is dispensed into the outlet. The predetermined dispensing time can be on the scale of sub seconds to less than a minute.

The methods can further comprise an operation of retrieving the dispensing tip from the specific dispensing location.

The methods can further comprise an operation of flowing at least part of the first reagent from the open landing area to one or more channels of the flow cell device. This operation of flowing the reagents can be driven passively without actively adding any mechanical force on the reagents. Alternatively, the operation can be facilitated by adding a mechanical force to transfer the reagent from one end of the channels in direct contact with the open landing area to the opposite end of the channels that is in contact with the outlet. For example, the force can be applied by a pump or a vacuum at the outlet. The sequencing reactions can occur when the first reagent flows through the channels.

The methods can further comprise an operation of cleaning residuals of the first reagent from the one or more channels by driving an air gap before dispensing any second reagent to the flow cell device. The air gap can be driven by a mechanical force applied by a pump or a vacuum at the outlet. The air gap may also help clean some of the residuals on the open landing area. The mechanical force can be adjusted so that the air gap can occupy about 30% to about 80% volume of each channel in a predetermined time window. Channels with a larger lumen may need a larger air gap for similar cleaning effect as compared to channels with smaller lumens.

The methods can further comprise an operation of washing the channels before dispensing any second reagents to achieve a cleaning effect.

The methods can further comprise an operation of dispensing a second reagent openly to the open landing area via a different dispensing tip from that of the first reagent when the second reagent is different from the first reagent.

Before dispensing the second reagent, the methods can further comprise an operation of confirming that the channels have been cleaned and a predetermined cleaning threshold has been met. For example, the predetermined cleaning threshold can be a contamination level that is required for the second reagent that is going to be administered.

The methods can further comprise an operation of facilitating cleaning of residuals of the first reagent off the open landing area by using a coating on at least part of the open landing area. The residuals of the first reagent on the open landing area may also contaminate the second reagent to be administered subsequent to the first reagent. Cleaning of such residuals can also help reduce contamination level of the second reagent and thus improve accuracy and reliability of the sequencing reactions based on the second reagent. The coating, e.g., liquid repelling or slippery, on the open landing area can passively facilitate transfer of the first reagent to the channels and reduce residuals on the open landing area.

The methods can further comprise an operation of cleaning residuals of the first reagent from at least part of the open landing area by driving the residuals through the cleaning outlet. An active mechanical force can be applied via the cleaning outlet, e.g., by a pump or a vacuum, to suck the residuals from the open landing area to the cleaning outlet. The active mechanical force can be combined with a passive second coating on the open landing area to facilitate cleaning of the open landing area before administration of the second reagent.

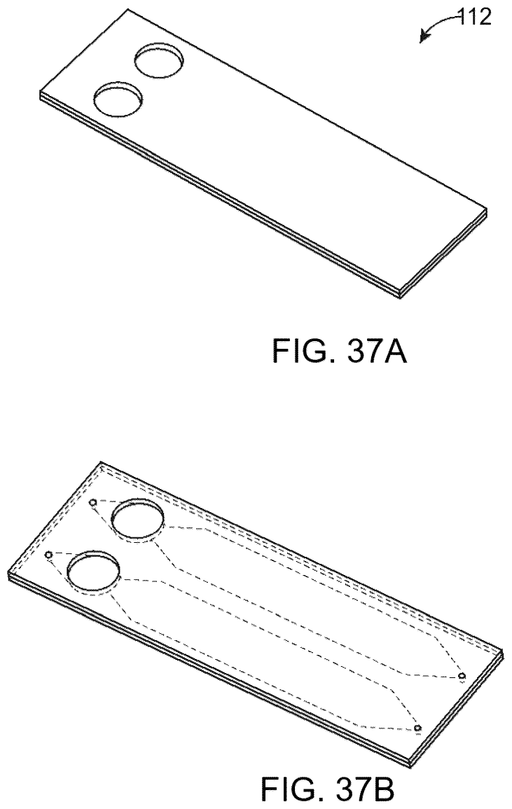

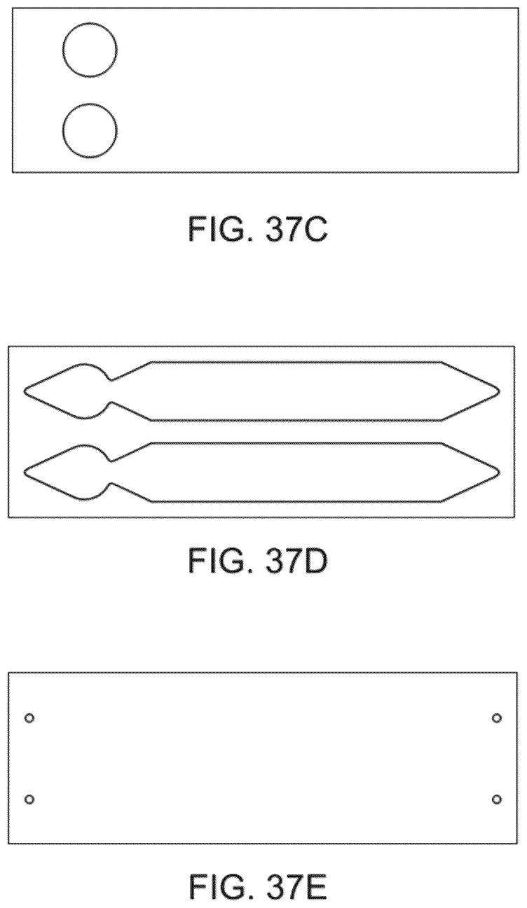

FIGS. 37A-37E and FIGS. 38A-38E show embodiments of the flow cell devices disclosed herein. Structural elements of the flow cell devices disclosed herein can have varying sizes. Such structural elements can include, but are not limited to, the inlet, the open landing area, the outlet, the tapered transition portion from the cleaning outlet to the open landing area or the inlet, the tapered transition portion from the inlet to the corresponding channel, and the tapered transition portion from the channel to the corresponding outlet. FIGS. 39A-39C shows embodiments of flow cell devices in which the sizes of the open landing area, the tapered transition portion from the cleaning outlet to the inlet or the open landing area, the tapered transition portion from the inlet or the opening landing area to the channel, or their combinations, are altered from the flow cell devices in FIGS. 37A-37E and FIGS. 38A-38E. FIGS. 39A-39C are non-limiting examples of the flow cell device disclosed herein. FIG. 39A is a top view of an embodiment of the flow cell device. FIG. 39B is a top view of another embodiment of the flow cell device. The flow device in FIG. 39A comprises a differently sized open landing area and inlet as compared to the flow cell device in FIG. 39B or FIGS. 37A-37E. The tapered transition portion from the cleaning outlet to the open landing area of the flow cell device in FIG. 39A is also altered from embodiments in FIG. 39B or FIGS. 37A-37E. FIG. 39C is a top view of yet another embodiment of the flow cell device. The flow device in FIG. 39C comprises a differently sized open landing area and inlet as compared to the flow cell device in FIGS. 38A-38E.

FIGS. 40A-40G show embodiments of the flow cell device disclosed herein. In these particular embodiments, as shown in FIG. 40A, the total thickness of the flow cell device is 2.07 mm. The top and the bottom substrates have thicknesses of 1 mm.

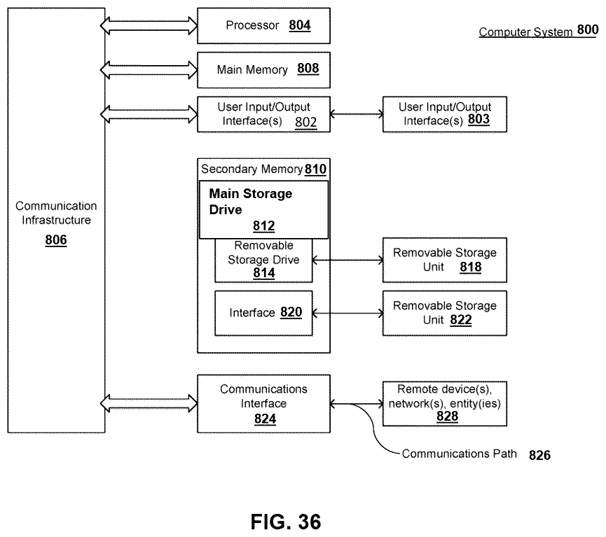

Various embodiments of the methods may be implemented, for example, using one or more computer systems, such as computer system 800 shown in FIG. 36. One or more computer systems 800 may be used, for example, to implement any of the embodiments discussed herein, as well as combinations and sub-combinations thereof. The flatness of surface A in FIG. 40A from its peak to valley, e.g., the difference between the highest and lowest points on the surface, is less than 0.02 mm. The flatness of surface B in FIG. 40B from its peak to valley, e.g., the difference between the highest and lowest points on the surface, is less than 0.02 mm. FIG. 40B shows that each channel edge to the edge of the flow cell device along x axis can be about 2.24 mm. The channels can have a width of 8.5 mm. The gap between the two channels along the x axis can be 3.5 mm. Each lane starts at 11 mm away from the closest edge of the flow cell device along the y axis. FIG. 40C shows that the cleaning outlet is 6.5 mm away from the edge of the flow cell device along the x axis, and the two cleaning outlets are 12 mm apart from each other along x axis. The cleaning outlets are 3 mm away from the closest edge of the flow cell device along the y axis. The diameter of the cleaning outlet is 0.81 mm. Alignment element “1” in FIG. 40C is configured to align the flow cell devices to the moving stage that can hold the flow cell device and move it relative to the optical system, which is positioned 11.25 mm from one edge of the flow cell device and 13.75 mm from the other edge of the flow cell device along the x axis, and positioned between the opening landing areas. As shown in FIG. 40C, the outlets are of the same dimension as the cleaning outlets. The total width of the flow cell device is 25 mm. The total length of the flow cell device is 75 mm. The cleaning outlets are 3 mm away from one edge of the flow cell device along the y axis. The outlets are 3 mm away from the opposite edge of the flow cell device along the y axis. FIG. 40G shows the middle substrate of 0.07 mm. FIGS. 40E and 40F show that the open landing area has a circular shape with a diameter of 8.52 mm. The tapered transition from the outlet to the body of the channel includes a curved portion that is a portion of a circular shape with a radius of 0.5 mm, and the angle defined between the tapered transition portion is 50.2 degrees.

In some embodiments, the curved portion of the tapered transition portion, as shown in FIG. 40E can be a portion of a circular shape with a radius in the range of about 0.2 mm to about 1.5 mm. In some embodiments, the curved portion can be a portion of a circular shape with a radius in the range of about 0.3 mm to about 0.9 mm. In some embodiments, the curved portion can be a portion of a circular shape with a radius in the range of about 0.4 mm to about 0.7 mm.

In some embodiments, the curved portion can be a portion of a circular shape with a radius in the range of 0.2 mm to 1.5 mm. In some embodiments, the curved portion can be a portion of a circular shape with a radius in the range of 0.3 mm to 0.9 mm. In some embodiments, the curved portion can be a portion of a circular shape with a radius in the range of 0.4 mm to 0.7 mm.

### Computer Systems

Computer system 800 may include one or more hardware processors 804. The hardware processor 804 can be central processing unit (CPU), graphic processing units (GPU), or their combination. Processor 804 may be connected to a bus or communication infrastructure 806.

Computer system 800 may also include user input/output interface(s) 803, such as monitors, keyboards, pointing devices, etc., which may communicate with communication infrastructure 806 through user input/output interface(s) 802. The user input/output interfaces 803 may be coupled to the user interface 124 in FIG. 1.

One or more of processors 804 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, vector processing, array processing, etc., as well as cryptography (including brute-force cracking), generating cryptographic hashes or hash sequences, solving partial hash-inversion problems, or producing results of other proof-of-work computations for some blockchain-based applications, or combinations thereof, for example. With capabilities of general-purpose computing on graphics processing units (GPGPU), the GPU may be particularly useful in at least the image recognition and machine learning aspects described herein.

Additionally, one or more of processors 804 may include a coprocessor or other implementation of logic for accelerating cryptographic calculations or other specialized mathematical functions, including hardware-accelerated cryptographic coprocessors. Such accelerated processors may further include instruction set(s) for acceleration using coprocessors, or other logic, or combinations thereof, to facilitate such acceleration.

Computer system 800 may also include a data storage device such as a main or primary memory 808, e.g., random access memory (RAM). Main memory 808 may include one or more levels of cache. Main memory 808 may have stored therein control logic (e.g., computer software), or data, or combinations thereof.

Computer system 800 may also include one or more secondary data storage devices or secondary memory 810. Secondary memory 810 may include, for example, a main storage drive 812, or a removable storage device or drive 814, or combinations thereof. Main storage drive 812 may be a hard disk drive or solid-state drive, for example. Removable storage drive 814 may be a floppy disk drive, a magnetic tape drive, a compact disk drive, an optical storage device, a tape backup device, or any other storage device/drive, or combinations thereof.

Removable storage drive 814 may interact with a removable storage unit 818.

Removable storage unit 818 may include a computer usable or readable storage device having stored thereon computer software, or data, or combinations thereof. The software can include control logic. The software may include instructions executable by the hardware processor(s) 804. Removable storage unit 818 may be a floppy disk, magnetic tape, compact disk, DVD, optical storage disk, and any other computer data storage device. Removable storage drive 814 may read from, or write to, or combinations thereof, removable storage unit 818.

Secondary memory 810 may include other methods, devices, components, instrumentalities or other approaches for allowing computer programs, or other instructions or data, or combinations thereof, to be accessed by computer system 800. Such methods, devices, components, instrumentalities or other approaches may include, for example, a removable storage unit 822 and an interface 820. Examples of the removable storage unit 822 and the interface 820 may include a program cartridge and cartridge interface (such as that found in video game devices), a removable memory chip (such as an EPROM or PROM) and associated socket, a memory stick and USB port, a memory card and associated memory card slot, or any other removable storage unit and associated interface, or combinations thereof.

Computer system 800 may further include a communication or network interface 824. The communication interface 824 may enable computer system 800 to communicate and interact with any combination of external devices, external networks, external entities, etc. (individually and collectively referenced by reference number 828). For example, communication interface 824 may allow computer system 800 to communicate with external or remote devices 828 over communication path 826, which may be wired, or wireless, or combinations thereof, and which may include any combination of LANs, WANs, the Internet, etc. Control logic, or data, or combinations thereof, may be transmitted to and from computer system 800 via communication path 826. In some embodiments, communication path 826 is the connection to the cloud 130, as depicted in FIG. 1. The external devices, etc. referred to by reference number 828 may be devices, networks, entities, etc. in the cloud 130.

Computer system 800 may also be any of a personal digital assistant (PDA), desktop workstation, laptop or notebook computer, netbook, tablet, smart phone, smart watch or other wearable, appliance, part of the Internet of Things (IoT), or embedded system, to name a few non-limiting examples, or any combination thereof.

It can be appreciated that the framework described herein may be implemented as a method, process, apparatus, system, or article of manufacture such as a non-transitory computer-readable medium or device. For illustration purposes, the present framework may be described in the context of distributed ledgers being publicly available, or at least available to untrusted third parties. One example as a modern use case is with blockchain-based systems. It can be appreciated, however, that the present framework may also be applied in other settings where sensitive or confidential information may need to pass by or through hands of untrusted third parties, and that this technology is in no way limited to distributed ledgers or blockchain uses.

Computer system 800 may be a client or server, accessing or hosting any applications, or data, or combinations thereof, through any delivery paradigm, including but not limited to: remote or distributed cloud computing solutions; local or on-premises software (e.g., “on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), database as a service (DBaaS), etc.); or a hybrid model including any combination of the foregoing examples or other services or delivery paradigms.

Any applicable data structures, file formats, and schemas may be derived from standards including but not limited to: JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with existing or open standards.

Any pertinent data, files, or databases, or combinations thereof, may be stored, retrieved, accessed, or transmitted, or combinations thereof, in human-readable formats such as numeric, textual, graphic, or multimedia formats, further including various types of markup language, among other possible formats. Alternatively, or in combination with the above formats, the data, files, or databases, or combinations thereof, may be stored, retrieved, accessed, or transmitted, or combinations thereof, in binary, encoded, compressed, or encrypted, or combinations thereof, formats, or any other machine-readable formats.

Interfacing or interconnection among various systems and layers may employ any number of mechanisms, such as any number of protocols, programmatic frameworks, floorplans, or application programming interfaces (API), including but not limited to Document Object Model (DOM), Discovery Service (DS), NSUserDefaults, Web Services Description Language (WSDL), Message Exchange Pattern (MEP), Web Distributed Data Exchange (WDDX), Web Hypertext Application Technology Working Group (WHATWG) HTML5 Web Messaging, Representational State Transfer (REST or RESTful web services), Extensible User Interface Protocol (XUP), Simple Object Access Protocol (SOAP), XML Schema Definition (XSD), XML Remote Procedure Call (XML-RPC), or any other mechanisms, open or proprietary, that may achieve similar functionality and results.

Such interfacing or interconnection may also make use of uniform resource identifiers (URI), which may further include uniform resource locators (URL) or uniform resource names (URN). Other forms of uniform, or unique, or combinations thereof, identifiers, locators, or names may be used, either exclusively or in combination with forms such as those set forth above.

Any of the above protocols or APIs may interface with or be implemented in any programming language, procedural, functional, or object-oriented, and may be compiled or interpreted. Non-limiting examples include C, C++, C#, Objective-C, Java, Scala, Clojure, Elixir, Swift, Go, Perl, PHP, Python, Ruby, JavaScript, WebAssembly, or virtually any other language, with any other libraries or schemas, in any kind of framework, runtime environment, virtual machine, interpreter, stack, engine, or similar mechanism, including but not limited to Node.js, V8, Knockout, jQuery, Dojo, Dijit, OpenUI5, AngularJS, Expressjs, Backbone.js, Ember.js, DHTMLX, Vue, React, Electron, and so on, among many other non-limiting examples.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 800, main memory 808, secondary memory 810, and removable storage units 818 and 822, as well as tangible articles of manufacture embodying any combination of the foregoing. Such control logic, when executed by one or more data processing devices (such as computer system 800), may cause such data processing devices to operate as described herein.

Based on the teachings contained in this disclosure, it may be apparent how to make and use embodiments of this disclosure using data processing devices, computer systems, or computer architectures, or combinations thereof, other than that shown in FIG. 8. In particular, embodiments may operate with software, hardware, or operating system implementations, or combinations thereof, other than those described herein.

### Optical Systems

The imager 116 in FIG. 1 can include one or more optical systems. Further disclosed herein are optical system design guidelines and high-performance fluorescence imaging methods and systems that provide improved optical resolution and image quality for fluorescence imaging-based genomics applications. The disclosed optical imaging system designs provide for larger fields-of-view, increased spatial resolution, improved modulation transfer, contrast-to-noise ratio, and image quality, higher spatial sampling frequency, faster transitions between image capture when repositioning the sample plane to capture a series of images (e.g., of different fields-of-view), and improved imaging system duty cycle, and thus, enable higher throughput image acquisition and analysis.

In some instances, improvements in imaging performance, e.g., for dual-side (flow cell) imaging applications, may be achieved by using an electro-optical phase plate in combination with an objective lens to compensate for the optical aberrations induced by the layer of fluid separating the upper (near) and lower (far) interior surfaces of a flow cell. In some instances, this design approach may also compensate for vibrations introduced by, e.g., a motion-actuated compensator that is moved in or out of the optical path depending on which surface of the flow cell is being imaged.

In some instances, improvements in imaging performance, e.g., for dual-side (flow cell) imaging applications comprising the use of thick flow cell walls (e.g., wall (or coverslip) thickness >700 μm) and fluid channels (e.g., fluid channel height or thickness of 50-200 μm) may be achieved even when using commercially-available, off-the-shelf objectives by using a tube lens design that corrects for the optical aberrations induced by the thick flow cell walls, or intervening fluid layer, or combinations thereof, in combination with the objective.

In some instances, improvements in imaging performance, e.g., for multichannel (e.g., two-color or four-color) imaging applications, may be achieved by using multiple tube lenses, one for each imaging channel, where each tube lens design has been optimized for the specific wavelength range used in that imaging channel.

Embodiments disclosed herein may comprise fluorescence imaging systems, said systems comprising: a) at least one light source configured to provide excitation light within one or more specified wavelength ranges; b) an objective lens configured to collect fluorescence arising from within a specified field-of-view of a sample plane upon exposure of the sample plane to the excitation light, wherein a numerical aperture of the objective lens is at least 0.1, at least 0.2, at least 0.3, at least 0.4, at least 0.5, at least 0.6, at least 0.7, at least 0.8, or at least 0.9 or a numerical aperture value falling within a range defined by any two of the foregoing; wherein a working distance of the objective lens is at least 400 micron (μm), at least 500 μm, at least 600 μm, at least 700 μm, at least 800 μm, at least 900 μm, at least 1000 μm, or a working distance falling within a range defined by any two of the foregoing; and wherein the field-of-view has an area of at least 0.1 mm2, at least 0.2 mm2, at least 0.5 mm2, at least 0.7 mm2, at least 1 mm2, at least 2 mm2, at least 3 mm2, at least 5 mm2, or at least 10 mm2, or a field of view falling within a range defined by any two of the foregoing; and c) at least one image sensor, wherein the fluorescence collected by the objective lens is imaged onto the image sensor, and wherein a pixel dimension for the image sensor is chosen such that a spatial sampling frequency for the fluorescence imaging system is at least twice an optical resolution of the fluorescence imaging system.

In some embodiments, the numerical aperture may be at least 0.75. In some embodiments, the numerical aperture is at least 1.0. In some embodiments, the working distance is at least 850 μm. In some embodiments, the working distance is at least 1,000 μm. In some embodiments, the field-of-view may have an area of at least 2.5 mm². In some embodiments, the field-of-view may have an area of at least 3 mm². In some embodiments, the spatial sampling frequency may be at least 2.5 times the optical resolution of the fluorescence imaging system. In some embodiments, the spatial sampling frequency may be at least 3 times the optical resolution of the fluorescence imaging system. In some embodiments, the system may further comprise an X-Y-Z translation stage such that the system is configured to acquire a series of two or more fluorescence images in an automated fashion, wherein each image of the series is or can be acquired for a different field-of-view. In some embodiments, a position of the sample plane may be simultaneously adjusted in an X direction, a Y direction, and a Z direction to match the position of an objective lens focal plane in between acquiring images for different fields-of-view. In some embodiments, the time required for the simultaneous adjustments in the X direction, Y direction, and Z direction may be less than 0.3 seconds, less than 0.4 seconds, less than 0.5 seconds, less than 0.7 seconds, or less than 1 second, or a time falling within a range defined by any two of the foregoing. In some embodiments, the system may further comprise an autofocus mechanism configured to adjust the focal plane position prior to acquiring an image of a different field-of-view if an error signal indicates that a difference in the position of the focal plane and the sample plane in the Z direction is greater than a specified error threshold. In some embodiments, the specified error threshold is 100 nm or greater. In some embodiments, the specified error threshold is 50 nm or less. In some embodiments, the system comprises three or more image sensors, and wherein the system is configured to image fluorescence in each of three or more wavelength ranges onto a different image sensor. In some embodiments, a difference in the position of a focal plane for each of the three or more image sensors and the sample plane is less than 100 nm. In some embodiments, a difference in the position of a focal plane for each of the three or more image sensors and the sample plane is less than 50 nm. In some embodiments, the total time required to reposition the sample plane, adjust focus if necessary, and acquire an image is less than 0.4 seconds per field-of-view. In some embodiments, the total time required to reposition the sample plane, adjust focus if necessary, and acquire an image is less than 0.3 seconds per field-of-view.

Also disclosed herein are fluorescence imaging systems for dual-side imaging of a flow cell comprising: a) an objective lens configured to collect fluorescence arising from within a specified field-of-view of a sample plane within the flow cell; b) at least one tube lens positioned between the objective lens and at least one image sensor, wherein the at least one tube lens is configured to correct an imaging performance metric for a combination of the objective lens, the at least one tube lens, and the at least one image sensor when imaging an interior surface of the flow cell, and wherein the flow cell has a wall thickness of at least 700 μm and a gap between an upper interior surface and a lower interior surface of at least 50 μm; wherein the imaging performance metric is substantially the same for imaging the upper interior surface or the lower interior surface of the flow cell without moving an optical compensator into or out of an optical path between the flow cell and the at least one image sensor, without moving one or more optical elements of the tube lens along the optical path, and without moving one or more optical elements of the tube lens into or out of the optical path.

In some embodiments, the objective lens may be a commercially-available microscope objective. In some embodiments, the commercially-available microscope objective may have a numerical aperture of at least 0.3. In some embodiments, the objective lens may have a working distance of at least 700 μm. In some embodiments, the objective lens may be corrected to compensate for a cover slip thickness (or flow cell wall thickness) of 0.17 mm or of greater or lesser thickness than 0.17 mm. In some embodiments, the optical system may be corrected to compensate for cover slip thickness, flow cell thickness, or distance between focal planes. In some embodiments, said correction may be made by inserting a corrective optic, such as a lens or optical assembly into the light path of the optical system. In some embodiments, said correction may be made without inserting a corrective optic, such as a lens or optical assembly into the light path of the optical system. In some embodiments, the fluorescence imaging system may further comprise an electro-optical phase plate positioned adjacent to the objective lens and between the objective lens and the tube lens, wherein the electro-optical phase plate may provide correction for optical aberrations caused by a fluid filling the gap between the upper interior surface and the lower interior surface of the flow cell. In some embodiments, the at least one tube lens may be a compound lens comprising three or more optical components. In some embodiments, the at least one tube lens is a compound lens comprising four optical components, which may comprise one or more of a first asymmetric convex-convex lens, a second convex-plano lens, a third asymmetric concave-concave lens, and a fourth asymmetric convex-concave lens which may be present in the order as listed above, or in any alternate order. In some embodiments, the at least one tube lens is configured to correct an imaging performance metric for a combination of the objective lens, the at least one tube lens, and the at least one image sensor when imaging an interior surface of a flow cell having a wall thickness of at least 1 mm. In some embodiments, the at least one tube lens is configured to correct an imaging performance metric for a combination of the objective lens, the at least one tube lens, and the at least one image sensor when imaging an interior surface of a flow cell having a gap of at least 100 μm. In some embodiments, the at least one tube lens is configured to correct an imaging performance metric for a combination of the objective lens, the at least one tube lens, and the at least one image sensor when imaging an interior surface of a flow cell having a gap of at least 200 μm. In some embodiments, the system comprises a single objective lens, two tube lenses, and two image sensors, and each of the two tube lenses is designed to provide optimal imaging performance at a different fluorescence wavelength. In some embodiments, the system comprises a single objective lens, three tube lenses, and three image sensors, and each of the three tube lenses is designed to provide optimal imaging performance at a different fluorescence wavelength. In some embodiments, the system comprises a single objective lens, four tube lenses, and four image sensors, and each of the four tube lenses is designed to provide optimal imaging performance at a different fluorescence wavelength. In some embodiments, the design of the objective lens or the at least one tube lens is configured to optimize the modulation transfer function in the mid to high spatial frequency range. In some embodiments, the imaging performance metric comprises a measurement of modulation transfer function (MTF) at one or more specified spatial frequencies, defocus, spherical aberration, chromatic aberration, coma, astigmatism, field curvature, image distortion, contrast-to-noise ratio (CNR), or any combination thereof. In some embodiments, the difference in the imaging performance metric for imaging the upper interior surface and the lower interior surface of the flow cell is less than 10%. In some embodiments, the difference in imaging performance metric for imaging the upper interior surface and the lower interior surface of the flow cell is less than 5%. In some embodiments, the use of the at least one tube lens provides for an at least equivalent or better improvement in the imaging performance metric for dual-side imaging compared to that for an existing system comprising an objective lens, a motion-actuated compensator, and an image sensor. In some embodiments, the use of the at least one tube lens provides for an at least 10% improvement in the imaging performance metric for dual-side imaging compared to that for an existing system comprising an objective lens, a motion-actuated compensator, and an image sensor.

Disclosed herein are illumination systems for use in imaging-based solid-phase genotyping and sequencing applications, the illumination system comprising: a) a light source; and b) a liquid light-guide configured to collect light emitted by the light source and deliver it to a specified field-of-illumination on a support surface comprising tethered biological macromolecules.

In some embodiments, the illumination system further comprises a condenser lens. In some embodiments, the specified field-of-illumination has an area of at least 2 mm². In some embodiments, the light delivered to the specified field-of-illumination is of uniform intensity across a specified field-of-view for an imaging system used to acquire images of the support surface. In some embodiments, the specified field-of-view has an area of at least 2 mm². In some embodiments, the light delivered to the specified field-of-illumination is of uniform intensity across the specified field-of-view when a coefficient of variation (CV) for light intensity is less than 10%. In some embodiments, the light delivered to the specified field-of-illumination is of uniform intensity across the specified field-of-view when a coefficient of variation (CV) for light intensity is less than 5%. In some embodiments, the light delivered to the specified field-of-illumination has a speckle contrast value of less than 0.1. In some embodiments, the light delivered to the specified field-of-illumination has a speckle contrast value of less than 0.05.

Imaging modules and systems: The disclosed optical systems, imaging systems, or modules may, in some instances, be stand-alone optical systems designed for imaging a sample or substrate surface. In some instances, they may comprise one or more processors or computers. In some instances, they may comprise one or more software packages that provide instrument control functionality, or image processing functionality, or combinations thereof. In some instances, in addition to optical components such as light sources (e.g., solid-state lasers, dye lasers, diode lasers, arc lamps, tungsten-halogen lamps, etc.), lenses, prisms, mirrors, dichroic reflectors, optical filters, optical bandpass filters, apertures, and image sensors (e.g., complementary metal oxide semiconductor (CMOS) image sensors and cameras, charge-coupled device (CCD) image sensors and cameras, etc.), they may also include mechanical, or optomechanical components, or combinations thereof, such as an X-Y translation stage, an X-Y-Z translation stage, an auto focusing mechanism, or a piezoelectric focusing mechanism, and the like. In some instances, they may function as modules, components, sub-assemblies, or sub-systems of larger systems designed for genomics applications (e.g., genetic testing, or nucleic acid sequencing applications, or combinations thereof). For example, in some instances, they may function as modules, components, sub-assemblies, or sub-systems of larger systems that further comprise light-tight, or other environmental control housings, temperature control modules, fluidics control modules, fluid dispensing robotics, pick-and-place robotics, one or more processors or computers, one or more local or cloud-based software packages (e.g., instrument/system control software packages, image processing software packages, data analysis software packages), data storage modules, data communication modules (e.g., Bluetooth, WiFi, intranet, or internet communication hardware and associated software), display modules, or any combination thereof.

### Methods for Sequencing

The present disclosure provides methods for sequencing immobilized or non-immobilized template molecules. The methods can be operated in system 100, for example, in sequencer 114. In some embodiments, the immobilized template molecules comprise a plurality of nucleic acid template molecules having one copy of a target sequence of interest. In some embodiments, nucleic acid template molecules having one copy of a target sequence of interest can be generated by conducting bridge amplification using linear library molecules. In some embodiments, the immobilized template molecules comprise a plurality of nucleic acid template molecules each having two or more tandem copies of a target sequence of interest (e.g., concatemers). In some embodiments, nucleic acid template molecules comprising concatemer molecules can be generated by conducting rolling circle amplification of circularized linear library molecules. In some embodiments, the non-immobilized template molecules comprise circular molecules. In some embodiments, methods for sequencing employ soluble (e.g., non-immobilized) sequencing polymerases or sequencing polymerases that are immobilized to a support.

In some embodiments, the sequencing reactions employ detectably labeled nucleotide analogs. In some embodiments, the sequencing reactions employ a two-stage sequencing reaction comprising binding detectably labeled multivalent molecules and incorporating nucleotide analogs. In some embodiments, the sequencing reactions employ non-labeled nucleotide analogs. In some embodiments, the sequencing reactions employ phosphate chain labeled nucleotides.

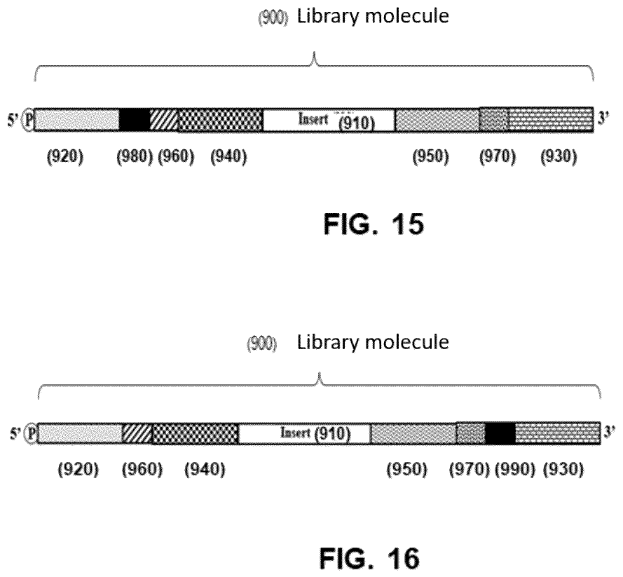

In some embodiments, the immobilized concatemers each comprise tandem repeat units of the sequence-of-interest (e.g., insert region) and any adaptor sequences. For example, as shown in FIG. 15, the tandem repeat unit comprises: (i) a left universal adaptor sequence having a binding sequence for a first surface primer (920) (e.g., surface pinning primer), (ii) a left universal adaptor sequence having a binding sequence for a first sequencing primer (940) (e.g., forward sequencing primer), (iii) a sequence-of-interest (910), (iv) a right universal adaptor sequence having a binding sequence for a second sequencing primer (950) (e.g., reverse sequencing primer), (v) a right universal adaptor sequence having a binding sequence for a second surface primer (930) (e.g., surface capture primer), and (vii) a left sample index sequence (960), or a right sample index sequence (970), or combinations thereof. In some embodiments, the tandem repeat unit further comprises a left unique identification sequence (980), or a right unique identification sequence (990), or combinations thereof. In some embodiments, the tandem repeat unit further comprises at least one binding sequence for a compaction oligonucleotide. In some embodiments, FIG. 15 and FIG. 16 show linear library molecules or a unit of a concatemer molecule. FIG. 15 shows a non-limiting example of a linear single stranded library molecule (900) which comprises: a surface pinning primer binding site (920); an optional left unique identification sequence (980); a left index sequence (960); a forward sequencing primer binding site (940); an insert region having a sequence of interest (910); a reverse sequencing primer binding site (950); a right index sequence (970); and a surface capture primer binding site (930). FIG. 16 shows a non-limiting example of a linear single stranded library molecule (900) which comprises: a surface pinning primer binding site (920); a left index sequence (960); a forward sequencing primer binding site (940); an insert region having a sequence of interest (910); a reverse sequencing primer binding site (950); a right index sequence (970); an optional right unique identification sequence (990); and a surface capture primer binding site (930).

The immobilized concatemer can self-collapse into a compact nucleic acid nanoball. Inclusion of one or more compaction oligonucleotides during the RCA reaction can further compact the size, or shape, or combinations thereof, of the nanoball. An increase in the number of tandem repeat units in a given concatemer increases the number of sites along the concatemer for hybridizing to multiple sequencing primers (e.g., sequencing primers having a universal sequence) which serve as multiple initiation sites for polymerase-catalyzed sequencing reactions. When the sequencing reaction employs detectably labeled nucleotides, or detectably labeled multivalent molecules (e.g., having nucleotide units), or combinations thereof, the signals emitted by the nucleotides or nucleotide units that participate in the parallel sequencing reactions along the concatemer yields an increased signal intensity for each concatemer. Multiple portions of a given concatemer can be simultaneously sequenced. Furthermore, a plurality of binding complexes can form along a particular concatemer molecule, each binding complex comprising a sequencing polymerase bound to a template/primer duplex and bound to a multivalent molecule, wherein the plurality of binding complexes remains stable without dissociation, resulting in increased persistence time which increases signal intensity and reduces imaging time.

### Methods for Sequencing Using Nucleotide Analogs

The present disclosure provides methods for sequencing any of the immobilized template molecules described herein, the methods comprising step (a): contacting a sequencing polymerase to (i) a nucleic acid template molecule and (ii) a nucleic acid sequencing primer, wherein the contacting is conducted under a condition suitable to bind the sequencing polymerase to the nucleic acid template molecule which is hybridized to the nucleic acid primer, wherein the nucleic acid template molecule hybridized to the nucleic acid primer forms the nucleic acid duplex. In some embodiments, the sequencing polymerase comprises a recombinant mutant sequencing polymerase that can bind and incorporate nucleotide analogs.

In some embodiments, in the methods for sequencing template molecules, the sequencing primer comprises a 3′ extendible end or a 3′ non-extendible end. In some embodiments, the plurality of nucleic acid template molecules comprises amplified template molecules (e.g., clonally amplified template molecules). In some embodiments, the plurality of nucleic acid template molecules comprises one copy of a target sequence of interest. In some embodiments, the plurality of nucleic acid molecules comprises two or more tandem copies of a target sequence of interest (e.g., concatemers). In some embodiments, the plurality of nucleic acid template molecules comprises the same target sequence of interest or different target sequences of interest. In some embodiments, the plurality of nucleic acid primers is in solution or is immobilized to a support. In some embodiments, when the plurality of nucleic acid template molecules, or the plurality of nucleic acid primers, or combinations thereof, are immobilized to a support, the binding with the first sequencing polymerase generates a plurality of immobilized first complexed polymerases. In some embodiments, the plurality of nucleic acid template molecules, or nucleic acid primers, or combinations thereof, are immobilized to 102-1015 different sites on a support. In some embodiments, the binding of the plurality of template molecules and nucleic acid primers with the plurality of first sequencing polymerases generates a plurality of first complexed polymerases immobilized to 102-1015 different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, or divalent cations, or combinations thereof) onto the support so that the plurality of immobilized complexed polymerases on the support are reacted with the solution of reagents in a massively parallel manner.

In some embodiments, the methods for sequencing further comprise step (b): contacting the sequencing polymerase with a plurality of nucleotides under a condition suitable for binding at least one nucleotide to the sequencing polymerase which is bound to the nucleic acid duplex and suitable for polymerase-catalyzed nucleotide incorporation which extends the sequencing primer by one nucleotide. In some embodiments, the sequencing polymerase is contacted with the plurality of nucleotides in the presence of at least one catalytic cation comprising magnesium, or manganese, or combinations thereof. In some embodiments, the plurality of nucleotides comprises at least one nucleotide analog having a chain terminating moiety at the sugar 2′ or 3′ position. In some embodiments, the chain terminating moiety is removable from the sugar 2′ or 3′ position to convert the chain terminating moiety to an OH or H group. In some embodiments, the plurality of nucleotides comprises at least one nucleotide that lacks a chain terminating moiety. In some embodiments, at least on nucleotide is labeled with a detectable reporter moiety (e.g., fluorophore) that emits a detectable signal. In some embodiments, the detectable reporter moiety comprises a fluorophore. In some embodiments, the fluorophore is attached to the nucleo-base. In some embodiments, the fluorophore is attached to the nucleo-base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleo-base. When the incorporated chain terminating nucleotide is detectably labeled, step (b) further comprises detecting the emitted signal from the incorporated chain terminating nucleotide. In some embodiments, step (b) further comprises identifying the nucleo-based of the incorporated chain terminating nucleotide.

In some embodiments, the methods for sequencing further comprise step (c): removing the chain terminating moiety from the incorporated chain terminating nucleotide to generate an extendible 3′OH group. In some embodiments, step (c) further comprises removing the detectable label from the incorporated chain terminating nucleotide. In some embodiments, the sequencing polymerase remains bound to the template molecule which is hybridized to the sequencing primer which is extended by one nucleo-base.

In some embodiments, the methods for sequencing further comprise step (d): repeating steps (b) and (c) at least once.

### Two-Stage Methods for Nucleic Acid Sequencing

The present disclosure provides a two-stage method for sequencing any of the immobilized template molecules described herein. In some embodiments, the first stage may comprise binding multivalent molecules to complexed polymerases to form multivalent-complexed polymerases and detecting the multivalent-complexed polymerases.

In some embodiments, the first stage comprises step (a): contacting a plurality of a first sequencing polymerase to (i) a plurality of nucleic acid template molecules and (ii) a plurality of nucleic acid sequencing primers, wherein the contacting is conducted under a condition suitable to bind the plurality of first sequencing polymerases to the plurality of nucleic acid template molecules and the plurality of nucleic acid primers thereby forming a plurality of first complexed polymerases each comprising a first sequencing polymerase bound to a nucleic acid duplex wherein the nucleic acid duplex comprises a nucleic acid template molecule hybridized to a nucleic acid primer. In some embodiments, the first polymerase comprises a recombinant mutant sequencing polymerase.

In some embodiments, in the methods for sequencing template molecules, the sequencing primer comprises an oligonucleotide having a 3′ extendible end or a 3′ non-extendible end. In some embodiments, the plurality of nucleic acid template molecules comprises amplified template molecules (e.g., clonally amplified template molecules). In some embodiments, the plurality of nucleic acid template molecules comprises one copy of a target sequence of interest. In some embodiments, the plurality of nucleic acid molecules comprises two or more tandem copies of a target sequence of interest (e.g., concatemers). In some embodiments, the nucleic acid template molecules in the plurality of nucleic acid template molecules comprise the same target sequence of interest or different target sequences of interest. In some embodiments, the plurality of nucleic acid template molecules, or the plurality of nucleic acid primers, or combinations thereof, are in solution or are immobilized to a support. In some embodiments, when the plurality of nucleic acid template molecules, or the plurality of nucleic acid primers, or combinations thereof, are immobilized to a support, the binding with the first sequencing polymerase generates a plurality of immobilized first complexed polymerases. In some embodiments, the plurality of nucleic acid template molecules, or nucleic acid primers, or combinations thereof, are immobilized to 102-1015 different sites on a support. In some embodiments, the binding of the plurality of template molecules and nucleic acid primers with the plurality of first sequencing polymerases generates a plurality of first complexed polymerases immobilized to 102-1015 different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, or divalent cations, or combinations thereof) onto the support so that the plurality of immobilized complexed polymerases on the support are reacted with the solution of reagents in a massively parallel manner.

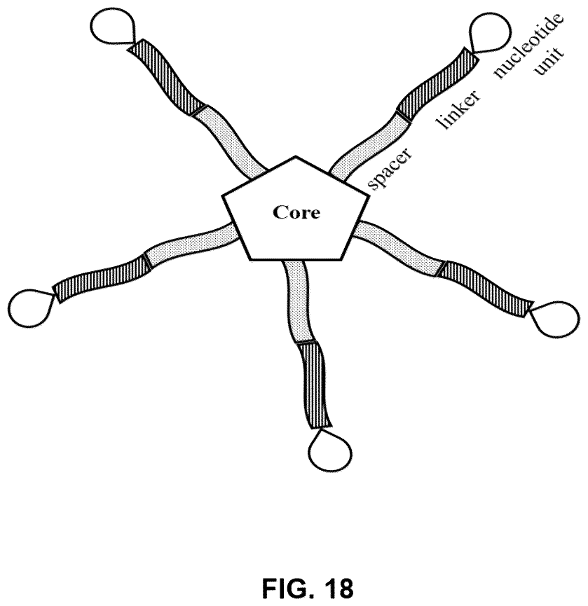

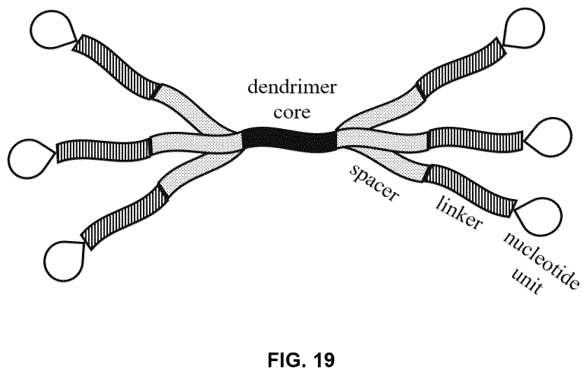

In some embodiments, the methods for sequencing further comprise step (b): contacting the plurality of first complexed polymerases with a plurality of multivalent molecules to form a plurality of multivalent-complexed polymerases (e.g., binding complexes). In some embodiments, individual multivalent molecules in the plurality of multivalent molecules comprise a core attached to multiple nucleotide arms and each nucleotide arm is attached to a nucleotide (e.g., nucleotide unit) (e.g., FIGS. 15-19). In some embodiments, the contacting of step (b) is conducted under a condition suitable for binding complementary nucleotide units of the multivalent molecules to at least two of the plurality of first complexed polymerases thereby forming a plurality of multivalent-complexed polymerases. In some embodiments, the condition is suitable for inhibiting polymerase-catalyzed incorporation of the complementary nucleotide units into the primers of the plurality of multivalent-complexed polymerases. In some embodiments, the plurality of multivalent molecules comprises at least one multivalent molecule having multiple nucleotide arms (e.g., FIGS. 15-18) each attached with a nucleotide analog (e.g., nucleotide analog unit), where the nucleotide analog includes a chain terminating moiety at the sugar 2′ position, or 3′ position, or combinations thereof. In some embodiments, the plurality of multivalent molecules comprises at least one multivalent molecule comprising multiple nucleotide arms each attached with a nucleotide unit that lacks a chain terminating moiety. In some embodiments, at least one of the multivalent molecules in the plurality of multivalent molecules is labeled with a detectable reporter moiety that emits a signal. In some embodiments, the detectable reporter moiety comprises a fluorophore. In some embodiments, the contacting of step (b) is conducted in the presence of at least one non-catalytic cation comprising strontium, barium, or calcium, or combinations thereof.

In some embodiments, the methods for sequencing further comprises step (c): detecting the plurality of multivalent-complexed polymerases. In some embodiments, the detecting includes detecting the signals emitted by the multivalent molecules that are bound to the complexed polymerases, where the complementary nucleotide units of the multivalent molecules are bound to the primers but incorporation of the complementary nucleotide units is inhibited. In some embodiments, the multivalent molecules are labeled with a detectable reporter moiety to permit detection. In some embodiments, the labeled multivalent molecules comprise a fluorophore attached to the core, linker, or nucleotide unit, or combinations thereof, of the multivalent molecules.

In some embodiments, the methods for sequencing further comprise step (d): identifying the nucleobase of the complementary nucleotide units that are bound to the plurality of first complexed polymerases, thereby determining the sequence of the template molecule. In some embodiments, the multivalent molecules are labeled with a detectable reporter moiety that corresponds to the particular nucleotide units attached to the nucleotide arms to permit identification of the complementary nucleotide units (e.g., nucleotide base adenine, guanine, cytosine, thymine or uracil) that are bound to the plurality of first complexed polymerases.

In some embodiments, the methods for sequencing further comprise step (e): dissociating the plurality of multivalent-complexed polymerases and removing the plurality of first sequencing polymerases and their bound multivalent molecules, and retaining the plurality of nucleic acid duplexes.

In some embodiments, the second stage of the two-stage sequencing method may comprise nucleotide incorporation. In some embodiments, the methods for sequencing further comprises step (f): contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a nucleic acid duplex. In some embodiments, the second sequencing polymerase comprises a recombinant mutant sequencing polymerase.

In some embodiments, the plurality of first sequencing polymerases of step (a) has an amino acid sequence that is 100% identical to the amino acid sequence as the plurality of the second sequencing polymerases of step (f). In some embodiments, the plurality of first sequencing polymerases of step (a) has an amino acid sequence that differs from the amino acid sequence of the plurality of the second sequencing polymerases of step (f).

In some embodiments, the methods for sequencing further comprise step (g): contacting the plurality of second complexed polymerases with a plurality of nucleotides, wherein the contacting is conducted under a condition suitable for binding complementary nucleotides from the plurality of nucleotides to at least two of the second complexed polymerases thereby forming a plurality of nucleotide-complexed polymerases. In some embodiments, the contacting of step (g) is conducted under a condition that is suitable for promoting polymerase-catalyzed incorporation of the bound complementary nucleotides into the primers of the nucleotide-complexed polymerases thereby extending the sequencing primer by one nucleo-base. In some embodiments, the incorporating the nucleotide into the 3′ end of the sequencing primer in step (g) comprises a primer extension reaction. In some embodiments, the contacting of step (g) is conducted in the presence of at least one catalytic cation comprising magnesium, or manganese, or combinations thereof. In some embodiments, the plurality of nucleotides comprises native nucleotides (e.g., non-analog nucleotides) or nucleotide analogs. In some embodiments, the plurality of nucleotides comprises a 2′, or 3′, or combinations thereof, chain terminating moiety which is removable or is not removable. In some embodiments, at least one of the nucleotides in the plurality is not labeled with a detectable reporter moiety. In some embodiments, the plurality of nucleotides is non-labeled. In some embodiments, the plurality of nucleotides comprises a plurality of nucleotides labeled with a detectable reporter moiety. In some embodiments, the detectable reporter moiety comprises a fluorophore. In some embodiments, the fluorophore is attached to the nucleotide base. In some embodiments, the fluorophore is attached to the nucleotide base with a linker which is cleavable/removable from the base or is not removable from the base. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, when the plurality of nucleotides in step (g) is detectably labeled, the methods for sequencing further comprise step (h): detecting the complementary nucleotides which are incorporated into the primers of the nucleotide-complexed polymerases. In some embodiments, the plurality of nucleotides is labeled with a detectable reporter moiety to permit detection. In some embodiments, when the plurality of nucleotides in step (g) is non-labeled, the detecting of step (h) is omitted.

In some embodiments, when the plurality of nucleotides in step (g) is detectably labeled, the methods for sequencing further comprise step (i): identifying the bases of the complementary nucleotides which are incorporated into the primers of the nucleotide-complexed polymerases. In some embodiments, the identification of the incorporated complementary nucleotides in step (i) can be used to confirm the identity of the complementary nucleotides of the multivalent molecules that are bound to the plurality of first complexed polymerases in step (d). In some embodiments, the identifying of step (i) can be used to determine the sequence of the nucleic acid template molecules. In some embodiments, when the plurality of nucleotides in step (g) is non-labeled, the identifying of step (i) is omitted.

In some embodiments, the methods for sequencing further comprise step (j): removing the chain terminating moiety from the incorporated nucleotide when step (g) is conducted by contacting the plurality of second complexed polymerases with a plurality of nucleotides that comprise at least one nucleotide having a 2′, or 3′, or combinations thereof, chain terminating moiety.

In some embodiments, the methods for sequencing further comprise step (k): repeating steps (a)-(j) at least once. In some embodiments, the sequence of the nucleic acid template molecules can be determined by detecting and identifying the multivalent molecules that bind the sequencing polymerases but do not incorporate into the 3′ end of the primer at steps (c) and (d). In some embodiments, the sequence of the nucleic acid template molecules can be determined (or confirmed) by detecting and identifying the nucleotide that incorporates into the 3′ end of the primer at steps (h) and (i).

In some embodiments, in any of the methods for sequencing nucleic acid molecules, the binding of the plurality of first complexed polymerases with the plurality of multivalent molecules forms at least one avidity complex, the method comprising the steps: (a) binding a first nucleic acid primer, a first sequencing polymerase, and a first multivalent molecule to a first portion of a concatemer template molecule thereby forming a first binding complex, wherein a first nucleotide unit of the first multivalent molecule binds to the first sequencing polymerase; and (b) binding a second nucleic acid primer, a second sequencing polymerase, and the first multivalent molecule to a second portion of the same concatemer template molecule thereby forming a second binding complex, wherein a second nucleotide unit of the first multivalent molecule binds to the second sequencing polymerase, wherein the first and second binding complexes which include the same multivalent molecule forms an avidity complex. In some embodiments, the first sequencing polymerase comprises any wild type or mutant polymerase described herein. In some embodiments, the second sequencing polymerase comprises any wild type or mutant polymerase described herein. The concatemer template molecule comprises tandem repeat sequences of a sequence of interest and at least one universal sequencing primer binding site. The first and second nucleic acid primers can bind to a sequencing primer binding site along the concatemer template molecule. For example, multivalent molecules are shown in FIGS. 15-18.

In some embodiments, in any of the methods for sequencing nucleic acid molecules, wherein the method includes binding the plurality of first complexed polymerases with the plurality of multivalent molecules to form at least one avidity complex, the method comprising the steps: (a) contacting the plurality of sequencing polymerases and the plurality of nucleic acid primers with different portions of a concatemer nucleic acid concatemer molecule to form at least first and second complexed polymerases on the same concatemer template molecule; (b) contacting a plurality of multivalent molecules to the at least first and second complexed polymerases on the same concatemer template molecule, under conditions suitable to bind a single multivalent molecule from the plurality to the first and second complexed polymerases, wherein at least a first nucleotide unit of the single multivalent molecule is bound to the first complexed polymerase which includes a first primer hybridized to a first portion of the concatemer template molecule thereby forming a first binding complex (e.g., first ternary complex), and wherein at least a second nucleotide unit of the single multivalent molecule is bound to the second complexed polymerase which includes a second primer hybridized to a second portion of the concatemer template molecule thereby forming a second binding complex (e.g., second ternary complex), wherein the contacting is conducted under a condition suitable to inhibit polymerase-catalyzed incorporation of the bound first and second nucleotide units in the first and second binding complexes, and wherein the first and second binding complexes which are bound to the same multivalent molecule forms an avidity complex; and (c) detecting the first and second binding complexes on the same concatemer template molecule, and (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule. In some embodiments, the plurality of sequencing polymerases comprise any wild type or mutant sequencing polymerase described herein. The concatemer template molecule comprises tandem repeat sequences of a sequence of interest and at least one universal sequencing primer binding site. The plurality of nucleic acid primers can bind to a sequencing primer binding site along the concatemer template molecule. For example, multivalent molecules are shown in FIGS. 15-18.

### Sequencing-by-Binding

The present disclosure provides methods for sequencing any of the immobilized template molecules described herein, wherein the sequencing methods comprise a sequencing-by-binding (SBB) procedure which employs non-labeled chain-terminating nucleotides. In some embodiments, the sequencing-by-binding (SBB) method comprises the steps of (a) sequentially contacting a primed template nucleic acid with at least two separate mixtures under ternary complex stabilizing conditions, wherein the at least two separate mixtures each include a polymerase and a nucleotide, whereby the sequentially contacting results in the primed template nucleic acid being contacted, under the ternary complex stabilizing conditions, with nucleotide cognates for first, second and third base types in the template; (b) examining the at least two separate mixtures to determine whether a ternary complex formed; and (c) identifying the next correct nucleotide for the primed template nucleic acid molecule, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed template nucleic acid after step (b), thereby producing an extended primer; and (e) repeating steps (a) through (d) at least once on the primed template nucleic acid that comprises the extended primer. For example, sequencing-by-binding methods are described in U.S. Pat. Nos. 10,246,744 and 10,731,141 (where the contents of both patents are hereby incorporated by reference in their entireties).

### Methods for Sequencing Using Phosphate-Chain Labeled Nucleotides

The present disclosure provides methods for sequencing using immobilized sequencing polymerases which bind non-immobilized template molecules, wherein the sequencing reactions are conducted with phosphate-chain labeled nucleotides. In some embodiments, the sequencing methods comprise step (a): providing a support having a plurality of sequencing polymerases immobilized thereon. In some embodiments, the sequencing polymerase comprises a processive DNA polymerase. In some embodiments, the sequencing polymerase comprises a wild type or mutant DNA polymerase, including, for example, a Phi29 DNA polymerase. In some embodiments, the support comprises a plurality of separate compartments and a sequencing polymerase that is immobilized to the bottom of a compartment. In some embodiments, the separate compartments comprise a silica bottom through which light can penetrate. In some embodiments, the separate compartments comprise a silica bottom configured with a nanophotonic confinement structure comprising a hole in a metal cladding film (e.g., aluminum cladding film). In some embodiments, the hole in the metal cladding has a small aperture, for example, approximately 70 nm. In some embodiments, the height of the nanophotonic confinement structure is approximately 100 nm. In some embodiments, the nanophotonic confinement structure comprises a zero mode waveguide (ZMW). In some embodiments, the nanophotonic confinement structure contains a liquid.

In some embodiments, the sequencing method further comprises step (b): contacting the plurality of immobilized sequencing polymerases with a plurality of single stranded circular nucleic acid template molecules and a plurality of oligonucleotide sequencing primers, under a condition suitable for individual immobilized sequencing polymerases to bind a single stranded circular template molecule, and suitable for individual sequencing primers to hybridize to individual single stranded circular template molecules, thereby generating a plurality of polymerase/template/primer complexes. In some embodiments, the individual sequencing primers hybridize to a universal sequencing primer binding site on the single stranded circular template molecule.

In some embodiments, the sequencing method further comprises step (c): contacting the plurality of polymerase/template/primer complexes with a plurality of phosphate chain labeled nucleotides each comprising an aromatic base, a five carbon sugar (e.g., ribose or deoxyribose), and a phosphate chain comprising 3-20 phosphate groups, where the terminal phosphate group is linked to a detectable reporter moiety (e.g., a fluorophore). The first, second and third phosphate groups can be referred to as alpha, beta and gamma phosphate groups. In some embodiments, a particular detectable reporter moiety which is attached to the terminal phosphate group corresponds to the nucleotide base (e.g., dATP, dGTP, dCTP, dTTP or dUTP) to permit detection and identification of the nucleo-base. In some embodiments, the plurality of polymerase/template/primer complexes is contacted with the plurality of phosphate chain labeled nucleotides under a condition suitable for polymerase-catalyzed nucleotide incorporation. In some embodiments, the sequencing polymerases are capable of binding a complementary phosphate chain labeled nucleotide and incorporating the complementary nucleotide opposite a nucleotide in a template molecule. In some embodiments, the polymerase-catalyzed nucleotide incorporation reaction cleaves between the alpha and beta phosphate groups thereby releasing a multi-phosphate chain linked to a fluorophore.

In some embodiments, the sequencing method further comprises step (d): detecting the fluorescent signal emitted by the phosphate chain labeled nucleotide that is bound by the sequencing polymerase and incorporated into the terminal end of the sequencing primer. In some embodiments, step (d) further comprises identifying the phosphate chain labeled nucleotide that is bound by the sequencing polymerase and incorporated into the terminal end of the sequencing primer.

In some embodiments, the sequencing method further comprises step (d): repeating steps (c)-(d) at least once. In some embodiments, sequencing methods that employ phosphate chain labeled nucleotides can be conducted according to the methods described in U.S. Pat. Nos. 7,170,050; 7,302,146; or 7,405,281, or combinations thereof, all of which are hereby incorporated by reference in their entireties.

### Sequencing Polymerases

The present disclosure provides methods for sequencing nucleic acid molecules, where any of the sequencing methods described herein employ at least one type of sequencing polymerase and a plurality of nucleotides or employ at least one type of sequencing polymerase and a plurality of nucleotides and a plurality of multivalent molecules. In some embodiments, the sequencing polymerase(s) is/are capable of incorporating a complementary nucleotide opposite a nucleotide in a template molecule. In some embodiments, the sequencing polymerase(s) is/are capable of binding a complementary nucleotide unit of a multivalent molecule opposite a nucleotide in a template molecule. In some embodiments, the plurality of sequencing polymerases comprises recombinant mutant polymerases.

Examples of suitable polymerases for use in sequencing with nucleotides, or multivalent molecules, or combinations thereof, include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including existing polymerases such as 9 degrees N, VENT, DEEP VENT, THERMINATOR, Pfu, KOD, Pfx, Tgo and RB69 polymerases.

### Nucleotides

The present disclosure provides methods for sequencing nucleic acid molecules, where any of the sequencing methods described herein employ at least one nucleotide. The nucleotides comprise a base, a sugar and at least one phosphate group. In some embodiments, at least one nucleotide in the plurality comprises an aromatic base, a five carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP, or dUTP, or combinations thereof. In some embodiments, at least one nucleotide in the plurality is not a nucleotide analog. In some embodiments, at least one nucleotide in the plurality comprises a nucleotide analog.

In some embodiments, in any of the methods for sequencing nucleic acid molecules described herein, at least one nucleotide in the plurality of nucleotides comprise a chain of one, two or three phosphorus atoms where the chain may be attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methyl phosphoramidite groups.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to 3′ sugar position where the sugar comprises a ribose or deoxyribose sugar moiety. In some embodiments, the chain terminating moiety is removable/cleavable from 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, silyl or acetal group. In some embodiments, the chain terminating moiety is cleavable/removable from the nucleotide, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄), with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride. In some embodiments, the chain terminating moiety may be cleavable/removable with nitrous acid. In some embodiments, a chain terminating moiety may be cleavable/removable using a solution comprising nitrite, such as, for example, a combination of nitrite with an acid such as acetic acid, sulfuric acid, or nitric acid. In some further embodiments, said solution may comprise an organic acid.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl groups are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP). In some embodiments, the chain terminating moiety comprising one or more of a 3′-O-amino group, a 3′-O-aminomethyl group, a 3′-O-methylamino group, or derivatives thereof may be cleaved with nitrous acid, through a mechanism utilizing nitrous acid, or using a solution comprising nitrous acid. In some embodiments, the chain terminating moiety comprising one or more of a 3′-O-amino group, a 3′-O-aminomethyl group, a 3′-O-methylamino group, or derivatives thereof may be cleaved using a solution comprising nitrite. In some embodiments, for example, nitrite may be combined with or contacted with an acid such as acetic acid, sulfuric acid, or nitric acid. In some further embodiments, for example, nitrite may be combined with or contacted with an organic acid such as, for example, formic acid, acetic acid, propionic acid, butyric acid, isobutyric acid, or the like. In some embodiments, the chain terminating moiety comprises a 3′-acetal moiety which can be cleaved with a palladium deblocking reagent (e.g., Pd(0)).

In some embodiments, in any of the methods for sequencing nucleic acid molecules described herein, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, 3-O-benzyl, and 3′-O-benzyl, 3-acetal moiety, or derivatives thereof.

In some embodiments, in any of the methods for sequencing nucleic acid molecules described herein, the plurality of nucleotides comprises a plurality of nucleotides labeled with a detectable reporter moiety. In some embodiments, the detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, in any of the methods for sequencing nucleic acid molecules described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine)palladium(0) (Pd(PPh₃)₄), with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thio, disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, in any of the methods for sequencing nucleic acid molecules described herein, the cleavable linker on the nucleotide base comprises cleavable moiety including an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing nucleic acid molecules described herein, the chain terminating moiety (e.g., at the sugar 2′ position, or sugar 3′ position, or combinations thereof) and the cleavable linker on the nucleotide base have the same or different cleavable moieties. In some embodiments, the chain terminating moiety (e.g., at the sugar 2′ position, or sugar 3′ position, or combinations thereof) and the detectable reporter moiety linked to the base are chemically cleavable/removable with the same chemical agent. In some embodiments, the chain terminating moiety (e.g., at the sugar 2′ position, or sugar 3′ position, or combinations thereof) and the detectable reporter moiety linked to the base are chemically cleavable/removable with different chemical agents.

### Multivalent Molecules

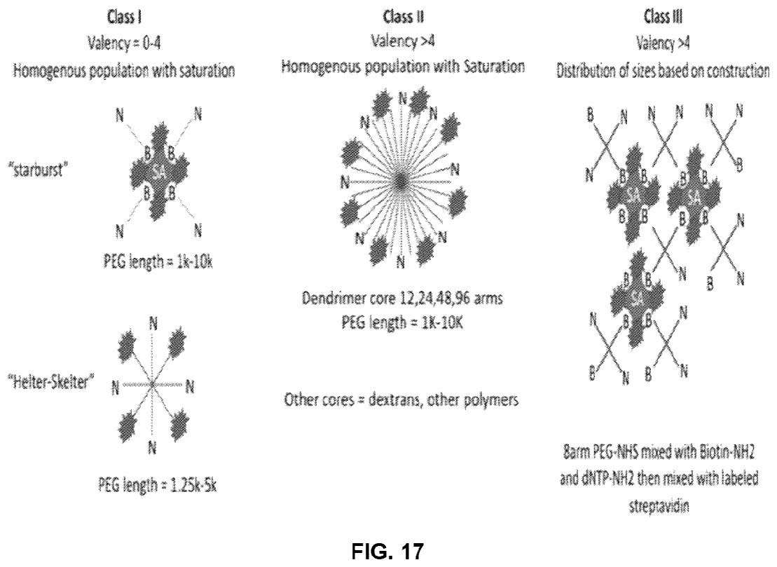

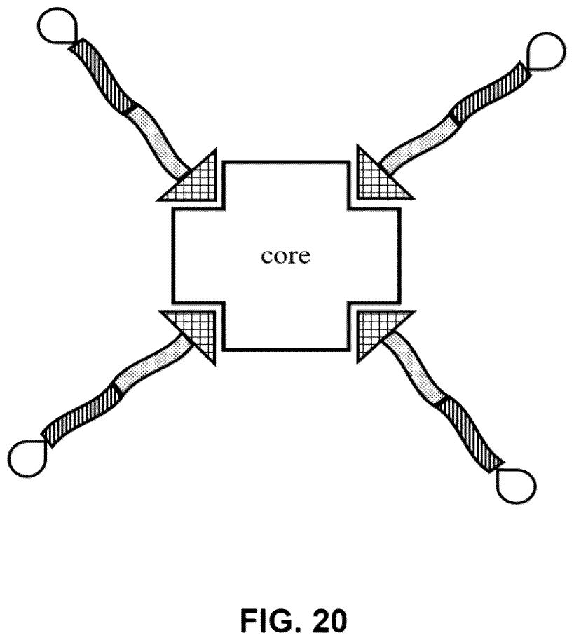

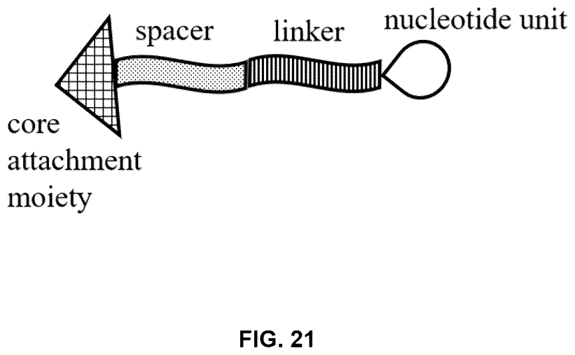

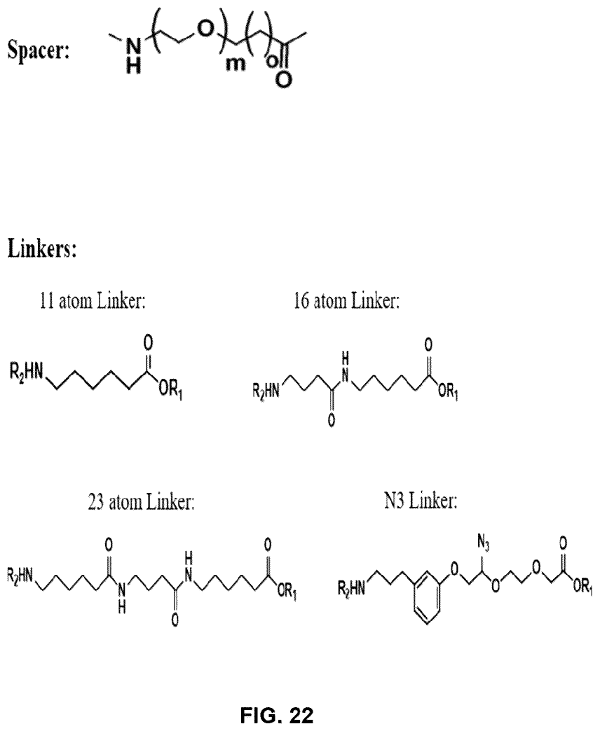

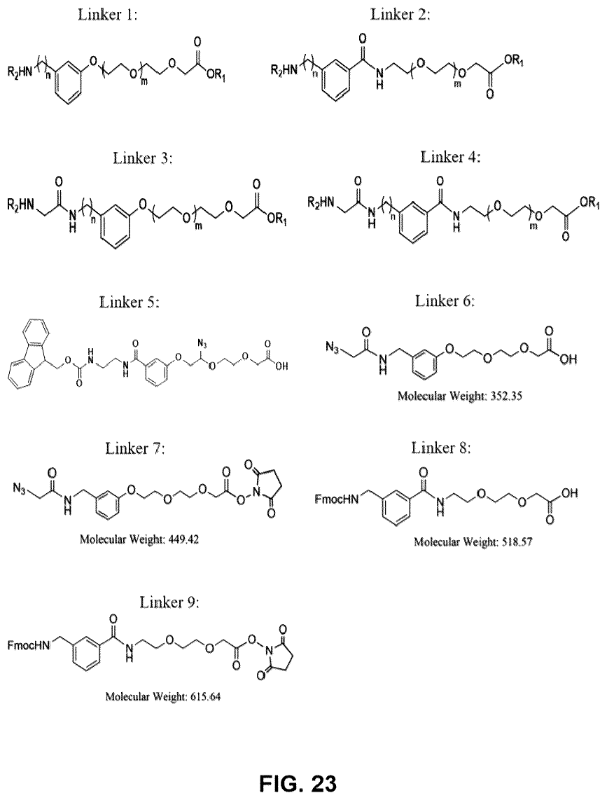

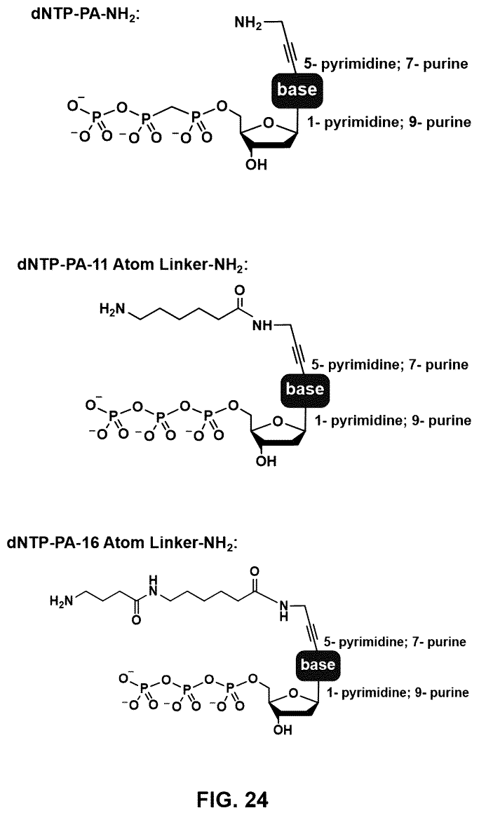

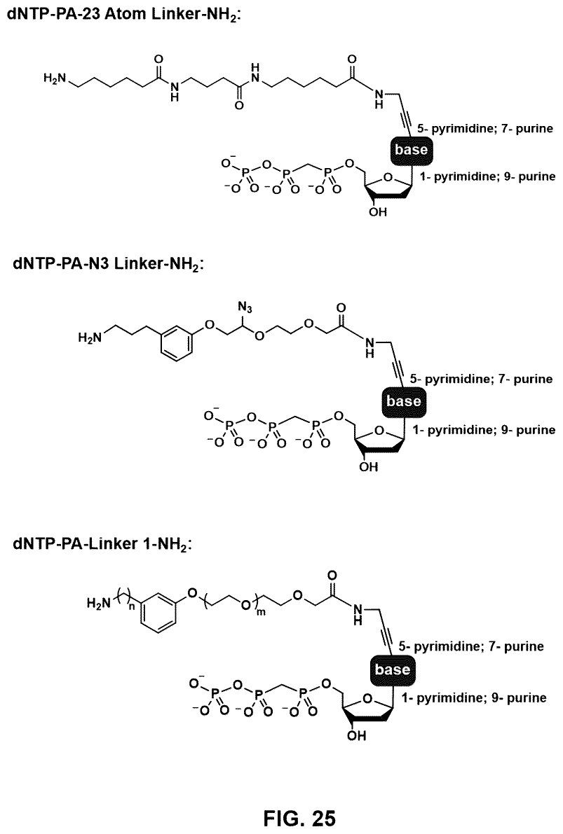

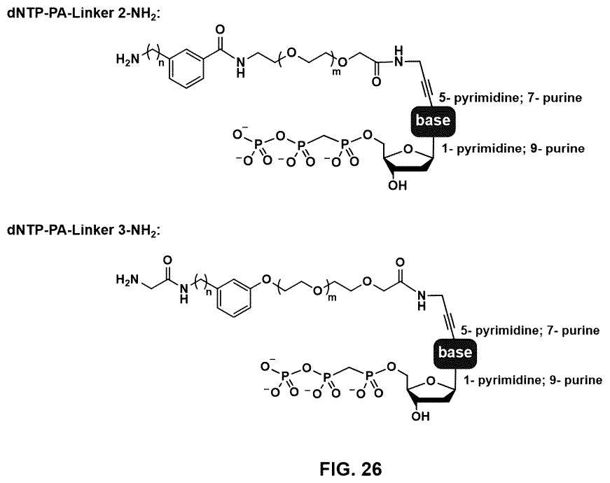

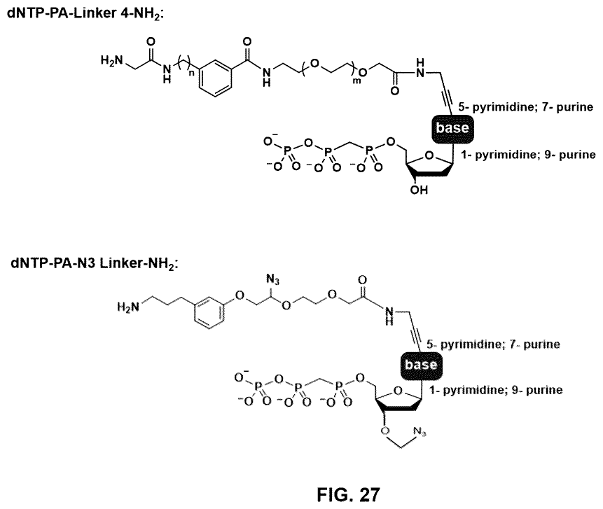

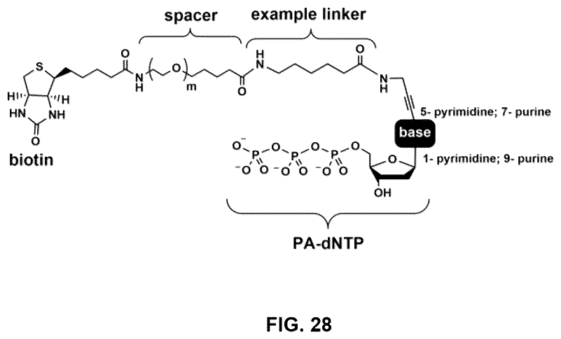

The present disclosure provides methods for sequencing nucleic acid molecules, where any of the sequencing methods described herein employ at least one multivalent molecule. In some embodiments, the multivalent molecule comprises a plurality of nucleotide arms attached to a core and having any configuration including a starburst, helter skelter, or bottle brush configuration (e.g., FIG. 17). The multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, wherein the linker is attached to the nucleotide unit. In some embodiments, the nucleotide unit comprises a base, sugar and at least one phosphate group, and the linker is attached to the nucleotide unit through the base. FIG. 17 shows non-limiting examples of various configurations of multivalent molecules. Left (Class I) of FIG. 17 are schematics of multivalent molecules having a “starburst” or “helter-skelter” configuration. Center (Class II) of FIG. 17 is a schematic of a multivalent molecule having a dendrimer configuration. Right (Class III) of FIG. 17 is a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’. In some embodiments, the linker comprises an aliphatic chain or an oligo ethylene glycol chain where both linker chains having 2-6 subunits. In some embodiments, the linker also includes an aromatic moiety. An example of a nucleotide arm is shown in FIG. 21. Examples of multivalent molecules are shown in FIGS. 17-20. An example of a spacer is shown in FIG. 22 (top) and examples of linkers are shown in FIG. 22 (bottom) and FIG. 23. Examples of nucleotides attached to a linker are shown in FIGS. 24-27. An example of a biotinylated nucleotide arm is shown in FIG. 28. FIG. 28 is a schematic of a non-limiting example of the chemical structure of a biotinylated nucleotide-arm. In FIG. 28, the nucleotide unit is connected to the linker via a propargyl amine attachment at the 5 position of a pyrimidine base or the 7 position of a purine base.

In some embodiments, a multivalent molecule comprises a core attached to multiple nucleotide arms, wherein the multiple nucleotide arms have the same type of nucleotide unit which is selected from a group consisting of dATP, dGTP, dCTP, dTTP and dUTP.

In some embodiments, a multivalent molecule comprises a core attached to multiple nucleotide arms, where each arm includes a nucleotide unit. In some embodiments, the nucleotide unit comprises an aromatic base, a five carbon sugar (e.g., ribose or deoxyribose), and one or more phosphate groups (e.g., 1-10 phosphate groups). In some embodiments, the plurality of multivalent molecules can comprise one type of multivalent molecule having one type of nucleotide unit selected from a group consisting of dATP, dGTP, dCTP, dTTP and dUTP. The plurality of multivalent molecules can comprise a mixture of any combination of two or more types of multivalent molecules, where individual multivalent molecules in the mixture comprise nucleotide units selected from a group consisting of dATP, dGTP, dCTP, dTTP, or dUTP, or combinations thereof.

In some embodiments, the nucleotide unit comprises a chain of one, two or three phosphorus atoms where the chain may be attached to the 5′ carbon of the sugar moiety via an ester or phosphoramide linkage. In some embodiments, at least one nucleotide unit is a nucleotide analog having a phosphorus chain in which the phosphorus atoms are linked together with intervening O, S, NH, methylene or ethylene. In some embodiments, the phosphorus atoms in the chain include substituted side groups including O, S or BH3. In some embodiments, the chain includes phosphate groups substituted with analogs including phosphoramidate, phosphorothioate, phosphordithioate, and O-methyl phosphoramidite groups.

In some embodiments, the multivalent molecule comprises a core attached to multiple nucleotide arms, wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to 3′ sugar position where the sugar comprises a ribose or deoxyribose sugar moiety. In some embodiments, the chain terminating moiety is removable/cleavable from 3′ sugar position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide unit, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh3)4), with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP), or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the nucleotide unit comprising a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker and nucleotide unit, and wherein the core, linker, or nucleotide unit, or combinations thereof, are labeled with a detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core comprises a streptavidin-type or avidin-type moiety which includes an avidin protein, as well as any derivatives, analogs and other non-native forms of avidin that can bind to at least one biotin moiety. Other forms of avidin moieties include native and recombinant avidin and streptavidin as well as derivatized molecules, e.g. non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN, CAPTAVIDIN, NEUTRA VIDIN and NEUTRALITE AVIDIN.

In some embodiments, any of the methods for sequencing nucleic acid molecules described herein can include forming a binding complex, where the binding complex comprises (i) a polymerase, a nucleic acid template molecule duplexed with a primer, and a nucleotide, or the binding complex comprises (ii) a polymerase, a nucleic acid template molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. The binding complex may have a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing, or combinations thereof. The binding complex (e.g., ternary complex) remains stable until subjected to a condition that causes dissociation of interactions between any of the polymerase, template molecule, primer, or the nucleotide unit or the nucleotide, or combinations thereof. For example, a dissociating condition comprises contacting the binding complex with any one or any combination of a detergent, EDTA, or water, or combinations thereof. In some embodiments, the present disclosure provides said method wherein the binding complex is deposited on, attached to, or hybridized to, a surface showing a contrast to noise ratio in the detecting step of greater than 20. In some embodiments, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

### Compaction Oligonucleotides

A compaction oligonucleotide comprises a single-stranded linear oligonucleotide having a 5′ region that can hybridize to a first portion of a concatemer molecule and the compaction oligonucleotide having a 3′ region that can hybridize to a second portion of the concatemer molecule (e.g., the same concatemer molecule). In some embodiments, hybridization of the compaction oligonucleotides to individual concatemer molecules causes the concatemer molecule to collapse or fold into a DNA nanoball which is more compact in shape and size compared to a non-collapsed DNA molecule. A spot image of a DNA nanoball can be represented as a Gaussian spot and the size can be measured as a full width half maximum (FWHM). A smaller spot size as indicated by a smaller FWHM may correlate with an improved image of the spot. In some embodiments, the FWHM of a DNA nanoball spot can be about 10 μm or smaller. The DNA nanoball can be a compact nucleic acid structure having a full width half maximum (FWHM) that is smaller compared to a concatemer that is not collapsed/folded into a DNA nanoball.

In some embodiments, compaction oligonucleotides comprise single stranded oligonucleotides comprising DNA, RNA, or a combination of DNA and RNA. The compaction oligonucleotides can be any length, including 20-150 nucleotides, or 30-100 nucleotides, or 40-80 nucleotides in length.

In some embodiments, the compaction oligonucleotides comprise a 5′ region and a 3′ region, and optionally an intervening region between 5′ and 3′ regions. The intervening region can be any length, for example about 2-20 nucleotides in length. In some embodiments, the intervening region comprises a homopolymer having consecutive identical bases (e.g., AAA, GGG, CCC, TTT or UUU). In some embodiments, the intervening region comprises a non-homopolymer sequence.

The 5′ region of the compaction oligonucleotides can be wholly complementary or partially complementary along its length to a first portion of a concatemer molecule. The 3′ region of the compaction oligonucleotides can be wholly complementary or partially complementary along its length to a second portion of a concatemer molecule. The 5′ region of the compaction oligonucleotides can hybridize to a first universal sequence portion of a concatemer molecule. The 3′ region of the compaction oligonucleotides can hybridize to a second universal sequence portion of a concatemer molecule. The 5′ and 3′ regions of the compaction oligonucleotide can hybridize to the concatemer to pull together distal portions of the concatemer causing compaction of the concatemer to form a DNA nanoball.

The 5′ region of the compaction oligonucleotide can have the same sequence as 3′ region. The 5′ region of the compaction oligonucleotide can have a sequence that is different from the 3′ region. The 3′ region of the compaction oligonucleotide can have a sequence that is a reverse sequence of 5′ region.

### Supports and Low Non-Specific Coatings

In some embodiments, the flow cell devices disclosed herein can include a support, e.g., a solid support as disclosed herein. The present disclosure provides pairwise sequencing compositions and methods which employ a support comprising a plurality of oligonucleotide surface primers immobilized thereon. In some embodiments, the support is passivated with a low non-specific binding coating. The surface coatings described herein may exhibit very low non-specific binding to reagents that may be used for nucleic acid capture, amplification and sequencing workflows, such as dyes, nucleotides, enzymes, and nucleic acid primers. The surface coatings exhibit low background fluorescence signals or high contrast-to-noise (CNR) ratios compared to existing surface coatings.

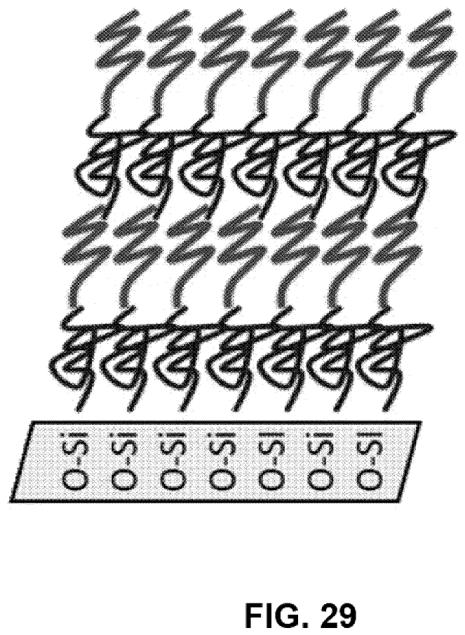

The low non-specific binding coating may comprise one layer or multiple layers (FIG. 29). FIG. 29 is a schematic of a non-limiting example of the flow cell devices in which the support comprises a glass substrate and alternating layers of hydrophilic coatings which are covalently or non-covalently adhered to the glass, and which further comprises chemically-reactive functional groups that serve as attachment sites for oligonucleotide primers. In some embodiments, the plurality of surface primers is immobilized to the low non-specific binding coating. In some embodiments, at least one surface primer is embedded within the low non-specific binding coating. The low non-specific binding coating may enable improved nucleic acid hybridization and amplification performance. The supports may comprise a substrate (or support structure), one or more layers of a covalently or non-covalently attached low-binding, chemical modification layers, e.g., silane layers, polymer films, and one or more covalently or non-covalently attached surface primers that can be used for tethering single-stranded nucleic acid library molecules to the support. In some embodiments, the formulation of the coating, e.g., the chemical composition of one or more layers, the coupling chemistry used to cross-link the one or more layers to the support, or to each other, or combinations thereof, and the total number of layers, may be varied such that non-specific binding of proteins, nucleic acid molecules, and other hybridization and amplification reaction components to the coating are minimized or reduced relative to a comparable monolayer. The formulation of the coating described herein may be varied such that non-specific hybridization on the coating is minimized or reduced relative to a comparable monolayer. The formulation of the coating may be varied such that non-specific amplification on the coating is minimized or reduced relative to a comparable monolayer. The formulation of the coating may be varied such that specific amplification rates, or yields, or combinations thereof, on the coating are maximized. Amplification levels suitable for detection are achieved in no more than 2, 3, 4, 5, 6, 7, 8, 9, 10, 15, 20, 25, 30, or more than 30 amplification cycles in some cases disclosed herein.

The support structure that comprises the one or more chemically-modified layers, e.g., layers of a low non-specific binding polymer, may be independent or integrated into another structure or assembly. For example, in some embodiments, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments, the support structure comprises the interior surface (such as the lumen surface) of a capillary etched into a planar chip.

The attachment chemistry used to graft a first chemically-modified layer to the surface of the support may be dependent on both the material from which the surface is fabricated and the chemical nature of the layer. In some embodiments, the first layer may be covalently attached to the surface. In some embodiments, the first layer may be non-covalently attached, e.g., adsorbed to the support through non-covalent interactions such as electrostatic interactions, hydrogen bonding, or van der Waals interactions between the support and the molecular components of the first layer. In either case, the support may be treated prior to attachment or deposition of the first layer. Any of a variety of existing surface preparation techniques may be used to clean or treat the surface. For example, glass or silicon surfaces may be acid-washed using a Piranha solution (a mixture of sulfuric acid (H2SO₄) and hydrogen peroxide (H2O2)), base treatment in KOH and NaOH, or cleaned using an oxygen plasma treatment method, or combinations thereof.

Silane chemistries constitute non-limiting approaches for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding coatings include, but are not limited to, (3-Aminopropyl) trimethoxysilane (APTMS), (3-Aminopropyl)triethoxysilane (APTES), any of a variety of PEG-silanes (e.g., comprising molecular weights of 1K, 2K, 5K, 10K, 20K, etc.), amino-PEG silane (e.g., comprising a free amino functional group), maleimide-PEG silane, biotin-PEG silane, and the like.

Any of a variety of existing molecules including, but not limited to, amino acids, peptides, nucleotides, oligonucleotides, other monomers or polymers, or combinations thereof may be used in creating the one or more chemically-modified layers on the support, where the choice of components used may be varied to alter one or more properties of the layers, e.g., the surface density of functional groups, or tethered oligonucleotide primers, or combinations thereof, the hydrophilicity/hydrophobicity of the layers, or the three three-dimensional nature (e.g., “thickness”) of the layer. Examples of polymers that may be used to create one or more layers of low non-specific binding material in any of the disclosed coatings include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface, or to cross-link the layers to each other, or combinations thereof, include, but are not limited to, biotin-streptavidin interactions (or variations thereof), his tag-Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the support. Alternatively, the surface coating may be patterned, such that the chemical modification layers are confined to one or more discrete regions of the support. For example, the coating may be patterned using photolithographic techniques to create an ordered array or random pattern of chemically-modified regions on the support. Alternately, or in combination, the coating may be patterned using, e.g., contact printing techniques, or ink-jet printing techniques, or combinations thereof. In some embodiments, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In some embodiments, the low nonspecific binding coatings comprise hydrophilic polymers that are non-specifically adsorbed or covalently grafted to the support. Passivation may be performed utilizing poly(ethylene glycol) (PEG, also known as polyethylene oxide (PEO) or polyoxyethylene) or other hydrophilic polymers with different molecular weights and end groups that are linked to a support using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting coating. In some embodiments, surface primers with different nucleotide sequences, or base modifications (or other biomolecules, e.g., enzymes or antibodies), or combinations thereof, may be tethered to the resulting layer at various surface densities. In some embodiments, for example, both surface functional group density and surface primer concentration may be varied to attain a surface primer density range. Additionally, surface primer density can be controlled by diluting the surface primers with other molecules that carry the same functional group. For example, amine-labeled surface primers can be diluted with amine-labeled polyethylene glycol in a reaction with an NHS-ester coated surface to reduce the final primer density. Surface primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of an existing concentration.

In some embodiments, the low nonspecific binding coatings comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl) acrylamide-co-acrylamide (PAZAM).

In order to scale primer surface density and add additional dimensionality to hydrophilic or amphoteric coatings, supports comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the support significantly. Existing PEG coating approaches use monolayer primer deposition, which may be reported for single molecule applications, but do not yield high copy numbers for nucleic acid amplification applications. As described herein “layering” can be accomplished using existing crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple steps.

Examples of materials from which the support structure may be fabricated include, but are not limited to, glass, fused-silica, silicon, a polymer microporous styrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic support structures are contemplated.

The support structure may be rendered in any of a variety of existing geometries and dimensions, and may comprise any of a variety of existing materials. For example, the support structure may be locally planar (e.g., comprising a microscope slide or the surface of a microscope slide). Globally, the support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). In some embodiments, the surface of the support structure used for nucleic acid hybridization and amplification may be a solid, non-porous surface. In some embodiments, the surface of the support structure used for nucleic acid hybridization and amplification may be porous, such that the coatings described herein penetrate the porous surface, and nucleic acid hybridization and amplification reactions performed thereon may occur within the pores.

The support structure that comprises the one or more chemically-modified layers, e.g., layers of a low non-specific binding polymer, may be independent or integrated into another structure or assembly. For example, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments the support structure comprises the interior surface (such as the lumen surface) of a capillary etched into a planar chip.

As noted, the low non-specific binding supports of the present disclosure exhibit reduced non-specific binding of proteins, nucleic acids, and other components of the hybridization formulation, or amplification formulation, or combinations thereof, used for solid-phase nucleic acid amplification. The degree of non-specific binding exhibited by a given support surface may be assessed either qualitatively or quantitatively. For example, exposure of the surface to fluorescent dyes (e.g., cyanins such as Cy3, or Cy5, etc., fluoresceins, coumarins, rhodamines, etc. or other dyes disclosed herein), fluorescently-labeled nucleotides, fluorescently-labeled oligonucleotides, or fluorescently-labeled proteins (e.g. polymerases), or combinations thereof, under a standardized set of conditions, followed by a specified rinse protocol and fluorescence imaging, may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, or fluorescently-labeled proteins (e.g. polymerases), or combinations thereof, under a standardized set of conditions, followed by a specified rinse protocol and fluorescence imaging, may be used as a quantitative tool for comparison of non-specific binding on supports comprising different surface formulations-provided that care has been taken to ensure that the fluorescence imaging is performed under conditions where fluorescence signal is linearly related (or related in a predictable manner) to the number of fluorophores on the support surface (e.g., under conditions where signal saturation, or self-quenching, or combinations thereof, of the fluorophore is not an issue) and suitable calibration standards are used. In some embodiments, other existing techniques, for example, radioisotope labeling and counting methods, may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed by detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other existing detectable label. In some embodiments, the degree of non-specific binding exhibited by a given support surface formulation may thus be assessed in terms of the number of non-specifically bound protein molecules (or nucleic acid molecules or other molecules) per unit area. In some embodiments, the low-binding supports of the present disclosure may exhibit non-specific protein binding (or non-specific binding of other specified molecules, (e.g., cyanins such as Cy3, or Cy5, etc., fluoresceins, coumarins, rhodamines, etc. or other dyes disclosed herein)) of less than 0.001 molecule per μm2, less than 0.01 molecule per μm2, less than 0.1 molecule per μm2, less than 0.25 molecule per μm2, less than 0.5 molecule per μm2, less than 1 molecule per μm2, less than 10 molecules per μm2, less than 100 molecules per μm2, or less than 1,000 molecules per μm2. A given support surface of the present disclosure may exhibit non-specific binding falling anywhere within this range, for example, of less than 86 molecules per μm2. For example, some modified surfaces disclosed herein exhibit nonspecific protein binding of less than 0.5 molecule/μm² following contact with a 1 μM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm2. In independent nonspecific binding assays, 1 uM labeled Cy3 SA (ThermoFisher), 1 μM Cy5 SA dye (ThermoFisher), 10 μM Aminoallyl-dUTP-ATTO-647N (Jena Biosciences), 10 μM Aminoallyl-dUTP-ATTO-Rhol 1 (Jena Biosciences), 10 μM Aminoallyl-dUTP-ATTO-Rhol 1 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) were incubated on the low binding coated supports at 37° C. for 15 minutes in a 384 well plate format. Each well was rinsed 2-3× with 50 μl of deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (e.g., inverted fluorescence microscope) (Olympus Corp., Center Valley, Pa.) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, N.Y.), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm2. In some embodiments, the coated support was immersed in a buffer (e.g., 25 mM ACES, pH 7.4) while the image was acquired.

In some embodiments, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surface disclosed herein may range from about 0 degrees to about 30 degrees. In some embodiments, the water contact angle for the hydrophilic, low-binding support surfaced disclosed herein may be no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases, the contact angle may not be more than 40 degrees. A given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash steps may be performed in less than 60, 50, 40, 30, 20, 15, 10, or less than 10 seconds. For example, adequate wash steps may be performed in less than 30 seconds.

Some low-binding surfaces of the present disclosure exhibit significant improvement in stability or durability to prolonged exposure to solvents and elevated temperatures, or to repeated cycles of solvent exposure or changes in temperature. For example, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents, or elevated temperatures, or combinations thereof (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes, or changes in temperature, or combinations thereof (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100-fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100-fold greater than a signal of an adjacent amplified nucleic acid population region of the surface.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer may be attached or tethered to the support surface. In some embodiments, the one or more type of adapters or primers may comprise spacer sequences, adapter sequences for hybridization to adapter-ligated target library nucleic acid sequences, forward amplification primers, reverse amplification primers, sequencing primers, or molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface.

In some embodiments, the tethered adapter, or primer sequences, or combinations thereof, may range in length from about 10 nucleotides to about 100 nucleotides. In some embodiments, the tethered adapter, or primer sequences, or combinations thereof, may be at least 10, at least 20, at least 30, at least 40, at least 50, at least 60, at least 70, at least 80, at least 90, or at least 100 nucleotides in length. In some embodiments, the tethered adapter, or primer sequences, or combinations thereof, may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter, or primer sequences, or combinations thereof, may range from about 20 nucleotides to about 80 nucleotides. The length of the tethered adapter, or primer sequences, or combinations thereof, may have any value within this range, e.g., about 24 nucleotides.

In some embodiments, the resultant surface density of primers (e.g., capture primers) on the low binding support surfaces of the present disclosure may range from about 100 primer molecules per μm² to about 100,000 primer molecules per μm². In some embodiments, the resultant surface density of primers on the low binding support surfaces of the present disclosure may range from about 1,000 primer molecules per μm² to about 1,000,000 primer molecules per μm². In some embodiments, the surface density of primers may be at least 1,000, at least 10,000, at least 100,000, or at least 1,000,000 molecules per μm². In some embodiments, the surface density of primers may be at most 1,000,000, at most 100,000, at most 10,000, or at most 1,000 molecules per μm². Any of the lower and upper values described in this paragraph may be combined to form a range included within the present disclosure, for example, in some embodiments the surface density of primers may range from about 10,000 molecules per μm² to about 100,000 molecules per μm². The surface density of primer molecules may have any value within this range, e.g., about 455,000 molecules per μm². In some embodiments, the surface density of target library nucleic acid sequences initially hybridized to adapter or primer sequences on the support surface may be less than or equal to that indicated for the surface density of tethered primers. In some embodiments, the surface density of clonally-amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000/μm², while also comprising at least a second region having a substantially different local density.

In some embodiments, the performance of nucleic acid hybridization, or amplification reactions, or combinations thereof, using the disclosed reaction formulations and low-binding supports may be assessed using fluorescence imaging techniques, where the contrast-to-noise ratio (CNR) of the images provides a key metric in assessing amplification specificity and non-specific binding on the support. CNR is commonly defined as: CNR=(Signal−Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times may be minimized). At high CNR, the imaging time required to reach accurate discrimination (and thus accurate base-calling in the case of sequencing applications) can be drastically reduced even with moderate improvements in CNR. Improved CNR in imaging data on the imaging integration time provides a method for more accurately detecting features such as clonally-amplified nucleic acid colonies on the support surface.

In most ensemble-based sequencing approaches, the background term may be measured as the signal associated with interstitial regions. In addition to interstitial background (Bᵢₙₜₑᵣ), intrastitial background (Bᵢₙₜᵣₐ) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bᵢₙₜₑᵣ background signal arises from a variety of sources; a few examples include auto-fluorescence from consumable flow cells, non-specific adsorption of detection molecules that yield spurious fluorescence signals that may obscure the signal from the ROI, and the presence of non-specific DNA amplification products (e.g., those arising from primer dimers). In existing next generation sequencing (NGS) applications, this background signal in the current field-of-view (FOV) may be averaged over time and subtracted. The signal arising from individual DNA colonies (e.g., (Signal)-B (interstial) in the FOV) yields a discernable feature that can be classified. In some embodiments, the intrastitial background (B (intrastitial)) can contribute a confounding fluorescence signal that is not specific to the target of interest, but is present in the same ROI, thus making it far more difficult to average and subtract.

Nucleic acid amplification on the low-binding coated supports described herein may decrease the B (interstitial) background signal by reducing non-specific binding, may lead to improvements in specific nucleic acid amplification, and may lead to a decrease in non-specific amplification that can impact the background signal arising from both the interstitial and intrastitial regions. In some embodiments, the disclosed low-binding coated supports, optionally used in combination with the disclosed hybridization, or amplification reaction formulations, or combinations thereof, may lead to improvements in CNR by a factor of 2, 5, 10, 100, 250, 500 or 1000-fold over those achieved using existing supports and hybridization, amplification, or sequencing protocols, or combinations thereof. Although described here in the context of using fluorescence imaging as the read-out or detection mode, the same principles apply to the use of the disclosed low-binding coated supports and nucleic acid hybridization and amplification formulations for other detection modes as well, including both optical and non-optical detection modes.

The headings provided herein are not limitations of the various aspects of the disclosure, which aspects can be understood by reference to the specification as a whole.

### Definitions

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to existing methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000), which is hereby incorporated by reference. See also Ausubel et al., Current Protocols in Molecular Biology, Greene Publishing Associates (1992), which is hereby incorporated by reference. The nomenclatures utilized in connection with, and the laboratory procedures and techniques described herein are those well-known and commonly used in the art.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken to mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, the terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of”, or “consisting essentially of”, or combinations thereof, are also provided.

As used herein, the terms “about,” “approximately,” and “substantially” refer to a value or composition that is within an acceptable error range for the particular value or composition as determined by one of ordinary skill in the art, which will depend in part on how the value or composition is measured or determined, e.g., the limitations of the measurement system. For example, “about,” “approximately,” or “substantially” can mean within one or more than one standard deviation per the practice in the art. Alternatively, “about” or “approximately” can mean a range of up to 10% (e.g., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about,” “approximately,” and “substantially” can be assumed to be within an acceptable error range for that particular value or composition. Also, where ranges, or subranges, or combinations thereof, of values are provided, the ranges, or subranges, or combinations thereof, can include the endpoints of the ranges, or subranges, or combinations thereof.

The term “glass” refers to silica-based material, including silicate, borosilicate, fused silica, fused quartz, glass, quartz, or lead glass.

The term “contrast-to-noise ratio” (CNR) used herein refers to a measure used to determine image quality, which is useful for accurately determining which base is present at each position in a target nucleic acid sequence, as well as the read length, reproducibility, and throughput of a sequencing system. In the context of a sequencing system disclosed herein, CNR may be calculated with the following equation: CNR=(Signal−Background)/(Noise), and where Background=(Bintrastitial+Binterstitial), wherein Bintrastitial refers to the “intrastitial” background that exists within a region on a surface occupied by a DNA colony, and Binterstitial is the “interstitial” background between the DNA colonies.

The term “polony” used herein refers to a nucleic acid library molecule that can be clonally amplified in-solution or on-support to generate an amplicon that can serve as a template molecule for sequencing. In some embodiments, a linear library molecule can be circularized to generate a circularized library molecule, and the circularized library molecule can be clonally amplified in-solution or on-support to generate a concatemer. In some embodiments, the concatemer can serve as a nucleic acid template molecule which can be sequenced. The concatemer is sometimes referred to as a polony. In some embodiments, a polony includes a nucleotide strand.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to post-translation modification such as proteolytic cleavage, cleavage due to ribosomal skipping, hydroxylation, methylation, lipidation, acetylation, SUMOylation, ubiquitination, glycosylation, phosphorylation, or disulfide bond formation, or combinations thereof. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins.

The term “polymerase” and its variants, as used herein, comprises any enzyme that can catalyze polymerization of nucleotides (including analogs thereof) into a nucleic acid strand. Such nucleotide polymerization may occur in a template-dependent fashion. A polymerase may comprise one or more active sites at which nucleotide binding, or catalysis of nucleotide polymerization, or combinations thereof, can occur. In some embodiments, a polymerase includes other enzymatic activities, such as for example, 3′ to 5′ exonuclease activity or 5′ to 3′ exonuclease activity. In some embodiments, a polymerase has strand displacing activity. A polymerase can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). In some embodiments, a polymerase can be isolated from a cell, or generated using recombinant DNA technology or chemical synthesis methods. In some embodiments, a polymerase can be expressed in prokaryote, eukaryote, viral, or phage organisms. In some embodiments, a polymerase can be post-translationally modified proteins or fragments thereof. A polymerase can be derived from a prokaryote, eukaryote, virus or phage. A polymerase may comprise DNA-directed DNA polymerase and RNA-directed DNA polymerase.

As used herein, the term “fidelity” refers to the accuracy of DNA polymerization by template-dependent DNA polymerase. The fidelity of a DNA polymerase may be measured by the error rate (the frequency of incorporating an inaccurate nucleotide, e.g., a nucleotide that is not complementary to the template nucleotide). The accuracy or fidelity of DNA polymerization is maintained by both the polymerase activity and 3′-5′ exonuclease activity of a DNA polymerase.

As used herein, the term “binding complex” refers to a complex formed by binding together a nucleic acid duplex, a polymerase, and a free nucleotide or a nucleotide unit of a multivalent molecule, where the nucleic acid duplex comprises a nucleic acid template molecule hybridized to a nucleic acid primer. In the binding complex, the free nucleotide or nucleotide unit may or may not be bound to the 3′ end of the nucleic acid primer at a position that is opposite a complementary nucleotide in the nucleic acid template molecule. A “ternary complex” is an example of a binding complex which is formed by binding together a nucleic acid duplex, a polymerase, and a free nucleotide or nucleotide unit of a multivalent molecule, where the free nucleotide or nucleotide unit is bound to the 3′ end of the nucleic acid primer (as part of the nucleic acid duplex) at a position that is opposite a complementary nucleotide in the nucleic acid template molecule.

The term “persistence time” and related terms refers to the length of time that a binding complex remains stable without dissociation of any of the components, where the components of the binding complex include a nucleic acid template and nucleic acid primer, a polymerase, or a nucleotide unit of a multivalent molecule or a free (e.g., unconjugated) nucleotide, or combinations thereof. The nucleotide unit or the free nucleotide can be complementary or non-complementary to a nucleotide residue in the template molecule. The nucleotide unit or the free nucleotide can bind to the 3′ end of the nucleic acid primer at a position that is opposite a complementary nucleotide residue in the nucleic acid template molecule. The persistence time is indicative of the stability of the binding complex and strength of the binding interactions. Persistence time can be measured by observing the onset, or duration, or combinations thereof, of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One label, for example, is a fluorescent label. The binding complex (e.g., ternary complex) remains stable until subjected to a condition that causes dissociation of interactions between any of the polymerase, template molecule, primer, or the nucleotide unit or the nucleotide, or combinations thereof. For example, a dissociating condition comprises contacting the binding complex with any one or any combination of a detergent, EDTA, or water, or combinations thereof.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids can be single-stranded or double-stranded. Nucleic acids comprise polymers of nucleotides, where the nucleotides include natural or non-natural bases, or sugars, or combinations thereof. Nucleic acids comprise naturally-occurring internucleosidic linkages, for example phosphodiester linkages. Nucleic acids comprise non-natural internucleoside linkages, including phosphorothioate, phosphorothiolate, or peptide nucleic acid (PNA) linkages. In some embodiments, nucleic acids comprise a one type of polynucleotides or a mixture of two or more different types of polynucleotides.

The term “primer” and related terms used herein refers to an oligonucleotide, either natural or synthetic, that is capable of hybridizing with a DNA, or RNA, or combinations thereof, polynucleotide template to form a duplex molecule. Primers may have any length and may range from 4-50 nucleotides. A primer may comprise a 5′ end and a 3′ end. The 3′ end of the primer can include a 3′ OH moiety which serves as a nucleotide polymerization initiation site in a polymerase-mediated primer extension reaction. Alternatively, 3′ end of the primer can lack a 3′ OH moiety, or can include a terminal 3′ blocking group that inhibits nucleotide polymerization in a polymerase-mediated reaction. Any one nucleotide, or more than one nucleotide, along the length of the primer can be labeled with a detectable reporter moiety. A primer can be in solution (e.g., a soluble primer) or can be immobilized to a support (e.g., a capture primer).

The term “template nucleic acid”, “template polynucleotide”, “target nucleic acid” “target polynucleotide”, “template strand” and other variations refer to a nucleic acid strand that serves as the base nucleic acid molecule for generating a complementary nucleic acid strand. The template nucleic acid can be single-stranded or double-stranded, or the template nucleic acid can have single-stranded or double-stranded portions. The sequence of the template nucleic acid can be partially or wholly complementary to the sequence of the complementary strand. The template nucleic acid can be obtained from a naturally-occurring source, recombinant form, or chemically synthesized to include any type of nucleic acid analog. The template nucleic acid can be linear, circular, or can come in other forms. The template nucleic acids can include an insert region having an insert sequence which is also known as a sequence of interest. The template nucleic acids can also include at least one adaptor sequence. The template nucleic acid can be a concatemer having two or tandem copies of a sequence of interest and at least one adaptor sequence. The insert region can be isolated in any form, including chromosomal, genomic, organellar (e.g., mitochondrial, chloroplast or ribosomal), recombinant molecules, cloned, amplified, cDNA, RNA such as precursor mRNA or mRNA, oligonucleotides, whole genomic DNA, obtained from fresh frozen paraffin embedded tissue, needle biopsies, cell free circulating DNA, or any type of nucleic acid library. The insert region can be isolated from any source including from organisms such as prokaryotes, eukaryotes (e.g., humans, plants and animals), fungus, viruses cells, tissues, normal or diseased cells or tissues, body fluids including blood, urine, serum, lymph, tumor, saliva, anal and vaginal secretions, amniotic samples, perspiration, semen, environmental samples, culture samples, or synthesized nucleic acid molecules prepared using recombinant molecular biology or chemical synthesis methods. The insert region can be isolated from any organ, including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs. The template nucleic acid can be subjected to nucleic acid analysis, including sequencing and composition analysis.

When used in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization can comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary. Complementary nucleic acid strands may not need to hybridize with each other across their entire length. The complementary base pairing can be the standard A-T or C-G base pairing or can be other forms of base-pairing interactions. Duplex nucleic acids can include mismatched base-paired nucleotides.

The term “nucleotides” and related terms refers to a molecule comprising an aromatic base, a five carbon sugar (e.g., ribose or deoxyribose), and at least one phosphate group. Canonical or non-canonical nucleotides are consistent with use of the term. The phosphate in some embodiments comprises a monophosphate, diphosphate, or triphosphate, or corresponding phosphate analog. In some embodiments, the nucleotide comprises 1, 2, 3, 4, 5, 6, 7, 8, 9 or 10 phosphate groups. The term “nucleoside” refers to a molecule comprising an aromatic base and a sugar.

Nucleotides (and nucleosides) may comprise a hetero cyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick, or Hoogstein hydrogen bonds, or combinations thereof, with an appropriate complementary base. Examples of bases include, but are not limited to, purines and pyrimidines such as: 2-aminopurine, 2,6-diaminopurine, adenine (A), ethenoadenine, N⁶-Δ²-isopentenyladenine (6iA), N⁶-Δ²-isopentenyl-2-methylthioadenine (2ms6iA), N⁶-methyladenine, guanine (G), isoguanine, N²-dimethylguanine (dmG), 7-methylguanine (7mG), 2-thiopyrimidine, 6-thioguanine (6sG), hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional bases can be found in Fasman, 1989, in “Practical Handbook of Biochemistry and Molecular Biology”, pp. 385-394, CRC Press, Boca Raton, Fla., which is hereby incorporated by reference.

Nucleotides (and nucleosides) may comprise a sugar moiety, such as carbocyclic moiety (Ferraro and Gotor 2000 Chem. Rev. 100:4319-48, which is hereby incorporated by reference), acyclic moieties (Martinez, et al., 1999 Nucleic Acids Research 27:1271-1274; Martinez, et al., 1997 Bioorganic & Medicinal Chemistry Letters vol. 7:3013-3016, which is hereby incorporated by reference), and other sugar moieties (Joeng, et al., 1993 J. Med. Chem. 36:2627-2638; Kim, et al., 1993 J. Med. Chem. 36:30-7; Eschenmosser 1999 Science 284:2118-2124; and U.S. Pat. No. 5,558,991, which is hereby incorporated by reference). The sugar moiety comprises: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides comprise a chain of one, two or three phosphorus atoms where the chain may be attached to the 5′ carbon of the sugar moiety via an ester or phosphoramide linkage. In some embodiments, the nucleotide is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methyl phosphoramidite groups.

When used in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation can be conducted with natural nucleotides, or nucleotide analogs, or combinations thereof. Nucleotide incorporation may occur in a template-dependent fashion. Any suitable method of extending a nucleic acid molecule may be used, including primer extension catalyzed by a DNA polymerase or RNA polymerase.

The term “reporter moiety”, “reporter moieties” or related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. Reporter moieties may be selected so that each absorbs excitation radiation, or emits fluorescence, or combinations thereof, at a wavelength distinguishable from the other reporter moieties to permit monitoring the presence of different reporter moieties in the same reaction or in different reactions. Two or more different reporter moieties can be selected having spectrally distinct emission profiles or having minimal overlapping spectral emission profiles. Reporter moieties can be linked (e.g., operably linked) to nucleotides, nucleosides, nucleic acids, enzymes (e.g., polymerases or reverse transcriptases), or support (e.g., surfaces).

A reporter moiety (or label) comprises a fluorescent label or a fluorophore. Examples of fluorescent moieties which may serve as fluorescent labels or fluorophores include, but are not limited to fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, all of which are hereby incorporated by reference in their entireties, or derivatives thereof, or any combination thereof. Cyanine dyes may exist in either sulfonated or non-sulfonated forms and consist of two indolenin, benzo-indolium, pyridium, thiozolium, or quinolinium, or combinations thereof, groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6Iohexyl)-2-((1E,3E)-5-((E)-1-(6-((2,5-dioxopyrrolidin-1-yl)oxy)-6-oxohexyl)-3,3-dimethyl-5-indolin-2-ylidene) penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin-2-ylidene) penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene) hepta-1,3,5-trien-1-yl]-3H-indolium or 1-(5-carboxypentyl)-2-[(1E,3E,5E,7Z)-7-(1-ethyl-5-sulfo-1,3-dihydro-2H-indol-2-ylidene) hepta-1,3,5-trien-1-yl]-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

In some embodiments, the reporter moiety can be a FRET pair, such that multiple classifications can be performed under a single excitation and imaging step. As used herein, FRET may comprise excitation exchange (Forster) transfers, or electron-exchange (Dexter) transfers.

The terms “linked”, “joined”, “attached”, and variants thereof comprise any type of fusion, bond, adherence or association between any combination of compounds or molecules that is of sufficient stability to withstand use in the particular procedure. The procedure can include but is not limited to: nucleotide transient-binding; nucleotide incorporation; de-blocking; washing; removing; flowing; detecting; imaging, or identifying, or combinations thereof. Such linkage can comprise, for example, covalent, ionic, hydrogen, dipole-dipole, hydrophilic, hydrophobic, or affinity bonding, bonds or associations involving van der Waals forces, mechanical bonding, and the like. In some embodiments, such linkage occurs intramolecularly, for example linking together the ends of a single-stranded or double-stranded linear nucleic acid molecule to form a circular molecule. In some embodiments, such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998), all of which are hereby incorporated by reference in their entireties.

The term “operably linked” and “operably joined” or related terms as used herein refers to juxtaposition of components. The juxtapositioned components can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription, or translation, or combinations thereof, initiation sequence, transcription, or translation, or combinations thereof, termination sequence, polypeptide secretion signal sequences, and the like. In some embodiments, the host cell regulatory sequence controls expression of the level, timing or location, or combinations thereof, of the transgene.

The term “adaptor” and related terms refers to oligonucleotides that can be operably linked (appended) to a target polynucleotide, where the adaptor confers a function to the co-joined adaptor-target molecule. Adaptors comprise DNA, RNA, chimeric DNA/RNA, or analogs thereof. Adaptors can include at least one ribonucleoside residue. Adaptors can be single-stranded, double-stranded, or have single-stranded portions, or double-stranded portions, or combinations thereof. Adaptors can be configured to be linear, stem-looped, hairpin, or Y-shaped forms. Adaptors can be any length, including 4-100 nucleotides or longer. Adaptors can have blunt ends, overhang ends, or a combination of both. Overhang ends include 5′ overhang and 3′ overhang ends. The 5′ end of a single-stranded adaptor, or one strand of a double-stranded adaptor, can have a 5′ phosphate group or lack a 5′ phosphate group. Adaptors can include a 5′ tail that does not hybridize to a target polynucleotide (e.g., tailed adaptor), or adaptors can be non-tailed. An adaptor can include a sequence that is complementary to at least a portion of a primer, such as an amplification primer, a sequencing primer, or a capture primer (e.g., soluble or immobilized capture primers). Adaptors can include a random sequence or degenerate sequence. Adaptors can include at least one inosine residue. Adaptors can include at least one phosphorothioate, phosphorothiolate, or phosphoramidate, or combinations thereof, linkage. Adaptors can include a barcode sequence which can be used to distinguish polynucleotides (e.g., insert sequences) from different sample sources in a multiplex assay. Adaptors can include a unique identification sequence (e.g., unique molecular index (UMI); or a unique molecular tag) that can be used to uniquely identify a nucleic acid molecule to which the adaptor is appended. In some embodiments, a unique identification sequence can be used to increase error correction and accuracy, reduce the rate of false-positive variant calls, or increase sensitivity of variant detection, or combinations thereof. Adaptors can include at least one restriction enzyme recognition sequence, including any one or any combination of two or more selected from a group consisting of type I, type II, type III, type IV, type Hs or type IIB.

The term “universal sequence”, “universal adaptor sequences” and related terms refers to a sequence in a nucleic acid molecule that is common among two or more polynucleotide molecules. For example, adaptors having the same universal sequence can be joined to a plurality of polynucleotides so that the population of co-joined molecules carry the same universal adaptor sequence. Examples of universal adaptor sequences include an amplification primer sequence, a sequencing primer sequence or a capture primer sequence (e.g., soluble or support-immobilized capture primers).

In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support comprises a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

In some embodiments, the surface of the support is coated with one or more compounds to produce a passivated layer on the support. In some embodiments, the support comprises a low non-specific binding surface that enable improved nucleic acid hybridization and amplification performance on the support. The support may comprise one or more layers of a covalently or non-covalently attached low-binding, chemical modification layers, e.g., silane layers, polymer films, and one or more covalently or non-covalently attached oligonucleotides that may be used for immobilizing a plurality of nucleic acid template molecules to the support.

In some embodiments, the degree of hydrophilicity (or “wettability” with aqueous solutions) of the surface coatings may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In some cases, the contact angle is no more than 40 degrees. A given hydrophilic, low-binding support surface of the present disclosure may exhibit a water contact angle having a value of anywhere within this range.

The present disclosure provides a plurality (e.g., two or more) of nucleic acid templates immobilized to a support. In some embodiments, the immobilized plurality of nucleic acid templates has the same sequence or has different sequences. In some embodiments, individual nucleic acid template molecules in the plurality of nucleic acid templates are immobilized to a different site on the support. In some embodiments, two or more individual nucleic acid template molecules in the plurality of nucleic acid templates are immobilized to a site on the support. In some embodiments, the support comprises a plurality of sites arranged in an array. The term “array” refers to a support comprising a plurality of sites located at pre-determined locations on the support to form an array of sites. The sites can be discrete and separated by interstitial regions. In some embodiments, the pre-determined sites on the support can be arranged in one dimension in a row or a column, or arranged in two dimensions in rows and columns. In some embodiments, the plurality of pre-determined sites is arranged on the support in an organized fashion. In some embodiments, the plurality of pre-determined sites is arranged in any organized pattern, including rectilinear, hexagonal patterns, grid patterns, patterns having reflective symmetry, patterns having rotational symmetry, or the like. The pitch between different pairs of sites can be that same or can vary. In some embodiments, the support can have nucleic acid template molecules immobilized at a plurality of sites at a surface density of about 102-1015 sites per mm2, or more, to form a nucleic acid template array. In some embodiments, the support comprises at least 102 sites, at least 103 sites, at least 104 sites, at least 105 sites, at least 106 sites, at least 107 sites, at least 108 sites, at least 109 sites, at least 1010 sites, at least 1011 sites, at least 1012 sites, at least 1013 sites, at least 1014 sites, at least 1015 sites, or more, where the sites are located at pre-determined locations on the support. In some embodiments, a plurality of pre-determined sites on the support (e.g., 102-1015 sites or more) are immobilized with nucleic acid templates to form a nucleic acid template array. In some embodiments, the nucleic acid templates that are immobilized at a plurality of pre-determined sites by hybridization to immobilized surface capture primers, or the nucleic acid templates, are covalently attached to the surface capture primers. In some embodiments, the nucleic acid templates that are immobilized at a plurality of pre-determined sites are, for example, immobilized at 102-1015 sites or more. In some embodiments, the nucleic acid templates that are immobilized at a plurality of sites on the support comprise linear or circular nucleic acid template molecules or a mixture of both linear and circular molecules. In some embodiments, the immobilized nucleic acid templates are clonally-amplified to generate immobilized nucleic acid polonies at the plurality of pre-determined sites. In some embodiments, individual immobilized nucleic acid template molecules comprise one copy of a target sequence of interest, or comprise concatemers having two or more tandem copies of a target sequence of interest.

In some embodiments, a support comprising a plurality of sites located at random locations on the support is referred to herein as a support having randomly located sites thereon. In some embodiments, the location of the randomly located sites on the support are not pre-determined. In some embodiments, the plurality of randomly-located sites is arranged on the support in a disordered fashion, or unpredictable fashion, or combinations thereof. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some embodiments, a plurality of randomly located sites on the support (e.g., 10²-10¹⁵ sites or more) is immobilized with nucleic acid templates to form a support immobilized with nucleic acid templates. In some embodiments, the nucleic acid templates that are immobilized at a plurality of randomly located sites by hybridization to immobilized surface capture primers, or the nucleic acid templates, are covalently attached to the surface capture primer. In some embodiments, the nucleic acid templates that are immobilized at a plurality of randomly located sites are, for example, immobilized at 102-1015 sites or more. In some embodiments, the nucleic acid templates that are immobilized at a plurality of sites on the support comprise linear or circular nucleic acid template molecules or a mixture of both linear and circular molecules. In some embodiments, the immobilized nucleic acid templates are clonally-amplified to generate immobilized nucleic acid polonies at the plurality of randomly located sites. In some embodiments, individual immobilized nucleic acid template molecules comprise one copy of a target sequence of interest or comprise concatemers having two or more tandem copies of a target sequence of interest.

In some embodiments, with respect to nucleic acid template molecules immobilized to pre-determined or random sites on the support, the plurality of immobilized nucleic acid template molecules on the support are in fluid communication with each other to permit flowing a solution of reagents (e.g., enzymes including polymerases, multivalent molecules, nucleotides, divalent cations, or buffers and the like, or combinations thereof) onto the support so that the plurality of immobilized nucleic acid template molecules on the support can be reacted with the reagents in a massively parallel manner. In some embodiments, the fluid communication of the plurality of immobilized nucleic acid template molecules can be used to conduct nucleotide binding assays, or conduct nucleotide polymerization reactions (e.g., primer extension or sequencing), or combinations thereof, on the plurality of immobilized nucleic acid template molecules, and to conduct detection and imaging for massively parallel sequencing. In some embodiments, the term “immobilized” and related terms refer to nucleic acid molecules or enzymes (e.g., polymerases) that are attached to the support at pre-determined or random locations, where the nucleic acid molecules or enzymes are attached directly to a support through covalent bonds or non-covalent interactions, or the nucleic acid molecules or enzymes are attached to a coating on the support.

When used in reference to a low binding surface coating, one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxyethyl methacrylate) (branched PHEMA), branched poly(oligo (ethylene glycol) methyl ether methacrylate) (branched POEGMA), branched polyglutamic acid (branched PGA), branched poly-lysine, branched poly-glucoside, and dextran.

In some embodiments, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branched.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some embodiments, e.g., wherein at least one layer of a multi-layered surface comprises a branched polymer, the number of covalent bonds between a branched polymer molecule of the layer being deposited and molecules of the previous layer may range from about one covalent linkage per molecule and about 32 covalent linkages per molecule. In some embodiments, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some embodiments, the number of layers is at least 1, at least 2, at least 3, at least 4, at least 5, at least 6, at least 7, at least 8, at least 9, or at least 10. In some embodiments, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some embodiments the number of layers may range from about 2 to about 4. In some embodiments, all of the layers may comprise the same material. In some embodiments, each layer may comprise a different material. In some embodiments, the plurality of layers may comprise a plurality of materials. In some embodiments at least one layer may comprise a branched polymer. In some embodiments, all of the layers may comprise a branched polymer.

One or more layers of low non-specific binding material may in some cases be deposited on, or conjugated to, or combinations thereof, the substrate surface using a polar protic solvent, a polar or polar aprotic solvent, a nonpolar solvent, or any combination thereof. In some embodiments, the solvent used for layer deposition, or coupling, or combinations thereof, may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino) propanesulfonic acid (MOPS), etc.), or any combination thereof. In some embodiments, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some embodiments, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

The term “branched polymer” and related terms refers to a polymer having a plurality of functional groups that help conjugate a biologically active molecule such as a nucleotide, and the functional group can be either on the side chain of the polymer or directly attached to a central core or central backbone of the polymer. The branched polymer can have a linear backbone with one or more functional groups coming off the backbone for conjugation. The branched polymer can also be a polymer having one or more sidechains, wherein the side chain has a site suitable for conjugation. Examples of the functional group include but are limited to hydroxyl, ester, amine, carbonate, acetal, aldehyde, aldehyde hydrate, alkenyl, acrylate, methacrylate, acrylamide, active sulfone, hydrazide, thiol, alkanoic acid, acid halide, isocyanate, isothiocyanate, maleimide, vinylsulfone, dithiopyridine, vinylpyridine, iodoacetamide, epoxide, glyoxal, dione, mesylate, tosylate, and tresylate.

As used herein, the term “clonally amplified” and its variants refers to a nucleic acid template molecule that has been subjected to one or more amplification reactions either in-solution or on-support. In the case of in-solution amplified template molecules, the resulting amplicons are distributed onto the support. Prior to amplification, the template molecule may comprise a sequence of interest and at least one universal adaptor sequence. In some embodiments, clonal amplification comprises the use of a polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, single-stranded binding (SSB) protein-dependent amplification, or any combination thereof.

As used herein, the term “sequencing” and its variants comprise obtaining sequence information from a nucleic acid strand, which may be by determining the identity of at least some nucleotides (including their nucleobase components) within the nucleic acid template molecule. While in some embodiments, “sequencing” a given region of a nucleic acid molecule includes identifying each and every nucleotide within the region that is sequenced, in some embodiments, “sequencing” comprises methods whereby the identity of some of the nucleotides in the region is determined, while the identity of some nucleotides remains undetermined or incorrectly determined. Any suitable method of sequencing may be used. In an embodiment, sequencing can include label-free or ion based sequencing methods. In some embodiments, sequencing can include labeled or dye-containing nucleotide or fluorescent based nucleotide sequencing methods. In some embodiments, sequencing can include polony-based sequencing or bridge sequencing methods. In some embodiments, sequencing includes massively parallel sequencing platforms that employ sequence-by-synthesis, sequence-by-hybridization or sequence-by-binding procedures. Examples of massively parallel sequence-by-synthesis procedures include polony sequencing, pyrosequencing (e.g., from 454 Life Sciences; U.S. Pat. Nos. 7,211,390, 7,244,559 and 7,264,929, all of which are hereby incorporated by reference), chain-terminator sequencing (e.g., from Illumina; U.S. Pat. No. 7,566,537; Bentley 2006 Current Opinion Genetics and Development 16:545-552; and Bentley, et al., 2008 Nature 456:53-59, all of which are hereby incorporated by reference), ion-sensitive sequencing (e.g., from Ion Torrent), probe-anchor ligation sequencing (e.g., Complete Genomics), DNA nanoball sequencing, nanopore DNA sequencing. Examples of single molecule sequencing include Heliscope single molecule sequencing, and single molecule real time (SMRT) sequencing from Pacific Biosciences (Levene, et al., 2003 Science 299 (5607): 682-686; Eid, et al., 2009 Science 323 (5910): 133-138; U.S. Pat. Nos. 7,170,050; 7,302,146; and 7,405,281, all of which are hereby incorporated by reference). An example of sequence-by-hybridization includes SOLID sequencing (e.g., from Life Technologies; WO 2006/084132, which is hereby incorporated by reference). An example of sequence-by-binding includes Omniome sequencing (e.g., U.S. Pat. No. 10,246,744, which is hereby incorporated by reference).

It is to be appreciated that the Detailed Description section, and not any other section, is intended to be used to interpret the claims. Other sections may set forth one or more but not all embodiments as contemplated by the inventor(s), and thus, are not intended to limit this disclosure or the appended claims in any way.

While this disclosure describes embodiments for fields and applications, it can be understood that the disclosure is not limited thereto. Other embodiments and modifications thereto are possible and are within the scope and spirit of this disclosure. For example, and without limiting the generality of this paragraph, embodiments are not limited to the software, hardware, firmware, or entities illustrated in the figures or described herein, or any combination thereof. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries may be defined as long as the specified functions and relationships (or equivalents thereof) are appropriately performed. Also, alternative embodiments may perform functional blocks, steps, operations, methods, etc. using orderings different from those described herein.

References herein to “one embodiment,” “an embodiment,” “an example embodiment,” “some embodiments,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it may be within the knowledge of persons skilled in the relevant art(s) to incorporate such feature, structure, or characteristic into other embodiments whether or not explicitly mentioned or described herein.

Additionally, some embodiments may be described using the expression “coupled” and “connected” along with their derivatives. These terms are not necessarily intended as synonyms for each other. For example, some embodiments may be described using the terms “connected”, or “coupled”, or combinations thereof, to indicate that two or more elements are in direct physical or electrical contact with each other. The term “coupled,” however, may also mean that two or more elements are not in direct contact with each other, but yet still co-operate or interact with each other.

While embodiments of the present inventive concepts have been shown and described herein, it will be obvious to those skilled in the art that such embodiments are provided by way of example only. The breadth and scope of this disclosure may not be limited by any of the above-described embodiments. Numerous variations, changes, and substitutions will now occur to those skilled in the art without departing from the inventive concepts. It may be understood that various alternatives to the embodiments of the inventive concepts described herein may be employed in practicing the inventive concepts. It is intended that the following claims define the scope of the inventive concepts and that methods and structures within the scope of these claims and their equivalents be covered thereby.

### Embodiments

Embodiment 1 A flow cell system comprising:
a flow cell device comprising:
a support comprising one or more substrates;
one or more channels defined by the one or more substrates, wherein the one or more channels are configured to allow fluids and a gas gap between the fluids to flow therethrough;
an inlet in the one or more substrates, the inlet in fluidic connection with the one or more channels, wherein the inlet comprises an open landing area in one substrate of the one or more substrates; and
an outlet in the one or more substrates,
wherein the one or more channels run from the inlet to the outlet.

Embodiment 2 A flow cell system comprising:
a flow cell device comprising:
a support comprising one or more substrates;
one or more channels defined by the one or more substrates;
an inlet in the one or more substrates, the inlet in fluidic connection with the one or more channels, the inlet comprising an open landing area in one substrate of the one or more substrates, wherein the open landing area is at least partly covered with a coating; and an outlet in the one or more substrates,
wherein the one or more channels run from the inlet to the outlet.

Embodiment 3 A flow cell system comprising:
a flow cell device comprising:
a support comprising one or more substrates;
one or more channels defined by between the one or more substrates;
an inlet in the one or more substrates, the inlet in fluidic connection with the one or more channels, wherein the inlet comprises an open landing area in one substrate of the one or more substrates;
a cleaning outlet in the one or more substrates, wherein the cleaning outlet is in fluidic connection with the inlet; and
an outlet in the one or more substrates, wherein the one or more channels run from the inlet to the outlet.

Embodiment 4 The flow cell system of any one of the preceding embodiments, wherein the one or more channels are configured to allow the gas gap to flow therethrough between allowing a first reagent and a second reagent to flow therethrough.

Embodiment 5 The flow cell system of any one of the preceding embodiments, wherein the one or more channels are configured to allow the gas gap to flow therethrough during DNA sequencing.

Embodiment 6 The flow cell system of any one of the preceding embodiments, wherein the one or more channels are configured to allow the gas gap to flow therethrough from the inlet.

Embodiment 7 The flow cell system of any one of the preceding embodiments, wherein the one or more channels are configured to allow the gas gap to flow therethrough to facilitate reducing contamination of the second reagent by the first reagent to DNA sequencing.

Embodiment 8 The flow cell system of any one of the preceding embodiments, wherein the one or more channels are configured to allow the gas gap to flow therethrough to reduce a minimum amount of the first reagent, the second reagent, or a washing reagent for DNA sequencing.

Embodiment 9 The flow cell system of any one of the preceding embodiments, wherein each of the one of the one or more channels comprises a surface.

Embodiment 10 The flow cell system of any one of the preceding embodiments, wherein the surface is an inner surface.

Embodiment 11 The flow cell system of any one of the preceding embodiments, wherein the surface is an exterior surface.

Embodiment 12 The flow cell system of any one of the preceding embodiments, wherein the surface comprises an interior top surface, an interior bottom surface, or both.

Embodiment 13 The flow cell system of any one of the preceding embodiments, wherein the surface comprises an exterior top surface, an exterior bottom surface, or both.

Embodiment 14 The flow cell system of any one of the preceding embodiments, wherein the surface is a planar surface.

Embodiment 15 The flow cell system of any one of the preceding embodiments, wherein the surface is passivated.

Embodiment 16 The flow cell system of any one of the preceding embodiments, wherein the surface is passivated with a coating that immobilize surface capture primers, nucleic acid template molecules, or both for capturing polynucleotides.

Embodiment 17 The flow cell system of any one of the preceding embodiments, wherein the surface comprises polynucleotides captured thereon.

Embodiment 18 The flow cell system of any one of the preceding embodiments, wherein the gas gap is configured to dry at least part of the surface of the one or more channels.

Embodiment 19 The flow cell system of any one of the preceding embodiments, wherein the gas gap does not impair chemical functions of the surface.

Embodiment 20 The flow cell system of any one of the preceding embodiments, wherein the coating of the surface comprises at least one hydrophilic polymer coating layer.

Embodiment 21 The flow cell system of any one of the preceding embodiments, wherein the coating of the surface comprises a plurality of oligonucleotide molecules attached to at least one hydrophilic polymer coating layer.

Embodiment 22 The flow cell system of any one of the preceding embodiments, wherein the surface comprises at least one discrete region that comprises a plurality of clonally-amplified sample nucleic acid molecules that have been annealed to a plurality of attached oligonucleotide molecules.

Embodiment 23 The flow cell system of any one of the preceding embodiments, wherein the at least one hydrophilic polymer coating layer has a water contact angle of no more than about 50 degrees.

Embodiment 24 The flow cell system of any one of the preceding embodiments, wherein at least one of the plurality of clonally-amplified sample nucleic acid molecules comprises a concatemer annealed to at least one of the plurality of attached oligonucleotide.

Embodiment 25 The flow cell system of any one of the preceding embodiments, wherein the at least one hydrophilic polymer coating layer comprises PEG.

Embodiment 26 The flow cell system of any one of the preceding embodiments, wherein the surface further comprises a second hydrophilic polymer coating layer.

Embodiment 27 The flow cell system of any one of the preceding embodiments, wherein the at least one hydrophilic polymer coating layer comprises a branched hydrophilic polymer.

Embodiment 28 The flow cell system of any one of the preceding embodiments, wherein the branched hydrophilic polymer comprises at least 8 branches.

Embodiment 29 The flow cell system of any one of the preceding embodiments, wherein the at least one of the plurality of the clonally-amplified sample nucleic acid molecules comprises a single-stranded multimeric nucleic acid molecule comprising repeats of a regularly occurring monomer unit.

Embodiment 30 The flow cell system of any one of the preceding embodiments, wherein the single-stranded multimeric nucleic acid molecule is at least 10 kilobases in length.

Embodiment 31 The flow cell system of any one of the preceding embodiments, wherein the at least one of the plurality of the clonally-amplified sample nucleic acid molecules further comprises a double-stranded monomeric copy of the regularly occurring monomer unit.

Embodiment 32 The flow cell system of any one of the preceding embodiments, wherein the plurality of oligonucleotide molecules is present at about a uniform surface density across the surface.

Embodiment 33 The flow cell system of any one of the preceding embodiments, wherein the plurality of oligonucleotide molecules is present at a local surface density of at least 100,000 molecules/μm2 at a first position on the surface, and at a second local surface density at a second position on the surface.

Embodiment 34 The flow cell system of any one of the preceding embodiments, wherein the coating comprises:
a first layer comprising a monolayer of polymer molecules tethered to the surface of the substrate;
a second layer comprising a second monolayer of polymer molecules tethered to the polymer molecules of the first layer; and
a third layer comprising a third monolayer of polymer molecules tethered to the polymer molecules of the second layer, wherein at least one of the first layer, the second layer, or the third layer comprises branched polymer molecules.

Embodiment 35 The flow cell system of any one of the preceding embodiments, wherein the third layer further comprises oligonucleotides tethered to the polymer molecules of the third layer.

Embodiment 36 The flow cell system of any one of the preceding embodiments, wherein the oligonucleotides tethered to the polymer molecules of the third layer are distributed at a plurality of depths throughout the third layer.

Embodiment 37 The flow cell system of any one of the preceding embodiments, wherein the coating further comprises:
a fourth layer comprising branched polymer molecules tethered to the polymer molecules of the third layer, and
a fifth layer comprising polymer molecules tethered to the branched polymer molecules of the fourth layer.

Embodiment 38 The flow cell system of any one of the preceding embodiments, wherein the polymer molecules of the fifth layer further comprise oligonucleotides tethered to the polymer molecules of the fifth layer.

Embodiment 39 The flow cell system of any one of the preceding embodiments, wherein the oligonucleotides tethered to the polymer molecules of the fifth layer are distributed at a plurality of depths throughout the fifth layer.

Embodiment 40 The flow cell system of any one of the preceding embodiments, wherein the at least one hydrophilic polymer coating layer comprises a molecule selected from the group consisting of: polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxyethyl methacrylate) (PHEMA), poly(oligo (ethylene glycol) methyl ether methacrylate) (POEGMA), polyglutamic acid (PGA), poly-lysine, poly-glucoside, streptavidin, and dextran.

Embodiment 41 The flow cell system of any one of the preceding embodiments, wherein when the clonally-amplified sample nucleic acid molecules or complementary sequences thereof are labeled with Cyanine dye-3, an image of the surface exhibits a ratio of fluorescence intensities for the clonally-amplified, Cyanine dye-3-labeled sample nucleic acid molecules, or complementary sequences thereof, and nonspecific Cyanine dye-3 dye adsorption background (Bᵢₙₜₑᵣ) of at least 3:1.

Embodiment 42 The flow cell system of any one of the preceding embodiments, wherein the image of the surface exhibits a ratio of fluorescence intensities for clonally amplified, Cyanine dye-3-labeled sample nucleic acid molecules, or complementary sequences thereof, and a combination of nonspecific Cyanine dye-3 dye adsorption background and nonspecific amplification background (Bᵢₙₜₑᵣ+Bᵢₙₜᵣₐ) of at least 3:1.

Embodiment 43 The flow cell system of any one of the preceding embodiments, wherein when the clonally-amplified sample nucleic acid molecules or complementary sequences thereof are labeled with Cyanine dye-3, the image of the surface exhibits a ratio of fluorescence intensities for clonally-amplified, Cyanine dye-3-labeled sample nucleic acid molecules, or complementary sequences thereof, and nonspecific dye adsorption background (Binter) of at least 5:1.

Embodiment 44 The flow cell system of any one of the preceding embodiments, wherein the image of the surface exhibits a ratio of fluorescence intensities for clonally-amplified, Cyanine dye-3-labeled sample nucleic acid molecules, or complementary sequences thereof, and a combination of nonspecific Cyanine dye-3 dye adsorption background and nonspecific amplification background (Binter+Bintra) of at least 5:1.

Embodiment 45 The flow cell system of any one of the preceding embodiments, wherein when the clonally-amplified sample nucleic acid molecules or complementary sequences thereof are labeled with Cyanine dye-3, the fluorescence image of the surface exhibits a contrast-to-noise ratio (CNR) of at least 20 when the fluorescence image is acquired using an inverted microscope equipped with a 20× objective, NA=0.75, dichroic mirror optimized for 532 nm light, a bandpass filter optimized for Cyanine dye-3 emission, and a camera under non-signal saturating conditions, while the surface is immersed in a buffer.

Embodiment 46 The flow cell system of any one of the preceding embodiments, wherein the plurality of oligonucleotide molecules is present at a surface density of at least 1,000 molecules/m².

Embodiment 47 The flow cell system of any one of the preceding embodiments, wherein the first reagent is configured to wet the surface of the one or more channels.

Embodiment 48 The flow cell system of any one of the preceding embodiments, wherein the second reagent is configured to rewet the surface of the one or more channels after at least partly drying the surface by the gas gap.

Embodiment 49 The flow cell system of any one of the preceding embodiments, wherein the flow cell system further comprises:
a fluidic control device comprising:
a first pump coupled with the outlet; and
a dispenser that is configured to openly dispense one or more reagents to the inlet.

Embodiment 50 The flow cell system of any one of the preceding embodiments, wherein the first pump or a second pump is configured to introduce the gas gap via the inlet and flow the gas gap at least partly through the one or more channels.

Embodiment 51 The flow cell system of any one of the preceding embodiments, wherein the gas gap comprises air.

Embodiment 52 The flow cell system of any one of the preceding embodiments, wherein the gas gap comprises dry air.

Embodiment 53 The flow cell system of any one of the preceding embodiments, wherein the gas gap comprises one or more inert gases.

Embodiment 54 The flow cell system of any one of the preceding embodiments, wherein the gas gap comprises one or more active gases.

Embodiment 55 The flow cell system of any one of the preceding embodiments, wherein the first or the second reagent comprise liquid.

Embodiment 56 The flow cell system of any one of the preceding embodiments, wherein the first or the second reagent is deprived of air bubbles that are greater than a predetermined size.

Embodiment 57 The flow cell system of any one of the preceding embodiments, wherein the coating comprises a liquid-repelling coating.

Embodiment 58 The flow cell system of any one of the preceding embodiments, wherein the coating comprises an omniphobic coating.

Embodiment 59 The flow cell system of any one of the preceding embodiments, wherein the coating comprises a slippery liquid-infused porous surface (SLIPS).

Embodiment 60 The flow cell system of any one of the preceding embodiments, wherein the coating comprises a slippery omniphobic covalently attached liquid (SOCAL) coating.

Embodiment 61 The flow cell system of any one of the preceding embodiments, wherein the coating comprises a liquid-like polymer brush surface that is covalently attached to the one or more substrates.

Embodiment 62 The flow cell system of any one of the preceding embodiments, wherein the coating is formed by impregnating lubricants in one or more porous surfaces.

Embodiment 63 The flow cell system of any one of the preceding embodiments, wherein the lubricants comprise a liquid with a surface energy below about 20 mJ/m².

Embodiment 64 The flow cell system of any one of the preceding embodiments, wherein the lubricants comprise a silicone oil.

Embodiment 65 The flow cell system of any one of the preceding embodiments, wherein the coating comprises a surface energy that is below about 20 mJ/m².

Embodiment 66 The flow cell system of any one of the preceding embodiments, wherein the coating is formed by acid-catalyzed graft polycondensation of one or more saline monomers.

Embodiment 67 The flow cell system of any one of the preceding embodiments, wherein the one or more saline monomers comprises dimethyldimethoxysilane.

Embodiment 68 The flow cell system of any one of the preceding embodiments, wherein the open landing area is in fluidic connection with the one or more channels.

Embodiment 69 The flow cell system of any one of the preceding embodiments, wherein the open landing area is in fluidic connection with one corresponding channel of the one or more channels.

Embodiment 70 The flow cell system of any one of the preceding embodiments, wherein the open landing area is in fluidic connection with two or more of the one or more channels.

Embodiment 71 The flow cell system of any one of the preceding embodiments, wherein the open landing area is on a bottom substrate of the one or more substrates.

Embodiment 72 The flow cell system of any one of the preceding embodiments, wherein the inlet comprises a hole in a top substrate of the one or more substrates.

Embodiment 73 The flow cell system of any one of the preceding embodiments, wherein the hole in the top substrate is positioned above at least part of the open landing area.

Embodiment 74 The flow cell system of any one of the preceding embodiments, wherein the dispenser is configured to openly dispense the one or more reagents through the hole to the open landing area.

Embodiment 75 The flow cell system of any one of the preceding embodiments, wherein the dispenser is configured to openly dispense the one or more reagents from a tip of the dispenser to the open landing area.

Embodiment 76 The flow cell system of any one of the preceding embodiments, wherein the dispenser is configured to openly dispense the one or more reagents from the tip of the dispenser to the open landing area, without any tubing in between.

Embodiment 77 The flow cell system of any one of the preceding embodiments, wherein at least part of the tip of the dispenser is in contact with the open landing area.

Embodiment 78 The flow cell system of any one of the preceding embodiments, wherein the tip of the dispenser is not in contact with the open landing area.

Embodiment 79 The flow cell system of any one of the preceding embodiments, wherein the flow cell device further comprises a cleaning outlet in the one or more substrates.

Embodiment 80 The flow cell system of any one of the preceding embodiments, wherein the cleaning outlet is in fluidic connection with the inlet.

Embodiment 81 The flow cell system of any one of the preceding embodiments, wherein the cleaning outlet is in fluidic connection with the open landing area.

Embodiment 82 The flow cell system of any one of the preceding embodiments, wherein the cleaning outlet is positioned underneath the open landing area.

Embodiment 83 The flow cell system of any one of the preceding embodiments, wherein the cleaning outlet is in a top or bottom substrate of the one or more substrates.

Embodiment 84 The flow cell system of any one of the preceding embodiments, wherein the cleaning outlet is a side port on the one or more substrates.

Embodiment 85 The flow cell system of any one of the preceding embodiments, wherein the cleaning outlet is configured to be coupled with the first pump or the second pump.

Embodiment 86 The flow cell system of any one of the preceding embodiments, wherein the one or more channels comprises microfluidic channels.

Embodiment 87 The flow cell system of any one of the preceding embodiments, wherein the surface is coated with fluorescent beads that are chemically immobilized to the surface.

Embodiment 88 The flow cell system of any one of the preceding embodiments, wherein the fluorescent beads are covalently attached to the surface.

Embodiment 89 The flow cell system of any one of the preceding embodiments, wherein a gap between the interior top surface and the interior bottom surface is about 150 μm, 130 μm, 120 μm, 110 μm, 100 μm, 90 μm, 80 μm, 70 μm, 60 μm, 50 μm, or 40 μm.

Embodiment 90 The flow cell system of any one of the preceding embodiments, wherein a height of the one or more channels is about 150 μm, 130 μm, 120 μm, 110 μm, 100 μm, 90 μm, 80 μm, 70 μm, 60 μm, 50 μm, or 40 μm.

Embodiment 91 The flow cell system of any one of the preceding embodiments, wherein the polynucleotides captured thereon are configured to be imaged in a sequencing cycle.

Embodiment 92 The flow cell system of any one of the preceding embodiments, wherein the one or more substrates comprises a top substrate and a bottom substrate.

Embodiment 93 The flow cell system of any one of the preceding embodiments, wherein the one or more channels are defined between the top substrate and the bottom substrate.

Embodiment 94 The flow cell system of any one of the preceding embodiments, wherein the one or more channels are defined at least partly in a top surface of the bottom substrate.

Embodiment 95 The flow cell system of any one of the preceding embodiments, wherein the one or more channels are defined at least partly in a bottom surface of the top substrate.

Embodiment 96 The flow cell system of any one of the preceding embodiments, wherein the one or more substrates further comprises a middle substrate.

Embodiment 97 The flow cell system of any one of the preceding embodiments, wherein the one or more channels are defined at least partly in the middle substrate.

Embodiment 98 The flow cell system of any one of the preceding embodiments, wherein the one or more substrates comprise glass or plastic.

Embodiment 99 The flow cell system of any one of the preceding embodiments, wherein at least part of the support is transparent.

Embodiment 100 The flow cell system of any one of the preceding embodiments, wherein at least part of the one or more substrates is transparent.

Embodiment 101 The flow cell system of any one of the preceding embodiments, wherein the support is solid.

Embodiment 102 The flow cell system of any one of the preceding embodiments, wherein the one or more channels comprises 1, 2, 3, 4, 5, 6, 7, or 8 channels.

Embodiment 103 The flow cell system of any one of the preceding embodiments, wherein the one or more channels comprises 2, 4, 6, 8, or 10 channels.

Embodiment 104 The flow cell system of any one of the preceding embodiments, wherein each channel of the one or more channels comprises a lane length of less than about 70 mm, 75 mm, 80 mm, or 90 mm.

Embodiment 105 The flow cell system of any one of the preceding embodiments, wherein each channel of the one or more channels comprises a lane width of less than about 10 mm, 8 mm, 7 mm, 6 mm, 5 mm, 4 mm, or 3 mm.

Embodiment 106 The flow cell system of any one of the preceding embodiments, wherein covering at least a portion of the open landing area with a second coating comprises covering at least a portion of the open landing area with a slippery coating.

Embodiment 107 The flow cell system of any one of the preceding embodiments, wherein covering at least a portion of the open landing area with the second coating comprises covering at least a portion of the open landing area with a liquid-repelling coating.

Embodiment 108 The flow cell system of any one of the preceding embodiments, wherein covering at least a portion of the open landing area with the second coating comprises covering at least a portion of the open landing area with an omniphobic coating.

Embodiment 109 The flow cell system of any one of the preceding embodiments, wherein covering at least a portion of the open landing area with the second coating comprises covering at least a portion of the open landing area with a slippery liquid-infused porous surface (SLIPS).

Embodiment 110 The flow cell system of any one of the preceding embodiments, wherein covering at least a portion of the open landing area with the second coating comprises covering at least a portion of the open landing area with a slippery omniphobic covalently attached liquid (SOCAL) coating.

Embodiment 111 The flow cell system of any one of the preceding embodiments, wherein covering at least a portion of the open landing area with the second coating comprises covering at least a portion of the open landing area with a liquid-like polymer brush surface that is covalently attached to the one or more substrates.

Embodiment 112 The flow cell system of any one of the preceding embodiments, wherein covering at least a portion of the open landing area with the second coating comprises impregnating lubricants in porous surfaces to generate the coating with a surface energy below about 20 mJ/m2.

Embodiment 113 The flow cell system of any one of the preceding embodiments, wherein covering at least a portion of the open landing area with the second coating comprises acid-catalyzed graft polycondensation of one or more saline monomers.

Embodiment 114 The flow cell system of any one of the preceding embodiments, wherein the one or more saline monomers comprises dimethyldimethoxysilane.

Embodiment 115 The flow cell system of any one of the preceding embodiments, wherein cleaning at least part of the first reagent from at least part of the one or more channels is during DNA sequencing.

Embodiment 116 The flow cell system of any one of the preceding embodiments, wherein the at least part of the first reagent is remained in the one or more channels.

Embodiment 117 The flow cell system of any one of the preceding embodiments, wherein the first reagent and the second reagent are different.

Embodiment 118 The flow cell system of any one of the preceding embodiments, wherein at least part of the one or more channels comprises more than about 40% of a corresponding volume or length of each of the one or more channels.

Embodiment 119 The flow cell system of any one of the preceding embodiments, wherein at least part of the one or more channels comprises more than about half of a corresponding volume or length of each of the one or more channels.

Embodiment 120 The flow cell system of any one of the preceding embodiments, wherein at least part of the one or more channels comprises more than about 60% of a corresponding volume or length of each of the one or more channels.

Embodiment 121 The flow cell system of any one of the preceding embodiments, wherein at least part of the one or more channels comprises more than about 70% of a corresponding volume or length of each of the one or more channels.

Embodiment 122 The flow cell system of any one of the preceding embodiments, wherein at least part of the one or more channels comprises more than about 80% of a corresponding volume or length of each of the one or more channels.

Embodiment 123 The flow cell system of any one of the preceding embodiments, wherein the cleaning outlet is configured to allow residuals of a first reagent on the open landing area to flow therethrough.

Embodiment 124 The flow cell system of any one of the preceding embodiments, wherein the flow cell system further comprises:
a fluidic control device comprising:
a first pump in fluidic connection with the cleaning outlet;
the first pump or a second pump in fluidic connection with the outlet; and
a dispenser that is configured to openly dispense the one or more reagents to the inlet.

Embodiment 125 The flow cell system of any one of the preceding embodiments, wherein the first pump is configured to clean the open landing area by driving residuals of a first reagent off the open landing area to flow through the cleaning outlet.

Embodiment 126 The flow cell system of any one of the preceding embodiments, wherein the residuals of the first reagent on the open landing area comprises meniscus of the first reagent.

Embodiment 127 A method for manufacturing flow cell devices, comprising:
obtaining one or more substrates;
generating one or more channels in the one or more substrates, wherein the one or more channels are configured to allow fluids and a gas gap between the fluids to flow therethrough;
forming an inlet comprising a hole in one of the one or more substrates and an open landing area, wherein the inlet is in fluidic connection with the one or more channels;
forming an outlet that is in fluidic connection with the one or more channels;
coating at least a portion of a surface of the one or more channels with a first coating, wherein the surface is configured to be dried and rewet during DNA sequencing; and
fixedly coupling the one of one or more substrates together.

Embodiment 128 A method for manufacturing flow cell devices, comprising: obtaining one or more substrates;
generating one or more channels in the one or more substrates;
forming an inlet comprising a hole in one of the one or more substrates and an open landing area, wherein the inlet is in fluidic connection with the one or more channels;
coating at least a portion of a surface of the one or more channels with a first coating;
covering at least a portion of the open landing area with a second coating; and
fixedly coupling the one of one or more substrates together.

Embodiment 129 A method for sequencing with flow cell devices, comprising:
dispensing a first reagent openly to an open landing area of an inlet of the flow cell device;
flowing at least part of the first reagent from the open landing area to one or more channels of the flow cell device;
cleaning first residuals of the first reagent from the one or more channels by driving a gas gap between fluids from the inlet and through at least part of the one or more channels; and
dispensing a second reagent openly to the open landing area.

Embodiment 130 A method for sequencing with flow cell devices, comprising:
dispensing a first reagent openly to an open landing area of an inlet of the flow cell device; flowing at least part of the first reagent from the open landing area to one or more channels of the flow cell device;
facilitating cleaning of residuals of the first reagent off the open landing area by using a coating on at least part of the open landing area; and
dispensing a second reagent openly to the open landing area.

Embodiment 131 A method for manufacturing flow cell devices, comprising: obtaining one or more substrates;
forming an inlet comprising a hole in one of the one or more substrates and an open landing area;
generating one or more channels in the one or more substrates;
forming an outlet in the one or more substrates, wherein the inlet and outlet are in fluidic connection with the one or more channels;
forming a cleaning outlet in the one or more substrates, wherein the cleaning outlet is in fluidic connection with the inlet, and wherein the cleaning outlet is closer to the inlet than to the outlet; and
fixedly coupling the one of one or more substrates together.

Embodiment 132 A method for sequencing with flow cell devices, comprising:
dispensing a first reagent openly to an open landing area of an inlet of the flow cell device; flowing at least part of the first reagent from the open landing area to one or more channels of the flow cell device;
cleaning residuals of the first reagent from at least part of the open landing area by driving the residuals through a cleaning outlet; and
dispensing a second reagent openly to the open landing area.

Embodiment 133 The method of any one of the preceding embodiments, wherein the one or more channels are configured to allow the gas gap to flow therethrough between allowing the first reagent and the second reagent to flow therethrough.

Embodiment 134 The method of any one of the preceding embodiments, wherein the one or more channels are configured to allow the gas gap to flow therethrough during DNA sequencing.

Embodiment 135 The method of any one of the preceding embodiments, wherein the one or more channels are configured to allow the gas gap to flow therethrough from the inlet.

Embodiment 136 The method of any one of the preceding embodiments, wherein the one or more channels are configured to allow the gas gap to flow therethrough to facilitate reducing contamination of the second reagent by the first reagent in DNA sequencing.

Embodiment 137 The method of any one of the preceding embodiments, wherein the one or more channels are configured to allow the gas gap to flow therethrough to reduce a minimum amount of the first reagent, the second reagent, or a washing reagent for DNA sequencing.

Embodiment 138 The method of any one of the preceding embodiments, wherein each of the one of the one or more channels comprises a surface.

Embodiment 139 The method of any one of the preceding embodiments, wherein the surface is an inner surface.

Embodiment 140 The method of any one of the preceding embodiments, wherein the surface is an exterior surface.

Embodiment 141 The method of any one of the preceding embodiments, wherein the surface comprises an interior top surface, an interior bottom surface, or both.

Embodiment 142 The method of any one of the preceding embodiments, wherein the surface comprises an exterior top surface, an exterior bottom surface, or both.

Embodiment 143 The method of any one of the preceding embodiments, wherein the surface is a planar surface.

Embodiment 144 The method of any one of the preceding embodiments, wherein the surface is passivated.

Embodiment 145 The method of any one of the preceding embodiments, wherein the surface is passivated with a coating that immobilizes surface capture primers, nucleic acid template molecules, or both for capturing polynucleotides.

Embodiment 146 The method of any one of the preceding embodiments, wherein the surface comprises polynucleotides captured thereon.

Embodiment 147 The method of any one of the preceding embodiments, wherein the gas gap is configured to dry at least part of the surface of the one or more channels.

Embodiment 148 The method of any one of the preceding embodiments, wherein the gas gap does not impair chemical functions of the surface.

Embodiment 149 The method of any one of the preceding embodiments, wherein the coating of the surface comprises at least one hydrophilic polymer coating layer.

Embodiment 150 The method of any one of the preceding embodiments, wherein the coating of the surface comprises a plurality of oligonucleotide molecules attached to at least one hydrophilic polymer coating layer.

Embodiment 151 The method of any one of the preceding embodiments, wherein the surface comprises at least one discrete region that comprises a plurality of clonally-amplified sample nucleic acid molecules that have been annealed to a plurality of attached oligonucleotide molecules.

Embodiment 152 The method of any one of the preceding embodiments, wherein the at least one hydrophilic polymer coating layer has a water contact angle of no more than about 50 degrees.

Embodiment 153 The method of any one of the preceding embodiments, wherein at least one of the plurality of clonally-amplified sample nucleic acid molecules comprises a concatemer annealed to at least one of the plurality of attached oligonucleotide.

Embodiment 154 The method of any one of the preceding embodiments, wherein the at least one hydrophilic polymer coating layer comprises PEG.

Embodiment 155 The method of any one of the preceding embodiments, wherein the surface further comprises a second hydrophilic polymer coating layer.

Embodiment 156 The method of any one of the preceding embodiments, wherein the at least one hydrophilic polymer coating layer comprises a branched hydrophilic polymer.

Embodiment 157 The method of any one of the preceding embodiments, wherein the branched hydrophilic polymer comprises at least 8 branches.

Embodiment 158 The method of any one of the preceding embodiments, wherein the at least one of the plurality of the clonally-amplified sample nucleic acid molecules comprises a single-stranded multimeric nucleic acid molecule comprising repeats of a regularly occurring monomer unit.

Embodiment 159 The method of any one of the preceding embodiments, wherein the single-stranded multimeric nucleic acid molecule is at least 10 kilobases in length.

Embodiment 160 The method of any one of the preceding embodiments, wherein at least one of the plurality of the clonally-amplified sample nucleic acid molecules further comprises a double-stranded monomeric copy of the regularly occurring monomer unit.

Embodiment 161 The method of any one of the preceding embodiments, wherein the plurality of oligonucleotide molecules is present at about a uniform surface density across the surface.

Embodiment 162 The method of any one of the preceding embodiments, wherein the plurality of oligonucleotide molecules is present at a local surface density of at least 100,000 molecules/μm2 at a first position on the surface, and at a second local surface density at a second position on the surface.

Embodiment 163 The method of any one of the preceding embodiments, wherein the coating comprises:
a first layer comprising a monolayer of polymer molecules tethered to the surface of the substrate;
a second layer comprising a second monolayer of polymer molecules tethered to the polymer molecules of the first layer; and
a third layer comprising a third monolayer of polymer molecules tethered to the polymer molecules of the second layer, wherein at least one of the first layer, the second layer, or the third layer comprises branched polymer molecules.

Embodiment 164 The method of any one of the preceding embodiments, wherein the third layer further comprises oligonucleotides tethered to the polymer molecules of the third layer.

Embodiment 165 The method of any one of the preceding embodiments, wherein the oligonucleotides tethered to the polymer molecules of the third layer are distributed at a plurality of depths throughout the third layer.

Embodiment 166 The method of any one of the preceding embodiments, wherein the coating further comprises:
a fourth layer comprising branched polymer molecules tethered to the polymer molecules of the third layer, and
a fifth layer comprising polymer molecules tethered to the branched polymer molecules of the fourth layer.

Embodiment 167 The method of any one of the preceding embodiments, wherein the polymer molecules of the fifth layer further comprise oligonucleotides tethered to the polymer molecules of the fifth layer.

Embodiment 168 The method of any one of the preceding embodiments, wherein the oligonucleotides tethered to the polymer molecules of the fifth layer are distributed at a plurality of depths throughout the fifth layer.

Embodiment 169 The method of any one of the preceding embodiments, wherein the at least one hydrophilic polymer coating layer comprises a molecule selected from the group consisting of: polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxyethyl methacrylate) (PHEMA), poly(oligo (ethylene glycol) methyl ether methacrylate) (POEGMA), polyglutamic acid (PGA), poly-lysine, poly-glucoside, streptavidin, and dextran.

Embodiment 170 The method of any one of the preceding embodiments, wherein when the clonally-amplified sample nucleic acid molecules or complementary sequences thereof are labeled with Cyanine dye-3, an image of the surface exhibits a ratio of fluorescence intensities for the clonally-amplified, Cyanine dye-3-labeled sample nucleic acid molecules, or complementary sequences thereof, and nonspecific Cyanine dye-3 dye adsorption background (Bᵢₙₜₑᵣ) of at least 3:1.

Embodiment 171 The method of any one of the preceding embodiments, wherein the image of the surface exhibits a ratio of fluorescence intensities for clonally amplified, Cyanine dye-3-labeled sample nucleic acid molecules, or complementary sequences thereof, and a combination of nonspecific Cyanine dye-3 dye adsorption background and nonspecific amplification background (Bᵢₙₜₑᵣ+Bᵢₙₜᵣₐ) of at least 3:1.

Embodiment 172 The method of any one of the preceding embodiments, wherein when the clonally-amplified sample nucleic acid molecules or complementary sequences thereof are labeled with Cyanine dye-3, the image of the surface exhibits a ratio of fluorescence intensities for clonally-amplified, Cyanine dye-3-labeled sample nucleic acid molecules, or complementary sequences thereof, and nonspecific dye adsorption background (Bᵢₙₜₑᵣ) of at least 5:1.

Embodiment 173 The method of any one of the preceding embodiments, wherein the image of the surface exhibits a ratio of fluorescence intensities for clonally-amplified, Cyanine dye-3-labeled sample nucleic acid molecules, or complementary sequences thereof, and a combination of nonspecific Cyanine dye-3 dye adsorption background and nonspecific amplification background (Bᵢₙₜₑᵣ+Bᵢₙₜᵣₐ) of at least 5:1.

Embodiment 174 The method of any one of the preceding embodiments, wherein when the clonally-amplified sample nucleic acid molecules or complementary sequences thereof are labeled with Cyanine dye-3, the fluorescence image of the surface exhibits a contrast-to-noise ratio (CNR) of at least 20 when the fluorescence image is acquired using an inverted microscope equipped with a 20× objective, NA=0.75, dichroic mirror optimized for 532 nm light, a bandpass filter optimized for Cyanine dye-3 emission, and a camera under non-signal saturating conditions, while the surface is immersed in a buffer.

Embodiment 175 The method of any one of the preceding embodiments, wherein the plurality of oligonucleotide molecules is present at a surface density of at least 1,000 molecules/m2.

Embodiment 176 The method of any one of the preceding embodiments, wherein the first reagent is configured to wet the surface of the one or more channels.

Embodiment 177 The method of any one of the preceding embodiments, wherein the second reagent is configured to rewet the surface of the one or more channels after at least partly drying the surface by the gas gap.

Embodiment 178 The method of any one of the preceding embodiments, wherein the flow cell system further comprises:
a fluidic control device comprising:
a first pump coupled with the outlet; and
a dispenser that is configured to openly dispense one or more reagents to the inlet.

Embodiment 179 The method of any one of the preceding embodiments, wherein the first pump or a second pump is configured to introduce the gas gap via the inlet and flow the gas gap at least partly through the one or more channels.

Embodiment 180 The method of any one of the preceding embodiments, wherein the gas gap comprises air.

Embodiment 181 The method of any one of the preceding embodiments, wherein the gas gap comprises dry air.

Embodiment 182 The method of any one of the preceding embodiments, wherein the gas gap comprises one or more inert gases.

Embodiment 183 The method of any one of the preceding embodiments, wherein the gas gap comprises one or more active gases.

Embodiment 184 The method of any one of the preceding embodiments, wherein the first or second reagent comprise liquid.

Embodiment 185 The method of any one of the preceding embodiments, wherein the first or the second reagent is deprived of air bubbles that are greater than a predetermined size.

Embodiment 186 The method of any one of the preceding embodiments, wherein the coating comprises a liquid-repelling coating.

Embodiment 187 The method of any one of the preceding embodiments, wherein the coating comprises an omniphobic coating.

Embodiment 188 The method of any one of the preceding embodiments, wherein the coating comprises a slippery liquid-infused porous surface (SLIPS).

Embodiment 189 The method of any one of the preceding embodiments, wherein the coating comprises a slippery omniphobic covalently attached liquid (SOCAL) coating.

Embodiment 190 The method of any one of the preceding embodiments, wherein the coating comprises a liquid-like polymer brush surface that is covalently attached to the one or more substrates.

Embodiment 191 The method of any one of the preceding embodiments, wherein the coating is formed by impregnating lubricants in one or more porous surfaces.

Embodiment 192 The method of any one of the preceding embodiments, wherein the lubricants comprise a liquid with a surface energy below about 20 mJ/m².

Embodiment 193 The method of any one of the preceding embodiments, wherein the lubricants comprise a silicone oil.

Embodiment 194 The method of any one of the preceding embodiments, wherein the coating comprises a surface energy that is below about 20 mJ/m².

Embodiment 195 The method of any one of the preceding embodiments, wherein the coating is formed by acid-catalyzed graft polycondensation of one or more saline monomers.

Embodiment 196 The method of any one of the preceding embodiments, wherein the one or more saline monomers comprises dimethyldimethoxysilane.

Embodiment 197 The method of any one of the preceding embodiments, wherein the open landing area is in fluidic connection with the one or more channels.

Embodiment 198 The method of any one of the preceding embodiments, wherein the open landing area is in fluidic connection with one of the one or more channels.

Embodiment 199 The method of any one of the preceding embodiments, wherein the open landing area is on a bottom substrate of the one or more substrates.

Embodiment 200 The method of any one of the preceding embodiments, wherein the inlet comprises a hole in a top substrate of the one or more substrates.

Embodiment 201 The method of any one of the preceding embodiments, wherein the hole in the top substrate is positioned above at least part of the open landing area.

Embodiment 202 The method of any one of the preceding embodiments, wherein the dispenser is configured to openly dispense the one or more reagents through the hole to the open landing area.

Embodiment 203 The method of any one of the preceding embodiments, wherein the dispenser is configured to openly dispense the one or more reagents from a tip of the dispenser to the open landing area.

Embodiment 204 The method of any one of the preceding embodiments, wherein the dispenser is configured to openly dispense the one or more reagents from the tip of the dispenser to the open landing area, without any tubing in between.

Embodiment 205 The method of any one of the preceding embodiments, wherein at least part of the tip of the dispenser is in contact with the open landing area.

Embodiment 206 The method of any one of the preceding embodiments, wherein the tip of the dispenser is not in contact with the open landing area.

Embodiment 207 The method of any one of the preceding embodiments, wherein the flow cell device further comprises a cleaning outlet in the one or more substrates.

Embodiment 208 The method of any one of the preceding embodiments, wherein the cleaning outlet is in fluidic connection with the inlet.

Embodiment 209 The method of any one of the preceding embodiments, wherein the cleaning outlet is in fluidic connection with the open landing area.

Embodiment 210 The method of any one of the preceding embodiments, wherein the cleaning outlet is in a top or a bottom substrate of the one or more substrates.

Embodiment 211 The method of any one of the preceding embodiments, wherein the cleaning outlet is a side port on the one or more substrates.

Embodiment 212 The method of any one of the preceding embodiments, wherein the cleaning outlet is configured to be coupled with the first pump or the second pump.

Embodiment 213 The method of any one of the preceding embodiments, wherein the one or more channels comprises microfluidic channels.

Embodiment 214 The method of any one of the preceding embodiments, wherein the surface is coated with fluorescent beads that are chemically immobilized to the surface.

Embodiment 215 The method of any one of the preceding embodiments, wherein the fluorescent beads are covalently attached to the surface.

Embodiment 216 The method of any one of the preceding embodiments, wherein a gap between the interior top surface and the interior bottom surface is about 150 μm, 130 μm, 120 μm, 110 μm, 100 μm, 90 μm, 80 μm, 70 μm, 60 μm, 50 μm, or 40 μm.

Embodiment 217 The method of any one of the preceding embodiments, wherein a height of the one or more channels is about 150 μm, 130 μm, 120 μm, 110 μm, 100 μm, 90 μm, 80 μm, 70 μm, 60 μm, 50 μm, or 40 μm.

Embodiment 218 The method of any one of the preceding embodiments, wherein the polynucleotides captured thereon are configured to be imaged in a sequencing cycle.

Embodiment 219 The method of any one of the preceding embodiments, wherein the one or more substrates comprises a top substrate and a bottom substrate.

Embodiment 220 The method of any one of the preceding embodiments, wherein the one or more channels are defined between the top substrate and the bottom substrate.

Embodiment 221 The method of any one of the preceding embodiments, wherein the one or more channels are defined at least partly in a top surface of the bottom substrate.

Embodiment 222 The method of any one of the preceding embodiments, wherein the one or more channels are defined at least partly in a bottom surface of the top substrate.

Embodiment 223 The method of any one of the preceding embodiments, wherein the one or more substrates further comprises a middle substrate.

Embodiment 224 The method of any one of the preceding embodiments, wherein the one or more channels are defined at least partly in the middle substrate.

Embodiment 225 The method of any one of the preceding embodiments, wherein the one or more substrates comprises glass or plastic.

Embodiment 226 The method of any one of the preceding embodiments, wherein at least part of the support is transparent.

Embodiment 227 The method of any one of the preceding embodiments, wherein at least part of the one or more substrates is transparent.

Embodiment 228 The method of any one of the preceding embodiments, wherein the support is solid.

Embodiment 229 The method of any one of the preceding embodiments, wherein the one or more channels comprises 1, 2, 3, 4, 5, 6, 7, or 8 channels.

Embodiment 230 The method of any one of the preceding embodiments, wherein the one or more channels comprises 2, 4, 6, 8, or 10 channels.

Embodiment 231 The method of any one of the preceding embodiments, wherein each channel of the one or more channels comprises a lane length of less than about 70 mm, 75 mm, 80 mm, or 90 mm.

Embodiment 232 The method of any one of the preceding embodiments, wherein each channel of the one or more channels comprises a lane width of less than about 10 mm, 8 mm, 7 mm, 6 mm, 5 mm, 4 mm, or 3 mm.

Embodiment 233 The method of any one of the preceding embodiments, wherein covering at least a portion of the open landing area with the second coating comprises covering at least a portion of the open landing area with a slippery coating.

Embodiment 234 The method of any one of the preceding embodiments, wherein covering at least a portion of the open landing area with the second coating comprises covering at least a portion of the open landing area with a liquid-repelling coating.

Embodiment 235 The method of any one of the preceding embodiments, wherein covering at least a portion of the open landing area with the second coating comprises covering at least a portion of the open landing area with an omniphobic coating.

Embodiment 236 The method of any one of the preceding embodiments, wherein covering at least a portion of the open landing area with the second coating comprises covering at least a portion of the open landing area with a slippery liquid-infused porous surface (SLIPS).

Embodiment 237 The method of any one of the preceding embodiments, wherein covering at least a portion of the open landing area with the second coating comprises covering at least a portion of the open landing area with a slippery omniphobic covalently attached liquid (SOCAL) coating.

Embodiment 238 The method of any one of the preceding embodiments, wherein covering at least a portion of the open landing area with the second coating comprises covering at least a portion of the open landing area with a liquid-like polymer brush surface that is covalently attached to the one or more substrates.

Embodiment 239 The method of any one of the preceding embodiments, wherein covering at least a portion of the open landing area with the second coating comprises impregnating lubricants in porous surfaces to generate the coating with a surface energy below about 20 mJ/m².

Embodiment 240 The method of any one of the preceding embodiments, wherein covering at least a portion of the open landing area with the second coating comprises acid-catalyzed graft polycondensation of one or more saline monomers.

Embodiment 241 The method of any one of the preceding embodiments, wherein the one or more saline monomers comprises dimethyldimethoxysilane.

Embodiment 242 The method of any one of the preceding embodiments, wherein cleaning at least part of the first reagent from at least part of the one or more channels is during DNA sequencing.

Embodiment 243 The method of any one of the preceding embodiments, wherein the at least part of the first reagent is remained in the one or more channels.

Embodiment 244 The method of any one of the preceding embodiments, wherein the first reagent and the second reagent are different.

Embodiment 245 The method of any one of the preceding embodiments, wherein at least part of the one or more channels comprises more than about 40% of a corresponding volume or length of each of the one or more channels.

Embodiment 246 The method of any one of the preceding embodiments, wherein at least part of the one or more channels comprises more than about half of a corresponding volume or length of each of the one or more channels.

Embodiment 247 The method of any one of the preceding embodiments, wherein at least part of the one or more channels comprises more than about 60% of a corresponding volume or length of each of the one or more channels.

Embodiment 248 The method of any one of the preceding embodiments, wherein at least part of the one or more channels comprises more than about 70% of a corresponding volume or length of each of the one or more channels.

Embodiment 249 The method of any one of the preceding embodiments, wherein at least part of the one or more channels comprises more than about 80% of a corresponding volume or length of each of the one or more channels.

Embodiment E 250 The method of any one of the preceding embodiments, further comprising: driving residuals of the first reagent or the second reagent off the open landing area via a cleaning outlet of the flow cell device.

Embodiment 251 The method of any one of the preceding embodiments, wherein the cleaning outlet is configured to allow residuals of the first reagent on the open landing area to flow therethrough.

Embodiment 252 The method of any one of the preceding embodiments, wherein the flow cell system further comprises:
a fluidic control device comprising:
a first pump in fluidic connection with the cleaning outlet;
the first pump or the second pump in fluidic connection with the outlet; and
a dispenser that is configured to openly dispense the one or more reagents to the inlet.

Embodiment 253 The method of any one of the preceding embodiments, wherein the first pump is configured to clean the open landing area by driving residuals of the first reagent off the open landing area to flow through the cleaning outlet.

Embodiment 254 The method of any one of the preceding embodiments, further comprising: cleaning at least part of the first reagent from at least part of the one or more channels by driving the gas gap between fluids from the inlet and through at least part of the one or more channels.

Embodiment 255 The method of any one of the preceding embodiments, wherein the residuals of the first reagent on the open landing area comprises meniscus of the first reagent.

1. A flow cell device comprising:
(a) a support comprising one or more substrates, wherein the one or more substrates comprise an inlet and an outlet, wherein the inlet comprises an open landing area; and
(b) one or more channels defined by the one or more substrates, wherein the one or more channels are in fluidic connection with the inlet and the outlet, wherein the one or more channels are configured to allow a fluid or a gas gap between the fluid and another fluid to flow through the one or more channels.

2. The flow cell device of claim 1, wherein the open landing area is at least partly covered with a surface coating.

3. The flow cell device of claim 1, wherein the one or more channels extend: from the inlet to the outlet or along a first direction and between the inlet and the outlet.

4-7. (canceled)

8. The flow cell device of claim 1, wherein the fluid comprises a first reagent and the another fluid comprises a second reagent, wherein the one or more channels are configured to:
allow the gas gap to flow through the one or more channels during a DNA sequencing run to;
facilitate reducing contamination of the second reagent by the first reagent in the DNA sequencing run; or
reduce a minimum amount of the first reagent, the second reagent, or a washing reagent used for the DNA sequencing run.

9. (canceled)

10. The flow cell device of claim 1, wherein the one or more channels comprise one or more surfaces.

11-16. (canceled)

17. The flow cell device of claim 10, wherein the one or more surfaces is passivated with a coating that immobilizes a surface capture primer, a nucleic acid template molecule, or both, for capturing a polynucleotide.

18-59. (canceled)

60. The flow cell device of claim 17, wherein the coating comprises a liquid-like polymer brush surface that is covalently attached to the one or more substrates.

61-66. (canceled)

67. The flow cell device of claim 1, wherein the open landing area is in fluidic connection with the one or more channels.

68-71. (canceled)

72. The flow cell device of claim 1, wherein the inlet comprises a hole in a top substrate of the one or more substrates, wherein the hole in the top substrate is positioned above at least part of the open landing area, and wherein the flow cell device is configured to allow a dispenser to openly dispense one or more reagents through the hole to the open landing area.

73-77. (canceled)

78. The flow cell device of claim 1, further comprising a cleaning outlet in the one or more substrates.

79. The flow cell device of claim 78, wherein the cleaning outlet is in fluidic connection with the inlet or the open landing area.

80-82. (canceled)

83. The flow cell device of claim 78, wherein the cleaning outlet comprises a side port on the one or more substrates, wherein the side port:
(a) extends at least along a direction that is perpendicular or nearly perpendicular to an x direction;
(b) extends at least along a direction that is perpendicular or nearly perpendicular to a y direction;
(c) extends at least along a direction that is perpendicular or nearly perpendicular to a z direction;
(d) extends at least along a direction that is oblique to an x direction;
(e) extends at least along a direction that is oblique to a y direction; or
(f) extends at least along a direction that is oblique to a z direction.

84. The flow cell device of claim 78, wherein the cleaning outlet is configured to be coupled with a first pump or a second pump.

85-88. (canceled)

89. The flow cell device of claim 1, wherein a height of the one or more channels is about 150 μm, 130 μm, 120 μm, 110 μm, 100 μm, 90 μm, 80 μm, 70 μm, 60 μm, 50 μm, or 40 μm.

90-107. (canceled)

108. The flow cell device of claim 2, wherein at least a portion of the open landing area is covered with a second surface coating comprising a slippery liquid-infused porous surface (SLIPS).

109. The flow cell device of claim 2, wherein at least a portion of the open landing area is covered with a second surface coating comprising a slippery omniphobic covalently attached liquid (SOCAL) coating.

110. The flow cell device of claim 2, wherein at least a portion of the open landing area is covered with a second surface coating comprising a liquid-like polymer brush surface that is covalently attached to the one or more substrates.

111. The flow cell device of claim 2, wherein at least a portion of the open landing area is covered with a second surface coating comprising impregnating a lubricant in a porous surface to generate the second surface coating with a surface energy below about 20 mJ/m².

112. The flow cell device of claim 2, wherein at least a portion of the open landing area is covered with a second surface coating comprising acid-catalyzed graft polycondensation of one or more saline monomers.

113. The flow cell device of claim 112, wherein the one or more saline monomers comprise dimethyldimethoxysilane.

114. The flow cell device of claim 8, wherein the flow cell device is configured to allow cleaning at least part of the first reagent from at least part of the one or more channels during a DNA sequencing run.

115. The flow cell device of claim 8, wherein the flow cell device is configured to allow at least part of the first reagent to remain in the one or more channels.

116. The flow cell device of claim 8, wherein the first reagent and the second reagent are different.

117. The flow cell device of claim 1, wherein a volume of the air gap fills at least part of the one or more channels, and wherein said at least part of the one or more channels comprise more than about 40% of a corresponding volume or length of each of the one or more channels.

118-220. (canceled)

221. A flow cell system comprising:
(a) the flow cell device of claim 1;
(b) a fluidic control device.

222-227. (canceled)

228. A method for preparing a flow cell for DNA sequencing reactions, comprising:
(a) providing the flow cell comprising (i) an inlet and an outlet, wherein the inlet comprises an open landing area for receiving one or more reagents, and (ii) one or more channels disposed between the inlet and the outlet for performing the sequencing reactions;
(b) openly dispensing a first reagent of the one or more reagents to the open landing area to flow at least part of the first reagent from the open landing area to the one or more channels;
(c) introducing a gas into the one or more channels;
(d) openly dispensing a second reagent of the one or more reagents to the open landing area to flow at least part of the second reagent from the open landing area to the one or more channels, thereby removing a residual amount of the first reagent from the one or more channels.

229. (canceled)

230. A method for sequencing with a flow cell device, comprising:
(a) providing the flow cell comprising (i) an inlet and an outlet, wherein the inlet comprises an open landing area for receiving one or more reagents, and (ii) one or more channels disposed between the inlet and the outlet for performing the sequencing reactions;
(b) openly dispensing a first reagent of the one or more reagents to the open landing area to flow at least part of the first reagent from the open landing area to the one or more channels;
(c) removing a residual amount of the first reagent from at least part of the open landing area by flowing the residual amount of the first reagent through a cleaning outlet of the flow cell device; and
(d) openly dispensing a second reagent of the one or more reagents to the open landing area to flow at least part of the second reagent from the open landing area to the one or more channels.

231. A method for manufacturing a flow cell device, comprising:
obtaining one or more substrates;
generating one or more channels in the one or more substrates;
forming an inlet comprising a hole in one of the one or more substrates and an open landing area, wherein the inlet is in fluidic connection with the one or more channels;
forming an outlet that is in fluidic connection with the one or more channels;
coating at least a portion of a surface of the one or more channels with a first coating; and
fixedly coupling the one of one or more substrates together.

232. (canceled)

233. The method of claim 231, wherein the method further comprises
forming a cleaning outlet in the one or more substrates, wherein the cleaning outlet is in fluidic connection with the inlet, and wherein the cleaning outlet is closer to the inlet than to the outlet.

234. The method of claim 231, wherein the one or more channels are configured to allow a fluid or a gas gap between the fluid and another fluid to flow through the one or more channels, wherein the one or more channels are configured to allow the gas gap to flow through the one or more channels between allowing the first reagent and the second reagent to flow through the one or more channels.

235-236. (canceled)

237. The method of claim 234, wherein the one or more channels are configured to allow the gas gap to flow through the one or more channels to facilitate reducing contamination of the second reagent by the first reagent in a DNA sequencing run.

238. The method of claim 234, wherein the one or more channels are configured to allow the gas gap to flow through the one or more channels to reduce a minimum amount of the first reagent, the second reagent, or a washing reagent required for a DNA sequencing run.

239-356. (canceled)
